SEQUENCE LISTING (4 sequences)

SEQ ID NO: 1 - DNA, 24 bp, synthetic construct
  misc_feature 1..24: note = Description of sequence: Component of compound NSC-617145
  source 1..24: mol_type = other DNA
gcactggccgtcgttttacggtcg

SEQ ID NO: 2 - DNA, 23 bp, synthetic construct
  misc_feature 1..23: note = Description of sequence: Component of compound NSC-617145
  source 1..23: mol_type = other DNA
tccaagtaaaacgacggccagtg

SEQ ID NO: 3 - DNA, 2208 bp, Homo sapiens
  misc_feature 1..2208: note = Description of sequence: Gene sequence of WRN-BV08
  source 1..2208: mol_type = genomic DNA
atgaacgagggcgaagaagacgacgacaaggacttcctgtggcctgcccctaacgaagaacaagtgacatgcctgaagatgtacttcggacacagtagcttcaagcctgtgcaatggaaggtcatccactccgtgctggaagaaagaagggacaacgtggctgtgatggctaccggatacggtaagtccctgtgcttccagtaccctcccgtgtacgtgggcaagatcggtctggtgatctcccctctgatctctctgatggaggaccaggtgctgcaattgaagatgtccaacatccccgcttgcttcctgggttccgctcaaagtgagaacgtgctgacagacatcaagctgggcaagtaccgcatcgtgtacgtgacccctgagtactgctccggtaacatgggtctgctgcaacagctggaggctgacatcggaatcaccctgatcgctgtggacgaggctcactgcatctccgagtggggacacgacttccgcgactccttccgtaagctgggatccttgaagaccgctctccctatggtgcctatcgtggccctgaccgccactgcttcctcctccatccgcgaggacatcgtgcgttgcctgaacctgcgcaaccctcagatcacttgcaccggtttcgaccgccctaacttgtacctcgaggtgcgtcgcaagaccggtaacatcctccaggacctgcagcctttcctggtcaagacctcctcccactgggaatttgagggccctaccatcatctactgcccttcccgcaagatgacccagcaagtcaccggcgagctgcgcaagctcaacctctcctgcggtacctaccacgctggtatgtccttctccacccgcaaggacatccaccaccgcttcgtccgtgacgaaatccaatgcgtcatcgctaccatcgctttcggaatgggcatcaacaaggctgacatccgccaggtgatccactacggcgcccccaaggacatggaatcctactaccaggaaatcggtcgcgccggtcgcgacggtctgcagtcttcctgtcacgtgctgtgggcccccgctgacatcaacctgaaccgccacctgctgaccgaaatccgcaacgagaagttccgcctgtacaagctcaagatgatggctaagatggagaagtacctgcactcctcccgctgtcgccgtcagatcatcctctcccacttcgaggacaagcaagtgcaaaaggctagcctgggtatcatgggcaccgaaaagtgttgtgacaactgccgctcccgcctcgaccactgctactccatggacgacagcgaggacacctcctgggacttcggtcctcaagctttcaagctcttgtccgctgtggacatcctgggcgagaagttcggtatcggtctccccatcctcttcctgcgtggtagcaactcccaacgcctggctgaccagtaccgccgccactccctcttcggtaccggtaaggaccagaccgagtcctggtggaaggctttctctcgccaactgatcaccgaaggtttcctggtggaggtgtcccgctacaacaagttcatgaagatctgcgctctcactaagaagggaaggaactggctgcacaaggctaacactgagtcccaatccctcatcctgcaggctaacgaggagctgtgccctaagaagttcctgctgccttcctccaagaccgtgtcctccggaacaaaggaacactgctacaaccaagtccctgtggagctctccaccgagaagaagtccaacctggagaagctgtacagctacaagccttgcgacaagatcagctccggttccaacatcagcaagaagtccatcatggtgcaatcccctgaaaaggcctactccagctcccaacctgtcatctccgctcaagagcaagagacccagatcgtgctgtacggtaagctggtcgaagcccgccaaaagcacgctaacaagatggacgtccctcccgctatcctcgccaccaacaagatcctcgtggatatggctaagatgcgccccaccaccgtcgagaacgtgaagcgcatcgacggtgtctccgagggtaaggccgctatgctggctcctctgctggaagtgatcaagcacttctgccagaccaactccgtgcagaccgacctgttcagtagtgagaacctgtacttccaaggccaccatcatcatcatcatcaccactaa

SEQ ID NO: 4 - protein, 735 aa, Homo sapiens
  REGION 1..735: note = Description of sequence: Protein sequence of WRN-BV08
MNEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKFLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSENLYFQGHHHHHHHH

(19) United States
(12) Patent Application Publication
(10) Pub. No.: US 2026/0308173 A1
(43) Pub. Date: Oct. 8, 2026

(54) OXAZOLOPYRIDIN-7(4H)-ONE WRN INHIBITORS

(71) Applicant: NIMBUS WADJET, INC., Boston, MA (US)

(72) Inventors: Derun Li, Boston, MA (US); Angela V. West, Franklin, MA (US); Justin Caravella, Cambridge, MA (US); Nathan E. Genung, Boston, MA (US); Florian Bartels, Berlin (DE); Robert Lee Dow, Groton, CT (US); Silvana Marcel Leit De Moradei, Burlington, MA (US); Nikolay Sitnikov, Berlin, DE (US)

(21) Appl. No.: 19/491,004

(22) PCT Filed: Jun. 10, 2024
(86) PCT No.: PCT/US2024/033276

§ 371 Date: Dec. 8, 2025

Related U.S. Application Data
(60) Provisional application No. 63/507,362, filed on Jun. 9, 2023.
(60) Provisional application No. 63/518,298, filed on Aug. 8, 2023.
(60) Provisional application No. 63/613,651, filed on Dec. 21, 2023.

(51) Int. Cl. C07D 498/04; A61K 31/496; A61K 31/498; A61K 31/506; A61K 31/5377; A61P 35/00; C07D 519/00

(52) U.S. Cl. CPC C07D 498/04; A61K 31/496; A61K 31/498; A61K 31/506; A61K 31/5377; A61P 35/00; C07D 519/00

(57) ABSTRACT

The present disclosure is directed to oxazolopyridin-7(4H)-one based compounds, including compounds of Formula (I): (Formula (I)) and pharmaceutically acceptable salts thereof, and compositions thereof, as well as methods of treatment of cancers such as those involving WRN protein.

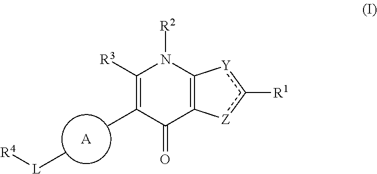

### RELATED APPLICATIONS

This application claims priority to and the benefit of U.S. Provisional Application No. 63/507,362 filed Jun. 9, 2023; U.S. Provisional Application No. 63/518,298 filed Aug. 8, 2023; and U.S. Provisional Application No. 63/613,651 filed Dec. 21, 2023; the contents of each of which are incorporated by reference in their entireties.

### FIELD OF INVENTION

The invention provides bicyclic compounds and compositions, the use thereof and methods using the compounds, for inhibiting Werner Syndrome RecQ DNA helicase (WRN) and methods of treating disease using said compounds, in particular the use in treating cancer, and in particular the treatment of cancer characterized as microsatellite instability-high (MSI-H) or mismatch repair deficient (dMMR), including colorectal, gastric and endometrial cancer. The invention also provides the use of said compounds as research chemicals, intermediate compounds, combinations, processes and formulations.

### SEQUENCE LISTING

This application contains a Sequence Listing which has been submitted in xml format via EFS and is hereby incorporated by reference. The ST.26 copy, created on Jun. 8, 2023, is named 407274-80WRP3_ST26.xml and is 8,751 bytes in size.

### BACKGROUND

Loss of DNA mismatch repair is a common initiating event in cancer development occurring in 10-30% of colorectal, endometrial, ovarian and gastric cancers (Aaltonen, L. A. et al. Clues to the pathogenesis of familial colorectal cancer, Science 260, 812-816 (1993), Bonneville R et al., Landscape of Microsatellite Instability Across 39 Cancer Types. JCO Precis Oncol. 1: PO.17.00073 (2017)). Cancers that are deficient in mismatch repair (dMMR) have a high mutational burden, and frequent deletion and insertion events in repetitive DNA tracts, a phenotype known as microsatellite instability (MSI). While progress has been made in the treatment of microsatellite instability high (MSI-H) cancers, and the demonstration that pembrolizumab (anti-PD1) treatment led to significantly longer progression-free survival than chemotherapy when received as first-line therapy for MSI-H-dMMR metastatic colorectal cancer (CRC) which resulted in the recent approval of pembrolizumab as first-line treatment of these cancers, there is still a significant unmet medical need in CRC and other MSI-H indications (André T., et al. Pembrolizumab in Microsatellite-Instability-High Advanced Colorectal Cancer. N Engl J Med 383(23):22072218(2020)). Several large-scale functional genomics screens across large panels of cell lines, including Novartis with 398 cell lines from the Cancer Cell Line Encyclopedia (CCLE) (McDonald E. R. et al., Project DRIVE: A Compendium of Cancer Dependencies and Synthetic Lethal Relationships Uncovered by Large-Scale, Deep RNAi Screening. Cell 170(3):577-592 (2017)), have identified the Werner Syndrome RecQ helicase (WRN) as being selectively required for the survival of cell lines with defective mismatch repair that have become MSI-H (Behan, F. M. et al. Prioritization of cancer therapeutic targets using CRISPR-Cas9 screens. Nature 568, 511-516(2019), Chan, E. M. et al. WRN helicase is a synthetic lethal target in microsatellite unstable cancers. Nature 568, 551-556(2019). Kategaya, L., Perumal, S. K., Hager, J. H. & Belmont, L. D. Werner syndrome helicase is required for the survival of cancer cells with microsatellite instability. iScience 13, 488-497(2019), Lieb, S. et al. Werner syndrome helicase is a selective vulnerability of microsatellite instability-high tumor cells. eLife 8, e43333(2019)). WRN is synthetically lethal with MSI cancers. Depletion of WRN leads to anti-proliferative effects and results in activation of multiple DNA damage signaling markers, induction of cell cycle arrest and apoptosis in MSI-H cancer models but not cancer cells with an intact MMR pathway (otherwise known as microsatellite stable or MSS). The anti-proliferative effects of WRN depletion could not be rescued with a helicase deficient WRN construct, demonstrating that helicase activity of WRN is required for MSI-H viability. These findings indicate that WRN helicase provides a DNA repair and maintenance function that is essential for cell survival in MSI cancers. Recently, the mechanism of WRN dependence has been elucidated. It has been shown that dinucleotide TA repeats are selectively unstable in MSI cells and undergo large scale expansions. These expanded TA repeats form secondary DNA structures that require the WRN helicase for unwinding (van Wietmarschen, N. et al. Repeat expansions confer WRN dependence in microsatellite-unstable cancers. Nature 586, 292-298, 2020). In the absence of WRN (or upon WRN helicase inhibition), expanded TA repeats in MSI cells are subject to nuclease cleavage and chromosome breakage. Thus, inhibiting the WRN helicase is an attractive strategy for the treatment of MSI-H cancers.

### SUMMARY

There remains a need for new treatments and therapies for the treatment of cancer, and in particular cancers characterized as microsatellite instability-high (MSI-H) or mismatch repair deficient (dMMR), including colorectal, gastric or endometrial cancer. The invention provides compounds, pharmaceutically acceptable salts thereof, pharmaceutical compositions thereof and combinations thereof, said compounds being inhibitors of Werner Syndrome RecQ DNA Helicase (WRN). The invention further provides methods of treating, preventing, or ameliorating a disease or condition, comprising administering to a subject in need thereof an effective amount of a WRN inhibitor. The invention also provides compounds, pharmaceutically acceptable salts thereof, pharmaceutical compositions thereof and combinations thereof, said compounds being useful for the treatment of cancer, in particular cancers characterized as microsatellite instability-high (MSI-H) or mismatch repair deficient (dMMR). Also provided are compounds that bind to, and/or inhibit WRN, and are therefore useful as research chemicals, e.g., as a chemical probe, and as tool compounds. Various embodiments of the invention are described herein.

In one aspect, the disclosure provides a compound of Formula I, or a pharmaceutically acceptable salt thereof:

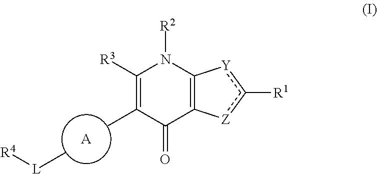

wherein R¹, R², R³, R⁴, Y, Z, -L-, and Ring A are as described and defined herein.

In another aspect, the invention provides a pharmaceutical composition comprising a compound of Formula I of the present invention and one or more pharmaceutically acceptable carriers.

In another aspect, the invention provides a combination, in particular a pharmaceutical combination, comprising a compound of Formula I of the present invention and one or more therapeutically active agents.

In another aspect, the invention provides a compound of Formula I of the present invention for use as a medicament, in particular for the treatment of a disorder or disease which can be treated by WRN inhibition.

In another aspect, the invention provides a compound of Formula I of the present invention for use in the treatment of cancer, particularly wherein the cancer is characterized as microsatellite instability-high (MSI-H) or mismatch repair deficient (dMMR).

In another aspect, the invention provides a method of treating a disorder or disease which can be treated by WRN inhibition in a subject, comprising administering to the subject a therapeutically effective amount of a compound of Formula I of the present invention.

In another aspect, the invention provides a method of treating cancer in a subject, more particularly wherein the cancer is characterized as microsatellite instability-high (MSI-H) or mismatch repair deficient (dMMR), comprising administering to the subject a therapeutically effective amount of a compound of Formula I of the present invention.

In another aspect, the invention provides the use of a compound of Formula I of the present invention in the manufacture of a medicament for the treatment of a disorder or disease which can be treated by WRN inhibition.

In another aspect, the invention provides a compound of Formula I of the present invention for use as a research chemical, for example as a chemical probe or as a tool compound.

In another aspect, the invention provides a solid form, process or intermediate as described herein.

### DETAILED DESCRIPTION

### 1. General Description of Certain Embodiments of the Invention

In one aspect, the disclosure provides a compound of Formula I, or a pharmaceutically acceptable salt thereof:

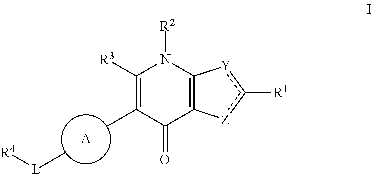

wherein Ring A represents:
   a) a 4-7 membered saturated or partially unsaturated bivalent monocyclic ring system selected from carbocyclylene and heterocyclylene (having 1-4 heteroatoms independently selected from nitrogen, oxygen, and sulfur); or
   b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
wherein Ring A is substituted with 0-4 independently selected R^{A} substituents;
each of Y and Z is selected from N and O, wherein Y is N and Z is O or Y is O and Z is N, wherein [symbol] denotes a single or double bond and the 5-membered ring comprising Z and Y is aromatic;
L is a linker selected from —C(O)—, —S(O)—, —S(O)₂—, and

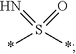

R¹ is selected from one of groups a) to e):
   a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A};
   b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A};
   c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A};
   d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}; and
   e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹—CH₂NR¹⁰R¹¹, —SO₂R¹², wherein the C₁-C₆aliphatic, C₃-C₇cycloalkyl, or C₁-C₆alkylene-O—C₁-C₆alkyl is substituted with 0-5 independently selected R^{A};
R¹⁰ is H, C₁-C₆aliphatic, haloC₁-C₆alkyl, C₃-C₆cycloalkyl, haloC₃-C₆cycloalkyl, —C(O)C₃-C₆cycloalkyl, —C(O)C₁-C₆alkyl, or a 5-6 membered heteroaryl (having 1-4 heteroatoms independently selected from nitrogen, oxygen, and sulfur) optionally substituted with 1 or 2 groups independently selected from R^{A};
R¹¹ is H, C₁-C₆aliphatic, or C₃-C₆cycloalkyl, or R¹⁰ and R¹¹ may combine to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R;
R² is C(R^{B})₂C(O)N(R)R^{2A},
R^{B} is independently selected at each occurrence from hydrogen, —CH₃, and —CH₂CH₃, or two R^{B} taken together with the carbon to which they are attached form a cyclopropyl ring;
R^{2A} is phenyl or pyridyl, each of which is optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen; or
R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH;
R³ is hydrogen, C₁-C₄aliphatic, C₃-C₆cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, —OR, —C(O)NR¹⁰R¹¹, or N(R)C(O)R;
each R^{3A} is independently selected at each occurrence from C₁-C₄alkyl;
R⁴ is phenyl or a 5-6 membered heteroaryl (having 1-4 heteroatoms independently selected from nitrogen, oxygen, and sulfur) wherein said phenyl or 5-6 membered heteroaryl are substituted with 0-5 R^{A} or two substituents on adjacent atoms of said phenyl or 5-6 membered heteroaryl together with said adjacent atoms form a 4-7 membered carbocyclyl, a 4-7 membered heterocyclyl, or a 5-6 membered heteroaryl (having 1-4 heteroatoms independently selected from nitrogen, oxygen, and sulfur) that is fused to the phenyl or 5-6 membered heteroaryl wherein said 4-7 membered carbocyclyl, 4-7 membered heterocyclyl, or 5-6 membered heteroaryl are substituted with 0-3 R^{A}, or
R⁴ is a C₁-C₄alkyl, C₁-C₄alkoxy, or C₃-C₆cycloalkyl, each of which is substituted with 0-3 groups independently selected from halogen, —CN, —OH, oxo, NH₂, C₁-C₄alkyl, C₁-C₄alkoxy, optionally substituted 5-6 membered heterocyclyl, and optionally substituted 5-6 membered heterocyclyloxy; each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

For clarity, it is understood that [symbol] above denotes a single or double bond and that if Y is O then

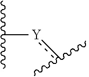

is

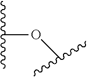 [SMILES: *O*]

and if Y is N then

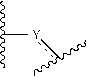

is

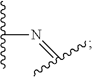 [SMILES: *N=*]

and if Z is O then

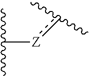

is

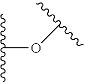 [SMILES: *O*]

and if Z is N then

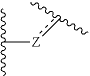

is

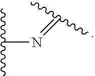 [SMILES: *N=*]

In another aspect, the invention provides a method of treating a disorder or disease which can be treated by WRN inhibition in a subject, comprising administering to the subject a therapeutically effective amount of a compound of Formula I of the present invention, or a pharmaceutically acceptable salt thereof.

In another aspect, the disclosure provides a compound of Formula I′, or a pharmaceutically acceptable salt thereof:

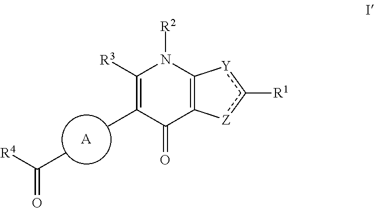

wherein R⁴ is selected from one of a), b), and c):
a) R⁴ is a Ring B that is selected from the group consisting of:

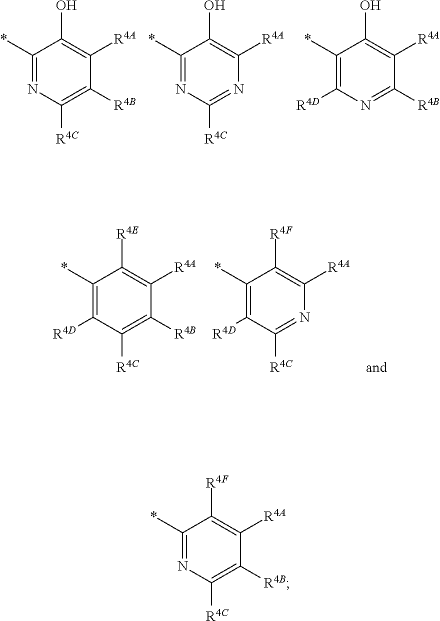

wherein * is a point of attachment to L in Formula I;
and wherein:
any substituents that are present on Ring E selected from R^{4A}, R^{4B}, R^{4C}, R^{4D}, R^{4E}, and R^{4F} are each independently selected from hydrogen; halogen; —OH; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form 4-7 membered optionally substituted carbocyclyl, 4-7 membered optionally substituted heterocyclyl, or 5-6 membered optionally substituted heteroaryl (having 1-4 heteroatoms independently selected from nitrogen, oxygen, and sulfur) that is fused to Ring E; and any substituents that are present on Ring E selected from R^{4C}, R^{4D}, R^{4E}, and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4B} and R^{4C}, along with their intervening atoms, join to form 4-7 membered optionally substituted carbocyclyl, 4-7 membered optionally substituted heterocyclyl, or 5-6 membered optionally substituted heteroaryl (having 1-4 heteroatoms independently selected from nitrogen, oxygen, and sulfur) that is fused to Ring E; and any substituents that are present on Ring E selected from R^{4A}, R^{4D}, R^{4E}, and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form 4-7 membered optionally substituted carbocyclyl, 4-7 membered optionally substituted heterocyclyl, or 5-6 membered optionally substituted heteroaryl (having 1-4 heteroatoms independently selected from nitrogen, oxygen, and sulfur) that is fused to Ring E; and any substituents that are present on Ring E selected from R^{4A}, R^{4B}, R^{4E} and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} and R^{4A}, along with their intervening atoms, join to form 5-6 membered optionally substituted heteroaryl (having 1-4 heteroatoms independently selected from nitrogen, oxygen, and sulfur) that is fused to Ring E; and R^{4B}, R^{4C}, and R^{4D} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4F} and R^{4A}, along with their intervening atoms, join to form optionally substituted 5-6 membered heteroaryl (having 1-4 heteroatoms independently selected from nitrogen, oxygen, and sulfur) that is fused to Ring E; and R^{4B} and R^{4C} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴;
R¹³ is independently selected at each occurrence from hydrogen and C₁-C₄alkyl optionally substituted with —OH, —OCH₃, or —OCH₂CH₃; and
R¹⁴ is hydrogen; or NR¹³R¹⁴ forms a heterocyclic ring selected from azetidinyl, pyrrolidinyl, and piperidinyl, said heterocyclic ring optionally substituted with —CH₃; or
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy; or
c) R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl, and optionally substituted 5-6 membered heterocyclyloxy.

In another aspect, the invention provides a method of treating a disorder or disease which can be treated by WRN inhibition in a subject, comprising administering to the subject a therapeutically effective amount of a compound of Formula I′ of the present invention, or a pharmaceutically acceptable salt thereof.

In one aspect, the disclosure provides a compound of Formula I″, or a pharmaceutically acceptable salt thereof:

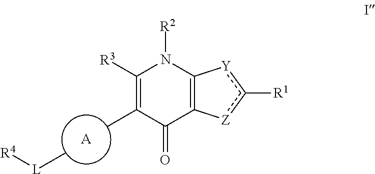

wherein Ring A represents:
   a) a 4-7 membered saturated or partially unsaturated bivalent monocyclic ring system selected from carbocyclylene and heterocyclylene (having 1-4 heteroatoms independently selected from nitrogen, oxygen, and sulfur); or
   b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
wherein Ring A is substituted with 0-4 independently selected R^{A} substituents;
each of Y and Z is selected from N and O, wherein Y is N and Z is O or Y is O and Z is N, wherein [symbol] denotes a single or double bond and the 5-membered ring comprising Z and Y is aromatic;
L is a linker selected from —C(O)—, —S(O)—, —S(O)₂—, and

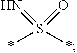

R¹ is selected from one of groups a) to e):
   a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A};
   b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A};
   c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A};
   d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}, and
   e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹, —CH₂NR¹⁰R¹¹, —SO₂R¹², wherein the C₁-C₆aliphatic, C₃-C₇cycloalkyl, or C₁-C₆alkylene-O—C₁-C₆alkyl is substituted with 0-5 independently selected R^{A};
R¹⁰ is H, C₁-C₆aliphatic, haloC₁-C₆alkyl, C₃-C₆cycloalkyl, haloC₃-C₆cycloalkyl, —C(O)C₃-C₆cycloalkyl, —C(O)C₁-C₆alkyl, or a 5-6 membered heteroaryl (having 1-4 heteroatoms independently selected from nitrogen, oxygen, and sulfur) optionally substituted with 1 or 2 groups independently selected from R^{A};
R¹¹ is H, C₁-C₆aliphatic, or C₃-C₆cycloalkyl, or R¹⁰ and R¹¹ may combine to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R;
R² is selected from C(R^{B})₂C(O)N(R)R^{2A}, C(R^{B})₂C(R^{B})₂C(O)N(R)R^{2A}, C(R^{B})₂C(R^{B})₂N(R)C(O) N(R)R^{2A}, and C(R^{B})₂C(R^{B})₂N(R)C(O)R^{2A},
R^{B} is independently selected at each occurrence from hydrogen, —CH₃, and —CH₂CH₃, or two R^{B} taken together with the carbon to which they are attached form a cyclopropyl ring;
R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring, or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen, and wherein 2 substituents on the same atom of said saturated or partially unsaturated monocyclic ring, or saturated or partially unsaturated bridged, fused, or spirocyclic ring form a cyclic group selected from:
   an optionally substituted 3-7 membered saturated or partially unsaturated carbocyclyl, and
   an optionally substituted 4-7 membered saturated or partially unsaturated heterocyclyl having 1-3 heteroatoms independently selected from nitrogen, oxygen, and sulfur, or
R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH;
R³ is hydrogen, C₁-C₄aliphatic, C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, —OR, —C(O)NR¹⁰R¹¹, or N(R)C(O)R;
each R^{3A} is independently selected at each occurrence from C₁-C₄alkyl;
R⁴ is phenyl or a first 5-6 membered heteroaryl (having 1-4 heteroatoms independently selected from nitrogen, oxygen, and sulfur) wherein said phenyl or said first 5-6 membered heteroaryl are substituted with 0-5 R^{A} or two substituents on adjacent atoms of said phenyl or said first 5-6 membered heteroaryl together with said adjacent atoms form a 4-7 membered carbocyclyl, a 4-7 membered heterocyclyl, or a second 5-6 membered heteroaryl (having 1-4 heteroatoms independently selected from nitrogen, oxygen, and sulfur) that is fused to the phenyl or first 5-6 membered heteroaryl wherein said 4-7 membered carbocyclyl, 4-7 membered heterocyclyl, or second 5-6 membered heteroaryl are substituted with 0-3 R^{A}, or
R⁴ is a C₁-C₄alkyl, C₁-C₄alkoxy, or C₃-C₆cycloalkyl, each of which is substituted with 0-3 groups independently selected from halogen, —CN, —OH, oxo, NH₂, C₁-C₄alkyl, C₁-C₄alkoxy, optionally substituted 5-6 membered heterocyclyl, and optionally substituted 5-6 membered heterocyclyloxy;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

In another aspect, the invention provides a method of treating a disorder or disease which can be treated by WRN inhibition in a subject, comprising administering to the subject a therapeutically effective amount of a compound of Formula I″ of the present invention, or a pharmaceutically acceptable salt thereof.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 5-12 ring members and 0-4 heteroatoms independently selected from nitrogen, oxygen, or sulfur unless otherwise specified, a bridged bicyclic group is optionally substituted with one or more substituents as set forth for aliphatic groups. Additionally or alternatively, any substitutable nitrogen of a bridged bicyclic group is optionally substituted. The term “alkyl” refers to a C₁-12 straight or branched saturated aliphatic group. In certain instances, alkyl refers to a C₁-8 straight or branched saturated aliphatic group or a C₁-6 straight or branched saturated aliphatic group. The term “lower alkyl” refers to a C₁-4 straight or branched alkyl group.

Exemplary lower alkyl groups are methyl (—CH₃), ethyl (—CH₂CH₃), propyl, isopropyl (also referred to interchangeably herein as 2-propyl, iPr, ⁱPr and i-Pr), butyl, isobutyl (also referred to interchangeably herein as 2-butyl, iBu, ⁱBu and i-Bu) and tert-butyl (also referred to interchangeably herein as 2-methyl-2-butyl, tBu, ^{t}Bu and t-Bu).

The term “alkenyl” refers to a C₂₋₁₂ straight or branched partially unsaturated aliphatic group comprising at least one unsaturated carbon carbon double bond. In certain instances, alkenyl refers to a C₂₋₈ or a C₂₋₆ straight or branched partially unsaturated aliphatic group comprising at least one unsaturated carbon carbon double bond. The term “lower alkenyl” refers to a C₂₋₄ straight or branched partially unsaturated aliphatic group comprising at least one unsaturated carbon carbon double bond. Alkenyl groups include both cis (Z) and trans (E) regioisomers. Exemplary lower alkenyl groups are vinyl, allyl, 2-propenyl, and butenyl isomers (—CH₂CH₂CH═CH₂, —CH₂CH═CHCH₃, and —CH═CHCH₂CH₃).

The term “alkynyl” refers to a C₂₋₁₂ straight or branched partially unsaturated aliphatic group comprising at least one unsaturated carbon carbon triple bond. In certain instances, alkynyl refers to a C₂₋₈ or a C₂₋₆ straight or branched partially unsaturated aliphatic group comprising at least one unsaturated carbon carbon triple bond. The term “lower alkynyl” refers to a C₂₋₄ straight or branched partially unsaturated aliphatic group comprising at least one unsaturated carbon carbon triple bond. Exemplary lower alkynyl groups are ethynyl, 1-propynyl, 2-propynyl, 1-butynyl, 2-butynyl, and 3-butynyl.

The term “haloalkyl” refers to a straight or branched alkyl group that is substituted with one or more halogen atoms. The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

As used herein, the term “bivalent C₁₋₈ (or C₁₋₆ i.e., C₁-C₆) saturated or unsaturated, straight or branched, hydrocarbon chain,” refers to bivalent alkylene, alkenylene, and alkynylene chains that are straight or branched as defined herein.

As used herein, the term “bivalent,” to describe a cyclic (and noncyclic) group refers to, for example, bivalent carbocyclylene, phenylene, heterocyclylene, and heteroarylene that are bivalent moieties of carbocycles, phenyls, heterocycles, and heteroaryls described herein. Non-limiting examples include

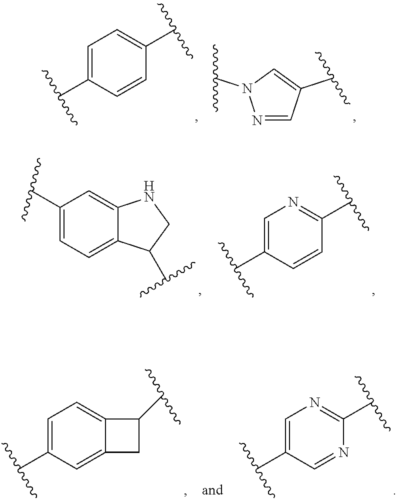 [SMILES: *c1ccc(*)cc1 ; *c1cnn(*)c1 ; *c1ccc2c(c1)NCC2* ; *c1ccc(*)nc1 ; *c1ccc2c(c1)CC2* ; *c1cnc(*)nc1]

“Carbocyclylene” as used herein refers to a carbocyclic or cycloalkyl moiety that is bivalent as described above (i.e., attached at two different points to the rest of the compound). Non-limiting examples include cyclopropylene, cyclobutylene, cyclopentylene, or cyclohexylene as shown below.

| | | Different examples | Different examples |
|---|---|---|---|
| cyclopropylene | cyclobutylene | of cyclopentylene | of cyclohexylene |
| 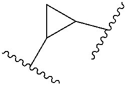 [SMILES: *C1CC1*] | 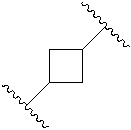 [SMILES: *C1CC(*)C1] | 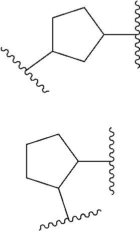 [SMILES: *C1CCC(*)C1 ; *C1CCCC1*] | 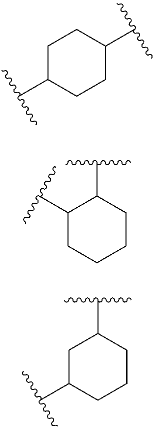 |

A carbocyclylene may be saturated as in the examples shown above or partially unsaturated as in the examples shown below.

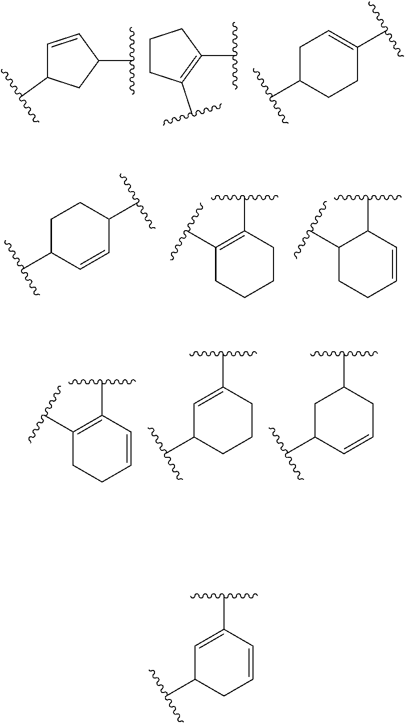

A carbocyclylene may be multi-cyclic, for example, bicyclic or tricyclic. Such multi-cyclic carbocyclylene systems may be saturated or partially unsaturated (while one ring of the bicyclic system may be aromatic it is to be understood that multi-cyclic ring systems that are not in their entirety aromatic may also fall under the definition of carbocyclylene). The rings may form bridged, fused, or spiro systems. Non-limiting examples are shown below.

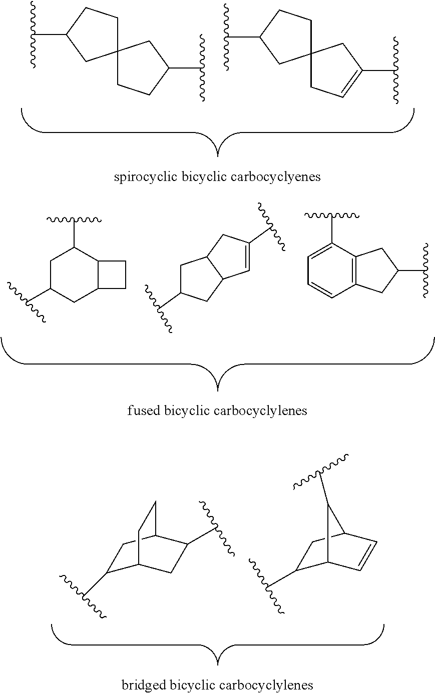

“Heterocyclylene” as used herein refers to a heterocyclic or heterocyclyl moiety that is bivalent as described above (i.e., attached at two different points to the rest of the compound) and may also be saturated or partially unsaturated. Non-limiting examples include those shown below. Heterocyclylene is understood to include bicyclic heterocyclylene systems. Non-limiting examples of bicyclic heterocyclylene moieties are also shown below and said bicyclic systems may be spirocyclic, fused, or bridged and may be saturated or partially unsaturated.

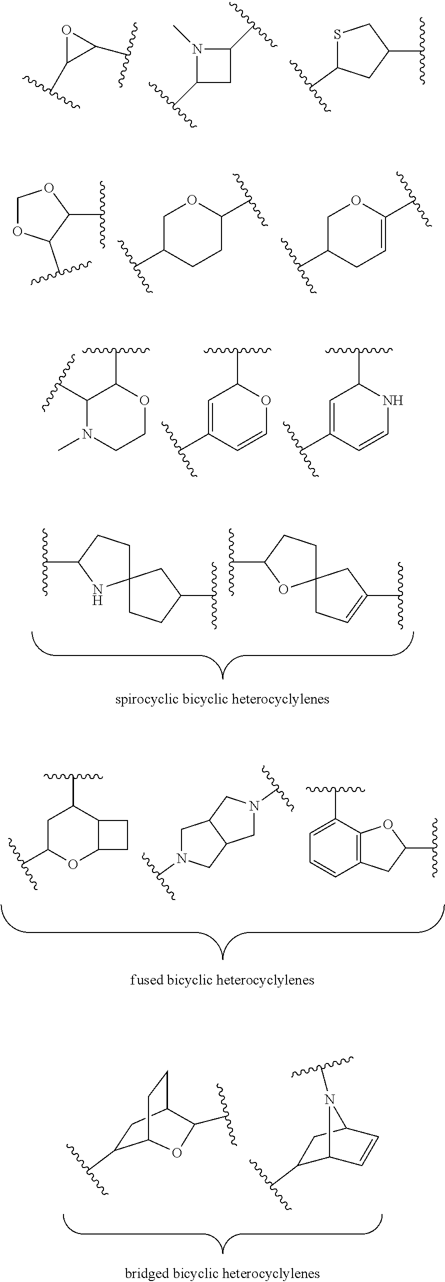

“Phenylene” as used herein refers to a phenyl moiety that is bivalent as described above (i.e., attached at two different points to the rest of the compound). Examples are shown below.

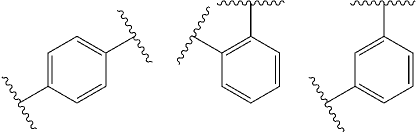 [SMILES: *c1ccc(*)cc1 ; *c1ccccc1* ; *c1cccc(*)c1]

“Arylene” as used herein refers to an mono or multi-cyclic aryl (i.e., phenyl or a multi-cyclic aryl) moiety that is bivalent as described above (i.e., attached at two different points to the rest of the compound), wherein the arylene group contains no heteroatoms. Examples are shown below.

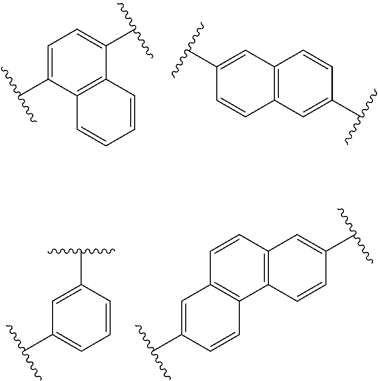

“Heteroarylene,” as used herein refers to a mono or multi-cyclic aryl ring system that contains at least one heteroatom wherein the ring system is bivalent as described above (i.e., attached at two different points to the rest of the compound). Examples are shown below.

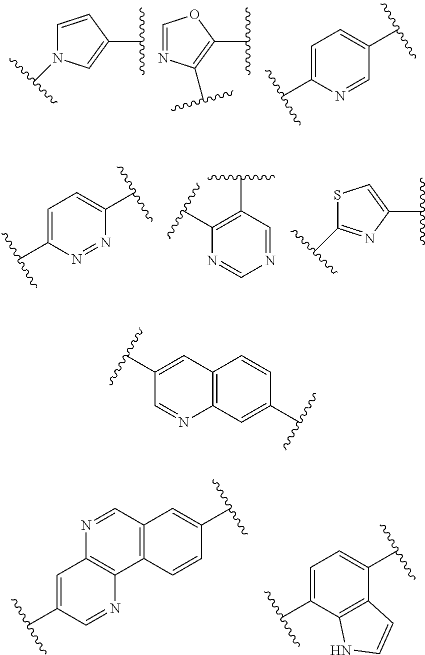

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

“Carbocyclyl (or heterocyclyl, aryl, phenyl, or heteroaryl) fused to” another phenyl, aryl, heteroaryl, carbocyclyl, or heterocyclyl, for example, a “phenyl or pyridyl” as used herein, may be referred to as “partially unsaturated” without said “carbocyclyl (or heterocyclyl, aryl, phenyl, or heteroaryl) fused to” the other ring requiring further unsaturation besides the carbon carbon bond which it shares with the ring to which it is fused (i.e., the “phenyl or pyridyl”). This is illustrated below.

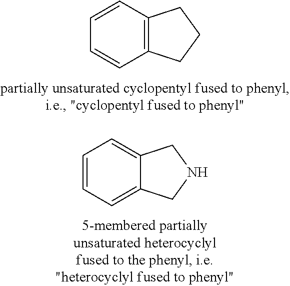 [SMILES: c1ccc2c(c1)CCC2 ; c1ccc2c(c1)CNC2]

A further example below shows a carbocyclyl moiety fused to a Ring B as defined in the embodiments herein. Said carbocyclyl does not explicitly require a descriptor of “partially unsaturated” to describe said carbocyclyl because it shares two carbons with the aromatic pyridine to which it is fused. Such language is used herein to describe such systems, for example, “R^{4A} and R^{4B}, along with their intervening atoms, join to form 4-7 membered carbocyclyl that is fused to Ring B” as shown in the image below. As such, “Ring B” may refer to a monocyclic ring (i.e., the pyridine shown below and its substituents which do not form a fused ring), without any further fused rings created by its substituents (i.e., R^{4A} and R^{4B}). Any further fused ring created by the substituents of Ring B is described as being “fused to Ring B.” Likewise, R^{4A} and R^{4B}, along with their intervening atoms, join to form 4-7 membered carbocyclyl or heterocyclyl (having 1-4 heteroatoms independently selected from nitrogen, oxygen, and sulfur) that is fused to Ring B (not pictured), is subject to the same interpretation.

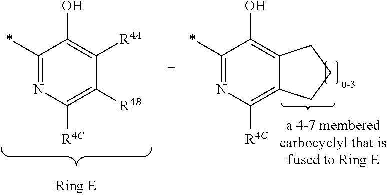

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “cubanyl” refers to a substituent of cubane as shown below.

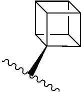

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, 9 or 10 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyrazinyl, pyrazolyl, pyridazinyl, pyridyl, pyrimidinyl, triazinyl, thienyl, furanyl, pyrrolyl, imidazolyl, pyrazolyl, triazolyl (i.e., 1,2,3-triazolyl), 1,2,4-triazolyl, 1,2,5-triazolyl, 1,3,4-triazolyl, tetrazolyl, oxazolyl, isoxazolyl, oxadiazolyl, 1,2,3-oxadiazolyl, 1,2,4-oxadiazolyl, 1,2,5-oxadiazolyl, 1,3,4-oxadiazolyl, thiazolyl, isothiazolyl, thiadiazolyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, indolizinyl, isoindolin-1-only, 1,2-dihydro-3H-pyrrolo[3,4-c]pyridin-3-onyl, 2,3-dihydro-1H-pyrrolo[3,4-c]pyridin-1-onyl, imidazo[1,2-a]pyridyl, imidazo[1,5-a]pyridyl, pyrazolo[1,5-a]pyridyl, pyrrolo[1,2-b]pyridazinyl, pyrrolo[1,2-a]pyrimidinyl, imidazo[1,2-b]pyridazinyl, imidazo[1,2-a]pyrimidinyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, oxetanyl, azetidinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

“Arylene” or “heteroarylene,” as used herein (i.e., phenylene), refers to any bivalent aryl or heterocyclyl described herein, that is a bisradical substituted at each of two substitutable positions of the ring system as described in detail supra.

“Heterocyclyloxy,” as used herein, refers to an —OR group wherein the R is a heterocyclyl. Nonlimiting examples are shown below.

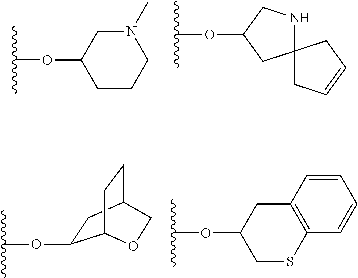

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄B(OR^{∘})₂; —(CH₂)₀₋₄R^{∘}; —(CH₂)₀₋₄OR^{∘}; —O(CH₂)₀₋₄R^{∘}; —O—(CH₂)⁰⁻⁴C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘)}₂; —(CH₂)₀₋₄N(R^{∘})C(O)R^{∘}; —N(R^{∘})C(S)R^{∘}; (CH₂)₀₋₄N(R^{∘})C(O)NR^{∘})₂; —N(R^{∘})C(S)NR^{∘})₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}2; —N(R^{∘})N(R^{∘})C(O)OR^{∘}); —N(R^{∘})C(NR^{∘})N(R^{∘)}₂; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)₀₋₄OP(O)R^{∘}₂; —(CH₂)₀₋₄OP(O)(OR^{∘})₂; —SiR^{∘}₃; —(C₁₋₄ straight or branched) alkylene)O—N(R^{∘)}₂; or —(C₁-4 straight or branched) alkylene)C(O)O—N(R^{∘)}₂, wherein each R^{∘} may be substituted as defined below and is independently hydrogen, C_{1 6} aliphatic, —SO₂—C₁₋₄ aliphatic (i.e., —SO₂CH₃)—CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, (haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁-4 straight or branched alkylene) C(O)OR^{●}, or —SSR^{●} wherein each R^{●} is unsubstituted or where preceded by “halo” is substituted only with one or more halogens, and is independently selected from C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents on a saturated carbon atom of an “optionally substituted” group, which includes instances of R^{∘} (or the ring formed by taking two independent occurrences of R^{∘} together with their intervening atoms), include the following: ═O, ═S, ═NNR*₂, ═NNHC(O)R*, ═NNHC(O)OR*, ═NNHS(O)₂R*, ═NR*, —NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, Z and E conformational isomers and Rₐ (or M) and Sₐ (or P) atropisomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C - or ¹⁴C-enriched carbon are within the scope of this invention. Such compounds are useful, for example, as analytical tools, as probes in biological assays, or as therapeutic agents in accordance with the present invention. In certain embodiments, Ring A of a provided compound may be substituted with one or more deuterium atoms.

The structures as drawn represent relative configurations, unless labeled as absolute configurations. The invention contemplates individual enantiomers and racemic mixtures.

### 3. Description of Exemplary Embodiments

In one aspect, the disclosure provides a compound of Formula I, or a pharmaceutically acceptable salt thereof:

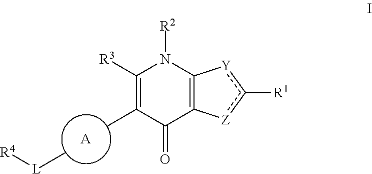

wherein Ring A represents:
   a) a 4-7 membered saturated or partially unsaturated bivalent monocyclic ring system selected from carbocyclylene and heterocyclylene (having 1-4 heteroatoms independently selected from nitrogen, oxygen, and sulfur); or
   b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
wherein Ring A is substituted with 0-4 independently selected R^{A} substituents;
each of Y and Z is selected from N and O, wherein Y is N and Z is O or Y is O and Z is N, [symbol] denotes a single or double bond, and the 5-membered ring comprising Z and Y is aromatic;
L is a linker selected from —C(O)—, —S(O)—, —S(O)₂—, and

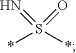

R¹ is selected from one of groups a) to e):
   a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A},
   b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A},
   c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A},
   d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}, and
   e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹. —CH₂NR¹⁰R¹¹, —SO₂R¹², wherein the C₁-C₆aliphatic, C₃-C₇cycloalkyl, or C₁-C₆alkylene-O—C₁-C₆alkyl is substituted with 0-5 independently selected R^{A};
R¹⁰ is H, C₁-C₆aliphatic, haloC₁-C₆alkyl, C₃-C₆cycloalkyl, haloC₃-C₆cycloalkyl, —C(O)C₃-C₆cycloalkyl, —C(O) C₁-C₆alkyl, or a 5-6 membered heteroaryl (having 1-4 heteroatoms independently selected from nitrogen, oxygen, and sulfur) optionally substituted with 1 or 2 groups independently selected from R^{A},
R¹¹ is H, C₁-C₆aliphatic, or C₃-C₆cycloalkyl, or R¹⁰ and R¹¹ may combine to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R;
R² is C(R^{B})₂C(O)N(R)R^{2A};
R^{B} is independently selected at each occurrence from hydrogen, —CH₃, and —CH₂CH₃, or two R^{B} taken together with the carbon to which they are attached form a cyclopropyl ring;
R^{2A} is phenyl or pyridyl, each of which is optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen; or
R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH;
R³ is hydrogen, C₁-C₄aliphatic, C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, —OR, —C(O)NR¹⁰R¹¹, or N(R)C(O)R;
each R^{3A} is independently selected at each occurrence from C₁-C₄alkyl;
R⁴ is phenyl or a 5-6 membered heteroaryl (having 1-4 heteroatoms independently selected from nitrogen, oxygen, and sulfur) wherein said phenyl or 5-6 membered heteroaryl are substituted with 0-5 R^{A} or two substituents on adjacent atoms of said phenyl or 5-6 membered heteroaryl together with said adjacent atoms form a 4-7 membered carbocyclyl, a 4-7 membered heterocyclyl, or a 5-6 membered heteroaryl (having 1-4 heteroatoms independently selected from nitrogen, oxygen, and sulfur) that is fused to the phenyl or 5-6 membered heteroaryl wherein said 4-7 membered carbocyclyl, 4-7 membered heterocyclyl, or 5-6 membered heteroaryl are substituted with 0-3 R^{A}, or
R⁴ is a C₁-C₄alkyl, C₁-C₄alkoxy, or C₃-C₆cycloalkyl, each of which is substituted with 0-3 groups independently selected from halogen, —CN, —OH, oxo, NH₂, C₁-C₄alkyl, C₁-C₄alkoxy, optionally substituted 5-6 membered heterocyclyl, and optionally substituted 5-6 membered heterocyclyloxy;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

In one aspect, the disclosure provides a compound of Formula IX, or a pharmaceutically acceptable salt thereof:

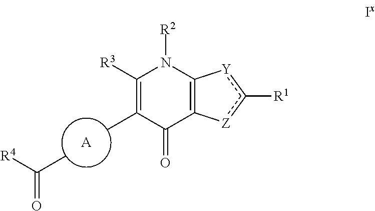

wherein Ring A represents:
   a) a 4-7 membered saturated or partially unsaturated bivalent monocyclic ring system selected from carbocyclylene and heterocyclylene (having 1-4 heteroatoms independently selected from nitrogen, oxygen, and sulfur); or
   b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
wherein Ring A is substituted with 0-4 independently selected R^{A} substituents;
each of Y and Z is selected from N and O, wherein Y is N and Z is O or Y is O and Z is N, [symbol] denotes a single or double bond, and the 5-membered ring comprising Z and Y is aromatic;
R¹ is selected from one of groups a) to e):
   a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A};
   b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A},
   c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A},
   d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}; and
   e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹. —CH₂NR¹⁰R¹¹, —SO₂R¹², wherein the C₁-C₆aliphatic, C₃-C₇cycloalkyl, or C₁-C₆alkylene-O—C₁-C₆alkyl is substituted with 0-5 independently selected R^{A};
R¹⁰ is H, C₁-C₆aliphatic, haloC₁-C₆alkyl, C₃-C₆cycloalkyl, haloC₃-C₆cycloalkyl, —C(O)C₃-C₆cycloalkyl, —C(O)C₁-C₆alkyl, or a 5-6 membered heteroaryl (having 1-4 heteroatoms independently selected from nitrogen, oxygen, and sulfur) optionally substituted with 1 or 2 groups independently selected from R^{A};
R¹¹ is H, C₁-C₆aliphatic, or C₃-C₆cycloalkyl, or R¹⁰ and R¹¹ may combine to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R;
R² is C(R^{B})₂C(O)N(R)R^{2A};
R^{B} is independently selected at each occurrence from hydrogen, —CH₃, and —CH₂CH₃, or two R^{B} taken together with the carbon to which they are attached form a cyclopropyl ring;
R^{2A} is phenyl or pyridyl, each of which is optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen; or
R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH;
R³ is hydrogen, C₁-C₄aliphatic, C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, —OR, —C(O)NR¹⁰R¹¹, or N(R)C(O)R;
each R^{3A} is independently selected at each occurrence from C₁-C₄alkyl;
R⁴ is phenyl or a 5-6 membered heteroaryl (having 1-4 heteroatoms independently selected from nitrogen, oxygen, and sulfur) wherein said phenyl or 5-6 membered heteroaryl are substituted with 0-5 R^{A} or two substituents on adjacent atoms of said phenyl or 5-6 membered heteroaryl together with said adjacent atoms form a 4-7 membered carbocyclyl, a 4-7 membered heterocyclyl, or a 5-6 membered heteroaryl (having 1-4 heteroatoms independently selected from nitrogen, oxygen, and sulfur) that is fused to the phenyl or 5-6 membered heteroaryl wherein said 4-7 membered carbocyclyl, 4-7 membered heterocyclyl, or 5-6 membered heteroaryl are substituted with 0-3 R^{A}; or
R⁴ is a C₁-C₄alkyl, C₁-C₄alkoxy, or C₃-C₆cycloalkyl, each of which is substituted with 0-3 groups independently selected from halogen, —CN, —OH, oxo, NH₂, C₁-C₄alkyl, C₁-C₄alkoxy, optionally substituted 5-6 membered heterocyclyl, and optionally substituted 5-6 membered heterocyclyloxy;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

In one aspect, the disclosure provides a compound of Formula I′, or a pharmaceutically acceptable salt thereof:

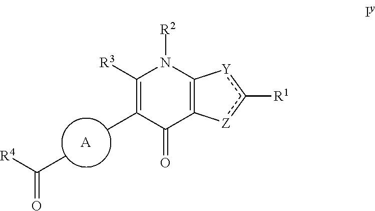

wherein Ring A represents:
   a) a 4-7 membered saturated or partially unsaturated bivalent monocyclic ring system selected from carbocyclylene and heterocyclylene (having 1-4 heteroatoms independently selected from nitrogen, oxygen, and sulfur); or
   b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
wherein Ring A is substituted with 0-4 independently selected R^{A} substituents;
each of Y and Z is selected from N and O, wherein Y is N and Z is O or Y is O and Z is N, [symbol] denotes a single or double bond, and the 5-membered ring comprising Z and Y is aromatic;
R¹ is selected from one of groups a) to e):
   a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A},
   b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A};
   c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A},
   d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}; and
   e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹, —CH₂NR¹⁰R¹¹, —SO₂R¹², wherein the C₁-C₆aliphatic, C₃-C₇cycloalkyl, or C₁-C₆alkylene-O—C₁-C₆alkyl is substituted with 0-5 independently selected R^{A};
R¹⁰ is H, C₁-C₆aliphatic, haloC₁-C₆alkyl, C₃-C₆cycloalkyl, haloC₃-C₆cycloalkyl, —C(O)C₃-C₆cycloalkyl, —C(O) C₁-C₆alkyl, or a 5-6 membered heteroaryl (having 1-4 heteroatoms independently selected from nitrogen, oxygen, and sulfur) optionally substituted with 1 or 2 groups independently selected from R^{A};
R¹¹ is H, C₁-C₆aliphatic, or C₃-C₆cycloalkyl, or R¹⁰ and R¹¹ may combine to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R;
R² is selected from C(R^{B})₂C(O)N(R)R^{2A}, C(R^{B})₂C(R^{B})₂C(O)N(R)R^{2A}, C(R^{B})₂C(R^{B})₂N(R)C(O) N(R)R^{2A}, and C(R^{B})₂C(R^{B})₂N(R)C(O)R^{2A};
R^{B} is independently selected at each occurrence from hydrogen, —CH₃, and —CH₂CH₃, or two R^{B} taken together with the carbon to which they are attached form a cyclopropyl ring;
R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen, and wherein 2 substituents on the same atom of said saturated or partially unsaturated monocyclic ring, or saturated or partially unsaturated bridged, fused, or spirocyclic ring form a cyclic group selected from:
   an optionally substituted 3-7 membered saturated or partially unsaturated carbocyclyl, and
   an optionally substituted 4-7 membered saturated or partially unsaturated heterocyclyl having 1-3 heteroatoms independently selected from nitrogen, oxygen, and sulfur; or
R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH;
R³ is hydrogen, C₁-C₄aliphatic, C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, —OR, —C(O)NR¹⁰R¹¹, or N(R)C(O)R;
each R^{3A} is independently selected at each occurrence from C₁-C₄alkyl;
R⁴ is phenyl or a first 5-6 membered heteroaryl (having 1-4 heteroatoms independently selected from nitrogen, oxygen, and sulfur) wherein said phenyl or said first 5-6 membered heteroaryl are substituted with 0-5 R^{A} or two substituents on adjacent atoms of said phenyl or said first 5-6 membered heteroaryl together with said adjacent atoms form a 4-7 membered carbocyclyl, a 4-7 membered heterocyclyl, or a second 5-6 membered heteroaryl (having 1-4 heteroatoms independently selected from nitrogen, oxygen, and sulfur) that is fused to the phenyl or first 5-6 membered heteroaryl wherein said 4-7 membered carbocyclyl, 4-7 membered heterocyclyl, or second 5-6 membered heteroaryl are substituted with 0-3 R⁴; or
R⁴ is a C₁-C₄alkyl, C₁-C₄alkoxy, or C₃-C₆cycloalkyl, each of which is substituted with 0-3 groups independently selected from halogen, —CN, —OH, oxo, NH₂, C₁-C₄alkyl, C₁-C₄alkoxy, optionally substituted 5-6 membered heterocyclyl, and optionally substituted 5-6 membered heterocyclyloxy;
each R is independently hydrogen, or an optionally substituted C₁-6aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

In one aspect, the disclosure provides a compound of Formula I″, or a pharmaceutically acceptable salt thereof:

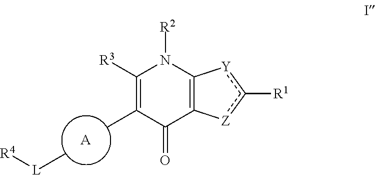

wherein Ring A represents:
   a) a 4-7 membered saturated or partially unsaturated bivalent monocyclic ring system selected from carbocyclylene and heterocyclylene (having 1-4 heteroatoms independently selected from nitrogen, oxygen, and sulfur); or
   b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
wherein Ring A is substituted with 0-4 independently selected R^{A} substituents;
each of Y and Z is selected from N and O, wherein Y is N and Z is O or Y is O and Z is N, [symbol] denotes a single or double bond, and the 5-membered ring comprising Z and Y is aromatic;
L is a linker selected from —C(O)—, —S(O)—, —S(O)₂—, and

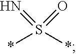

R¹ is selected from one of groups a) to e):
   a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A};
   b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A},
   c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A};
   d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}; and
   e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹. —CH₂NR¹⁰R¹¹, —SO₂R¹², wherein the C₁-C₆aliphatic, C₃-C₇cycloalkyl, or C₁-C₆alkylene-O—C₁-C₆alkyl is substituted with 0-5 independently selected R^{A};
R¹⁰ is H, C₁-C₆aliphatic, haloC₁-C₆alkyl, C₃-C₆cycloalkyl, haloC₃-C₆cycloalkyl, —C(O)C₃-C₆cycloalkyl, —C(O) C₁-C₆alkyl, or a 5-6 membered heteroaryl (having 1-4 heteroatoms independently selected from nitrogen, oxygen, and sulfur) optionally substituted with 1 or 2 groups independently selected from R^{A};
R¹¹ is H, C₁-C₆aliphatic, or C₃-C₆cycloalkyl, or R¹⁰ and R¹¹ may combine to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R;
R² is selected from C(R^{B})₂C(O)N(R)R^{2A}, C(R^{B})₂C(R^{B})₂C(O)N(R)R^{2A}, C(R^{B})₂C(R^{B})₂N(R)C(O) N(R)R^{2A}, and C(R^{B})₂C(R^{B})₂N(R)C(O)R^{2A};
R^{B} is independently selected at each occurrence from hydrogen, —CH₃, and —CH₂CH₃, or two R^{B} taken together with the carbon to which they are attached form a cyclopropyl ring;
R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen, and wherein 2 substituents on the same atom of said saturated or partially unsaturated monocyclic ring, or saturated or partially unsaturated bridged, fused, or spirocyclic ring form a cyclic group selected from:
   an optionally substituted 3-7 membered saturated or partially unsaturated carbocyclyl, and
   an optionally substituted 4-7 membered saturated or partially unsaturated heterocyclyl having 1-3 heteroatoms independently selected from nitrogen, oxygen, and sulfur; or
R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH;
R³ is hydrogen, C₁-C₄aliphatic, C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, —OR, —C(O)NR¹⁰R¹¹, or N(R)C(O)R;
each R^{3A} is independently selected at each occurrence from C₁-C₄alkyl;
R⁴ is phenyl or a first 5-6 membered heteroaryl (having 1-4 heteroatoms independently selected from nitrogen, oxygen, and sulfur) wherein said phenyl or said first 5-6 membered heteroaryl are substituted with 0-5 R^{A} or two substituents on adjacent atoms of said phenyl or said first 5-6 membered heteroaryl together with said adjacent atoms form a 4-7 membered carbocyclyl, a 4-7 membered heterocyclyl, or a second 5-6 membered heteroaryl (having 1-4 heteroatoms independently selected from nitrogen, oxygen, and sulfur) that is fused to the phenyl or first 5-6 membered heteroaryl wherein said 4-7 membered carbocyclyl, 4-7 membered heterocyclyl, or second 5-6 membered heteroaryl are substituted with 0-3 R^{A}, or
R⁴ is a C₁-C₄alkyl, C₁-C₄alkoxy, or C₃-C₆cycloalkyl, each of which is substituted with 0-3 groups independently selected from halogen, —CN, —OH, oxo, NH₂, C₁-C₄alkyl, C₁-C₄alkoxy, optionally substituted 5-6 membered heterocyclyl, and optionally substituted 5-6 membered heterocyclyloxy;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

In one aspect, the present invention provides a compound of Formula I, I′, I^{x}, I^{y}, or I″, wherein R⁴ is selected from one of a), b), and c):
a) R⁴ is a Ring B that is selected from the group consisting of:

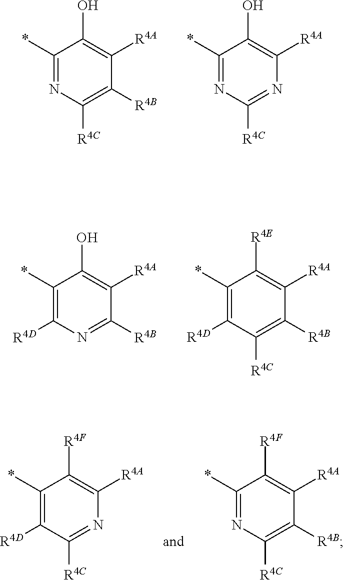

wherein * is a point of attachment to L in Formula I, I′, I^{x}, I^{y}, or I″;
and wherein:
   any substituents that are present on Ring B selected from R^{4A}, R^{4B}, R^{4C}, R^{4D}, R^{4E}, and R^{4F} are each independently selected from hydrogen; halogen; —OH; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4A} and R^{4B}, along with their intervening atoms, join to form 4-7 membered optionally substituted carbocyclyl, 4-7 membered optionally substituted heterocyclyl, or 5-6 membered optionally substituted heteroaryl (having 1-4 heteroatoms independently selected from nitrogen, oxygen, and sulfur) that is fused to Ring B; and any substituents that are present on Ring B selected from R^{4C}, R^{4D}, R^{4E}, and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4B} and R^{4C}, along with their intervening atoms, join to form 4-7 membered optionally substituted carbocyclyl, 4-7 membered optionally substituted heterocyclyl, or 5-6 membered heteroaryl (having 1-4 heteroatoms independently selected from nitrogen, oxygen, and sulfur) that is fused to Ring B; and any substituents that are present on Ring B selected from R^{4A}, R^{4D}, R^{4E}, and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form 4-7 membered optionally substituted carbocyclyl, 4-7 membered optionally substituted heterocyclyl, or 5-6 membered heteroaryl (having 1-4 heteroatoms independently selected from nitrogen, oxygen, and sulfur) that is fused to Ring B; and any substituents that are present on Ring B selected from R^{4A}, R^{4B}, R^{4E} and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} and R^{4A}, along with their intervening atoms, join to form 5-6 membered optionally substituted heteroaryl (having 1-4 heteroatoms independently selected from nitrogen, oxygen, and sulfur) that is fused to Ring B; and R^{4B}, R^{4C}, and R^{4D} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4F} and R^{4A}, along with their intervening atoms, join to form a 5-6 membered optionally substituted heteroaryl (having 1-4 heteroatoms independently selected from nitrogen, oxygen, and sulfur) that is fused to Ring B; and any substituents that are present on Ring B selected from R^{4B}, R^{4C}, and R^{4D} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen and C₁-C₄alkyl optionally substituted with —OH, —OCH₃, or —OCH₂CH₃; and
R¹⁴ is hydrogen; or NR¹³R¹⁴ forms a heterocyclic ring selected from azetidinyl, pyrrolidinyl, and piperidinyl, said heterocyclic ring optionally substituted with —CH₃; or
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy; or
c) R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl, and optionally substituted 5-6 membered heterocyclyloxy.

As described generally above, Ring A represents:
a) a 4-7 membered saturated or partially unsaturated bivalent monocyclic ring system selected from carbocyclylene and heterocyclylene (having 1-4 heteroatoms independently selected from nitrogen, oxygen, and sulfur); or
b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
   wherein Ring A is substituted with 0-4 independently selected R^{A} substituents.

In some embodiments, Ring A is a 4-7 membered saturated or partially unsaturated bivalent monocyclic ring system selected from carbocyclylene and heterocyclylene (having 1-4 heteroatoms independently selected from nitrogen, oxygen, and sulfur), wherein Ring A is substituted with 0-4 independently selected R^{A} substituents. In some embodiments, Ring A is a 4-7 membered saturated or partially unsaturated bivalent monocyclic carbocyclylene, wherein Ring A is substituted with 0-4 independently selected R^{A} substituents. In some embodiments, Ring A is a 4-7 membered saturated or partially unsaturated bivalent monocyclic heterocyclylene (having 1-4 heteroatoms independently selected from nitrogen, oxygen, and sulfur), wherein Ring A is substituted with 0-4 independently selected R^{A} substituents.

In some embodiments, Ring A is a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur), wherein Ring A is substituted with 0-4 independently selected R^{A} substituents. In some embodiments, Ring A is a 4-12 membered saturated or partially unsaturated bivalent bicyclic ring system that is fused, bridged, or spirocyclic and is a carbocyclylene, wherein Ring A is substituted with 0-4 independently selected R^{A} substituents. In some embodiments, Ring A is a 4-12 membered saturated or partially unsaturated bivalent bicyclic ring system that is fused, bridged, or spirocyclic and is a heterocyclylene (having 1-4 heteroatoms independently selected from nitrogen, oxygen, and sulfur), wherein Ring A is substituted with 0-4 independently selected R^{A} substituents.

In some embodiments, Ring A is a 4-12 membered saturated or partially unsaturated bivalent bicyclic ring system comprising 2 fused rings. In some embodiments, Ring A is a 4-12 membered saturated or partially unsaturated bivalent bicyclic ring system comprising a spirocyclic ring system. In some embodiments, Ring A is a 4-12 membered saturated or partially unsaturated bivalent bicyclic ring system comprising a bridged ring system.

In some embodiments, Ring A is selected from

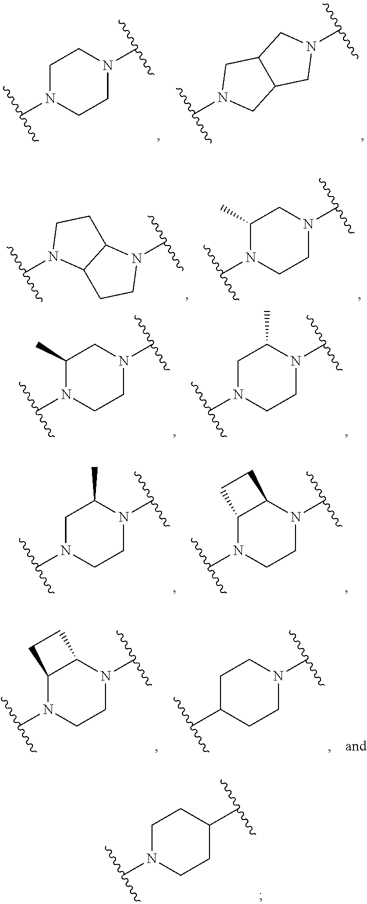

and wherein Ring A is substituted with 0-4 independently selected R^{A} substituents.

In some embodiments, Ring A is

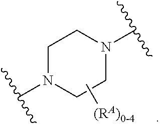

In some embodiments, Ring A is

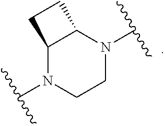 [SMILES: *N1CCN(*)[C@H]2CC[C@@H]21]

In some embodiments, Ring A is

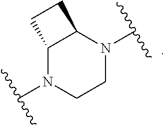 [SMILES: *N1CCN(*)[C@@H]2CC[C@H]21]

In some embodiments, Ring A is as selected from one of the substituents of Table 1, Table 1a, or Table 1b.

As described generally above, R¹ is selected from groups a)-e):
a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A};
b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A};
c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A};
d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}; and
e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹. —CH₂NR¹⁰R¹¹, —SO₂R¹², wherein the C₁-C₆aliphatic, C₃-C₇cycloalkyl, or C₁-C₆alkylene-O-C₁-C₆alkyl is substituted with 0-5 independently selected R^{A}.

In some embodiments, R¹ is a 5-6 membered monocyclic heteroaryl (having 1-4 heteroatoms independently selected from nitrogen, oxygen, and sulfur) optionally substituted with 1-3 groups independently selected from C₁-C₆alkyl, C₁-C₆alkoxy, C₃-C₆cycloalkyl, and C₃-C₆cycloalkoxy, wherein said 5-6 membered monocyclic heteroaryl is further substituted with 0-2 independently selected R^{A}. In some embodiments, R¹ is a 4- or 6-membered saturated or partially unsaturated monocyclic heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen. In some embodiments, R¹ is a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen. In some embodiments, R¹ is a 3-7 membered optionally substituted carbocyclyl. In some embodiments, R¹ is an optionally substituted C₂-C₄alkenyl. In some embodiments, R¹ is cyclopropyl substituted C₂-C₄alkenyl. In some embodiments, R¹ is methyl substituted C₂alkenyl.

In some embodiments, R¹ is a 6-membered partially unsaturated heterocyclyl (having 1 oxygen atom). In some embodiments, R¹ is a 6-membered heteroaryl (having 1 nitrogen atom), said heteroaryl may be optionally substituted with 1 or 2 groups independently selected from C₁-C₆alkyl, C₁-C₆alkoxy, C₃-C₆cycloalkyl, and C₃-C₆cycloalkoxy, wherein said heteroaryl is further substituted with 0-1 R^{A}, wherein R^{A} is an optionally substituted C₁₋₆ aliphatic group. In some embodiments, R¹ is a 6-membered heteroaryl (having 2 nitrogen atoms), said heteroaryl may be optionally substituted with 1 or 2 groups independently selected from C₁-C₆alkyl, C₁-C₆alkoxy, C₃-C₆cycloalkyl, and C₃-C₆cycloalkoxy, wherein said heteroaryl is further substituted with 0-1 R^{A}, wherein R^{A} is an optionally substituted C₁₋₆ aliphatic group.

In some embodiments, R¹ is a bicyclic 9-10 membered heteroaryl (having 1-4 heteroatoms independently selected from nitrogen, oxygen, and sulfur) optionally substituted with 1 or 2 groups independently selected from C₁-C₆alkyl, C₃-C₆cycloalkyl, C₁-C₆alkoxy, and C₃-C₆cycloalkoxy, wherein said bicyclic 9-10 membered heteroaryl is further substituted with 0-3 independently selected R^{A}.

In some embodiments, R¹ is a 5-membered heteroaryl (having 1-3 heteroatoms independently selected from nitrogen, oxygen, and sulfur) optionally substituted with 1 or 2 groups independently selected from C₁-C₆alkyl, C₁-C₆alkoxy, C₃-C₆cycloalkyl, and C₃-C₆cycloalkoxy, wherein said 5-membered heteroaryl is optionally further substituted with 0-2 independently selected R^{A}. In some embodiments, R¹ is a 5-membered heteroaryl (having 1-3 heteroatoms independently selected from nitrogen, oxygen, and sulfur) optionally substituted with 1 or 2 groups independently selected from C₁-C₆alkyl, C₁-C₆alkoxy, C₃-C₆cycloalkyl, and C₃-C₆cycloalkoxy. In some embodiments, R¹ is a 5-membered heteroaryl (having 2 nitrogen atoms) substituted with 1 or 2 groups independently selected from C₁-C₆alkoxy, C₃-C₆cycloalkyl, and C₃-C₆cycloalkoxy, wherein said 5-membered heteroaryl is optionally further substituted with 0-1 R^{A}, wherein R^{A} is hydroxyl substituted C₁-C₄alkyl.

In some embodiments, R¹ is a 5-6 membered heteroaryl (having 1-4 heteroatoms independently selected from nitrogen, oxygen, and sulfur) optionally substituted with one group of C₁-C₆alkoxy or C₃-C₆cycloalkyl, wherein said 5-6 membered heteroaryl is optionally further substituted with 0-3 independently selected R^{A}.

In some embodiments, R¹ is pyridyl substituted with C₁-C₄alkoxy and further substituted with 0-2 R^{A} substituents.

In some embodiments, R¹ is 5-membered heteroaryl (having 1 heteroatom independently selected from nitrogen, oxygen, and sulfur, and 0 or 1 additional ring nitrogen atoms), wherein said 5-membered heteroaryl is optionally substituted with C₁-C₆alkyl, or C₃-C₅cycloalkyl and further substituted with 0-2 R^{A} substituents.

In some embodiments, R¹ is
c) a 5-6 membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen; or
d) a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen.

In some embodiments, R¹ is a 5-6 membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen.

In some embodiments, R¹ is a 5-6 membered heteroaryl (having 1-4 heteroatoms independently selected from nitrogen, oxygen, and sulfur) substituted with 1 group selected from C₁-C₆alkoxy and C₃-C₆cycloalkyl, wherein said 5-6 membered heteroaryl is further substituted with 0-3 independently selected R^{A}.

In some embodiments, R¹ is

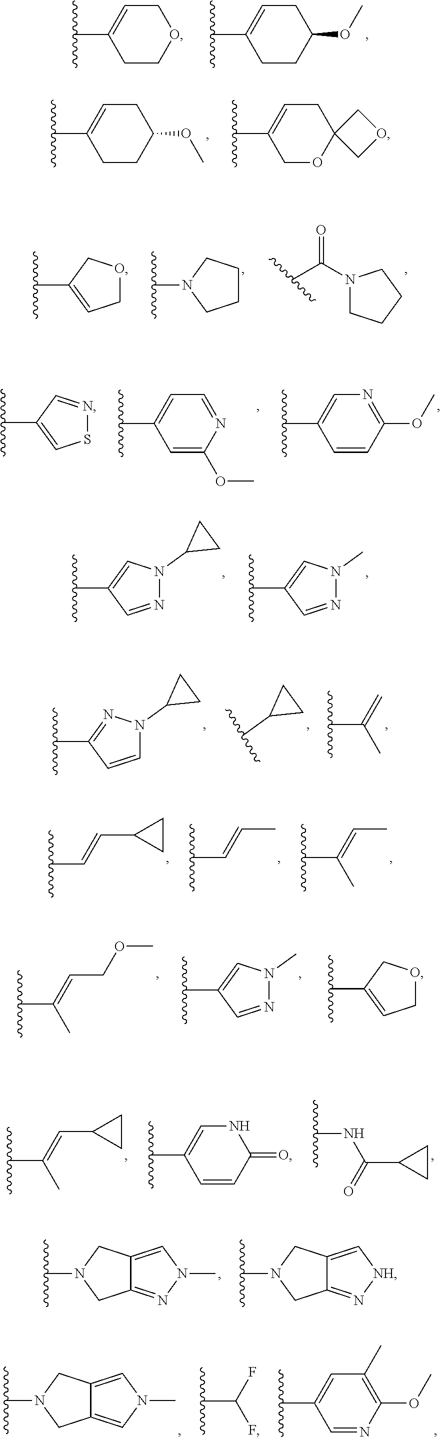

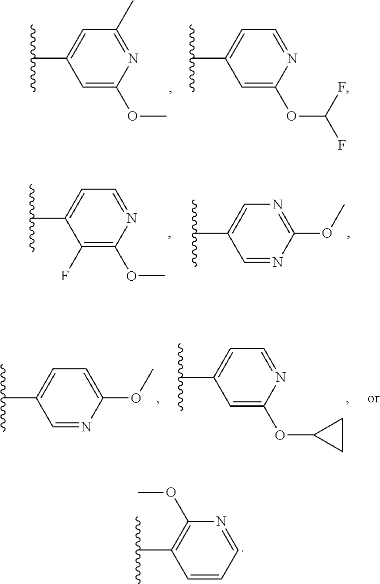

In some embodiments R¹ is

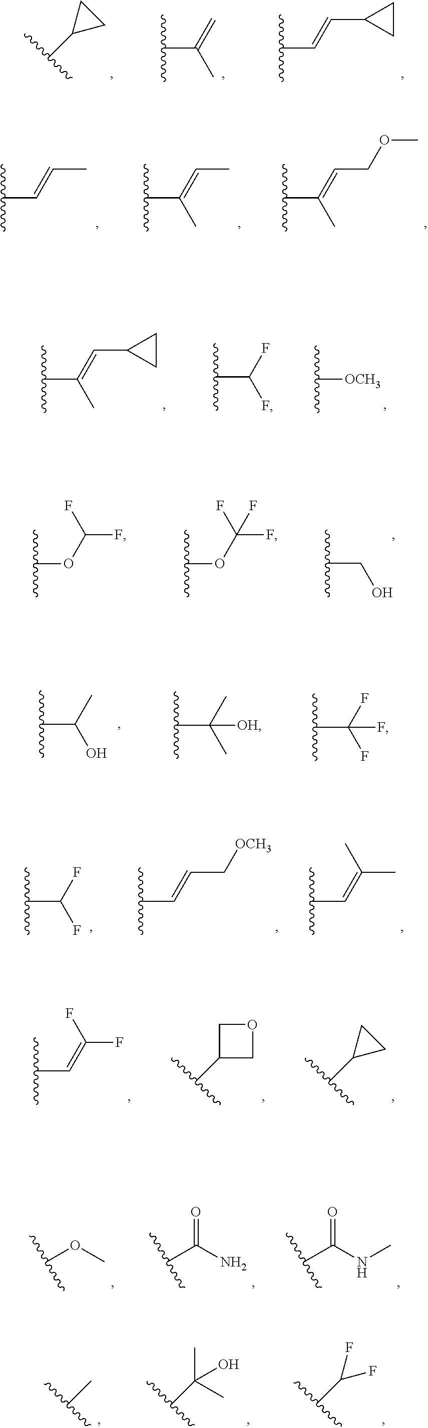

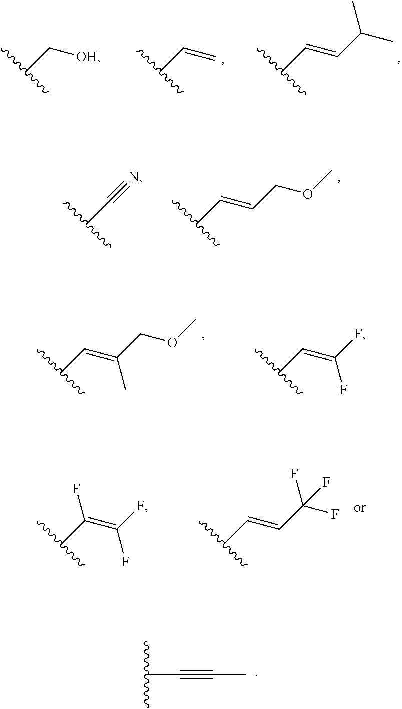

In some embodiments, R¹ is

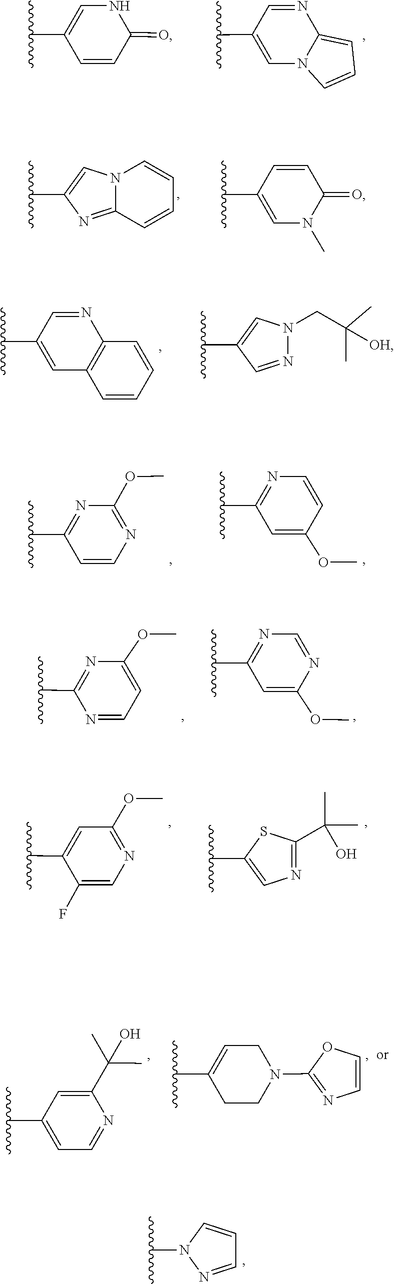

In some embodiments, R¹ is

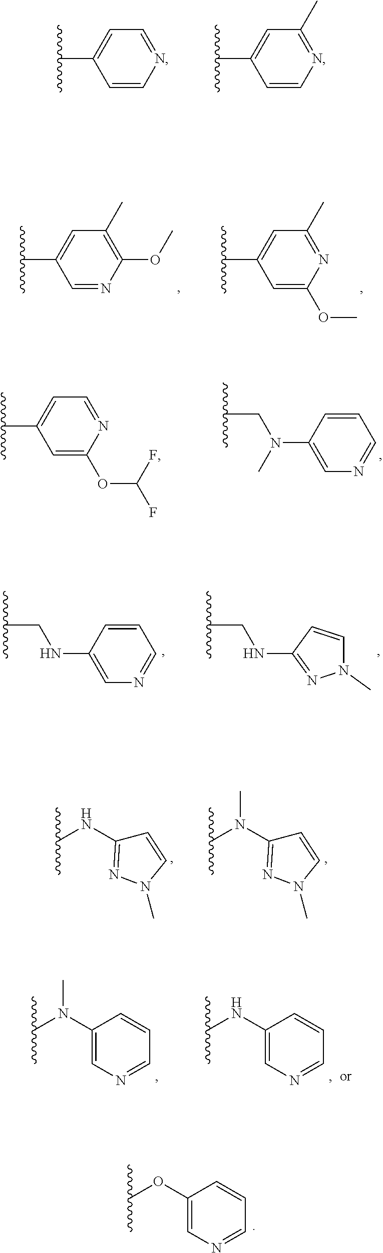

In some embodiments R¹ is

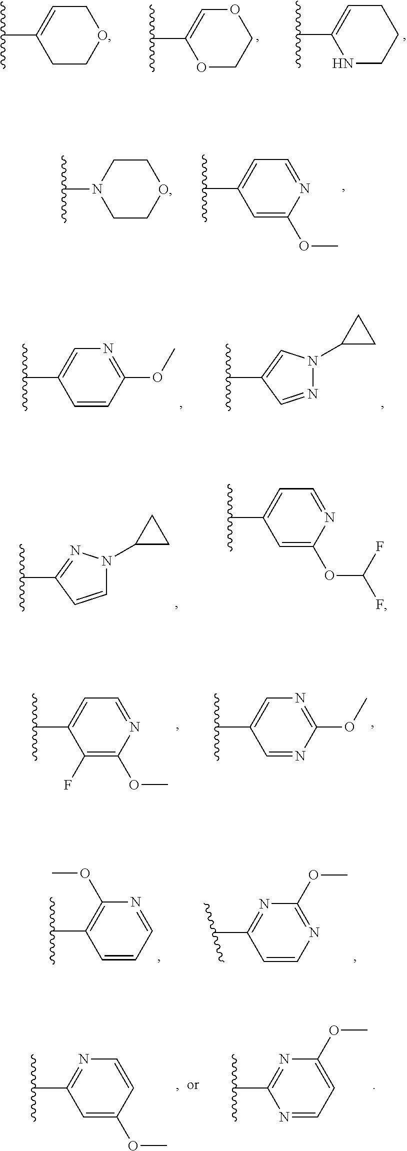

In some embodiments, R¹ is as selected from one of the substituents of Table 1, Table 1a, or Table 1b.

As described generally above, R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R.

In some embodiments, R^{A} is independently selected at each occurrence from the group consisting of halogen, —OR, or an optionally substituted C₁₋₆aliphatic group. In some embodiments, R^{A} is independently selected at each occurrence from a halogen. In some embodiments, R^{A} is independently selected at each occurrence from an —OR. In some embodiments R^{A} is an optionally substituted 5-6 membered heteroaryl ring (having 1-4 heteroatoms independently selected from nitrogen, oxygen, and sulfur). In some embodiments, R^{A} is an optionally substituted C₃-C₆cycloalkyl. In some embodiments, R^{A} is independently selected at each occurrence from an optionally substituted C₁₋₆aliphatic group.

In some embodiments, R^{A} is as selected from one of the substituents of Table 1, Table 1a, or Table 1b.

As described generally above, R² is C(R^{B})₂C(O)N(R)R^{2A}, C(R^{B})₂C(R^{B})₂C(O)N(R)R^{2A}, C(R^{B})₂C(R^{B})₂N(R)C(O)N(R)R^{2A}, or C(R^{B})₂C(R^{B})₂N(R)C(O)R^{2A}. In some embodiments, R² is C(R^{B})₂C(O)N(R)R^{2A}. In some embodiments, R² is C(R^{B})₂C(R^{B})₂C(O)N(R)R^{2A}. In some embodiments, R² is C(R^{B})₂C(R^{B})₂N(R)C(O)N(R)R^{2A}. In some embodiments, R² is C(R^{B})₂C(R^{B})₂N(R)C(O)R^{2A}. In some embodiments, R² is CH₂C(O)N(H)R^{2A}. In some embodiments, R² is CH₂CH₂C(O)N(H)R^{2A}. In some embodiments, R² is CH₂CH₂N(R)C(O)N(R)R^{2A}. In some embodiments, R² is CH₂CH₂N(H) C(O)R^{2A}. In some embodiments, R² is C(R^{B})₂C(O)N(H)R^{2A}, wherein R^{2A} is phenyl or bicyclo[1.1.1]pentyl optionally substituted with 1, 2, or 3 substituents independently selected from halogen, C₁-C₄alkyl, or haloC₁-C₄alkyl. In some embodiments, R² is C(R^{B})₂C(O)N(H)R^{2A}, wherein R^{2A} is phenyl optionally substituted with 1, 2, or 3 substituents independently selected from halogen, C₁-C₄alkyl, or haloC₁-C₄alkyl. In some embodiments, R² is C(R^{B})₂C(O)N(H)R^{2A}, wherein R^{2A} is bicyclo[1.1.1]pentyl optionally substituted with 1, 2, or 3 substituents independently selected from halogen, C₁-C₄alkyl, or haloC₁-C₄alkyl.

In some embodiments, R² is

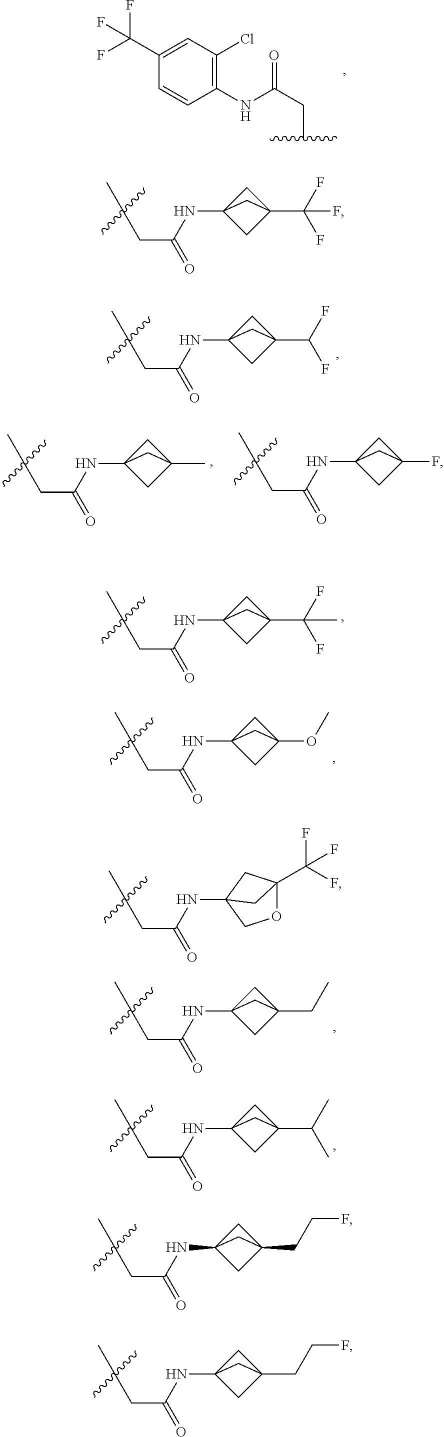

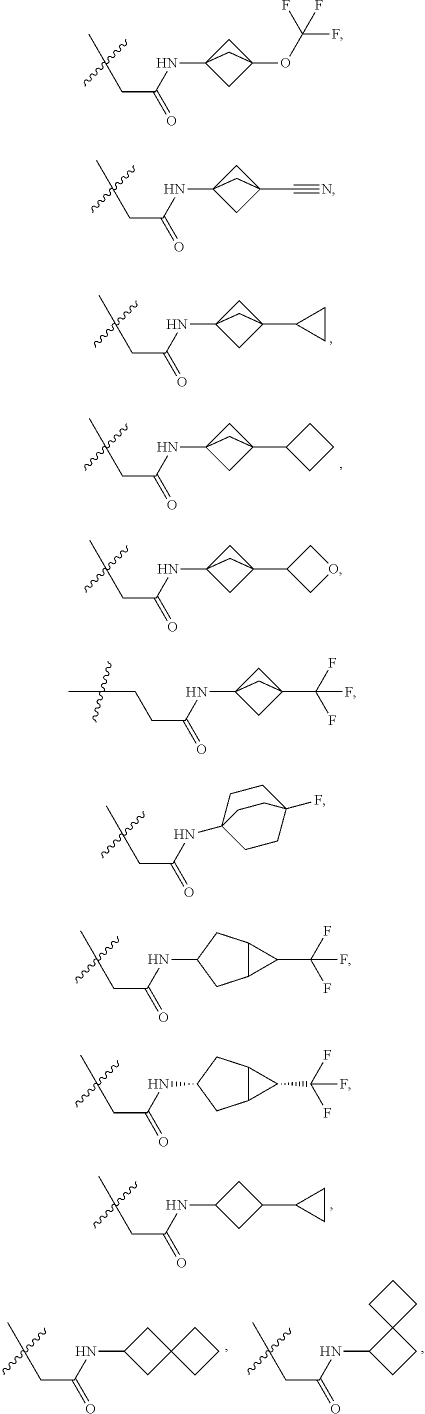

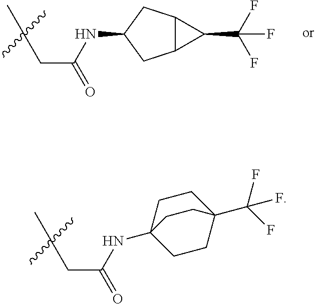 [SMILES: *CC(=O)N[C@H]1CC2C(C1)[C@@H]2C(F)(F)F ; *CC(=O)NC12CCC(C(F)(F)F)(CC1)CC2]

In some embodiments R² is

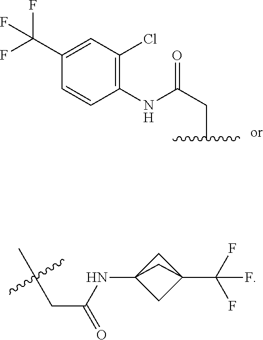 [SMILES: *CC(=O)Nc1ccc(C(F)(F)F)cc1Cl ; *CC(=O)NC12CC(C(F)(F)F)(C1)C2]

In some embodiments, R² is

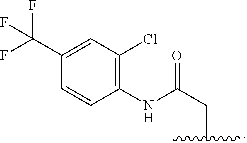 [SMILES: *CC(=O)Nc1ccc(C(F)(F)F)cc1Cl]

In some embodiments R² is

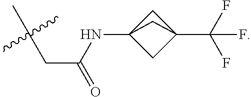 [SMILES: *CC(=O)NC12CC(C(F)(F)F)(C1)C2]

In some embodiments, R² is as selected from one of the substituents of Table 1, Table 1a, or Table 1b.

As described generally above, R^{B} is independently selected at each occurrence from hydrogen, —CH₃, and —CH₂CH₃, or two R^{B} taken together with the carbon to which they are attached form a cyclopropyl ring. In some embodiments, R^{B} is independently selected at each occurrence from hydrogen, —CH₃, and —CH₂CH₃. In some embodiments, R^{B} is hydrogen. In some embodiments, two R^{B} taken together with the carbon to which they are attached form a cyclopropyl ring.

In some embodiments, R^{B} is as selected from one of the substituents of Table 1, Table 1a, or Table 1b.

As described generally above, R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring, or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆-cycloalkoxy, haloC₃-C₆cycloalkoxy and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen, and wherein 2 substituents on the same atom of said saturated or partially unsaturated monocyclic ring or saturated or partially unsaturated bridged, fused, or spirocyclic ring form a cyclic group selected from:
an optionally substituted 3-7 membered saturated or partially unsaturated carbocyclyl, and
an optionally substituted 4-7 membered saturated or partially unsaturated heterocyclyl having 1-3 heteroatoms independently selected from nitrogen, oxygen, and sulfur, or
   R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH.

In some embodiments, there are 1-6 respective instances of wherein 2 substituents on the same 1^{st}, 2^{nd}, 3^{rd}, 4^{th}, 5^{th}, or 6^{th} atom of said saturated or partially unsaturated monocyclic ring, or said saturated or partially unsaturated bridged, fused, or spirocyclic ring form 1-6 of said cyclic groups. In some embodiments, there is one instance wherein 2 substituents on the same atom of said saturated or partially unsaturated monocyclic ring, or said saturated or partially unsaturated bridged, fused, or spirocyclic ring form one of said cyclic groups. In some embodiments, there 2 respective instances of wherein 2 substituents on the same 1^{st} and 2^{nd} atoms of said saturated or partially unsaturated monocyclic ring, or said saturated or partially unsaturated bridged, fused, or spirocyclic ring form both of said cyclic groups. In some embodiments, there are 3 respective instances of wherein 2 substituents on the same 1^{st}, 2^{nd}, and 3^{rd}, atoms of said saturated or partially unsaturated monocyclic ring, or said saturated or partially unsaturated bridged, fused, or spirocyclic ring form the three of said cyclic groups. In some embodiments, there are 4 respective instances of wherein 2 substituents on the same 1^{st}, 2^{nd}, 3^{rd}, and 4^{th} atoms of said saturated or partially unsaturated monocyclic ring, or said saturated or partially unsaturated bridged, fused, or spirocyclic ring form the four of said cyclic groups. In some embodiments, there are 5 respective instances of wherein 2 substituents on the same 1^{st}, 2^{nd}, 3^{rd}, 4^{th} and 5^{th} atoms of said saturated or partially unsaturated monocyclic ring, or said saturated or partially unsaturated bridged, fused, or spirocyclic ring form the five of said cyclic groups. In some embodiments, there 6 respective instances of wherein 2 substituents on the same 1^{st}, 2^{nd}, 3^{rd}, 4^{th} 5^{th}, and 6^{th} atoms of said saturated or partially unsaturated monocyclic ring, or said saturated or partially unsaturated bridged, fused, or spirocyclic ring form the six of said cyclic groups.

In some embodiments, R^{2A} is phenyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅, two substituents on adjacent atoms of the phenyl together with said adjacent atoms form a 4-7 membered carbocyclyl fused to the phenyl, and two substituents on adjacent atoms of the phenyl together with said adjacent atoms form a 4-7 membered heterocyclyl (having 1-4 heteroatoms independently selected from nitrogen, oxygen, and sulfur) fused to the phenyl.

In some embodiments, R^{2A} is phenyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅. In some embodiments, R^{2A} is phenyl optionally substituted with 1, 2, or 3 substituents independently selected from halogen, C₁-C₄alkyl, and haloC₁-C₄alkyl. In some embodiments, R^{2A} is phenyl optionally substituted with a halogen, C₁-C₄alkyl, and haloC₁-C₄alkyl. In some embodiments, R^{2A} is phenyl optionally substituted with 2 substituents independently selected from halogen, C₁-C₄alkyl, and haloC₁-C₄alkyl. In some embodiments, R^{2A} is phenyl optionally substituted with 3 substituents independently selected from halogen, C₁-C₄alkyl, and haloC₁-C₄alkyl.

In some embodiments, R^{2A} is pyridyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅, two substituents on adjacent atoms of the pyridyl together with said adjacent atoms form a 4-7 membered carbocyclyl fused to the pyridyl, and two substituents on adjacent atoms of the pyridyl together with said adjacent atoms form a 4-7 membered heterocyclyl (having 1-4 heteroatoms independently selected from nitrogen, oxygen, and sulfur) fused to the pyridyl.

In some embodiments, R^{2A} is pyridyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅. In some embodiments, R^{2A} is pyridyl optionally substituted with 1, 2, or 3 substituents independently selected from halogen, C₁-C₄alkyl, and haloC₁-C₄alkyl. In some embodiments, R^{2A} is pyridyl optionally substituted with a halogen, C₁-C₄alkyl, or haloC₁-C₄alkyl. In some embodiments, R²⁴ is pyridyl optionally substituted with 2 substituents independently selected from halogen, C₁-C₄alkyl, and haloC₁-C₄alkyl. In some embodiments, R^{2A} is pyridyl optionally substituted with 3 substituents independently selected from halogen, C₁-C₄alkyl, and haloC₁-C₄alkyl.

In some embodiments, R^{2A} is cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said cubanyl, partially unsaturated monocyclic ring, or saturated or partially unsaturated bridged, fused, or spirocyclic ring are each optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆cycloalkoxy and —SF₅.

In some embodiments, R^{2A} is bicyclo[1.1.1]pentyl optionally substituted with 1, 2, or 3 substituents independently selected from halogen, C₁-C₄alkyl, and haloC₁-C₄alkyl. In some embodiments, R^{2A} is bicyclo[2.2.2]octyl optionally substituted with 1, 2, or 3 substituents independently selected from halogen, C₁-C₄alkyl, and haloC₁-C₄alkyl. In some embodiments, R^{2A} is bicyclo[3.1.0]pentyl optionally substituted with 1, 2, or 3 substituents independently selected from halogen, C₁-C₄alkyl, and haloC₁-C₄alkyl. In some embodiments, R^{2A} is cubanyl optionally substituted with 1, 2, or 3 substituents independently selected from halogen, C₁-C₄alkyl, and haloC₁-C₄alkyl. In some embodiments, R^{2A} is 2-oxabicyclo[2.1.1]hexan-4-yl optionally substituted with 1, 2, or 3 substituents independently selected from halogen, C₁-C₄alkyl, and haloC₁-C₄alkyl.

In some embodiments, R^{2A} is a saturated or partially unsaturated bridged 5-, 6-, 7-, 8-, 9-, 10-, 11-, or 12-membered ring, which contains 0, 1, 2, 3, or 4 heteroatoms independently selected from nitrogen, oxygen, and sulfur; and wherein said bridged ring is optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆cycloalkoxy and —SF₅.

In some embodiments, R^{2A} is a saturated or partially unsaturated fused 5-, 6-, 7-, 8-, 9-, 10-, 11-, or 12-membered ring, which contains 0, 1, 2, 3, or 4 heteroatoms independently selected from nitrogen, oxygen, and sulfur; and wherein said fused ring is optionally substituted with 1, 2, or 3 substituents independently selected from halogen, C₁-C₄alkyl, haloC₁-C₄alkyl, C₃-C₆-cycloalkyl, haloC₃-C₆cycloalkyl, —OH, —CN, C₁-C₄alkoxy, haloC₁-C₄alkoxy, C₃-C₆cycloalkoxy, haloC₃-C₆cycloalkoxy and —SF₅.

In some embodiments, R^{2A} is a saturated or partially unsaturated spirocyclic 5-, 6-, 7-, 8-, 9-, 10-, 11-, or 12-membered ring, which contains 0, 1, 2, 3, or 4 heteroatoms independently selected from nitrogen, oxygen, and sulfur; and wherein said spirocyclic ring is optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆-cycloalkoxy, haloC₃-C₆cycloalkoxy and —SF₅.

In some embodiments, R^{2A} is bicyclo[1.1.1]pentyl optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅. In some embodiments, R^{2A} is bicyclo[1.1.1]pentyl optionally substituted with 1, 2, or 3 substituents independently selected from halogen, C₁-C₄alkyl, and haloC₁-C₄alkyl. In some embodiments, R^{2A} is bicyclo[1.1.1]pentyl optionally substituted with a halogen, C₁-C₄alkyl, or haloC₁-C₄alkyl. In some embodiments, R^{2A} is bicyclo[1.1.1]pentyl optionally substituted with 2 substituents independently selected from halogen, C₁-C₄alkyl, and haloC₁-C₄alkyl. In some embodiments, R^{2A} is bicyclo[1.1.1]pentyl optionally substituted with 3 substituents independently selected from halogen, C₁-C₄alkyl, and haloC₁-C₄alkyl.

In some embodiments, R^{2A} is Ring F selected from the group consisting of:

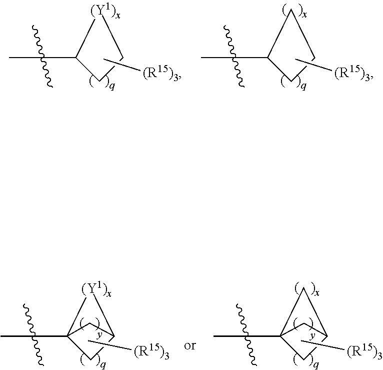

wherein x, y, and q are independently selected from 1, 2, or 3, Y¹ is independently selected from O, NR¹⁵, CHR¹⁵ or CR¹⁵R¹⁵; R¹⁵ is independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅.

In some embodiments, R^{2A} is Ring F of the following structure

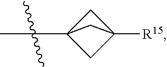

wherein R¹⁵ is selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅.

In some embodiments, R^{2A} is 2-benzimidazolyl, 2-naphthyl, or 3-quinolinyl, each of which is optionally substituted with 1, 2, or 3 substituents independently selected from halogen, C₁₋₄alkyl and —OH. In some embodiments, R^{2A} is 2-benzimidazolyl optionally substituted with 1, 2, or 3 substituents independently selected from halogen, C₁₋₄alkyl and —OH. In some embodiments, R^{2A} is 3-quinolinyl optionally substituted with 1, 2, or 3 substituents independently selected from halogen, C₁₋₄alkyl, and —OH.

In some embodiments, R^{2A} is phenyl comprising a —CF₃ substituent or pyridyl comprising a —CF₃ substituent.

In some embodiments, R^{2A} is bicyclo[1.1.1]pentyl comprising a —CF₃ substituent or bicyclo[1.1.1]pentyl comprising a —CHF₂ substituent.

In some embodiments, R^{2A} is as selected from one of the substituents of Table 1, Table 1a, or Table 1b.

As described generally above, R³ is hydrogen, C₁-C₄aliphatic, C₃-C₅cycloalkyl, C₁-C₄alkoxy, —NHR^{3A}, —N(R^{3A})₂ or C₁-C₄alkylthio each of which, besides hydrogen, is optionally substituted with —OH, 1-5 independently selected halogen, or C₁-C₄alkoxy.

In some embodiments, R³ is hydrogen. In some embodiments, R³ is C₁-C₄alkyl optionally substituted with —OH, 1-5 independently selected halogen, or C₁-C₄alkoxy. In some embodiments, R³ is C₁-C₄alkyl. In some embodiments, R³ is —CH₂CH₃. In some embodiments, R³ is —CH₃. In some embodiments, R³ is C₃-C₅cycloalkyl, C₁-C₄alkoxy, —NHR^{3A}, —N(R^{3A})₂ or C₁-C₄alkylthio optionally substituted with —OH, 1-5 independently selected halogen, or C₁-C₄alkoxy. In some embodiments, R³ is C₃-C₅cycloalkyl optionally substituted with —OH, 1-5 independently selected halogen, or C₁-C₄alkoxy. In some embodiments, R³ is C₁-C₄alkoxy optionally substituted with —OH, 1-5 independently selected halogen, or C₁-C₄alkoxy. In some embodiments, R³ is —NHR^{3A} optionally substituted with —OH, 1-5 independently selected halogen, or C₁-C₄alkoxy. In some embodiments, R³ is —N(R^{3A})₂ optionally substituted with —OH, 1-5 independently selected halogen, or C₁-C₄alkoxy. In some embodiments, R³ is C₁-C₄alkylthio optionally substituted with —OH, 1-5 independently selected halogen, or C₁-C₄alkoxy. In some embodiments, R³ is selected from the group consisting of C₁-C₄alkyl and C₃-C₅cycloalkyl.

In some embodiments, R³ is as selected from one of the substituents of Table 1, Table 1a, or Table 1b.

As described generally above, each R^{3A} is independently selected at each occurrence from C₁-C₄alkyl. In some embodiments, R^{3A} is —CH₃. In some embodiments, R^{3A} is —CH₂CH₃. In some embodiments, R^{3A} is propyl. In some embodiments, R^{3A} is butyl.

In some embodiments, R^{3A} is as selected from one of the substituents of Table 1, Table 1a, or Table 1b.

As described generally above L is a linker selected from —C(O)—, —S(O)—, —S(O)₂—, and

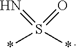 [SMILES: *S(*)(=N)=O]

In some embodiments, linker L is —C(O)—.

In some embodiments, linker L is —S(O)—.

In some embodiments, linker L is —S(O)₂—.

In some embodiments, linker L is

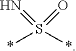

In some embodiments, linker L is as selected from one of the substituents of Table 1, Table 1a, or Table 1b.

As described generally above, R⁴ is selected from one of a), b), and c):
a) R⁴ is a Ring B that is selected from the group consisting of

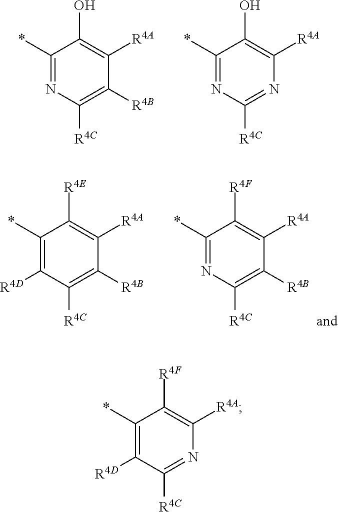

wherein * is a point of attachment to linker L that is bonded to Ring A in Formula I;
and wherein:
any substituents that are present on Ring B selected from R^{4A}, R^{4B}, R^{4C}, R^{4D}, R^{4E}, and R^{4F} are each independently selected from hydrogen; halogen; —OH; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form 4-7 membered optionally substituted carbocyclyl, 4-7 membered optionally substituted heterocyclyl, or 5-6 membered optionally substituted heteroaryl (having 1-4 heteroatoms independently selected from nitrogen, oxygen, and sulfur) that is fused to Ring B; and any substituents that are present on Ring B selected from R^{4C}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4B} and R^{4C}, along with their intervening atoms, join to form 4-7 membered optionally substituted carbocyclyl, 4-7 membered optionally substituted heterocyclyl, or 5-6 membered heteroaryl (having 1-4 heteroatoms independently selected from nitrogen, oxygen, and sulfur) that is fused to Ring B; and any substituents that are present on Ring B selected from R^{4A}, R^{4D}, R^{4F}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form 4-7 membered optionally substituted carbocyclyl, 4-7 membered optionally substituted heterocyclyl, or 5-6 membered heteroaryl (having 1-4 heteroatoms independently selected from nitrogen, oxygen, and sulfur) that is fused to Ring B; and any substituents that are present on Ring B selected from R^{4A}, R^{4B}, R^{4E} and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} is halogen or —OH, and R^{4A}, R^{4B}, R^{4C}, and RAD are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} and R^{4A}, along with their intervening atoms, join to form 5-6 membered optionally substituted heteroaryl (having 1-4 heteroatoms independently selected from nitrogen, oxygen, and sulfur) that is fused to Ring B; and R^{4B}, R^{4C}, and RAD are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4F} and R^{4A}, along with their intervening atoms, join to form a 5-6 membered optionally substituted heteroaryl (having 1-4 heteroatoms independently selected from nitrogen, oxygen, and sulfur) that is fused to Ring B; and any substituents that are present on Ring B selected from R^{4B}, R^{4C}, and RAD are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴;
R¹³ is independently selected at each occurrence from hydrogen and C₁-C₄alkyl optionally substituted with —OH, —OCH₃, or —OCH₂CH₃; and
R¹⁴ is hydrogen; or NR¹³R¹⁴ forms a heterocyclic ring selected from azetidinyl, pyrrolidinyl, and piperidinyl, said heterocyclic ring optionally substituted with —CH₃; or
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy; or
c) R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl, and optionally substituted 5-6 membered heterocyclyloxy;

In some embodiments, R⁴ is Ring B of the following structure:

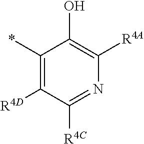

wherein * is a point of attachment to Linker L that is bonded to Ring A in Formula I;
and wherein:
   R^{4A}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4C} and R^{4D}, along with their intervening atoms, join to form 4-7 membered carbocyclyl or 4-7 membered heterocyclyl (having 1-4 heteroatoms independently selected from nitrogen, oxygen, and sulfur) that is fused to Ring B; and R^{4A} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR¹³R¹⁴; and
   R¹³ is independently selected at each occurrence from hydrogen and C₁-C₄alkyl optionally substituted with —OH, —OCH₃, or —OCH₂CH₃;
R¹⁴ is hydrogen or NR¹³R¹⁴ forms a heterocyclic ring selected from azetidinyl, pyrrolidinyl, and piperidinyl, said heterocyclic ring optionally substituted with —CH₃.

In some embodiments, R⁴ is Ring B of the following structure:

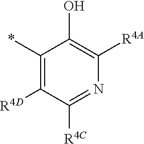

wherein * is a point of attachment to Linker L that is bonded to Ring A in Formula I;
and wherein:
   R^{4A} is —OCH₃, —OCH₂CH₃, or —OCHF₂;
   R^{4C} and R^{4D} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
   R¹³ is independently selected at each occurrence from hydrogen and C₁-C₄alkyl optionally substituted with —OH, —OCH₃, or —OCH₂CH₃;
   R¹⁴ is hydrogen or NR¹³R¹⁴ forms a heterocyclic ring selected from azetidinyl, pyrrolidinyl, and piperidinyl, said heterocyclic ring optionally substituted with —CH₃; or
   R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 substituents independently selected from halogen, —OH, —CN, C₁-C₄alkyl, haloC₁-C₄alkyl, C₃-C₆cycloalkyl, and C₁-C₄alkoxy.

In some embodiments, R⁴ is Ring B of the following structure:

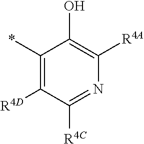

wherein * is a point of attachment to Linker L that is bonded to Ring A in Formula I;
and wherein:
   R^{4A} is —OCH₃, —OCH₂CH₃, or —OCHF₂;
   R^{4C} and R^{4D} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
   R¹³ is independently selected at each occurrence from hydrogen and C₁-C₄alkyl optionally substituted with —OH, —OCH₃, or —OCH₂CH₃; and
   R¹⁴ is hydrogen; or NR¹³R¹⁴ forms a heterocyclic ring selected from azetidinyl, pyrrolidinyl, and piperidinyl, said heterocyclic ring optionally substituted with —CH₃.

In some embodiments, R⁴ is Ring B of the following structure:

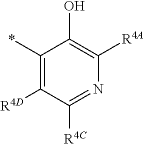

wherein * is a point of attachment to Linker L that is bonded to Ring A in Formula I;
and wherein:
   R^{4A}, R^{4C}, and R^{4D} are each independently selected from hydrogen; halogen; and C₁-C₄alkyl.

In some embodiments, R⁴ is Ring B of the following structure:

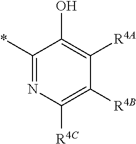

wherein * is a point of attachment to Linker L that is bonded to Ring A in Formula I;
and wherein:
   R^{4A}, R^{4B}, and R^{4C} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4A} and R^{4B}, along with their intervening atoms, join to form 4-7 membered carbocyclyl or heterocyclyl (having 1-4 heteroatoms independently selected from nitrogen, oxygen, and sulfur) that is fused to Ring B; and R^{4C} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR¹³R¹⁴; or
   R^{4B} and R^{4C}, along with their intervening atoms, join to form 4-7 membered carbocyclyl or heterocyclyl (having 1-4 heteroatoms independently selected from nitrogen, oxygen, and sulfur) that is fused to Ring B; and R⁴ is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR¹³R¹⁴; and
   R¹³ is independently selected at each occurrence from hydrogen and C₁-C₄alkyl optionally substituted with —OH, —OCH₃, or —OCH₂CH₃; and
   R¹⁴ is hydrogen; or NR¹³R¹⁴ forms a heterocyclic ring selected from azetidinyl, pyrrolidinyl, and piperidinyl, said heterocyclic ring optionally substituted with —CH₃.

In some embodiments, R⁴ is Ring B of the following structure:

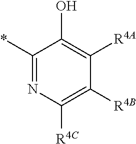

wherein * is a point of attachment to Linker L that is bonded to Ring A in Formula I;
and wherein:
   R^{4A} and R^{4B}, along with their intervening atoms, join to form 4-7 membered carbocyclyl or heterocyclyl (having 1-4 heteroatoms independently selected from nitrogen, oxygen, and sulfur) that is fused to Ring B; and
   R^{4C} is hydrogen.

In some embodiments, R⁴ is Ring B of the following structure:

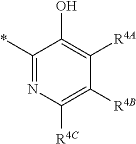

wherein * is a point of attachment to Linker L that is bonded to Ring A in Formula I;
and wherein:
   R^{4A} and R^{4B}, along with their intervening atoms, join to form 5-membered heterocyclyl (having 1 oxygen atom) that is fused to Ring B; and
   R^{4C} is hydrogen.

In some embodiments, R⁴ is Ring B of the following structure:

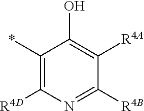

wherein * is a point of attachment to Linker L that is bonded to Ring A in Formula I;
and wherein:
   R^{4A}, R^{4B}, and R^{4D} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4A} and R^{4B}, along with their intervening atoms, join to form 4-7 membered carbocyclyl or heterocyclyl (having 1-4 heteroatoms independently selected from nitrogen, oxygen, and sulfur) that is fused to Ring B; and R^{4D} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR¹³R¹⁴; and
   R¹³ is independently selected at each occurrence from hydrogen and C₁-C₄alkyl optionally substituted with —OH, —OCH₃, or —OCH₂CH₃; and
   R¹⁴ is hydrogen or NR¹³R¹⁴ forms a heterocyclic ring selected from azetidinyl, pyrrolidinyl, and piperidinyl, said heterocyclic ring optionally substituted with —CH₃.

In some embodiments, R⁴ is Ring B of the following structure:

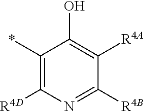

wherein * is a point of attachment to Linker L that is bonded to Ring A in Formula I;
and wherein:
   R^{4A} and R^{4D} are each hydrogen; and
   R^{4B} is C₁-C₄alkyl.

In some embodiments, R⁴ is Ring B of the following structure:

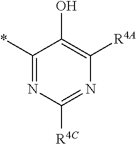

wherein * is a point of attachment to Linker L that is bonded to Ring A in Formula I;
and wherein:
   R^{4A} and R^{4C} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
   R¹³ is independently selected at each occurrence from hydrogen and C₁-C₄alkyl optionally substituted with —OH, —OCH₃, or —OCH₂CH₃; and
   R¹⁴ is hydrogen; or NR¹³R¹⁴ forms a heterocyclic ring selected from azetidinyl, pyrrolidinyl, and piperidinyl, said heterocyclic ring optionally substituted with —CH₃.

In some embodiments, R⁴ is Ring B of the following structure:

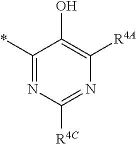

wherein * is a point of attachment to Linker L that is bonded to Ring A in Formula I;
and wherein:
   R^{4A} and R^{4C} are each independently selected from hydrogen and C₁-C₄alkyl.

In some embodiments, R⁴ is Ring B of the following structure:

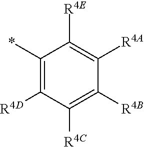

wherein * is a point of attachment to Linker L that is bonded to Ring A in Formula I;
and wherein:
   R^{4A}, R^{4B}, R^{4C}, R^{4D}, and R^{4E} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4A} and R^{4B}, along with their intervening atoms, join to form 4-7 membered carbocyclyl or heterocyclyl (having 1-4 heteroatoms independently selected from nitrogen, oxygen, and sulfur) that is fused to Ring B; and R^{4C}, R^{4D}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4C} and R^{4D}, along with their intervening atoms, join to form 4-7 membered carbocyclyl or heterocyclyl (having 1-4 heteroatoms independently selected from nitrogen, oxygen, and sulfur) that is fused to Ring B; and R^{4A}, R^{4B}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4E} is halogen or —OH, and R^{4A}, R^{4B}, R^{4C}, and RAD are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4E} and R^{4A}, along with their intervening atoms, join to form 5-6 membered heteroaryl (having 1-4 heteroatoms independently selected from nitrogen, oxygen, and sulfur) fused to Ring B; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
   R¹³ is independently selected at each occurrence from hydrogen and C₁-C₄alkyl optionally substituted with —OH, —OCH₃, or —OCH₂CH₃; and
   R¹⁴ is hydrogen; or NR¹³R¹⁴ forms a heterocyclic ring selected from azetidinyl, pyrrolidinyl, and piperidinyl, said heterocyclic ring optionally substituted with —CH₃.

In some embodiments, R⁴ is Ring B of the following structure:

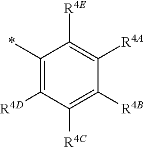

wherein * is a point of attachment to Linker L that is bonded to Ring A in Formula I;
and wherein:
   R^{4A}, R^{4B}, R^{4C}, R^{4D}, and R^{4E} are each independently selected from hydrogen; halogen; C₁-C₄alkyl; and C₁-C₄alkoxy; or
   R^{4C} and R^{4D}, along with their intervening atoms, join to form a 4-7 membered heterocyclyl (having 1-3 nitrogen atoms) fused to Ring B; and R^{4A}, R^{4B}, and R^{4E} are each hydrogen.

In some embodiments, R⁴ is Ring B of the following structure:

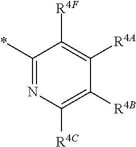

wherein * is a point of attachment to Linker L that is bonded to Ring A in Formula I;
and wherein:
   R^{4F} and R⁴, along with their intervening atoms, join to form 5-6 membered heteroaryl (having 1-4 heteroatoms independently selected from nitrogen, oxygen, and sulfur) fused to Ring B; and R^{4B} and R^{4C} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴;
   R¹³ is independently selected at each occurrence from hydrogen and C₁-C₄alkyl optionally substituted with —OH, —OCH₃, or —OCH₂CH₃; and
   R¹⁴ is hydrogen or NR¹³R¹⁴ may combine with the N to which they are attached to form a heterocyclic ring selected from azetidinyl, pyrrolidinyl, and piperidinyl, said heterocyclic ring optionally substituted with —CH₃.

In some embodiments, R⁴ is Ring B of the following structure:

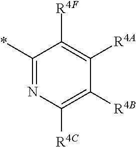

wherein * is a point of attachment to Linker L that is bonded to Ring A in Formula I;
and wherein:
   R^{4F} and R⁴, along with their intervening atoms, join to form 5-6 membered heteroaryl (having 1-2 nitrogen atoms) fused to Ring B; and R^{4B} and R^{4C} are each hydrogen.

In some embodiments, R⁴ is Ring B of the following structure:

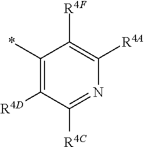

wherein * is a point of attachment to Linker L that is bonded to Ring A in Formula I; and wherein:
   R⁴, R^{4C}, R^{4D}, and R^{4F} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4C} and R^{4D}, along with their intervening atoms, join to form 4-7 membered optionally substituted carbocyclyl or optionally substituted heterocyclyl (having 1-4 heteroatoms independently selected from nitrogen, oxygen, and sulfur) that is fused to Ring B; and R⁴ and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
   R^{4F} and R^{4A}, along with their intervening atoms, join to form 5-6 membered optionally substituted heteroaryl (having 1-4 heteroatoms independently selected from nitrogen, oxygen, and sulfur) that is fused to Ring B; and R^{4C} and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
   R¹³ is independently selected at each occurrence from hydrogen and C₁-C₄alkyl optionally substituted with —OH, —OCH₃, or —OCH₂CH₃; and
   R¹⁴ is hydrogen; or NR¹³R¹⁴ may combine with the N to which they are attached to form a heterocyclic ring selected from azetidinyl, pyrrolidinyl, and piperidinyl, said heterocyclic ring optionally substituted with —CH₃.

In some embodiments, R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy.

In some embodiments, R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from OH, —CH₃, —CHF₂, cyclopropyl, and —OCH₃.

In some embodiments, R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl, and optionally substituted 5-6 membered heterocyclyloxy. In some embodiments, R⁴ is a C₁-C₄alkyl, substituted with 0-3 independently selected halogen, —CN, —OH, C₁-C₄alkyl, and C₁-C₄alkoxy. In some embodiments, R⁴ is a C₁-C₄alkoxy, substituted with 0-3 independently selected halogen, —CN, —OH, C₁-C₄alkyl, and C₁-C₄alkoxy. In some embodiments, R⁴ is a C₃-C₆cycloalkyl, substituted with 0-3 independently selected halogen, —CN, —OH, C₁-C₄alkyl, and C₁-C₄alkoxy.

In some embodiments, R⁴ is an isoxazolyl substituted with —OH or C₁-C₄alkoxy.

In some embodiments, R⁴ is a 5-membered heteroaryl (having 1 heteroatom independently selected from nitrogen, oxygen, and sulfur and 0, 1, 2, or 3 additional ring nitrogen atoms) selected from the group consisting of imidazolyl, pyrazolyl, tetrazolyl, thiazolyl, isothiazolyl, 1,2,5-thiadiazolyl, 1,3,4-thiadiazolyl, oxazolyl, isoxazolyl, 1,2,4-oxadiazolyl, 1,2,3-triazolyl, 1,2,4-triazolyl, wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy.

In some embodiments, R⁴ is

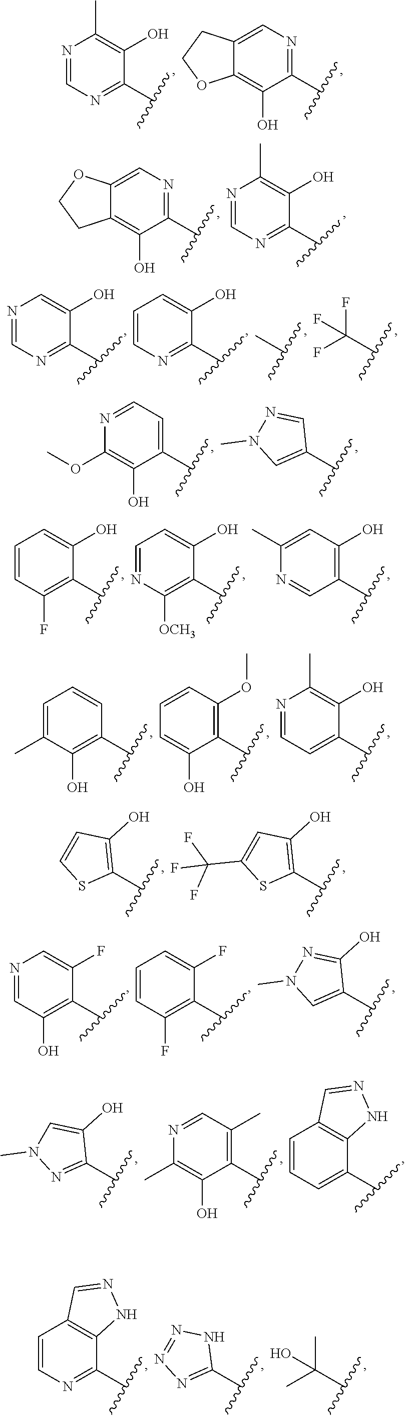

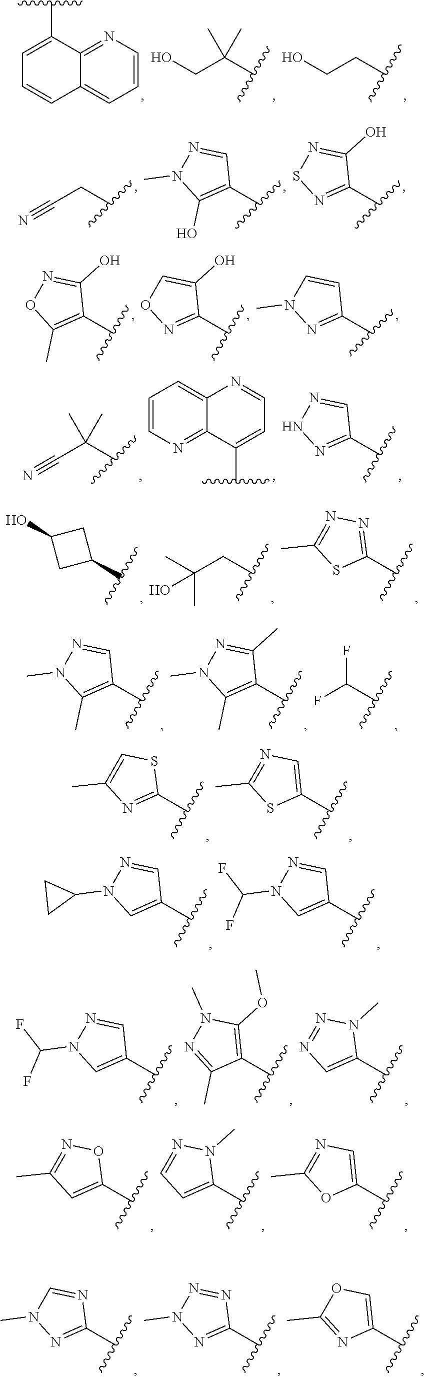

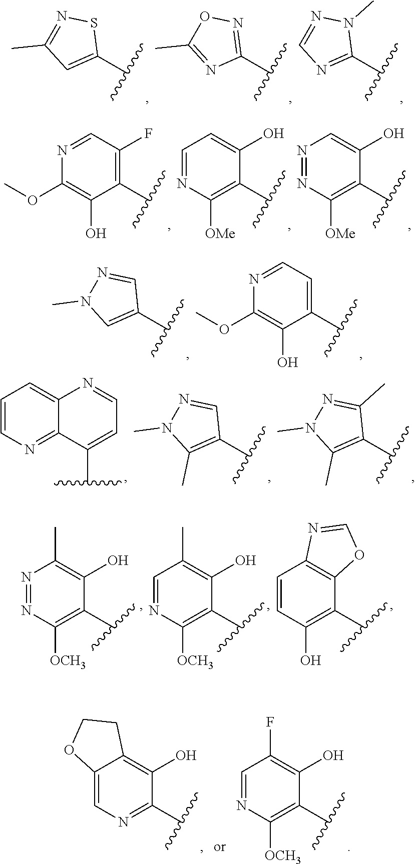

In some embodiments, R⁴ is

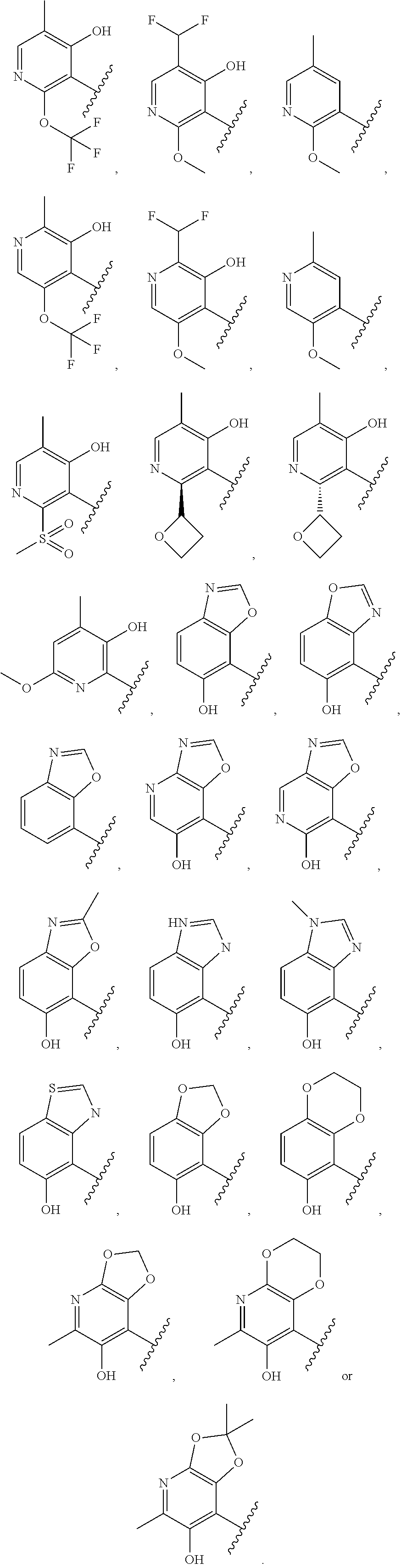

In some embodiments, R⁴ is as shown in a substituent of Table 1, Table 1a, or Table 1b.

As described generally above, each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

In some embodiments, each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur).

In some embodiments, two R groups on the same atom are taken together with the same atom to form an optionally substituted 4-7 membered saturated ring, partially unsaturated ring, or heteroaryl ring (wherein said 4-7 membered saturated ring, partially unsaturated ring, or heteroaryl ring, has 0-3 heteroatoms independently selected from nitrogen, oxygen, and sulfur).

In some embodiments, each R is independently hydrogen or a C₁₋₆alkyl. In some embodiments, R is hydrogen.

In some embodiments, each R is as selected from one or more of the substituents of Table 1, Table 1a, or Table 1b.

In some embodiments, the compound of Formula I is a compound of Formula I-a, or I-b or a pharmaceutically acceptable salt thereof:

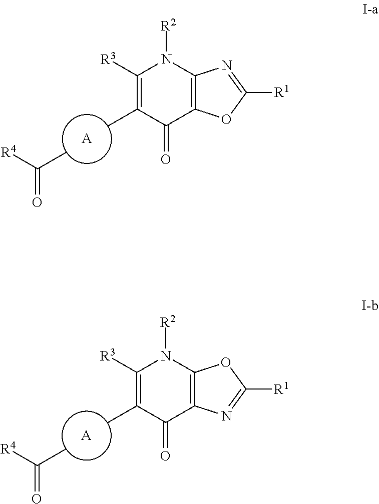

wherein Ring A, R¹, R², R³, and R⁴, are as defined herein, both singly and in combination.

In some embodiments, the compound of Formula I is a compound of Formula I-a, or I-b or a pharmaceutically acceptable salt thereof:

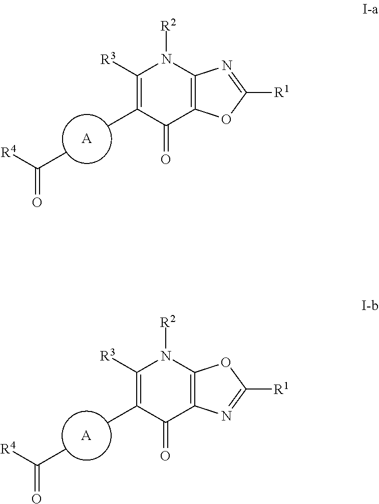

wherein Ring A, R¹, R², R³, and R⁴, are as defined herein, both singly and in combination and R^{2a} is selected from

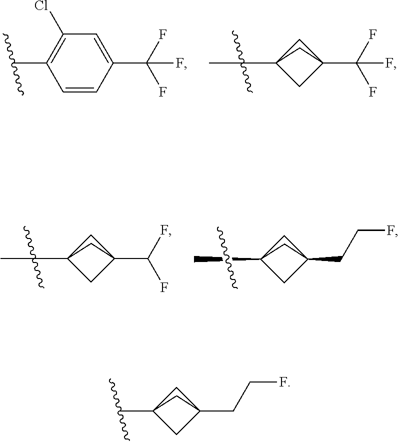

In some embodiments, the compound of Formula I is a compound of Formula I-a, or I-b or a pharmaceutically acceptable salt thereof:

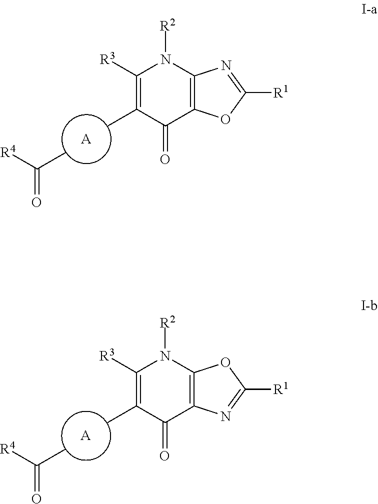

wherein R¹, R², R³, and R⁴, are as defined herein, both singly and in combination, Ring A is selected from

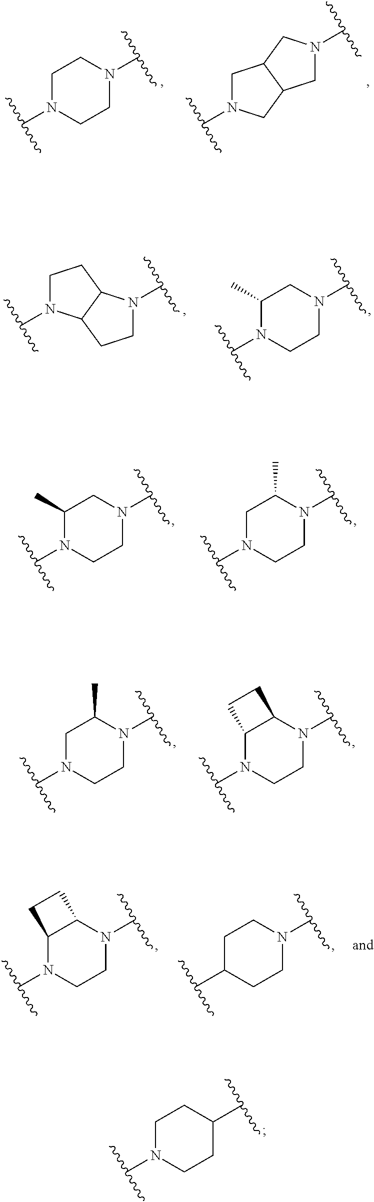

and R^{2a} is selected from

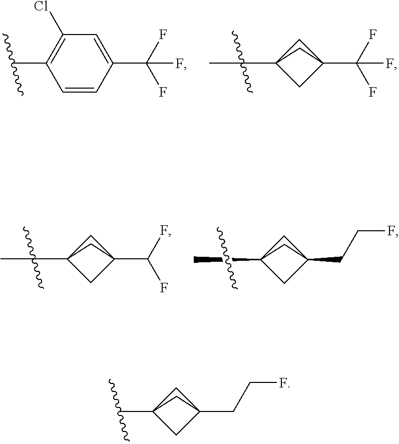

In some embodiments, the compound of Formula I is a compound of Formula II-a, II-b, or II-c or a pharmaceutically acceptable salt thereof:

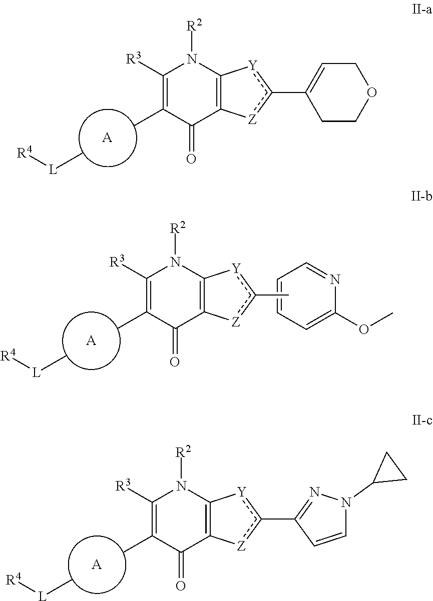

wherein Ring A, Linker L, R², R³, Y, Z and R⁴, are as defined herein, both singly and in combination.

In some embodiments, the compound of Formula I is a compound of Formula II-d, II-e, or II-f or a pharmaceutically acceptable salt thereof:

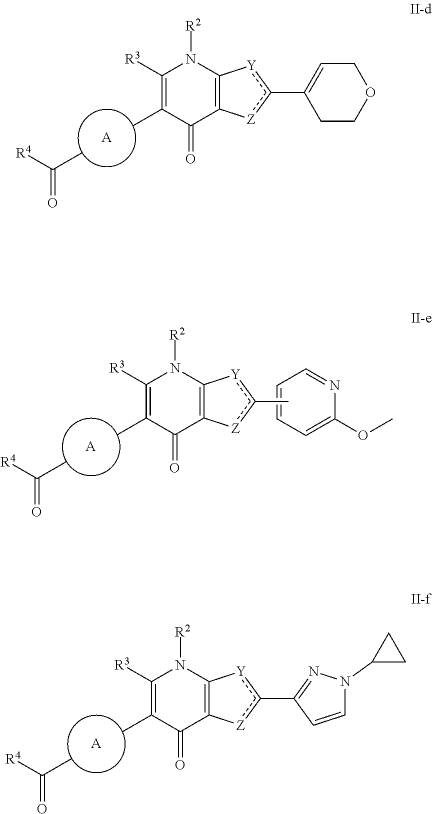

wherein Ring A, R², R³, Y, Z and R⁴, are as defined herein, both singly and in combination.

In some embodiments, the compound of Formula I is a compound of Formula III-a, III-b, or III-c or a pharmaceutically acceptable salt thereof:

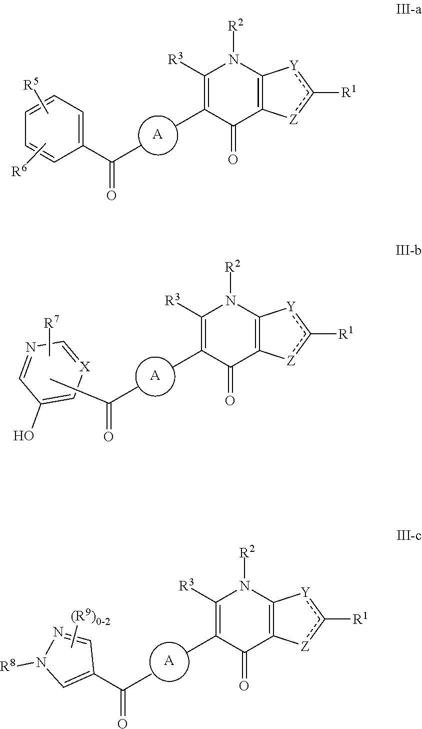

wherein Ring A, R¹, R², Y, Z and R³, are as defined herein, both singly and in combination, and
wherein:
X is CH, CR⁷, or N;
R⁵ is —OH or halogen;
R⁶ is halogen, C₁₋₄alkyl, or C₁₋₄alkoxy;
each R⁷ is independently hydrogen, halogen, C₁₋₄alkyl, or C₁₋₄alkoxy;
R⁸ is C₁₋₄alkyl;
each of the 0-2 instances of R⁹ is independently a hydrogen or C₁₋₄alkyl. In some embodiments:
X is CH or N;
R⁵ is —OH or fluoro;
R⁶ is fluoro, —CH₃, or —OCH₃;
each R⁷ is independently hydrogen, fluoro, —CH₃, or —OCH₃;
R⁸ is —CH₃;
each instance of R⁹ is independently a hydrogen or —CH₃.

In some embodiments, the compound of Formula I is a compound of Formula IV-a, IV-b, or IV-c or a pharmaceutically acceptable salt thereof:

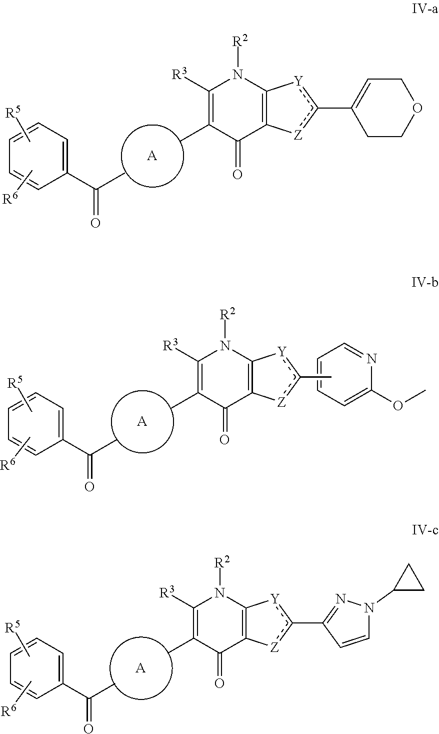

wherein Ring A, R², Y, Z and R³, are as defined herein, both singly and in combination and wherein:
R⁵ is —OH or halogen; and
R⁶ is halogen, C₁₋₄alkyl, or C₁₋₄alkoxy. In some embodiments: R⁵ is —OH or fluoro; and R⁶ is fluoro, —CH₃, or —OCH₃.

In some embodiments, the compound of Formula I is a compound of Formula V-a, V-b, or V-c or a pharmaceutically acceptable salt thereof:

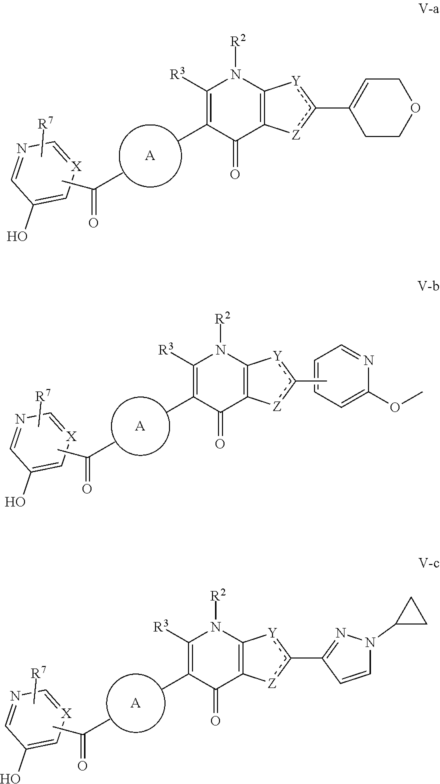

wherein Ring A, R², Y, Z and R³, are as defined herein, both singly and in combination, and wherein:
X is CH, CR⁷, or N; and each R⁷ is independently hydrogen, halogen, C₁₋₄alkyl, or C₁₋₄alkoxy. In some embodiments: X is CH or N; and R⁷ is hydrogen, fluoro, —CH₃, or —OCH₃.

In some embodiments, the compound of Formula I is a compound of Formula VI-a, VI-b, or VI-c or a pharmaceutically acceptable salt thereof:

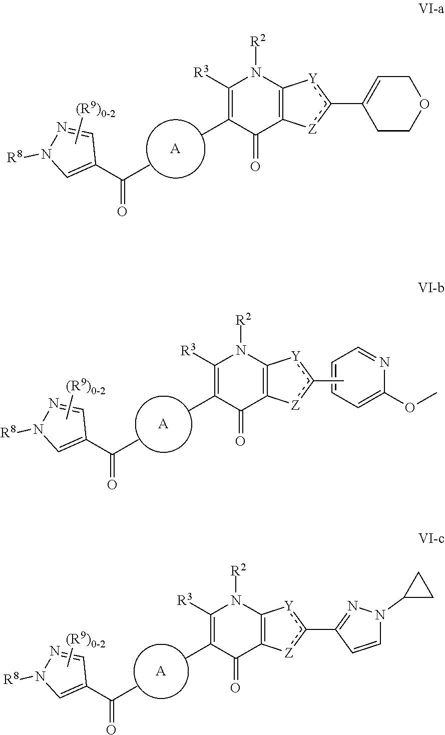

wherein Ring A, R², Y, Z and R³, are as defined herein, both singly and in combination, and wherein:
R⁸ is C₁₋₄alkyl; and each of the 0-2 instances of R⁹ are independently a hydrogen or C₁₋₄alkyl. In some embodiments: R⁸ is —CH₃; and each instance of R⁹ is independently a hydrogen or —CH₃.

In some embodiments, the compound of Formula I is a compound of Formula VII-a, or VII-b or a pharmaceutically acceptable salt thereof:

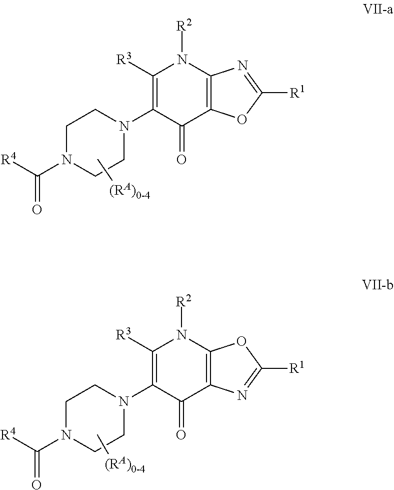

wherein R¹, R², R³, R^{A} and R⁴, are as defined herein, both singly and in combination.

In some embodiments, the compound of Formula I is a compound of Formula VII-a, or VII-b or a pharmaceutically acceptable salt thereof: wherein R¹, R², R³, R^{A} and R⁴, are as defined herein, both singly and in combination and R^{2a} is selected from

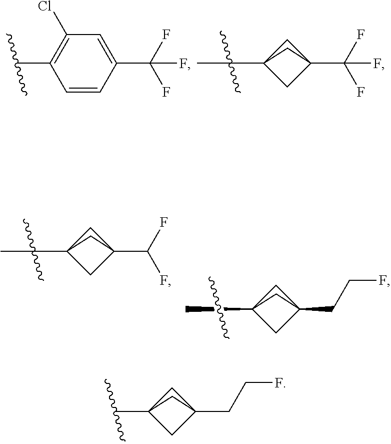

In some embodiments, the compound of Formula I is a compound of Formula VII-c, VII-d, or VII-e or a pharmaceutically acceptable salt thereof:

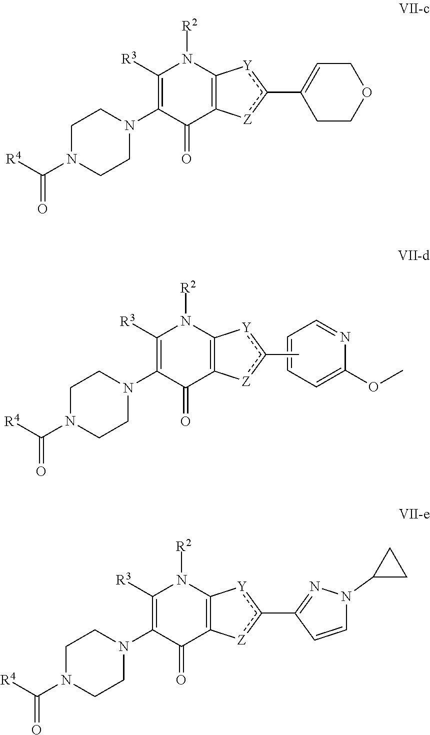

wherein R², R³, Y, Z and R⁴, are as defined herein, both singly and in combination.

In some embodiments, the compound of Formula I is a compound of Formula VIII-a, VIII-b, or VIII-c or a pharmaceutically acceptable salt thereof:

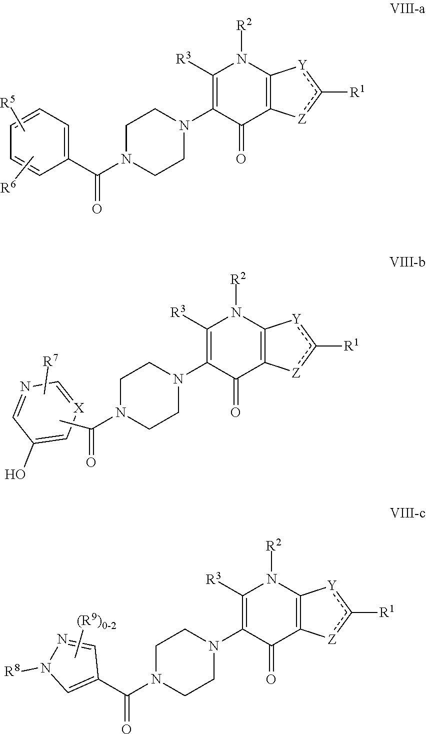

wherein R¹, R², Y, Z and R³, are as defined herein, both singly and in combination, and wherein:
X is CH, CR⁷, or N;
R⁵ is —OH or halogen;
R⁶ is halogen, C₁₋₄alkyl, or C₁₋₄alkoxy;
each R⁷ is independently hydrogen, halogen, C₁₋₄alkyl, or C₁₋₄alkoxy;
R⁸ is C₁₋₄alkyl; and
each of the 0-2 instances of R⁹ are independently a hydrogen or C₁₋₄alkyl. In some embodiments:
X is CH or N;
R⁵ is —OH or fluoro;
R⁶ is fluoro, —CH₃, or —OCH₃;
each R⁷ is independently hydrogen, fluoro, —CH₃, or —OCH₃;
R⁸ is —CH₃; and
each instance of R⁹ is independently a hydrogen or —CH₃.

In some embodiments, the compound of Formula I is a compound of Formula IX-a, IX-b, or IX-c or a pharmaceutically acceptable salt thereof:

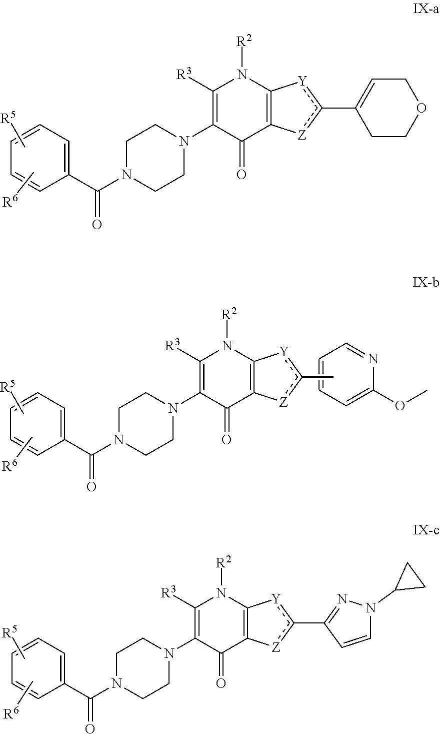

wherein Y, Z, R² and R³, are as defined herein, both singly and in combination and wherein:
R⁵ is —OH or halogen; and
R⁶ is halogen, C₁₋₄alkyl, or C₁₋₄alkoxy. In some embodiments: R⁵ is —OH or fluoro; and R⁶ is fluoro, —CH₃, or —OCH₃.

In some embodiments, the compound of Formula I is a compound of Formula X-a, X-b, or X-c or a pharmaceutically acceptable salt thereof:

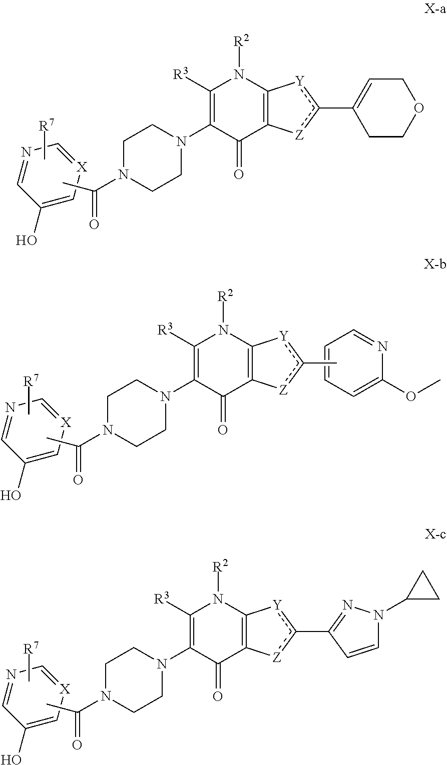

wherein Y, Z, R² and R³, are as defined herein, both singly and in combination, and wherein:
X is CH, CR⁷, or N; and
each R⁷ is independently hydrogen, halogen, C₁₋₄alkyl, or C₁₋₄alkoxy.

In some embodiments: X is CH or N; and each R⁷ is independently hydrogen, fluoro, —CH₃, or —OCH₃.

In some embodiments, the compound of Formula I is a compound of Formula XI-a, XI-b, or XI-c or a pharmaceutically acceptable salt thereof:

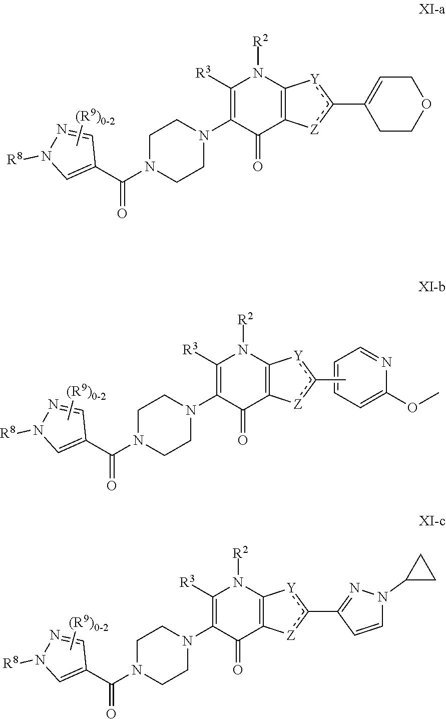

wherein Y, Z, R², and R³, are as defined herein, both singly and in combination, and wherein:
R⁸ is C₁₋₄alkyl; and
each of the 0-2 instances of R⁹ are independently a hydrogen or C₁₋₄alkyl. In some embodiments:
R⁸ is —CH₃; and each instance of R⁹ is independently a hydrogen or —CH₃.

In some embodiments, the compound of Formula I is a compound of Formula XII-a, XII-b, or XII-c or a pharmaceutically acceptable salt thereof:

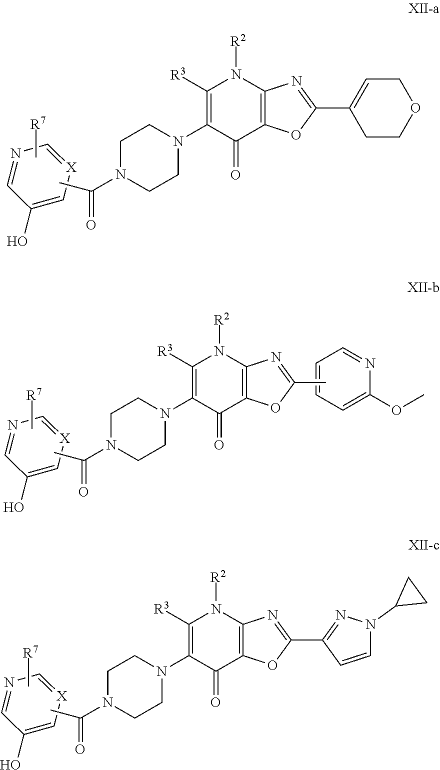

R² is C(R^{B})₂C(O)NHR^{2A};
R^{B} is independently selected at each occurrence from hydrogen and —CH₃;
R^{2A} is phenyl, pyridyl, or bicyclo[1.1.1]pentyl, each of which is optionally substituted with 1, or 2, or 3 substituents independently selected from halogen, C₁-C₄alkyl, haloC₁-C₄alkyl, C₃-C₆-cycloalkyl, haloC₃-C₆cycloalkyl, —OH, —CN, C₁-C₄alkoxy, haloC₁-C₄alkoxy, and —SF₅; or
R^{2A} is 2-benzimidazolyl, 2-naphthyl, or 3-quinolinyl, each of which is optionally substituted with 1, 2, or 3 substituents independently selected from halogen, C₁₋₄alkyl, and —OH;
R³ is C₁-C₄alkyl, C₃-C₅cycloalkyl, or C₁-C₄alkoxy, each of which is optionally substituted with —OH, 1-3 independently selected halogen, or C₁-C₄alkoxy; and
each R⁷ is independently hydrogen, halogen, C₁₋₄alkyl, or C₁₋₄alkoxy.

In some embodiments, the compound of Formula I is a compound of Formula XII-d, XII-e, or XII-f or a pharmaceutically acceptable salt thereof:

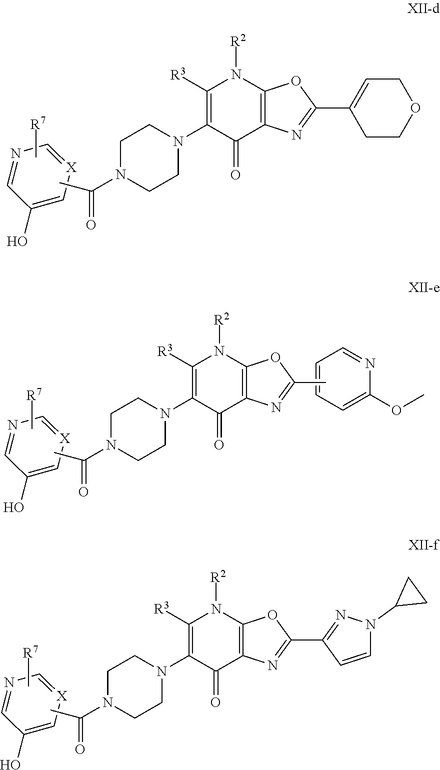

R² is C(R^{B})₂C(O)NHR^{2A};
R^{B} is independently selected at each occurrence from hydrogen and —CH₃;
R^{2A} is phenyl, pyridyl, or bicyclo[1.1.1]pentyl, each of which is optionally substituted with 1, 2, or 3 substituents independently selected from halogen, C₁-C₄alkyl, haloC₁-C₄alkyl, C₃-C₆-cycloalkyl, haloC₃-C₆cycloalkyl, —OH, —CN, C₁-C₄alkoxy, haloC₁-C₄alkoxy, and —SF₅; or
R^{2A} is 2-benzimidazolyl, 2-naphthyl, or 3-quinolinyl, each of which is optionally substituted with 1, 2, or 3 substituents independently selected from halogen, C₁₋₄alkyl, and —OH;
R³ is C₁-C₄alkyl, C₃-C₅cycloalkyl, or C₁-C₄alkoxy, each of which is optionally substituted with —OH, 1-3 independently selected halogen, or C₁-C₄alkoxy; and
each R⁷ is independently hydrogen, halogen, C₁₋₄alkyl, or C₁₋₄alkoxy.

In some embodiments, the compound of Formula I is selected from one of those depicted in Table 1, Table 1a, or Table 1b, or a pharmaceutically acceptable salt thereof. Table 1, Table 1a, or Table 1b identifies compounds by their IUPAC name and Table 2, Table 2a or Table 2b or lists the same compounds and shows their chemical structure. In the event of any discrepancy between Table 1's, Table 1a's, or Table 1b's, name for a compound and Table 2's, Table 2a's or Table 2b's structure for that same compound, Table 2's, Table 2a's or Table 2b's compound structures will dominate and identify the compound corresponding to each respective compound number (I-#) in Table 1, Table 1a, or Table 1b.

TABLE 1

| No. | IUPAC Name |
|---|---|
| I-1 | N-(2-chloro-4-(trifluoromethyl)phenyl)-2-(5-ethyl-6-(4-(5-hydroxy-6- |
| | methylpyrimidine-4-carbonyl)piperazin-1-y1)-2-(2-methoxypyridin-4-yl)-7- |
| | oxooxazolo[4,5-b]pyridin-4(7H)-yl)acetamide |
| I-2 | N-(2-chloro-4-(trifluoromethyl)phenyl)-2-(5-ethyl-6-(4-(5-hydroxy-6- |
| | methylpyrimidine-4-carbonyl)piperazin-1-yl)-2-(2-methoxypyridin-4-yl)-7- |
| | oxooxazolo[5,4-b]pyridin-4(7H)-yl)acetamide |
| I-3 | 2-(5-ethyl-6-(4-(5-hydroxy-6-methylpyrimidine-4-carbonyl)piperazin-1-yl)-2-(2- |
| | methoxypyridin-4-yl)-7-oxooxazolo[4,5-b]pyridin-4(7H)-yl)-N-(3- |
| | (trifluoromethyl)bicyclo[1.1.1]pentan-1-yl)acetamide |
| I-4 | 2-(5-ethyl-6-(4-(5-hydroxy-6-methylpyrimidine-4-carbonyl)piperazin-1-yl)-2-(2- |
| | methoxypyridin-4-yl)-7-oxooxazolo[5,4-b]pyridin-4(7H)-y1)-N-(3- |
| | (trifluoromethyl)bicyclo[1.1.1]pentan-1-yl)acetamide |
| I-5 | N-(2-chloro-4-(trifluoromethyl)phenyl)-2-(5-ethyl-6-(4-(6-hydroxybenzo[d]oxazole- |
| | 7-carbonyl)piperazin-1-y1)-2-(2-methoxypyridin-4-yl)-7-oxooxazolo[4,5-b]pyridin- |
| | 4(7H)-y1)acetamide |

TABLE la

| No. | IUPAC Name |
|---|---|
| I-2a | N-(2-chloro-4-(trifluoromethyl)phenyl)-2-(2-(3,6-dihydro-2H-pyran-4-yl)-5-ethyl- |
| | 6-(4-(5-hydroxy-6-methylpyrimidine-4-carbonyl)piperazin-1-yl)-7-oxooxazolo[4,5- |
| | b]pyridin-4(7H)-yl)acetamide |
| I-3a | N-(2-chloro-4-(trifluoromethyl)phenyl)-2-(5-ethyl-2-(2-methoxypyridin-4-y1)-6-(4- |
| | (1-methyl-1H-pyrazole-4-carbonyl)piperazin-1-yl)-7-oxooxazolo[4,5-b]pyridin- |
| | 4(7H)-yl)acetamide |
| I-4a | N-(2-chloro-4-(trifluoromethyl)phenyl)-2-(2-(1-cyclopropyl-1H-pyrazol-4-yl)-5- |
| | ethyl-6-(4-(5-fluoro-3-hydroxy-2-methoxyisonicotinoyl)piperazin-1-yl)-7- |
| | oxooxazolo[4,5-b]pyridin-4(7H)-yl)acetamide |
| I-5a | N-(2-chloro-4-(trifluoromethyl)phenyl)-2-(5-ethyl-6-(4-(5-fluoro-3-hydroxy-2- |
| | methoxyisonicotinoy1)piperazin-1-yl)-2-(2-methoxypyridin-4-y1)-7-oxooxazolo[4,5- |
| | b]pyridin-4(7H)-yl)acetamide |
| I-6a | N-(2-chloro-4-(trifluoromethyl)phenyl)-2-(2-(3,6-dihydro-2H-pyran-4-yl)-5-ethyl- |
| | 6-(4-(5-fluoro-3-hydroxy-2-methoxyisonicotinoyl)piperazin-1-yl)-7- |
| | oxooxazolo[4,5-b]pyridin-4(7H)-yl)acetamide |
| I-7a | N-(2-chloro-4-(trifluoromethyl)phenyl)-2-(5-ethyl-6-(4-(4-hydroxyisoxazole-3- |
| | carbonyl)piperazin-1-yl)-2-(2-methoxypyridin-4-yl)-7-oxooxazolo[4,5-b]pyridin- |
| | 4(7H)-yl)acetamide |
| I-8a | N-(2-chloro-4-(trifluoromethyl)phenyl)-2-(2-(3,6-dihydro-2H-pyran-4-yl)-5-ethyl- |
| | 6-(4-(4-hydroxyisoxazole-3-carbonyl)piperazin-1-yl)-7-oxooxazolo[4,5-b]pyridin- |
| | 4(7H)-yl)acetamide |
| I-9a | N-(2-chloro-4-(trifluoromethyl)phenyl)-2-(2-(1-cyclopropyl-1H-pyrazol-4-yl)-5- |
| | ethyl-6-(4-(3-hydroxy-2-methoxyisonicotinoyl)piperazin-1-yl)-7-oxooxazolo[4,5- |
| | b]pyridin-4(7H)-yl)acetamide |
| I-10a | N-(2-chloro-4-(trifluoromethyl)phenyl)-2-(5-ethyl-6-(4-(3-hydroxy-2- |
| | methoxyisonicotinoy1)piperazin-1-yl)-2-(2-methoxypyridin-4-y1)-7-oxooxazolo[4,5- |
| | b]pyridin-4(7H)-yl)acetamide |
| I-11a | N-(2-chloro-4-(trifluoromethyl)phenyl)-2-(2-(3,6-dihydro-2H-pyran-4-y1)-5-ethyl- |
| | 6-(4-(3-hydroxy-2-methoxyisonicotinoyl)piperazin-1-yl)-7-oxooxazolo[4,5- |
| | b]pyridin-4(7H)-yl)acetamide |
| I-12a | N-(2-chloro-4-(trifluoromethyl)phenyl)-2-(2-(3,6-dihydro-2H-pyran-4-y1)-5-ethyl- |
| | 6-(4-(1-methyl-1H-pyrazole-4-carbonyl)piperazin-1-yl)-7-oxooxazolo[4,5- |
| | b]pyridin-4(7H)-yl)acetamide |
| I-13a | N-(2-chloro-4-(trifluoromethyl)phenyl)-2-(2-(1-cyclopropyl-1H-pyrazol-4-y1)-5- |
| | ethyl-6-(4-(4-hydroxy-2-methoxynicotinoyl)piperazin-1-yl)-7-oxooxazolo[4,5- |
| | b]pyridin-4(7H)-yl)acetamide |
| I-14a | N-(2-chloro-4-(trifluoromethyl)phenyl)-2-(5-ethyl-6-(4-(4-hydroxy-2- |
| | methoxynicotinoyl)piperazin-1-y1)-2-(6-methoxypyridin-3-y1)-7-oxooxazolo[4,5- |
| | b]pyridin-4(7H)-yl)acetamide |
| I-15a | N-(2-chloro-4-(trifluoromethyl)phenyl)-2-(5-ethyl-6-(4-(4-hydroxy-2- |
| | methoxynicotinoyl)piperazin-1-y1)-2-(2-(2-hydroxypropan-2-yl)thiazol-5-yl)-7- |
| | oxooxazolo[4,5-b]pyridin-4(7H)-yl)acetamide |
| I-16a | N-(2-chloro-4-(trifluoromethyl)phenyl)-2-(5-ethyl-6-(4-(4-hydroxy-2- |
| | methoxynicotinoyl)piperazin-1-yl)-2-(2-methoxypyridin-4-yl)-7-oxooxazolo[4,5- |
| | b]pyridin-4(7H)-yl)acetamide |
| I-17a | N-(2-chloro-4-(trifluoromethyl)phenyl)-2-(2-(3,6-dihydro-2H-pyran-4-y1)-5-ethyl- |
| | 6-(4-(4-hydroxy-2-methoxynicotinoyl)piperazin-1-yl)-7-oxooxazolo[4,5-b]pyridin- |
| | 4(7H)-yl)acetamide |
| I-18a | N-(2-chloro-4-(trifluoromethyl)phenyl)-2-(5-ethyl-6-(4-(4-hydroxyisothiazole-3- |
| | carbonyl)piperazin-1-y1)-2-(2-methoxypyridin-4-y1)-7-oxooxazolo[4,5-b]pyridin- |
| | 4(7H)-yl)acetamide |
| I-19a | N-(2-chloro-4-(trifluoromethyl)phenyl)-2-(2-(3,6-dihydro-2H-pyran-4-y1)-5-ethyl- |
| | 6-(4-(4-hydroxyisothiazole-3-carbonyl)piperazin-1-y1)-7-oxooxazolo[4,5-b]pyridin- |
| | 4(7H)-yl)acetamide |
| I-20a | N-(2-chloro-4-(trifluoromethyl)phenyl)-2-(5-ethyl-6-(4-(7-hydroxy-2,3- |
| | dihydrofuro[3,2-c]pyridine-6-carbonyl)piperazin-1-yl)-2-(2-methoxypyridin-4-y1)- |
| | 7-oxooxazolo[4,5-b]pyridin-4(7H)-yl)acetamide |
| I-21a | N-(2-chloro-4-(trifluoromethyl)phenyl)-2-(2-(3,6-dihydro-2H-pyran-4-yl)-5-ethyl- |
| | 6-(4-(7-hydroxy-2,3-dihydrofuro[3,2-c]pyridine-6-carbonyl)piperazin-1-yl)-7- |
| | oxooxazolo[4,5-b]pyridin-4(7H)-yl)acetamide |
| I-22a | N-(2-chloro-4-(trifluoromethyl)phenyl)-2-(2-(1-cyclopropyl-1H-pyrazol-4-y1)-5- |
| | ethyl-6-(4-(5-hydroxy-6-methylpyrimidine-4-carbonyl)piperazin-1-y1)-7- |
| | oxooxazolo[4,5-b]pyridin-4(7H)-yl)acetamide |
| I-23a | N-(2-chloro-4-(trifluoromethyl)phenyl)-2-(5-ethyl-6-(4-(5-hydroxy-6- |
| | methylpyrimidine-4-carbonyl)piperazin-1-yl)-2-(6-methoxypyridin-3-yl)-7- |
| | oxooxazolo[4,5-b]pyridin-4(7H)-yl)acetamide |
| I-24a | N-(2-chloro-4-(trifluoromethyl)phenyl)-2-(5-ethyl-6-(4-(5-hydroxy-6- |
| | methylpyrimidine-4-carbonyl)piperazin-1-yl)-2-(2-(2-hydroxypropan-2-yl)thiazol- |
| | 5-y1)-7-oxooxazolo[4,5-b]pyridin-4(7H)-yl)acetamide |
| I-25a | N-(2-chloro-4-(trifluoromethyl)phenyl)-2-(5-ethyl-6-((1S,6S)-5-(5-hydroxy-6- |
| | methylpyrimidine-4-carbonyl)-2,5-diazabicyclo[4.2.0]octan-2-y1)-2-(2- |
| | methoxypyridin-4-yl)-7-oxooxazolo[4,5-b]pyridin-4(7H)-yl)acetamide |
| I-26a | N-(2-chloro-4-(trifluoromethyl)phenyl)-2-(2-(3,6-dihydro-2H-pyran-4-yl)-5-ethyl- |
| | 6-((1S,6S)-5-(5-hydroxy-6-methylpyrimidine-4-carbonyl)-2,5- |
| | diazabicyclo[4.2.0]octan-2-yl)-7-oxooxazolo[4,5-b]pyridin-4(7H)-yl)acetamide |
| I-27a | N-(2-chloro-4-(trifluoromethyl)phenyl)-2-(5-ethyl-6-(4-(4-hydroxy-2,3- |
| | dihydrofuro[2,3-c]pyridine-5-carbonyl)piperazin-1-yl)-2-(2-methoxypyridin-4-yl)- |
| | 7-oxooxazolo[4,5-b]pyridin-4(7H)-yl)acetamide |
| I-28a | N-(2-chloro-4-(trifluoromethyl)phenyl)-2-(2-(3,6-dihydro-2H-pyran-4-yl)-5-ethyl- |
| | 6-(4-(4-hydroxy-2,3-dihydrofuro[2,3-c]pyridine-5-carbonyl)piperazin-1-y1)-7- |
| | oxooxazolo[4,5-b]pyridin-4(7H)-yl)acetamide |
| I-29a | N-(2-chloro-4-(trifluoromethyl)phenyl)-2-(5-ethyl-6-(4-(3- |
| | hydroxypicolinoyl)piperazin-1-y1)-2-(2-methoxypyridin-4-yl)-7-oxooxazolo[4,5- |
| | b]pyridin-4(7H)-yl)acetamide |
| I-30a | N-(2-chloro-4-(trifluoromethyl)phenyl)-2-(2-(3,6-dihydro-2H-pyran-4-y1)-5-ethyl- |
| | 6-(4-(3-hydroxypicolinoyl)piperazin-1-yl)-7-oxooxazolo[4,5-b]pyridin-4(7H)- |
| | yl)acetamide |
| I-31a | N-(2-chloro-4-(trifluoromethyl)phenyl)-2-(5-ethyl-6-(4-(5-fluoro-4-hydroxy-2- |
| | methoxynicotinoyl)piperazin-1-y1)-2-(2-methoxypyridin-4-y1)-7-oxooxazolo[4,5- |
| | b]pyridin-4(7H)-yl)acetamide |
| I-32a | N-(2-chloro-4-(trifluoromethyl)phenyl)-2-(2-(3,6-dihydro-2H-pyran-4-yl)-5-ethyl- |
| | 6-((1R,6R)-5-(5-hydroxy-6-methylpyrimidine-4-carbonyl)-2,5- |
| | diazabicyclo[4.2.0]octan-2-y1)-7-oxooxazolo[4,5-b]pyridin-4(7H)-yl)acetamide |
| I-33a | N-(3-cyclopropylbicyclo[1.1.1]pentan-1-yl)-2-(2-(3,6-dihydro-2H-pyran-4-y1)-5- |
| | ethyl-6-(4-(5-hydroxy-6-methylpyrimidine-4-carbonyl)piperazin-1-yl)-7- |
| | oxooxazolo[4,5-b]pyridin-4(7H)-yl)acetamide |
| I-34a | 2-(2-(3,6-dihydro-2H-pyran-4-yl)-5-ethyl-6-(4-(4-hydroxy-2,3-dihydrofuro[2,3- |
| | c]pyridine-5-carbonyl)piperazin-1-yl)-7-oxooxazolo[4,5-b]pyridin-4(7H)-yl)-N-(3- |
| | (trifluoromethyl)bicyclo[1.1.1]pentan-1-yl)acetamide |
| I-35a | 2-(5-ethyl-6-(4-(5-fluoro-4-hydroxy-2-methoxynicotinoyl)piperazin-1-y1)-2-(2- |
| | methoxypyridin-4-yl)-7-oxooxazolo[4,5-b]pyridin-4(7H)-yl)-N-(3- |
| | (trifluoromethyl)bicyclo[1.1.1]pentan-1-yl)acetamide |
| I-36a | 2-(5-ethyl-6-(4-(6-hydroxybenzo[d]oxazole-7-carbonyl)piperazin-1-y1)-2-(2- |
| | methoxypyridin-4-yl)-7-oxooxazolo[4,5-b]pyridin-4(7H)-yl)-N-(3- |
| | (trifluoromethyl)bicyclo[1.1.1]pentan-1-yl)acetamide |
| I-37a | 2-(5-ethyl-6-(4-(4-hydroxy-2-methoxy-5-methylnicotinoyl)piperazin-1-yl)-2-(2- |
| | methoxypyridin-4-yl)-7-oxooxazolo[4,5-b]pyridin-4(7H)-yl)-N-(3- |
| | (trifluoromethyl)bicyclo[1.1.1]pentan-1-yl)acetamide |
| I-38a | 2-(5-ethyl-6-(4-(4-hydroxy-2-methoxy-5-methylnicotinoyl)piperazin-1-y1)-2-(2- |
| | methoxypyridin-4-yl)-7-oxooxazolo[4,5-b]pyridin-4(7H)-y1)-N-(3- |
| | (trifluoromethyl)bicyclo[1.1.1]pentan-1-yl)acetamide |
| I-39a | 2-(5-ethyl-6-((1R,6R)-5-(5-hydroxy-6-methylpyrimidine-4-carbonyl)-2,5- |
| | diazabicyclo[4.2.0]octan-2-y1)-2-(2-methoxypyridin-4-y1)-7-oxooxazolo[4,5- |
| | b]pyridin-4(7H)-y1)-N-(3-(trifluoromethyl)bicyclo[1.1.1]pentan-1-yl)acetamide |
| I-40a | 2-(2-(1-acetyl-1,2,3,6-tetrahydropyridin-4-y1)-5-ethyl-6-(4-(4-hydroxy-2,3- |
| | dihydrofuro[2,3-c]pyridine-5-carbonyl)piperazin-1-y1)-7-oxooxazolo[4,5-b]pyridin- |
| | 4(7H)-yl)-N-(3-(trifluoromethyl)bicyclo[1.1.1]pentan-1-yl)acetamide |
| I-41a | 2-(2-(dimethylamino)-5-ethyl-6-(4-(5-hydroxy-6-methylpyrimidine-4- |
| | carbonyl)piperazin-1-yl)-7-oxooxazolo[4,5-b]pyridin-4(7H)-yl)-N-(3- |
| | (trifluoromethyl)bicyclo[1.1.1]pentan-1-yl)acetamide |
| I-42a | 2-(2-(dimethylamino)-5-ethyl-6-(4-(6-hydroxybenzo[d]oxazole-7- |
| | carbonyl)piperazin-1-yl)-7-oxooxazolo[4,5-b]pyridin-4(7H)-yl)-N-(3- |
| | (trifluoromethyl)bicyclo[1.1.1]pentan-1-yl)acetamide |
| I-43a | 2-(5-ethyl-6-(4-(6-hydroxybenzo[d]oxazole-7-carbonyl)piperazin-1-y1)-7-oxo-2- |
| | (pyrrolidin-1-yl)oxazolo[4,5-b]pyridin-4(7H)-y1)-N-(3- |
| | (trifluoromethyl)bicyclo[1.1.1]pentan-1-y1)acetamide |
| I-44a | 2-(5-ethyl-6-(4-(4-hydroxy-2,3-dihydrofuro[2,3-c]pyridine-5-carbonyl)piperazin-1- |
| | y1)-2-(1-(2-methoxyacetyl)-1,2,3,6-tetrahydropyridin-4-yl)-7-oxooxazolo[4,5- |
| | b]pyridin-4(7H)-yl)-N-(3-(trifluoromethyl)bicyclo[1.1.1]pentan-1-yl)acetamide |
| I-45a | 2-(5-ethyl-6-(4-(6-hydroxybenzo[d]oxazole-7-carbonyl)piperazin-1-y1)-2- |
| | morpholino-7-oxooxazolo[4,5-b]pyridin-4(7H)-y1)-N-(3- |
| | (trifluoromethyl)bicyclo[1.1.1]pentan-1-yl)acetamide |
| I-46a | N-(3-cyclopropylbicyclo[1.1.1]pentan-1-y1)-2-(5-ethyl-6-(4-(6- |
| | hydroxybenzo[d]oxazole-7-carbonyl)piperazin-1-yl)-2-(2-methoxypyridin-4-yl)-7- |
| | oxooxazolo[4,5-b]pyridin-4(7H)-yl)acetamide |
| I-47a | N-(3-cyclopropylbicyclo[1.1.1]pentan-1-yl)-2-(2-(3,6-dihydro-2H-pyran-4-y1)-5- |
| | ethyl-6-(4-(4-hydroxy-2,3-dihydrofuro[2,3-c]pyridine-5-carbonyl)piperazin-1-yl)-7- |
| | oxooxazolo[4,5-b]pyridin-4(7H)-yl)acetamide |
| I-48a | N-(3-(1, 1-difluoroethyl)bicyclo[1.1.1]pentan-1-y1)-2-(2-(3,6-dihydro-2H-pyran-4- |
| | yl)-5-ethyl-6-(4-(4-hydroxy-2,3-dihydrofuro[2,3-c]pyridine-5-carbonyl)piperazin-1- |
| | y1)-7-oxooxazolo[4,5-b]pyridin-4(7H)-yl)acetamide |
| I-49a | 2-(2-(3,6-dihydro-2H-pyran-4-y1)-5-ethyl-6-(4-(4-hydroxy-2,3-dihydrofuro[2,3- |
| | c]pyridine-5-carbonyl)piperazin-1-yl)-7-oxooxazolo[4,5-b]pyridin-4(7H)-yl)-N-(3- |
| | methylbicyclo[1.1.1]pentan-1-yl)acetamide |
| I-50a | N-(3-(difluoromethyl)bicyclo[1.1.1]pentan-1-y1)-2-(2-(3,6-dihydro-2H-pyran-4-yl)- |
| | 5-ethyl-6-(4-(4-hydroxy-2,3-dihydrofuro[2,3-c]pyridine-5-carbonyl)piperazin-1-y1)- |
| | 7-oxooxazolo[4,5-b]pyridin-4(7H)-yl)acetamide |
| I-51a | 2-(2-(3,6-dihydro-2H-pyran-4-yl)-5-ethyl-6-(4-(5-hydroxy-6-methylpyrimidine-4- |
| | carbonyl)piperazin-1-yl)-7-oxooxazolo[4,5-b]pyridin-4(7H)-y1)-N-(1- |
| | (trifluoromethyl)-2-oxabicyclo[2.1.1]hexan-4-yl)acetamide |
| I-52a | 2-(5-ethyl-6-(4-(5-hydroxy-6-methylpyrimidine-4-carbonyl)piperazin-1-yl)-2-(2- |
| | methoxypyridin-4-y1)-7-oxooxazolo[4,5-b]pyridin-4(7H)-yl)-N-(3- |
| | (trifluoromethyl)cyclobutyl)acetamide |
| I-53a | 2-(2-(3,6-dihydro-2H-pyran-4-y1)-5-ethyl-6-(4-(4-hydroxy-2,3-dihydrofuro[2,3- |
| | c]pyridine-5-carbonyl)piperazin-1-yl)-7-oxooxazolo[4,5-b]pyridin-4(7H)-yl)-N-(3- |
| | methoxybicyclo[1.1.1]pentan-1-yl)acetamide |
| I-54a | 2-(2-(3,6-dihydro-2H-pyran-4-y1)-5-ethyl-6-(4-(4-hydroxy-2,3-dihydrofuro[2,3- |
| | c]pyridine-5-carbonyl)piperazin-1-y1)-7-oxooxazolo[4,5-b]pyridin-4(7H)-yl)-N-(3- |
| | fluorobicyclo[1.1.1]pentan-1-yl)acetamide |
| I-55a | 2-(2-(3,6-dihydro-2H-pyran-4-yl)-5-ethyl-6-((1R,6R)-5-(5-hydroxy-6- |
| | methylpyrimidine-4-carbonyl)-2,5-diazabicyclo[4.2.0]octan-2-y1)-7- |
| | oxooxazolo[4,5-b]pyridin-4(7H)-yl)-N-(3-(2-fluoroethyl)bicyclo[1.1.1]pentan-1- |
| | y1)acetamide |
| I-56a | 2-(2-(3,6-dihydro-2H-pyran-4-yl)-5-ethyl-6-(4-(4-hydroxy-2,3-dihydrofuro[2,3- |
| | c]pyridine-5-carbonyl)piperazin-1-yl)-7-oxooxazolo[4,5-b]pyridin-4(7H)-yl)-N-(3- |
| | (trifluoromethoxy)bicyclo[1.1.1]pentan-1-y1)acetamide |
| I-57a | N-(3-cyanobicyclo[1.1.1]pentan-1-yl)-2-(2-(3,6-dihydro-2H-pyran-4-yl)-5-ethyl-6- |
| | (4-(4-hydroxy-2,3-dihydrofuro[2,3-c]pyridine-5-carbonyl)piperazin-1-yl)-7- |
| | oxooxazolo[4,5-b]pyridin-4(7H)-yl)acetamide |
| I-58a | 2-(5-ethyl-6-((1R,6R)-5-(6-hydroxybenzo[d]oxazole-7-carbonyl)-2,5- |
| | diazabicyclo[4.2.0]octan-2-y1)-2-(2-methoxypyridin-4-y1)-7-oxooxazolo[4,5- |
| | b]pyridin-4(7H)-yl)-N-(3-(trifluoromethyl)bicyclo[1.1.1]pentan-1-yl)acetamide |
| I-59a | 2-(2-(dimethylamino)-5-ethyl-6-((1R,6R)-5-(6-hydroxybenzo[d]oxazole-7- |
| | carbonyl)-2,5-diazabicyclo[4.2.0]octan-2-y1)-7-oxooxazolo[4,5-b]pyridin-4(7H)-yl)- |
| | N-(3-(trifluoromethyl)bicyclo[1.1.1]pentan-1-y1)acetamide |
| I-60a | 2-(5-ethyl-6-((1R,6R)-5-(6-hydroxybenzo[d]oxazole-7-carbonyl)-2,5- |
| | diazabicyclo[4.2.0]octan-2-y1)-2-morpholino-7-oxooxazolo[4,5-b]pyridin-4(7H)- |
| | y1)-N-(3-(trifluoromethyl)bicyclo[1.1.1]pentan-1-yl)acetamide |
| I-61a | 2-(5-ethyl-6-(4-(5-hydroxybenzo[d]oxazole-4-carbonyl)piperazin-1-y1)-2-(2- |
| | methoxypyridin-4-yl)-7-oxooxazolo[4,5-b]pyridin-4(7H)-yl)-N-(3- |
| | (trifluoromethyl)bicyclo[1.1.1]pentan-1-yl)acetamide |
| I-62a | 2-(5-ethyl-6-(4-(6-hydroxybenzo[d]oxazole-7-carbonyl)piperazin-1-yl)-2-(2- |
| | methoxypyridin-4-yl)-7-oxooxazolo[4,5-b]pyridin-4(7H)-yl)-N-(3- |
| | ethylbicyclo[1.1.1]pentan-1-yl)acetamide |
| I-63a | 2-(5-ethyl-6-(4-(6-hydroxybenzo[d]oxazole-7-carbonyl)piperazin-1-yl)-2-(2- |
| | methoxypyridin-4-y1)-7-oxooxazolo[4,5-b]pyridin-4(7H)-y1)-N-(3- |
| | isopropylbicyclo[1.1.1]pentan-1-yl)acetamide |
| I-64a | 2-(5-ethyl-6-(4-(6-hydroxybenzo[d]oxazole-7-carbonyl)piperazin-1-y1)-2-(2- |
| | methoxypyridin-4-yl)-7-oxooxazolo[4,5-b]pyridin-4(7H)-yl)-N-(3-(2- |
| | fluoroethyl)bicyclo[1.1.1]pentan-1-yl)acetamide |
| I-65a | N-(3-cyclobutylbicyclo[1.1.1]pentan-1-yl)-2-(5-ethyl-6-(4-(6- |
| | hydroxybenzo[d]oxazole-7-carbonyl)piperazin-1-yl)-2-(2-methoxypyridin-4-yl)-7- |
| | oxooxazolo[4,5-b]pyridin-4(7H)-yl)acetamide |
| I-66a | 2-(5-ethyl-6-(4-(6-hydroxybenzo[d]oxazole-7-carbonyl)piperazin-1-y1)-2-(2- |
| | methoxypyridin-4-yl)-7-oxooxazolo[4,5-b]pyridin-4(7H)-yl)-N-(3-(oxetan-3- |
| | yl)bicyclo[1.1.1]pentan-1-yl)acetamide |
| I-67a | N-(2-(5-ethyl-6-(4-(6-hydroxybenzo[d]oxazole-7-carbonyl)piperazin-1-yl)-2-(2- |
| | methoxypyridin-4-y1)-7-oxooxazolo[4,5-b]pyridin-4(7H)-yl)ethyl)-3- |
| | (trifluoromethyl)bicyclo[1.1.1]pentane-1-carboxamide |
| I-68a | 2-(5-ethyl-6-(4-(6-hydroxybenzo[d]oxazole-7-carbonyl)piperazin-1-yl)-2-(2- |
| | methoxypyridin-4-yl)-7-oxooxazolo[4,5-b]pyridin-4(7H)-y1)-N-(4- |
| | (trifluoromethyl)bicyclo[2.2.2]octan-1-yl)acetamide |
| I-69a | 2-(5-ethyl-6-(4-(6-hydroxybenzo[d]oxazole-7-carbonyl)piperazin-1-y1)-2-(2- |
| | methoxypyridin-4-yl)-7-oxooxazolo[4,5-b]pyridin-4(7H)-yl)-N-(4- |
| | fluorobicyclo[2.2.2]octan-1-yl)acetamide |
| I-70a | 2-(5-ethyl-6-(4-(6-hydroxybenzo[d]oxazole-7-carbonyl)piperazin-1-y1)-2- |
| | morpholino-7-oxooxazolo[4,5-b]pyridin-4(7H)-y1)-N-((3r,6r)-6- |
| | (trifluoromethyl)bicyclo[3.1.0]hexan-3-yl)acetamide |
| I-71a | N-(3-cyclopropylbicyclo[1.1.1]pentan-1-yl)-2-(2-(3,6-dihydro-2H-pyran-4-yl)-5- |
| | ethyl-6-(4-(6-hydroxybenzo[d]oxazole-7-carbonyl)piperazin-1-yl)-7- |
| | oxooxazolo[4,5-b]pyridin-4(7H)-yl)acetamide |

TABLE 1b

| No. | IUPAC Name |
|---|---|
| I-2b | N-(2-chloro-4-(trifluoromethyl)phenyl)-2-(2-(3,6-dihydro-2H-pyran-4-y1)-5-ethyl- |
| | 6-(4-(5-hydroxy-6-methylpyrimidine-4-carbonyl)piperazin-1-yl)-7-oxooxazolo[5,4- |
| | b]pyridin-4(7H)-yl)acetamide |
| I-4b | N-(2-chloro-4-(trifluoromethyl)phenyl)-2-(5-ethyl-6-(4-(5-hydroxypyrimidine-4- |
| | carbonyl)piperazin-1-y1)-2-(2-methoxypyridin-4-y1)-7-oxooxazolo[5,4-b]pyridin- |
| | 4(7H)-yl)acetamide |
| I-5b | N-(2-chloro-4-(trifluoromethyl)phenyl)-2-(5-ethyl-6-(4-(3- |
| | hydroxypicolinoyl)piperazin-1-yl)-2-(2-methoxypyridin-4-yl)-7-oxooxazolo[5,4- |
| | b]pyridin-4(7H)-yl)acetamide |
| I-6b | N-(2-chloro-4-(trifluoromethyl)phenyl)-2-(5-ethyl-6-(4-(4-hydroxy-2- |
| | methoxynicotinoyl)piperazin-1-yl)-2-(2-methoxypyridin-4-yl)-7-oxooxazolo[5,4- |
| | b]pyridin-4(7H)-yl)acetamide |
| I-7b | N-(2-chloro-4-(trifluoromethyl)phenyl)-2-(5-ethyl-6-(4-(5-fluoro-4-hydroxy-2- |
| | methoxynicotinoyl)piperazin-1-yl)-2-(2-methoxypyridin-4-yl)-7-oxooxazolo[5,4- |
| | b]pyridin-4(7H)-y1)acetamide |
| I-8b | N-(2-chloro-4-(trifluoromethyl)phenyl)-2-(5-ethyl-6-(4-(4-hydroxyisoxazole-3- |
| | carbonyl)piperazin-1-y1)-2-(2-methoxypyridin-4-y1)-7-oxooxazolo[5,4-b]pyridin- |
| | 4(7H)-y1)acetamide |
| I-9b | N-(2-chloro-4-(trifluoromethyl)phenyl)-2-(5-ethyl-6-(4-(4-hydroxyisothiazole-3- |
| | carbonyl)piperazin-1-yl)-2-(2-methoxypyridin-4-y1)-7-oxooxazolo[5,4-b]pyridin- |
| | 4(7H)-yl)acetamide |
| I-10b | N-(2-chloro-4-(trifluoromethyl)phenyl)-2-(5-ethyl-6-(4-(3-hydroxy-2- |
| | methoxyisonicotinoyl)piperazin-1-y1)-2-(2-methoxypyridin-4-y1)-7-oxooxazolo[5,4- |
| | b]pyridin-4(7H)-yl)acetamide |
| I-11b | N-(2-chloro-4-(trifluoromethyl)phenyl)-2-(5-ethyl-6-(4-(5-fluoro-3-hydroxy-2- |
| | methoxyisonicotinoyl)piperazin-1-yl)-2-(2-methoxypyridin-4-y1)-7-oxooxazolo[5,4- |
| | b]pyridin-4(7H)-yl)acetamide |
| I-12b | N-(2-chloro-4-(trifluoromethyl)phenyl)-2-(2-(3,6-dihydro-2H-pyran-4-y1)-5-ethyl- |
| | 6-(4-(5-hydroxypyrimidine-4-carbonyl)piperazin-1-yl)-7-oxooxazolo[5,4-b]pyridin- |
| | 4(7H)-yl)acetamide |
| I-13b | N-(2-chloro-4-(trifluoromethyl)phenyl)-2-(2-(3,6-dihydro-2H-pyran-4-yl)-5-ethyl- |
| | 6-(4-(3-hydroxypicolinoyl)piperazin-1-yl)-7-oxooxazolo[5,4-b]pyridin-4(7H)- |
| | yl)acetamide |
| I-14b | N-(2-chloro-4-(trifluoromethyl)phenyl)-2-(2-(3,6-dihydro-2H-pyran-4-y1)-5-ethyl- |
| | 6-(4-(4-hydroxy-2-methoxynicotinoyl)piperazin-1-y1)-7-oxooxazolo[5,4-b]pyridin- |
| | 4(7H)-yl)acetamide |
| I-15b | N-(2-chloro-4-(trifluoromethyl)phenyl)-2-(2-(3,6-dihydro-2H-pyran-4-y1)-5-ethyl- |
| | 6-(4-(5-fluoro-4-hydroxy-2-methoxynicotinoyl)piperazin-1-yl)-7-oxooxazolo[5,4- |
| | b]pyridin-4(7H)-yl)acetamide |
| I-16b | N-(2-chloro-4-(trifluoromethyl)phenyl)-2-(2-(3,6-dihydro-2H-pyran-4-y1)-5-ethyl- |
| | 6-(4-(3-hydroxy-2-methoxyisonicotinoyl)piperazin-1-yl)-7-oxooxazolo[5,4- |
| | b]pyridin-4(7H)-yl)acetamide |
| I-17b | N-(2-chloro-4-(trifluoromethyl)phenyl)-2-(2-(3,6-dihydro-2H-pyran-4-yl)-5-ethyl- |
| | 6-(4-(5-fluoro-3-hydroxy-2-methoxyisonicotinoyl)piperazin-1-yl)-7- |
| | oxooxazolo[5,4-b]pyridin-4(7H)-y1)acetamide |
| I-18b | N-(2-chloro-4-(trifluoromethyl)phenyl)-2-(5-ethyl-6-((1S,6S)-5-(5-hydroxy-6- |
| | methylpyrimidine-4-carbonyl)-2,5-diazabicyclo[4.2.0]octan-2-y1)-2-(2- |
| | methoxypyridin-4-y1)-7-oxooxazolo[5,4-b]pyridin-4(7H)-yl)acetamide |
| I-19b | N-(2-chloro-4-(trifluoromethyl)phenyl)-2-(5-ethyl-6-((1R,6R)-5-(5-hydroxy-6- |
| | methylpyrimidine-4-carbonyl)-2,5-diazabicyclo[4.2.0]octan-2-y1)-2-(2- |
| | methoxypyridin-4-yl)-7-oxooxazolo[5,4-b]pyridin-4(7H)-yl)acetamide |
| I-20b | N-(2-chloro-4-(trifluoromethyl)phenyl)-2-(2-(3,6-dihydro-2H-pyran-4-y1)-5-ethyl- |
| | 6-((1R,6R)-5-(5-hydroxy-6-methylpyrimidine-4-carbonyl)-2,5- |
| | diazabicyclo[4.2.0]octan-2-yl)-7-oxooxazolo[5,4-b]pyridin-4(7H)-yl)acetamide |
| I-21b | N-(2-chloro-4-(trifluoromethyl)phenyl)-2-(2-(3,6-dihydro-2H-pyran-4-y1)-5-ethyl- |
| | 6-((1S,6S)-5-(5-hydroxy-6-methylpyrimidine-4-carbonyl)-2,5- |
| | diazabicyclo[4.2.0]octan-2-y1)-7-oxooxazolo[5,4-b]pyridin-4(7H)-yl)acetamide |
| I-22b | 2-(5-ethyl-6-(4-(6-hydroxybenzo[d]oxazole-7-carbonyl)piperazin-1-y1)-2-(2- |
| | methoxypyridin-4-yl)-7-oxooxazolo[5,4-b]pyridin-4(7H)-yl)-N-(3- |
| | (trifluoromethyl)bicyclo[1.1.1]pentan-1-yl)acetamide |
| I-23b | N-(3-cyclopropylbicyclo[1.1.1]pentan-1-yl)-2-(2-(3,6-dihydro-2H-pyran-4-yl)-5- |
| | ethyl-6-(4-(4-hydroxy-2,3-dihydrofuro[2,3-c]pyridine-5-carbonyl)piperazin-1-y1)-7- |
| | oxooxazolo[5,4-b]pyridin-4(7H)-yl)acetamide |
| I-24b | 2-(2-(1-acetyl-1,2,3,6-tetrahydropyridin-4-yl)-5-ethyl-6-(4-(4-hydroxy-2,3- |
| | dihydrofuro[2,3-c]pyridine-5-carbonyl)piperazin-1-yl)-7-oxooxazolo[5,4-b]pyridin- |
| | 4(7H)-yl)-N-(3-(trifluoromethyl)bicyclo[1.1.1]pentan-1-yl)acetamide |
| I-25b | N-(3-(1, 1-difluoroethyl)bicyclo[1.1.1]pentan-1-y1)-2-(2-(3,6-dihydro-2H-pyran-4- |
| | yl)-5-ethyl-6-(4-(4-hydroxy-2,3-dihydrofuro[2,3-c]pyridine-5-carbonyl)piperazin-1- |
| | y1)-7-oxooxazolo[5,4-b]pyridin-4(7H)-yl)acetamide |
| I-26b | 2-(2-(3,6-dihydro-2H-pyran-4-y1)-5-ethyl-6-(4-(4-hydroxy-2,3-dihydrofuro[2,3- |
| | c]pyridine-5-carbonyl)piperazin-1-yl)-7-oxooxazolo[5,4-b]pyridin-4(7H)-yl)-N-(3- |
| | methylbicyclo[1.1.1]pentan-1-yl)acetamide |
| I-27b | N-(3-(difluoromethyl)bicyclo[1.1.1]pentan-1-yl)-2-(2-(3,6-dihydro-2H-pyran-4-yl)- |
| | 5-ethyl-6-(4-(4-hydroxy-2,3-dihydrofuro[2,3-c]pyridine-5-carbonyl)piperazin-1-yl)- |
| | 7-oxooxazolo[5,4-b]pyridin-4(7H)-y1)acetamide |
| I-28b | 2-(2-(3,6-dihydro-2H-pyran-4-y1)-5-ethyl-6-(4-(5-hydroxy-6-methylpyrimidine-4- |
| | carbonyl)piperazin-1-yl)-7-oxooxazolo[5,4-b]pyridin-4(7H)-yl)-N-(1- |
| | (trifluoromethyl)-2-oxabicyclo[2.1.1]hexan-4-yl)acetamide |
| I-29b | 2-(5-ethyl-6-(4-(5-hydroxy-6-methylpyrimidine-4-carbonyl)piperazin-1-y1)-2-(2- |
| | methoxypyridin-4-yl)-7-oxooxazolo[5,4-b]pyridin-4(7H)-yl)-N-(3- |
| | (trifluoromethyl)cyclobutyl)acetamide |
| I-30b | 2-(2-(3,6-dihydro-2H-pyran-4-y1)-5-ethyl-6-(4-(4-hydroxy-2,3-dihydrofuro[2,3- |
| | c]pyridine-5-carbonyl)piperazin-1-yl)-7-oxooxazolo[5,4-b]pyridin-4(7H)-yl)-N-(3- |
| | methoxybicyclo[1.1.1]pentan-1-yl)acetamide |
| I-31b | 2-(2-(3,6-dihydro-2H-pyran-4-y1)-5-ethyl-6-(4-(4-hydroxy-2,3-dihydrofuro[2,3- |
| | c]pyridine-5-carbonyl)piperazin-1-y1)-7-oxooxazolo[5,4-b]pyridin-4(7H)-yl)-N-(3- |
| | fluorobicyclo[1.1.1]pentan-1-y1)acetamide |
| I-32b | 2-(2-(3,6-dihydro-2H-pyran-4-yl)-5-ethyl-6-((1R,6R)-5-(5-hydroxy-6- |
| | methylpyrimidine-4-carbonyl)-2,5-diazabicyclo[4.2.0]octan-2-y1)-7- |
| | oxooxazolo[5,4-b]pyridin-4(7H)-y1)-N-(3-(2-fluoroethyl)bicyclo[1.1.1]pentan-1- |
| | yl)acetamide |
| I-33b | 2-(2-(3,6-dihydro-2H-pyran-4-y1)-5-ethyl-6-(4-(4-hydroxy-2,3-dihydrofuro[2,3- |
| | c]pyridine-5-carbonyl)piperazin-1-yl)-7-oxooxazolo[5,4-b]pyridin-4(7H)-yl)-N-(3- |
| | (trifluoromethyl)bicyclo[1.1.1]pentan-1-yl)acetamide |
| I-34b | 2-(2-(3,6-dihydro-2H-pyran-4-yl)-5-ethyl-6-(4-(4-hydroxy-2,3-dihydrofuro[2,3- |
| | c]pyridine-5-carbonyl)piperazin-1-yl)-7-oxooxazolo[5,4-b]pyridin-4(7H)-yl)-N-(3- |
| | (trifluoromethoxy)bicyclo[1.1.1]pentan-1-yl)acetamide |
| I-35b | N-(3-cyanobicyclo[1.1.1]pentan-1-yl)-2-(2-(3,6-dihydro-2H-pyran-4-yl)-5-ethyl-6- |
| | (4-(4-hydroxy-2,3-dihydrofuro[2,3-c]pyridine-5-carbonyl)piperazin-1-y1)-7- |
| | oxooxazolo[5,4-b]pyridin-4(7H)-y1)acetamide |
| I-36b | 2-(2-(dimethylamino)-5-ethyl-6-(4-(6-hydroxybenzo[d]oxazole-7- |
| | carbonyl)piperazin-1-y1)-7-oxooxazolo[5,4-b]pyridin-4(7H)-y1)-N-(3- |
| | (trifluoromethyl)bicyclo[1.1.1]pentan-1-yl)acetamide |
| I-37b | 2-(5-ethyl-6-(4-(5-fluoro-4-hydroxy-2-methoxynicotinoyl)piperazin-1-y1)-2-(2- |
| | methoxypyridin-4-yl)-7-oxooxazolo[5,4-b]pyridin-4(7H)-yl)-N-(3- |
| | (trifluoromethyl)bicyclo[1.1.1]pentan-1-yl)acetamide |
| I-38b | 2-(5-ethyl-6-(4-(3-hydroxypicolinoyl)piperazin-1-y1)-2-(2-methoxypyridin-4-yl)-7- |
| | oxooxazolo[5,4-b]pyridin-4(7H)-y1)-N-(3-(trifluoromethyl)bicyclo[1.1.1]pentan-1- |
| | y1)acetamide |
| I-39b | 2-(5-ethyl-6-(4-(4-hydroxy-2-methoxy-5-methylnicotinoyl)piperazin-1-y1)-2-(2- |
| | methoxypyridin-4-yl)-7-oxooxazolo[5,4-b]pyridin-4(7H)-yl)-N-(3- |
| | (trifluoromethyl)bicyclo[1.1.1]pentan-1-yl)acetamide |
| I-40b | 2-(5-ethyl-6-((1R,6R)-5-(5-hydroxy-6-methylpyrimidine-4-carbonyl)-2,5- |
| | diazabicyclo[4.2.0]octan-2-y1)-2-(2-methoxypyridin-4-yl)-7-oxooxazolo[5,4- |
| | b]pyridin-4(7H)-y1)-N-(3-(trifluoromethyl)bicyclo[1.1.1]pentan-1-yl)acetamide |
| I-41b | 2-(2-(dimethylamino)-5-ethyl-6-(4-(5-hydroxy-6-methylpyrimidine-4- |
| | carbonyl)piperazin-1-y1)-7-oxooxazolo[5,4-b]pyridin-4(7H)-y1)-N-(3- |
| | (trifluoromethyl)bicyclo[1.1.1]pentan-1-yl)acetamide |
| I-42b | 2-(5-ethyl-6-(4-(6-hydroxybenzo[d]oxazole-7-carbonyl)piperazin-1-yl)-7-oxo-2- |
| | (pyrrolidin-1-yl)oxazolo[5,4-b]pyridin-4(7H)-yl)-N-(3- |
| | (trifluoromethyl)bicyclo[1.1.1]pentan-1-yl)acetamide |
| I-43b | 2-(5-ethyl-6-(4-(4-hydroxy-2,3-dihydrofuro[2,3-c]pyridine-5-carbonyl)piperazin-1- |
| | yl)-2-(1-(2-methoxyacetyl)-1,2,3,6-tetrahydropyridin-4-yl)-7-oxooxazolo[5,4- |
| | b]pyridin-4(7H)-y1)-N-(3-(trifluoromethyl)bicyclo[1.1.1]pentan-1-yl)acetamide |
| I-44b | 2-(5-ethyl-6-(4-(6-hydroxybenzo[d]oxazole-7-carbonyl)piperazin-1-y1)-2- |
| | morpholino-7-oxooxazolo[5,4-b]pyridin-4(7H)-y1)-N-(3- |
| | (trifluoromethyl)bicyclo[1.1.1]pentan-1-yl)acetamide |
| I-45b | N-(3-cyclopropylbicyclo[1.1.1]pentan-1-y1)-2-(5-ethyl-6-(4-(6- |
| | hydroxybenzo[d]oxazole-7-carbonyl)piperazin-1-yl)-2-(2-methoxypyridin-4-y1)-7- |
| | oxooxazolo[5,4-b]pyridin-4(7H)-yl)acetamide |
| I-46b | 2-(5-ethyl-6-((1R,6R)-5-(6-hydroxybenzo[d]oxazole-7-carbonyl)-2,5- |
| | diazabicyclo[4.2.0]octan-2-y1)-2-(2-methoxypyridin-4-yl)-7-oxooxazolo[5,4- |
| | b]pyridin-4(7H)-y1)-N-(3-(trifluoromethyl)bicyclo[1.1.1]pentan-1-yl)acetamide |
| I-47b | 2-(2-(dimethylamino)-5-ethyl-6-((1R,6R)-5-(6-hydroxybenzo[d]oxazole-7- |
| | carbonyl)-2,5-diazabicyclo[4.2.0]octan-2-y1)-7-oxooxazolo[5,4-b]pyridin-4(7H)-y1)- |
| | N-(3-(trifluoromethyl)bicyclo[1.1.1]pentan-1-yl)acetamide |
| I-48b | 2-(5-ethyl-6-((1R,6R)-5-(6-hydroxybenzo[d]oxazole-7-carbonyl)-2,5- |
| | diazabicyclo[4.2.0]octan-2-y1)-2-morpholino-7-oxooxazolo[5,4-b]pyridin-4(7H)- |
| | y1)-N-(3-(trifluoromethyl)bicyclo[1.1.1]pentan-1-yl)acetamide |
| I-49b | 2-(5-ethyl-6-(4-(5-hydroxybenzo[d]oxazole-4-carbonyl)piperazin-1-y1)-2-(2- |
| | methoxypyridin-4-yl)-7-oxooxazolo[5,4-b]pyridin-4(7H)-yl)-N-(3- |
| | (trifluoromethyl)bicyclo[1.1.1]pentan-1-yl)acetamide |
| I-50b | 2-(5-ethyl-6-(4-(6-hydroxybenzo[d]oxazole-7-carbonyl)piperazin-1-y1)-2-(2- |
| | methoxypyridin-4-yl)-7-oxooxazolo[5,4-b]pyridin-4(7H)-yl)-N-(3- |
| | ethylbicyclo[1.1.1]pentan-1-yl)acetamide |
| I-51b | 2-(5-ethyl-6-(4-(6-hydroxybenzo[d]oxazole-7-carbonyl)piperazin-1-yl)-2-(2- |
| | methoxypyridin-4-yl)-7-oxooxazolo[5,4-b]pyridin-4(7H)-yl)-N-(3- |
| | isopropylbicyclo[1.1.1]pentan-1-yl)acetamide |
| I-52b | 2-(5-ethyl-6-(4-(6-hydroxybenzo[d]oxazole-7-carbonyl)piperazin-1-y1)-2-(2- |
| | methoxypyridin-4-yl)-7-oxooxazolo[5,4-b]pyridin-4(7H)-yl)-N-(3-(2- |
| | fluoroethyl)bicyclo[1.1.1]pentan-1-yl)acetamide |
| I-53b | N-(3-cyclobutylbicyclo[1.1.1]pentan-1-yl)-2-(5-ethyl-6-(4-(6- |
| | hydroxybenzo[d]oxazole-7-carbonyl)piperazin-1-yl)-2-(2-methoxypyridin-4-yl)-7- |
| | oxooxazolo[5,4-b]pyridin-4(7H)-yl)acetamide |
| I-54b | 2-(5-ethyl-6-(4-(6-hydroxybenzo[d]oxazole-7-carbonyl)piperazin-1-y1)-2-(2- |
| | methoxypyridin-4-yl)-7-oxooxazolo[5,4-b]pyridin-4(7H)-yl)-N-(3-(oxetan-3- |
| | yl)bicyclo[1.1.1]pentan-1-yl)acetamide |
| I-55b | N-(2-(5-ethyl-6-(4-(6-hydroxybenzo[d]oxazole-7-carbonyl)piperazin-1-y1)-2-(2- |
| | methoxypyridin-4-y1)-7-oxooxazolo[5,4-b]pyridin-4(7H)-yl)ethyl)-3- |
| | (trifluoromethyl)bicyclo[ 1.1.1]pentane-1-carboxamide |
| I-56b | 2-(5-ethyl-6-(4-(6-hydroxybenzo[d]oxazole-7-carbonyl)piperazin-1-yl)-2-(2- |
| | methoxypyridin-4-y1)-7-oxooxazolo[5,4-b]pyridin-4(7H)-y1)-N-(4- |
| | (trifluoromethyl)bicyclo[2.2.2]octan-1-yl)acetamide |
| I-57b | 2-(5-ethyl-6-(4-(6-hydroxybenzo[d]oxazole-7-carbonyl)piperazin-1-y1)-2-(2- |
| | methoxypyridin-4-yl)-7-oxooxazolo[5,4-b]pyridin-4(7H)-yl)-N-(4- |
| | fluorobicyclo[2.2.2]octan-1-yl)acetamide |
| I-58b | 2-(5-ethyl-6-(4-(6-hydroxybenzo[d]oxazole-7-carbonyl)piperazin-1-y1)-2-(2- |
| | methoxypyridin-4-y1)-7-oxooxazolo[5,4-b]pyridin-4(7H)-yl)-N-((3r,6r)-6- |
| | (trifluoromethyl)bicyclo[3.1.0]hexan-3-yl)acetamide |
| I-59b | N-(3-cyclopropylbicyclo[1.1.1]pentan-1-y1)-2-(2-(3,6-dihydro-2H-pyran-4-yl)-5- |
| | ethyl-6-(4-(6-hydroxybenzo[d]oxazole-7-carbonyl)piperazin-1-yl)-7- |
| | oxooxazolo[5,4-b]pyridin-4(7H)-yl)acetamide |
| I-60b | N-(3-cyclopropylbicyclo[1.1.1]pentan-1-yl)-2-(2-(3,6-dihydro-2H-pyran-4-y1)-5- |
| | ethyl-6-(4-(5-hydroxy-6-methylpyrimidine-4-carbonyl)piperazin-1-y1)-7- |
| | oxooxazolo[5,4-b]pyridin-4(7H)-yl)acetamide |

### 4. Pharmaceutical Compositions, Methods of Treatment and Uses of Compounds

In another aspect, the present invention provides a pharmaceutical composition comprising a compound of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration, in particular oral administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Typical approaches to solubilize compounds for parenteral administration are the optimization of the pH or the use of co-solvents (e.g. PEG300, PEG400, propylene glycol, or ethanol). If these approaches are, for any reason, not feasible, the use of surfactants may be considered (e.g. Tween® 80 or Cremophor EL®). Cyclodextrins are established as safe solubilizing agents. Compounds with a high solubility in natural oils may be solubilized in parenteral fat emulsions.

There is also provided a pharmaceutical composition comprising a compound of Formula I as described herein, or a pharmaceutically acceptable salt thereof, and one or more pharmaceutically acceptable carriers.

### Uses

The compounds of Formula I of the present invention in free form or in pharmaceutically acceptable salt form, exhibit valuable pharmacological properties, e.g. WRN inhibiting properties, e.g. as indicated in vitro tests as provided in the next sections, and are therefore indicated for therapy, or for use as research chemicals, e.g. as a chemical probe, and as tool compounds.

Also provided is a compound of Formula I, as described herein. Said compound can be used as a research chemical, a compound herein comprising an added biotin moiety, for example a tool compound or chemical probe, in particular for research on WRN. In another embodiment there is provided the use of a compound of Formula I, as described herein, as a research chemical, for example tool compound or chemical probe, in particular for research on WRN.

There is also provided a compound of Formula I as described herein, or a pharmaceutically acceptable salt thereof, for use in the treatment of cancer. Cancers that may be treated by WRN inhibition include cancers that are characterized as microsatellite instability-high (MSI-H) or mismatch repair deficient (dMMR). In particular, a compound of Formula I as described herein, or a pharmaceutically acceptable salt thereof, may be useful in the treatment of a cancer that is characterized as microsatellite instability-high (MSI-H) or mismatch repair deficient (dMMR).

There is also provided a compound of Formula I as described herein, or a pharmaceutically acceptable salt thereof, for use as a medicament. In particular, said use is:
for the treatment of a disease that is treated by WRN inhibition,
for the treatment of cancer,
for the treatment of cancer that is characterized as microsatellite instability-high (MSI-H) or mismatch repair deficient (dMMR),
for the treatment of cancer that is characterized as microsatellite instability-high (MSI-H) or mismatch repair deficient (dMMR), such as colorectal, gastric, prostate, endometrial, adrenocortical, uterine, cervical, esophageal, breast, kidney and ovarian cancer,
for the treatment of cancer that is characterized as microsatellite instability-high (MSI-H) or mismatch repair deficient (dMMR) is selected from colorectal, gastric, prostate and endometrial cancer, or
for the treatment of cancer wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided a method of:
modulating WRN activity in a subject, wherein the method comprises administering to the subject a therapeutically effective amount of the compound of Formula I as described herein, or a pharmaceutically acceptable salt thereof,
inhibiting WRN in a subject, wherein the method comprises administering to the subject a therapeutically effective amount of the compound of Formula I as described herein, or a pharmaceutically acceptable salt thereof,
treating a disorder or disease which can be treated by WRN inhibition in a subject, comprising administering to the subject a therapeutically effective amount of the compound of Formula I as described herein, or a pharmaceutically acceptable salt thereof,
treating cancer in a subject, comprising administering to the subject a therapeutically effective amount of the compound of Formula I as described herein, or a pharmaceutically acceptable salt thereof,
treating cancer in a subject, comprising administering a compound of Formula I as described herein, wherein the cancer is characterized as microsatellite instability-high (MSI-H) or mismatch repair deficient (dMMR). In particular, the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer. More particularly, the cancer characterized as microsatellite instability-high (MSI-H) or mismatch repair deficient (dMMR) is selected from colorectal, gastric, prostate and endometrial cancer. Examples include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided the use of a compound of Formula I as described herein, or a pharmaceutically acceptable salt thereof:
in therapy,
in the manufacture of a medicament,
in the manufacture of a medicament for the treatment of cancer. In particular, said cancer is characterized as microsatellite instability-high (MSI-H) or mismatch repair deficient (dMMR),
in the manufacture of a medicament for treatment of a disease which may be treated by WRN inhibition,
   wherein in particular, the cancer is characterized by microsatellite instability-high (MSI-H) or mismatch repair deficient (dMMR), for example colorectal, gastric, prostate, endometrial, adrenocortical, uterine, cervical, esophageal, breast, kidney and ovarian cancer, in particular, colorectal, gastric, prostate or endometrial cancer, or uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

In some embodiments, the subject has or is identified as having a microsatellite instable (MSI-H) cancer, e.g., in reference to a control, e.g., a normal, subject. In one embodiment, the subject has MSI-H advanced solid tumors, a colorectal cancer (CRC), endometrial, uterine, stomach or other MSI-H cancer. In some embodiments, the subject has a colorectal (CRC), endometrial or stomach cancer, which cancer has or is identified as having a microsatellite instability (MSI-H), e.g., in reference to a control, e.g., a normal, subject. Such identification techniques are known in the art.

### Forms

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any formula given herein is intended to represent unlabeled forms as well as isotopically labeled forms of the compounds, in addition to the deuteration specifically claimed in Formula I. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen.

Further, incorporation of certain isotopes, particularly deuterium (i.e., 2H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as 3H, 11C, 13C, 14C, 15N, 18F 31P, 32P, 35S, 36CI, 123I, 124I, and 125I, respectively. Accordingly, it should be understood that the invention includes compounds that incorporate one or more of any of the aforementioned isotopes, including for example, radioactive isotopes, such as 3H and 14C, or those into which non-radioactive isotopes, such as 2H and 13C are present. Such isotopically labelled compounds are useful in metabolic studies (with 14C), reaction kinetic studies (with, for example 2H or 3H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

A “compound of the present invention” or a “compound of Formula I” includes a zwitterion thereof, a non-zwitterion thereof (non-charged form), or a pharmaceutically acceptable salt of said zwitterionic or non-zwitterionic form thereof. “Zwitterion” or “zwitterionic form” means a compound containing both positive and negatively charged functional groups.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to colorectal, gastric, endometrial, prostate, adrenocortical, uterine, cervical, esophageal, breast, kidney, ovarian cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“WRN inhibitor” or “WRN helicase inhibitor” as used herein means a compound that inhibits Werner Syndrome RecQ DNA helicase (WRN). The term “WRN” as used herein refers to the protein of Werner Syndrome RecQ DNA helicase. The term “WRN” includes mutants, fragments, variants, isoforms, and homologs of full-length wild-type WRN. In one embodiment, the protein is encoded by the WRN gene (Entrez gene ID 7486; Ensembl ID ENSG00000165392). Exemplary WRN sequences are available at the Uniprot database under accession number Q14191.

“Disease or condition mediated by WRN” includes a disease or condition, such as cancer, which is treated by WRN inhibition. In particular this may include cancers characterized as microsatellite instability-high (MSI-H) or mismatch repair deficient (dMMR).

“Microsatellite unstable cancer,” “microsatellite instability-high cancer,” “microsatellite high cancer” and “MSI-high cancer,” “MSIhi” and “MSI-H” when used herein, are used interchangeably, and describe cancers that have a high number of alterations in the length of simple repetitive genomic sequences within microsatellites.

The determination of MSI-H or dMMR tumor status for patients can be performed using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; and Kawakami et al. Curr Treat Options Oncol. 2015; 16(7):30).

Microsatellite instability can be found in colorectal cancer, gastric cancer and endometrial cancer in particular, but also in adrenocortical, uterine, cervical, esophageal, breast, kidney, prostate and ovarian cancers. Examples of microsatellite high cancers include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

A cancer that has “defective mismatch repair” (dMMR) or “dMMR character” includes cancer types associated with documented MLH1, PMS2, MSH2, MSH3, MSH6, MLH3, and PMS1 mutations or epigenetic silencing, microsatellite fragile sites, or other gene inactivation mechanisms, including but not limited to cancers of the lung, breast, kidney, large intestine, ovary, prostate, upper aerodigestive tract, stomach, endometrium, liver, pancreas, haematopoietic and lymphoid tissue, skin, thyroid, pleura, autonomic ganglia, central nervous system, soft tissue, pediatric rhabdoid sarcomas, melanomas and other cancers. A cell or cancer with “defective” mismatch repair has a significantly reduced (e.g., at least about 25%, 30%, 40%, 50%, 60%, 70%, 80% or 90% decrease) amount of mismatch repair. In some cases, a cell or cancer which is defective in mismatch repair will perform no mismatch repair.

As used herein, the term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy 22nd Ed. Pharmaceutical Press, 2013, pp. 1049-1070).

The terms “synthetic lethality,” and “synthetically lethal” are used to refer to reduced cell viability and/or a reduced rate of cell proliferation caused by a combination of mutations or approaches to cause loss of function (e.g., RNA interference or protein function inhibition) in two or more genes but not by the loss of function of only one of these genes.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In some embodiments, the methods of the invention comprise administration of a therapeutically effective amount of a compound herein.

In one embodiment, the term “a therapeutically effective amount” refers to the amount of the compound of the present invention that, when administered to a subject, is effective to (1) at least partially alleviate, prevent and/or ameliorate a condition, or a disorder or a disease (i) mediated by WRN, or (ii) associated with WRN activity, or (iii) characterized by activity (normal or abnormal) of WRN; or (2) reduce or inhibit the activity of WRN.

In another embodiment, the term “a therapeutically effective amount” refers to the amount of the compound of the present invention that, when administered to a cell, or a tissue, or a non-cellular biological material, or a medium, is effective to at least partially reducing or inhibiting the activity of WRN, or reducing WRN protein levels.

As used herein, the term “subject” refers to primates (e.g., humans, male or female), dogs, rabbits, guinea pigs, pigs, rats and mice. In certain embodiments, the subject is a primate, a rat or a mouse. In yet other embodiments, the subject is a human.

As used herein, the term “inhibit,” “inhibition” or “inhibiting” refers to the reduction or suppression of a given condition, symptom, or disorder, or disease, or a significant decrease in the baseline activity of a biological activity or process.

As used herein, the term “treat,” “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent,” “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

“May join” means joins or does not join.

“May be replaced by deuterium” means is replaced by deuterium, or is not replaced by deuterium.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### Isomeric Forms

Any asymmetric atom (e.g., carbon or the like) of the compound(s) of the present invention can be present in racemic or enantiomerically enriched, for example (R)-, (S)- or (R, S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of the (R)- or (S)-configuration. Substituents at atoms with unsaturated double bonds may, if possible, be present in cis-(Z)- trans-(E)- form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

Compounds of the invention, i.e. compounds of Formula I that contain groups capable of acting as donors and/or acceptors for hydrogen bonds may be capable of forming co-crystals with suitable co-crystal formers. These co-crystals may be prepared from compounds of Formula I by known co-crystal forming procedures. Such procedures include grinding, heating, co-subliming, co-melting, or contacting in solution compounds of Formula I with the co-crystal former under crystallization conditions and isolating co-crystals thereby formed. Suitable co-crystal formers include those described in WO 2004/078163. Hence the invention further provides co-crystals comprising a compound of Formula I.

Furthermore, the compounds of the present invention, including their salts, can also be obtained in the form of their hydrates, or include other solvents used for their crystallization.

The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

### Dosage Forms

The pharmaceutical composition or combination of the present invention may, for example, be in unit dosage of about 1-1000 mg of active ingredient(s) for a subject of about 50-70 kg.

### Combinations

“Combination” refers to either a fixed combination in one dosage unit form, or a combined administration where a compound of Formula I, or a pharmaceutically acceptable salt thereof, and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage.

The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The combinations described herein can include a compound of Formula I and one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the treatment.

There is also provided a combination comprising a compound of Formula I as described herein, or a pharmaceutically acceptable salt thereof, as described herein, and one or more additional therapeutically active agents. The additional therapeutic agent is, for example, a chemical compound, peptide, antibody, antibody fragment or nucleic acid, which is therapeutically active or enhances the therapeutic activity when administered to a patient in combination with a compound of the present disclosure. In particular, an additional therapeutically active agent is:
an anti-cancer agent,
a chemotherapy,
liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX0), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), in particular fluorouracil (5-FU) and irinotecan (Camptosar®).
a PD-1 inhibitor,
an anti-PD-1 antibody molecule,
spartalizumab, or
tislelizumab (BGB-A317, Beigene).

In certain embodiments, the additional therapeutically active agent is:
a chemotherapy selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate, or
a PD-1 inhibitor selected from spartalizumab (Novartis), nivolumab (Bristol-Myers Squibb), pembrolizumab (Merck & Co), pidilizumab (CureTech), MED10680 (Medimmune), cemiplimab (REGN2810, Regeneron), dostarlimab (TSR-042, Tesaro), PF-06801591 (Pfizer), tislelizumab (BGB-A317, Beigene), BGB-108 (Beigene), INCSHR1210 (Incyte), balstilimab (AGEN2035, Agenus), sintilimab (InnoVent), toripalimab (Shanghai Junshi Bioscience), camrelizumab (Jiangsu Hengrui Medicine Co.), AMP-224 (Amplimmune), penpulimab (Akeso Biopharma Inc), zimberelimab (Arcus Biosciences Inc), and prolgolimab (Biocad Ltd). In a further embodiment, the additional therapeutically active agent is the chemotherapy irinotecan (Camptosar®).

In another embodiment, the additional therapeutically active agent is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. In another embodiment, the additional therapeutically active agent is an anti-PD-1 antibody molecule.

In a further embodiment, the PD-1 inhibitor is an anti-PD-1 antibody molecule as described in US 2015/0210769, published on Jul. 30, 2015, entitled “Antibody Molecules to PD-1 and Uses Thereof.”

In another embodiment, there is provided a combination of a compound of Formula I or a pharmaceutically acceptable salt thereof, and a chemotherapy, and a PD-1 inhibitor. In particular, the chemotherapy and PD-1 inhibitor are selected from those described above. In some embodiments, the PD-1 inhibitor (e.g., tislelizumab) is administered at a flat dose of between about 100 mg to about 600 mg. In some embodiments, the PD-1 inhibitor is administered at a dose of between about 100 mg to about 500 mg. In some embodiments, the PD-1 inhibitor is administered at a dose of between about 100 mg to about 400 mg. In some embodiments, the PD-1 inhibitor is administered at a dose of between about 100 mg to about 300 mg. In some embodiments, the PD-1 inhibitor is administered at a dose of between about 100 mg to about 200 mg. In some embodiments, the PD-1 inhibitor is administered at a dose of between about 200 mg to about 600 mg. In some embodiments, the PD-1 inhibitor is administered at a dose of between about 200 mg to about 500 mg. In some embodiments, the PD-1 inhibitor is administered at a dose of between about 200 mg to about 400 mg. In some embodiments, the PD-1 inhibitor is administered at a dose of between about 200 mg to about 300 mg. In some embodiments, the PD-1 inhibitor is administered at a dose of between about 300 mg to about 600 mg. In some embodiments, the PD-1 inhibitor is administered at a dose of between about 300 mg to about 500 mg. In some embodiments, the PD-1 inhibitor is administered at a dose of between about 300 mg to about 400 mg. In some embodiments, the PD-1 inhibitor is administered at a dose of between about 400 mg to about 600 mg. In some embodiments, the PD-1 inhibitor is administered at a dose of between about 400 mg to about 500 mg. In some embodiments, the PD-1 inhibitor is administered at a dose of between about 500 mg to about 600 mg. In some embodiments, the PD-1 inhibitor is administered at a dose of between about 600 mg to about 700 mg. In some embodiments, the PD-1 inhibitor is administered at a dose of between about 700 mg to about 800 mg. In some embodiments, the PD-1 inhibitor is administered at a dose of between about 800 mg to about 900 mg. In some embodiments, the PD-1 inhibitor is administered at a dose of between about 900 mg to about 1000 mg.

In some embodiments, the PD-1 inhibitor (e.g., tislelizumab) is administered at a flat dose of about 100 mg. In some embodiments, the PD-1 inhibitor is administered at a dose of about 200 mg. In some embodiments, the PD-1 inhibitor is administered at a dose of about 300 mg. In some embodiments, the PD-1 inhibitor is administered at a dose of about 400 mg. In some embodiments, the PD-1 inhibitor is administered at a dose of about 500 mg. In some embodiments, the PD-1 inhibitor is administered at a dose of about 600 mg. In some embodiments, the PD-1 inhibitor is administered at a dose of about 700 mg. In some embodiments, the PD-1 inhibitor is administered at a dose of about 800 mg. In some embodiments, the PD-1 inhibitor is administered at a dose of about 900 mg. In some embodiments, the PD-1 inhibitor is administered at a dose of about 1000 mg.

In some embodiments, the PD-1 inhibitor (e.g., tislelizumab) is administered once every ten weeks. In some embodiments, the PD-1 inhibitor is administered once every nine weeks. In some embodiments, the PD-1 inhibitor is administered once every eight weeks. In some embodiments, the PD-1 inhibitor is administered once every seven weeks. In some embodiments, the PD-1 inhibitor is administered once every six weeks. In some embodiments, the PD-1 inhibitor is administered once every five weeks. In some embodiments, the PD-1 inhibitor is administered once every four weeks. In some embodiments, the PD-1 inhibitor is administered once every three weeks. In some embodiments, the PD-1 inhibitor is administered once every two weeks. In some embodiments, the PD-1 inhibitor is administered once every week.

In some embodiments, the PD-1 inhibitor (e.g., tislelizumab) is administered intravenously.

In some embodiments, the PD-1 inhibitor (e.g., tislelizumab) is administered over a period of about 20 minutes to 40 minutes (e.g., about 30 minutes). In some embodiments, the PD-1 inhibitor is administered over a period of about 30 minutes. In some embodiments, the PD-1 inhibitor is administered over a period of about an hour. In some embodiments, the PD-1 inhibitor is administered over a period of about two hours. In some embodiments, the PD-1 inhibitor is administered over a period of about three hours. In some embodiments, the PD-1 inhibitor is administered over a period of about four hours. In some embodiments, the PD-1 inhibitor is administered over a period of about five hours. In some embodiments, the PD-1 inhibitor is administered over a period of about six hours.

In some embodiments, the PD-1 inhibitor (e.g., tislelizumab) is administered at a dose between about 300 mg to about 500 mg (e.g., about 400 mg), intravenously, once every four weeks. In some embodiments, the PD-1 inhibitor is administered at a dose between about 200 mg to about 400 mg (e.g., about 300 mg), intravenously, once every three weeks. In some embodiments, tislelizumab is administered at a dose of 400 mg, once every four weeks. In some embodiments, tislelizumab is administered at a dose of 300 mg, once every three weeks.

In some embodiments, the PD-1 inhibitor (e.g., tislelizumab) is administered at a dose between about 300 mg to about 500 mg (e.g., about 400 mg), intravenously, over a period of about 20 minutes to about 40 minutes (e.g., about 30 minutes), once every two weeks. In some embodiments, the PD-1 inhibitor is administered at a dose between about 200 mg to about 400 mg (e.g., about 300 mg), intravenously, over a period of about 20 minutes to about 40 minutes (e.g., about 30 minutes), once every three weeks.

In some embodiments, the PD-1 inhibitor (e.g., tislelizumab) is administered at a dose of about 100 mg per week. For example, if a 10-week dose is given to a patient, then the PD-1 inhibitor (e.g., tislelizumab) can be given at 1000 mg. If a 9-week dose is given, then the PD-1 inhibitor (e.g., tislelizumab) can be given at 900 mg. If an 8-week dose is given, then the PD-1 inhibitor (e.g., tislelizumab) can be given at 800 mg. If a 7-week dose is given, then the PD-1 inhibitor (e.g., tislelizumab) can be given at 700 mg. If a 6-week dose is given, then the PD-1 inhibitor (e.g., tislelizumab) can be given at 600 mg. If a 5-week dose is given, then the PD-1 inhibitor (e.g., tislelizumab) can be given at 500 mg. If a 4-week dose is given, then the PD-1 inhibitor (e.g., tislelizumab) can be given at 400 mg. If a 3-week dose is given, then the PD-1 inhibitor (e.g., tislelizumab) can be given at 300 mg. If a 2-week dose is given, then the PD-1 inhibitor (e.g., tislelizumab) can be given at 200 mg. If a 1-week dose is given, then the PD-1 inhibitor (e.g., tislelizumab) can be given at 100 mg.

For example, if an anti-PD-1 antibody, such as tislelizumab is used, it can be administered at a dose of 200 mg as an intravenous infusion, once every three weeks. Alternatively, tislelizumab can be administered at a dose of 300 mg as an intravenous infusion, once every four weeks. If an anti-PD-1 antibody, such as tislelizumab is used, it can be administered at a dose of 300 mg as an intravenous infusion, once every three weeks. Alternatively, tislelizumab can be administered at a dose of 400 mg as an intravenous infusion, once every four weeks.

The above-mentioned compounds, which can be used in combination with a compound of the present invention, can be prepared and administered as described in the art, such as in the documents cited above.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by WRN. Products provided as a combined preparation include a composition comprising the compound of Formula I and the other therapeutic agent(s) together in the same pharmaceutical composition, or the compound of the present invention and the other therapeutic agent(s) in separate form, e.g. in the form of a kit.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of the present invention for treating a disease or condition mediated by WRN, wherein the medicament is prepared for administration with another therapeutic agent. The invention also provides the use of another therapeutic agent for treating a disease or condition mediated by WRN, wherein the medicament is administered with a compound of the present invention.

The invention also provides a compound of the present invention for use in treating a disease or condition mediated by WRN, wherein the compound of the present invention is prepared for administration with another therapeutic agent. The invention also provides another therapeutic agent for use in treating a disease or condition mediated by WRN, wherein the other therapeutic agent is prepared for administration with a compound of the present invention. The invention also provides a compound of the present invention for use in treating a disease or condition mediated by WRN, wherein the compound of the present invention is administered with another therapeutic agent. The invention also provides another therapeutic agent for use in a method of treating a disease or condition mediated by WRN, wherein the other therapeutic agent is administered with a compound of the present invention.

The invention also provides the use of a compound of the present invention for treating a disease or condition mediated by WRN, wherein the patient has previously (e.g. within 24 hours) been treated with another therapeutic agent. The invention also provides the use of another therapeutic agent for treating a disease or condition mediated by WRN, wherein the patient has previously (e.g. within 24 hours) been treated with compound of the present invention.

### 5. General Synthetic Methods of Producing Compounds of the Disclosure

Compounds of the present invention can generally be prepared by conventional techniques known to those skilled in the art or by processes analogous to those described in the accompanying synthetic schemes.

Referring to Scheme 1, amide synthesis achieved by coupling acyl chloride 2 (which contains the R¹ substituent of the present compounds) with primary amine 1 may produce 3. Intramolecular cyclization of 3 to produce oxazole 4 (the X and Y containing moiety of the present compounds) and subsequent amine protection provides 5. Butan-2one in the presence of strong base reacts with the ester of 5 to provide 6 which is subsequently brominated at the x keto position to furnish 7. Said bromine is displaced with a nucleophilic Ring A surrogate (protected piperidine in Step 7) to produce 10. 10 may undergo an intramolecular cyclization to produce an oxazolo[5,4-b]pyridin-7(4H)-one core of the present compounds in intermediate 11. Substituent R² may be introduced into the core of 11 via nucleophilic substitution of R² surrogate 12 to provide 13. Deprotection of 13 and amide coupling using R⁴ surrogate 15 may furnish compounds of the disclosure such as I-2.

Similar chemistry described in Scheme 1 may be used to form intermediates described infra and compounds of the disclosure. Those of skill in the art will readily be able to modify the order of introduction from Scheme 1 of substituents (e.g., R¹, R², R⁴, and Ring A) onto the compound intermediates in Scheme 2 by using alternative protecting group strategies and transition metal cross-couplings (see Step 1) well within the skill set of one having ordinary skill in the art.

Scheme 1: General synthetic methods of producing [5,4-b]pyridin-4(7H) intermediates of the disclosure such as 11

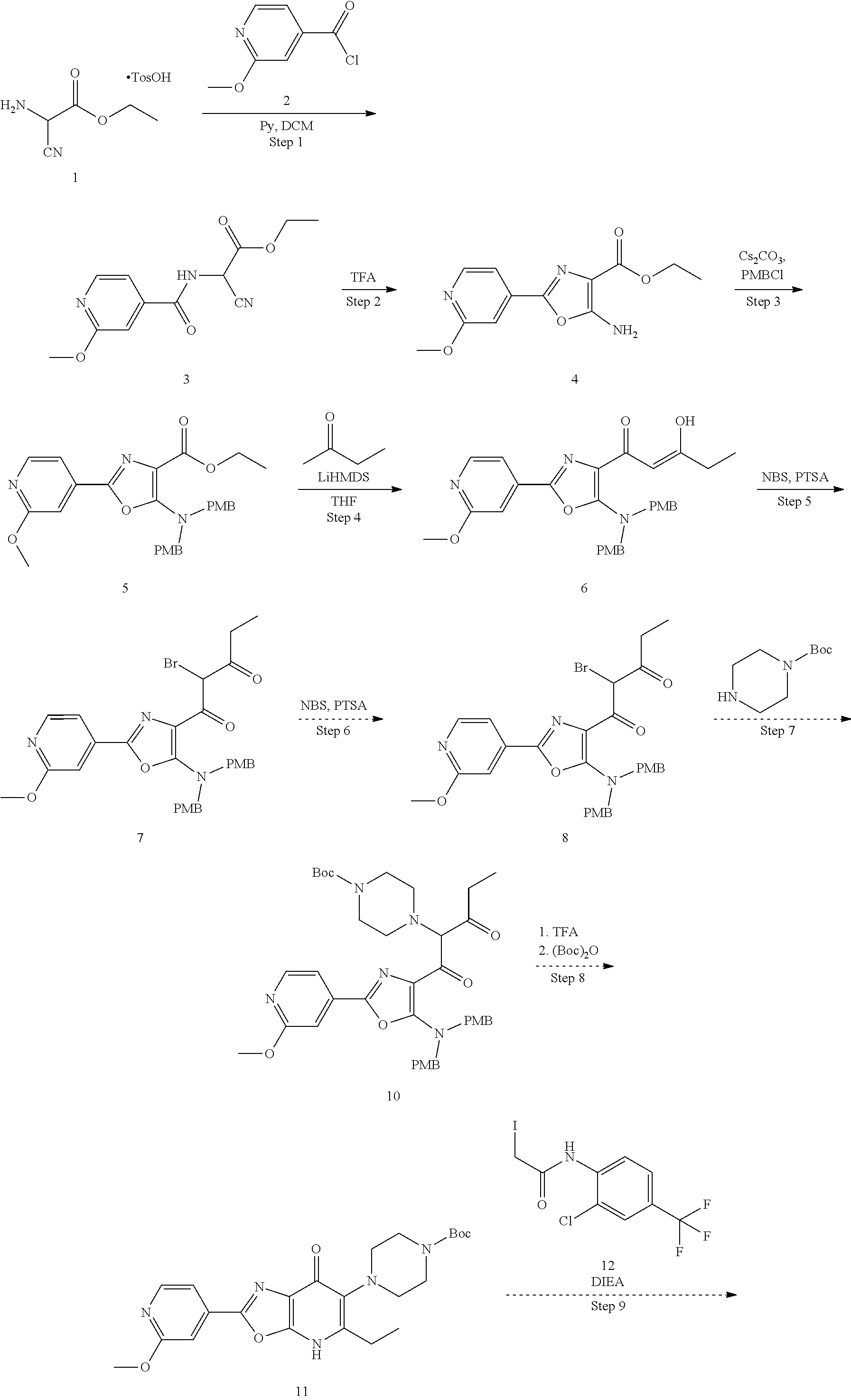

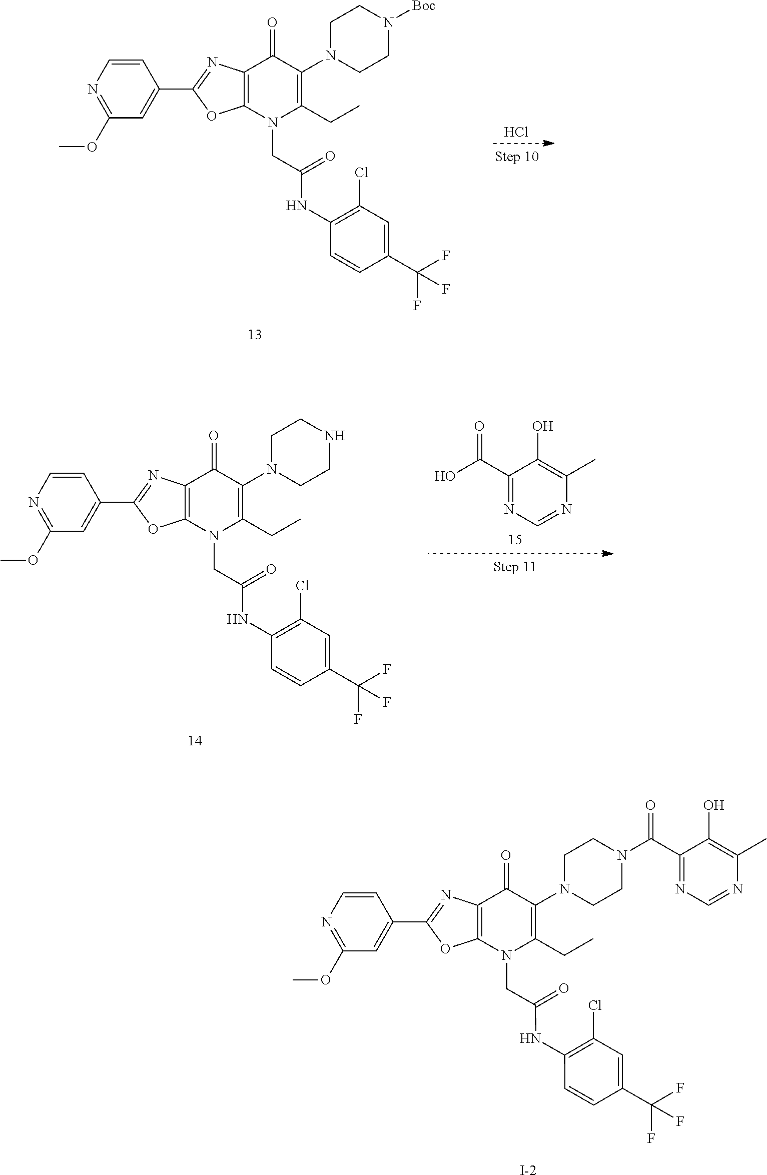

Scheme 2: General synthetic methods of producing [4,5-b]pyridin-4(7H) compounds of the disclosure

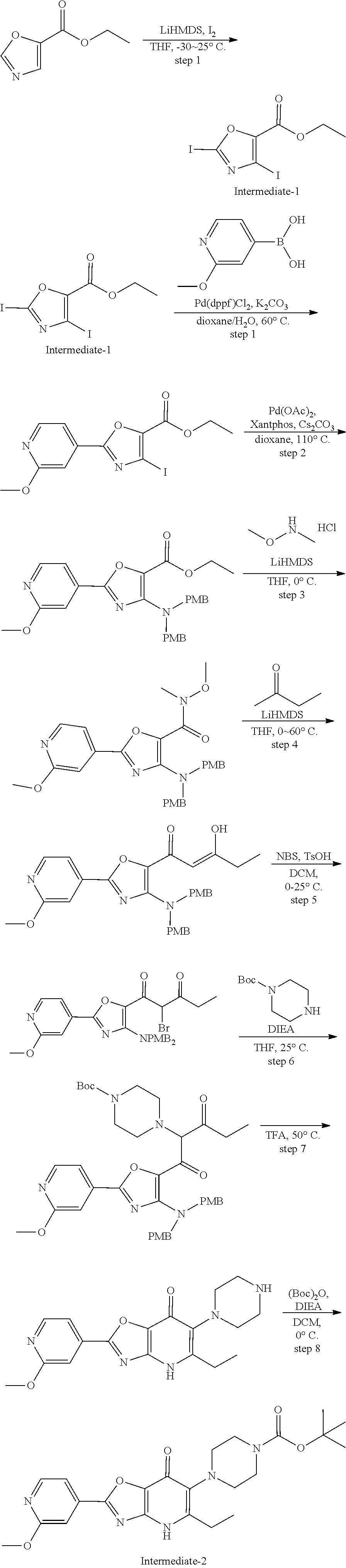

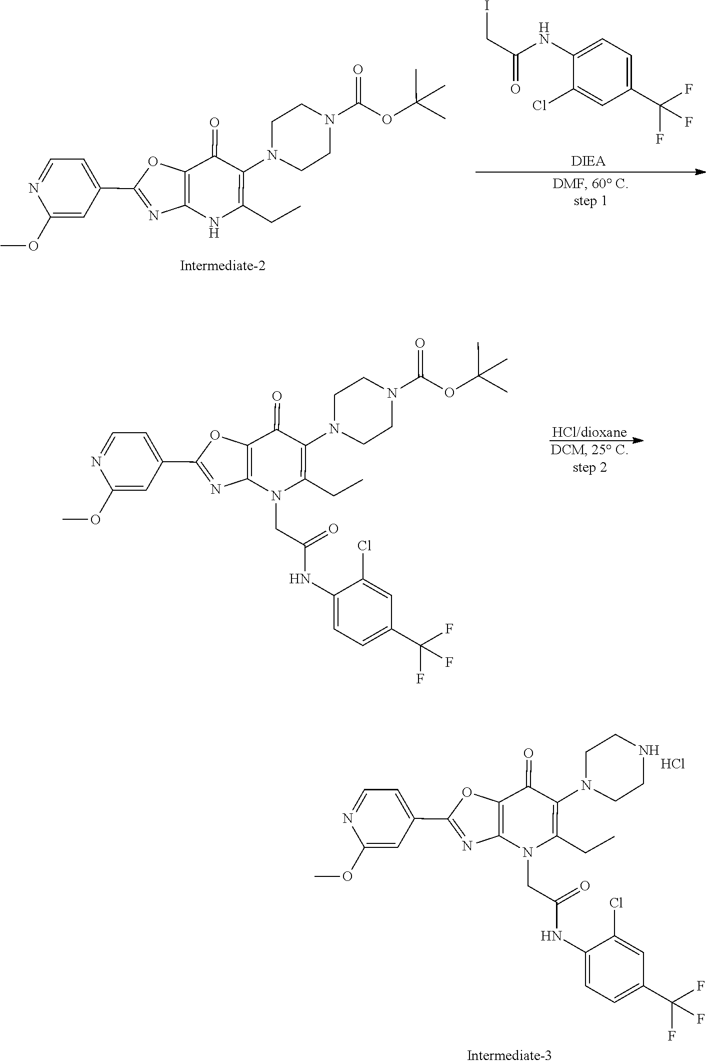

Those having ordinary skill in the art will be able to adapt such synthetic procedures to afford variably substituted compounds of Formula I for synthesis of the compounds of the disclosure.

### EXAMPLES

As depicted in the Examples below, in certain exemplary embodiments, compounds are prepared according to the procedures provided herein. It will be appreciated that, although the methods depict the synthesis of certain compounds of the present disclosure, the methods, and other methods known to one of ordinary skill in the art, can be applied to all compounds and subclasses and species of each of these compounds, as described herein.

### List of Abbreviations

H₂O: water
ACN: acetonitrile
THF: tetrahydrofuran
FA: formic acid
Na₂SO₄: sodium sulfate
EtOAc: ethyl acetate
HCl: hydrochloric acid
DCM: dichloromethane
pH: potential of hydrogen
(Boc)₂O: di-tert-butyl dicarbonate
POCl₃: phosphorus oxychloride
EDCI: N-(3-dimethylaminopropyl)-N′-ethylcarbodiimide hydrochloride
LCMS: Liquid chromatography-mass spectrometry
HPLC: High-performance liquid chromatography
NH₄Cl: ammonium chloride
K₃PO₄: tripotassium phosphate
TFA: trifluoroacetic acid
NBS: N-bromosuccinimide
DIEA: N,N-diisopropylethylamine
Pd(dppf)₂Cl₂—CH₂Cl₂:1,1′-Bis(diphenylphosphino) ferrocene-palladium (II) dichloride
dichloromethane complex
TsOH: 4-Methylbenzenesulfonic acid
DMSO-d₆: deuterated dimethyl sulfoxide
NMP: N-methyl-2-pyrrolidone
DBU: 1,8-Diazabicyclo[5.4.0]undec-7-ene
LiHMDS: Lithium hexamethyldisilazide
Prep-TLC: preparative thin layer chromatography
s: singlet
m: multiplet
d: doublet
dq: doublet of quartets
t: triplet
br: broad
N: normality
eq: equivalent
M: molar concentration
PE: Petroleum ether
aq.: aqueous
SOCl₂: thionyl chloride
MTBE: methyl tert-butyl ether
KI: potassium iodide
PMB-Cl: 4-methoxybenzyl chloride
t-BuOK: potassium t-butoxide
Cs₂CO₃: cesium carbonate
DMF: N, N-dimethylformamide
N₂: nitrogen
POCl₃: phosphoryl chloride
Na₂SO₄: sodium sulfate
CDCl₃: deuterated chloroform
CD₃OD: deuterated methanol

### Example 1: Synthesis of Compound of the Disclosure

### Synthesis of N-(2-chloro-4-(trifluoromethyl)phenyl)-2-(5-ethyl-6-(4-(5-hydroxy-6-methylpyrimidine-4-carbonyl)piperazin-1-yl)-2-(2-methoxypyridin-4-yl)-7-oxooxazolo[4,5-b]pyridin-4(7H)-yl)acetamide (I-1)

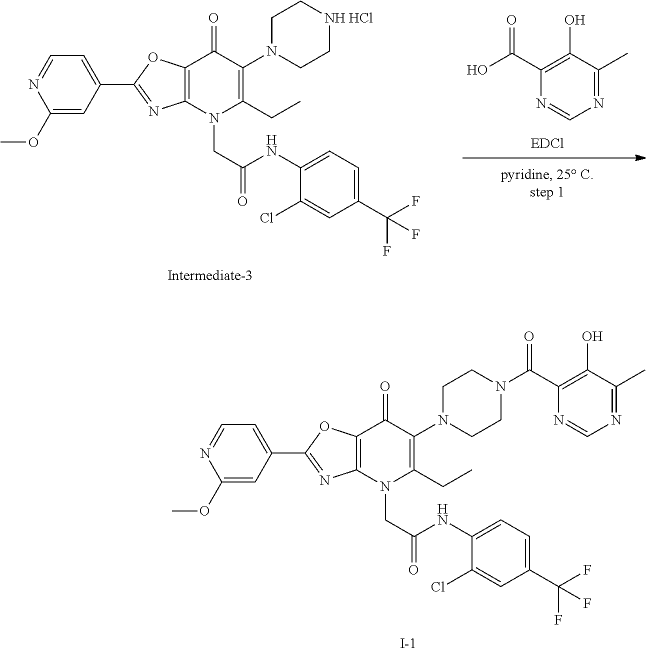

Intermediate-3 and other intermediates in the following synthetic procedures may be synthesized as described infra.

### Step 12. Synthesis of N-(2-chloro-4-(trifluoromethyl)phenyl)-2-(5-ethyl-6-(4-(5-hydroxy-6-methylpyrimidine-4-carbonyl)piperazin-1-yl)-2-(2-methoxypyridin-4-yl)-7-oxooxazolo[4,5-b]pyridin-4(7H)-yl)acetamide

To a solution of N-[2-chloro-4-(trifluoromethyl)phenyl]-2-[5-ethyl-2-(2-methoxy-4-pyridyl)-7-oxo-6-piperazin-1-yloxazolo[4,5-b]pyridin-4-yl]acetamide (18 mg, 29 μmol, 1 eq, HCl salt) and 5-hydroxy-6-methyl-pyrimidine-4-carboxylic acid (7 mg, 43 μmol, 1.5 eq) in pyridine (0.4 mL) was added EDCI (11 mg, 57 μmol, 2 eq). The mixture was stirred at 25° C. for 4 h. The reaction mixture was quenched with H₂O (10 mL). Then it was extracted with EtOAc (10 mL*3). The combined organic phase was washed with brine (15 mL*2), dried over anhydrous Na₂SO₄, filtered and concentrated in vacuum to give a residue. The residue was purified by Prep-TLC (SiO₂, DCM:MeOH=10:1) to afford the title compound as a white solid. ¹H NMR (400 MHz, CDCl₃) δ ppm 11.82 (s, 1H), 8.58 (s, 1H), 8.51 (d, 1H), 8.47 (s, 1H), 8.34 (d, 1H), 7.63 (d, 1H), 7.60 (d, 1H), 7.55 (d, 1H), 7.49 (s, 1H), 5.69-5.55 (m, 1H), 5.24 (s, 2H), 4.84-4.74 (m, 1H), 4.06-4.02 (m, 2H), 4.00 (s, 3H), 3.51-3.53 (m, 1H), 3.22-3.18 (q, 2H), 3.06-3.02 (m, 1H), 2.91-2.73 (m, 2H), 2.57 (s, 3H), 1.32 (t, 3H). LCMS: 727.2 [M+H]⁺.

### Synthesis of N-(2-chloro-4-(trifluoromethyl)phenyl)-2-(5-ethyl-6-(4-(5-hydroxy-6-methylpyrimidine-4-carbonyl)piperazin-1-yl)-2-(2-methoxypyridin-4-yl)-7-oxooxazolo[5,4-b]pyridin-4(7H)-yl)acetamide (I-2)

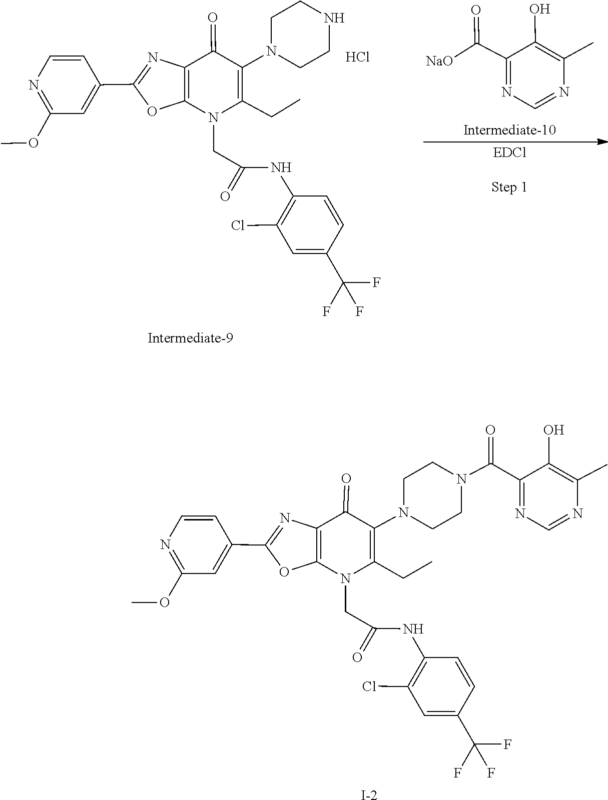

### Step 1. Synthesis of N-(2-chloro-4-(trifluoromethyl)phenyl)-2-(5-ethyl-6-(4-(5-hydroxy-6-methylpyrimidine-4-carbonyl)piperazin-1-yl)-2-(2-methoxypyridin-4-yl)-7-oxooxazolo[5,4-b]pyridin-4(7H)-yl)acetamide

To a solution of N-(2-chloro-4-(trifluoromethyl)phenyl)-2-(5-ethyl-2-(2-methoxypyridin-4-yl)-7-oxo-6-(piperazin-1-yl)oxazolo[5,4-b]pyridin-4(7H)-yl)acetamide hydrochloride (Intermediate-9) (45 mg, 72 μmol, 1 eq) and sodium 5-hydroxy-6-methylpyrimidine-4-carboxylate (Intermediate-10) (71 mg, 0.36 mmol, 5 eq) in pyridine (0.5 mL) was added EDCI (55 mg, 0.29 mmol, 4 eq). The reaction mixture was stirred at 25° C. for 1 h. The reaction mixture was poured into H₂O (10 mL) and was extracted with DCM (10 mL*3). The combined organic layers were washed with brine (10 mL), dried with anhydrous Na₂SO₄, filtered and concentrated under reduced pressure to give a residue. The residue was purified by Prep-TLC (SiO₂, DCM:MeOH=5:1) to afford the title compound.

¹H-NMR (400 MHz, CD₃OD) δ ppm 8.27 (d, 1H), 8.14 (d, 1H), 8.07 (s, 1H), 7.82 (s, 1H), 7.67-7.52 (m, 2H), 7.39 (s, 1H), 5-66-5.24 (m, 2H), 4.66-4.65 (m, 2H), 3.95 (s, 3H), 3.92-3.70 (m, 2H), 3.53-3.38 (m, 1H), 3.15 (q, 2H), 3.10-3.02 (m, 1H), 2.90-2.79 (m, 1H), 2.75-2.62 (m, 1H), 2.40 (s, 3H), 1.29 (t, 3H).

LCMS: 727.3 [M+H]⁺.

### Synthesis of 2-(5-ethyl-6-(4-(5-hydroxy-6-methylpyrimidine-4-carbonyl)piperazin-1-yl)-2-(2-methoxypyridin-4-yl)-7-oxooxazolo[4,5-b]pyridin-4(7H)-yl)-N-(3-(trifluoromethyl)bicyclo[1.1.1]pentan-1-yl)acetamide (I-3)

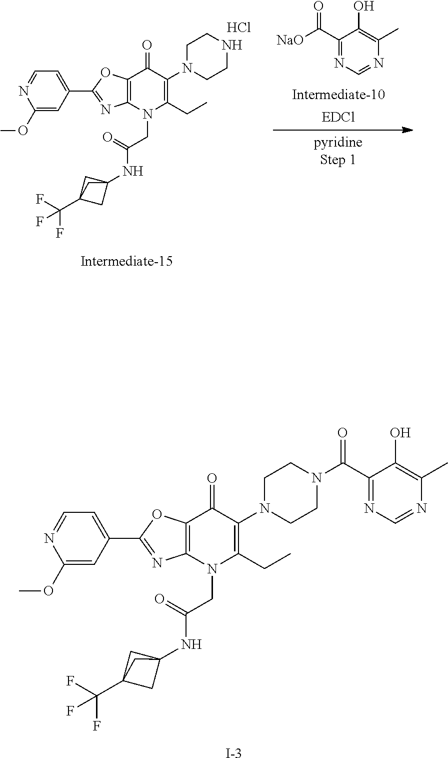

### Step 5. Synthesis of 2-(5-ethyl-6-(4-(5-hydroxy-6-methylpyrimidine-4-carbonyl)piperazin-1-yl)-2-(2-methoxypyridin-4-yl)-7-oxooxazolo[4,5-b]pyridin-4(7H)-yl)-N-(3-(trifluoromethyl)bicyclo[1.1.1]pentan-1-yl)acetamide

To a solution of 2-[5-ethyl-2-(2-methoxy-4-pyridyl)-7-oxo-6-piperazin-1-yl-oxazolo[4,5-b]pyridin-4-yl]-N-[3-(trifluoromethyl)-1 bicyclo[1.1.1]pentanyl]acetamide hydrochloride (Intermediate-15) (80 mg, 137 μmol, 1 eq) and sodium 5-hydroxy-6-methylpyrimidine-4-carboxylate (Intermediate-10) (54 mg, 274 μmol, 2 eq) in pyridine (1 mL) was added EDCI (79 mg, 412 μmol, 3 eq). The mixture was stirred at 25° C. for 4 h. To the reaction mixture was added H₂O (10 mL). Then the mixture was extracted with EtOAc (10 mL*3). The combined organic phase was washed with brine (15 mL*2), dried over anhydrous Na₂SO₄, filtered and concentrated in vacuum to give a residue. The residue was purified by Prep-TLC (SiO₂, DCM:MeOH=10:1) to afford the title compound.

¹H NMR (400 MHz, CDCl₃) δ ppm 11.82 (s, 1H), 8.57 (s, 1H), 8.31 (d, 1H), 7.54 (d, 1H), 7.43 (s, 1H), 6.81 (s, 1H), 5.63-5.60 (m, 1H), 5.00 (s, 2H), 4.84-4.73 (m, 1H), 4.04 (q, 2H), 3.99 (s, 3H), 3.55-3.42 (m, 1H), 3.04-3.02 (m, 3H), 2.89-2.75 (m, 2H), 2.57 (s, 3H), 2.34 (s, 6H), 1.26 (t, 3H).

LCMS: 683.2 [M+H]⁺.

### Synthesis of 2-(5-ethyl-6-(4-(5-hydroxy-6-methylpyrimidine-4-carbonyl)piperazin-1-yl)-2-(2-methoxypyridin-4-yl)-7-oxooxazolo[5,4-b]pyridin-4(7H)-yl)-N-(3-(trifluoromethyl)bicyclo[1.1.1]pentan-1-yl)acetamide (I-4)

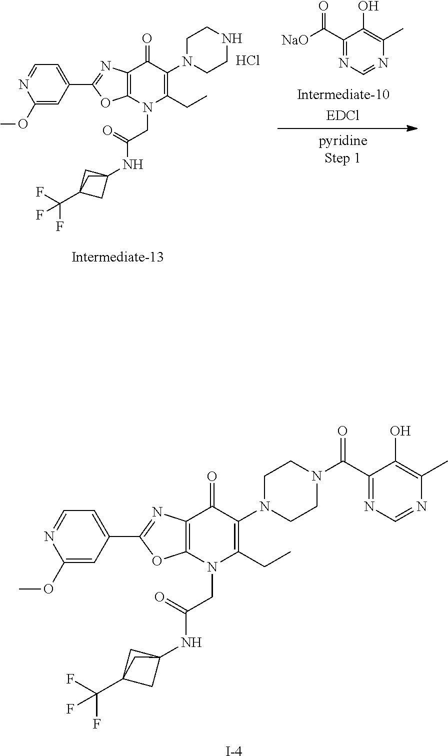

### Step 3. Synthesis of 2-(5-ethyl-6-(4-(5-hydroxy-6-methylpyrimidine-4-carbonyl)piperazin-1-yl)-2-(2-methoxypyridin-4-yl)-7-oxooxazolo[5,4-b]pyridin-4(7H)-yl)-N-(3-(trifluoromethyl)bicyclo[1.1.1]pentan-1-yl)acetamide

To a solution of 2-[5-ethyl-2-(2-methoxy-4-pyridyl)-7-oxo-6-piperazin-1-yl-oxazolo[5,4-b]pyridin-4-yl]-N-[3-(trifluoromethyl)-1-bicyclo[1.1.1]pentanyl]acetamide hydrochloride (Intermediate-13) (20 mg, 34 μmol, 1 eq) and sodium 5-hydroxy-6-methylpyrimidine-4-carboxylate (Intermediate-10) (34 mg, 172 μmol, 5 eq) in pyridine (0.2 mL) was added EDCI (26 mg, 137 μmol, 4 eq). The reaction mixture was stirred at 25° C. for 12 h. The reaction mixture was poured into water (10 mL) and extracted with DCM (10 mL*3). The combined organic layer was washed with brine (10 mL), dried over anhydrous Na₂SO₄, filtered and concentrated under reduced pressure to give a residue. The residue was purified by prep-TLC (SiO₂, MeOH/DCM=10:1) to afford the title compound.

¹H NMR (400 MHz, CD₃OD) δ ppm 8.29 (d, 1H), 8.14 (s, 1H), 7.57 (d, 1H), 7.40 (s, 1H), 5.07 (s, 2H), 4.66-4.63 (m, 1H), 3.97 (s, 3H), 3.94-3.79 (m, 2H), 3.72-3.59 (m, 1H), 3.48-3.44 (m, 1H), 3.19-3.08 (m, 1H), 3.04 (q, 2H), 2.-86-2.82 (m, 1H), 2.70-2.66 (m, 1H), 2.42 (s, 3H), 2.32 (s, 6H), 1.24 (t, 3H).

LCMS: 683.3 [M+H]⁺.

TABLE 2

| | | | (Mass_ | |
|---|---|---|---|---|
| | | | Observed) | (Retention_ |
| No. | | Method | [g/mol] | time) [min] |
| I-1 | 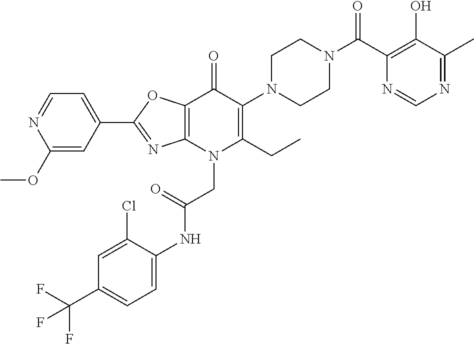 | LCMS 1 | 727.2 | 0.43 |
| I-2 | 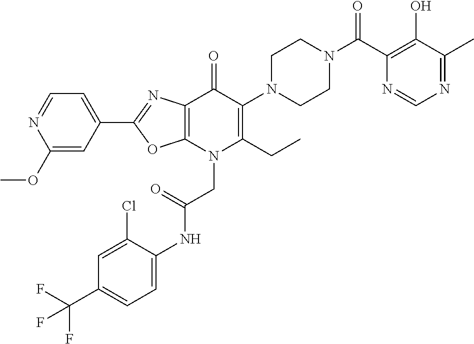 | LCMS 1 | 727.3 | 0.553 |
| I-3 | 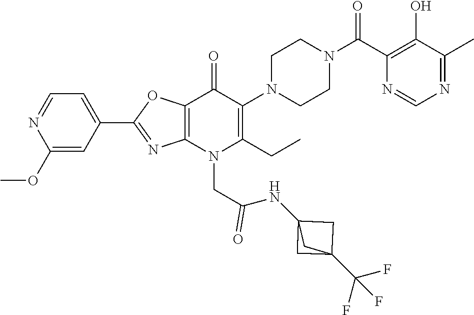 | LCMS 2 | 683.2 | 0.53 |
| I-4 | 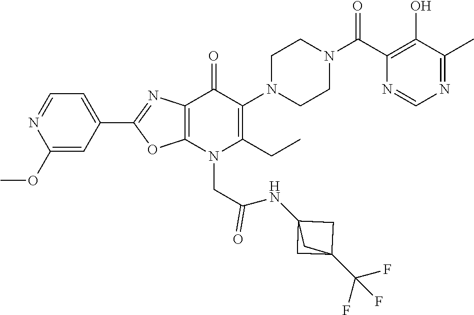 | LCMS 2 | 683.3 | 0.52 |
| I-5 | 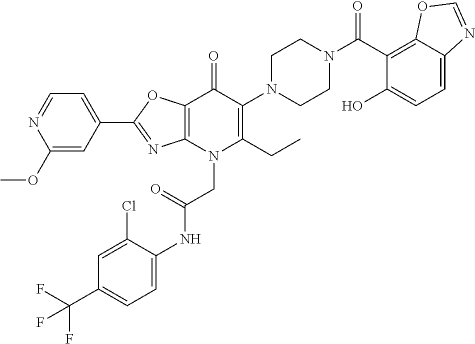 [SMILES: CCc1c(N2CCN(C(=O)c3c(O)ccc4ncoc34)CC2)c(=O)c2oc(-c3ccnc(OC)c3)nc2n1CC(=O)Nc1ccc(C(F)(F)F)cc1Cl] | LCMS 3 | 752.3 | 4.57 |

As can be appreciated by those skilled in the art, compounds of Table 2a may be accessed through Intermediate 1 (ethyl 2,4-diiodooxazole-5-carboxylate).

TABLE 2a

| | | Mass_ |
|---|---|---|
| | | Theoretical |
| No. | | [g/mol] |
| I-2a | 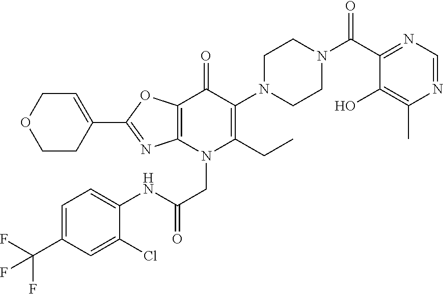 [SMILES: CCc1c(N2CCN(C(=O)c3ncnc(C)c3O)CC2)c(=O)c2oc(C3=CCOCC3)nc2n1CC(=O)Nc1ccc(C(F)(F)F)cc1Cl] | 701.20 |
| I-3a | 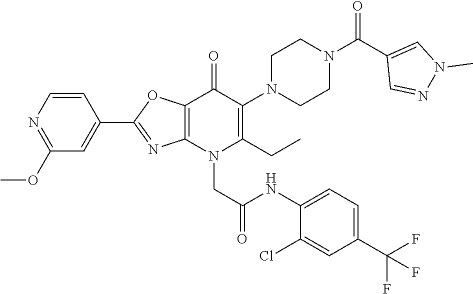 | 698.20 |
| I-4a | 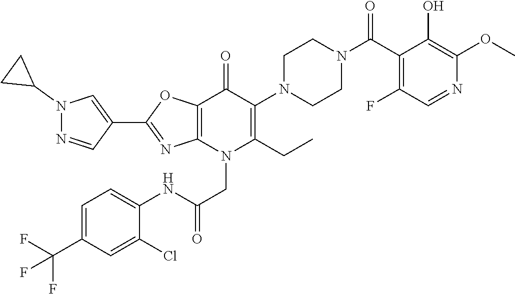 [SMILES: CCc1c(N2CCN(C(=O)c3c(F)cnc(OC)c3O)CC2)c(=O)c2oc(-c3cnn(C4CC4)c3)nc2n1CC(=O)Nc1ccc(C(F)(F)F)cc1Cl] | 758.20 |
| I-5a | 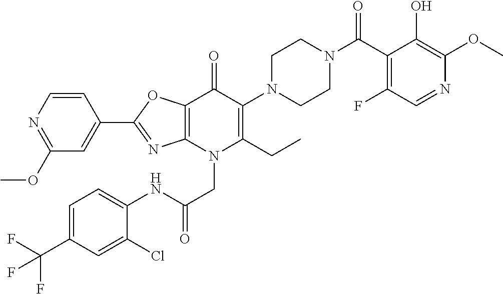 | 759.18 |
| I-6a | 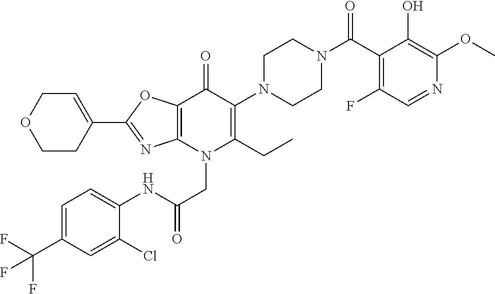 [SMILES: CCc1c(N2CCN(C(=O)c3c(F)cnc(OC)c3O)CC2)c(=O)c2oc(C3=CCOCC3)nc2n1CC(=O)Nc1ccc(C(F)(F)F)cc1Cl] | 734.19 |
| I-7a | 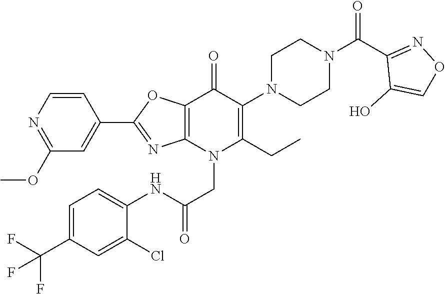 [SMILES: CCc1c(N2CCN(C(=O)c3nocc3O)CC2)c(=O)c2oc(-c3ccnc(OC)c3)nc2n1CC(=O)Nc1ccc(C(F)(F)F)cc1Cl] | 701.16 |
| I-8a | 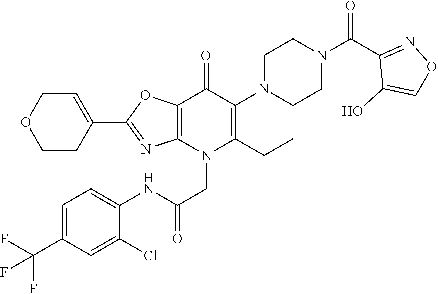 [SMILES: CCc1c(N2CCN(C(=O)c3nocc3O)CC2)c(=O)c2oc(C3=CCOCC3)nc2n1CC(=O)Nc1ccc(C(F)(F)F)cc1Cl] | 676.17 |
| I-9a | 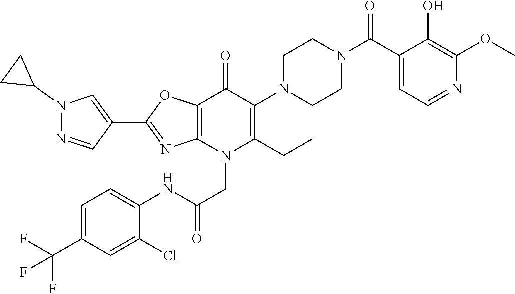 [SMILES: CCc1c(N2CCN(C(=O)c3ccnc(OC)c3O)CC2)c(=O)c2oc(-c3cnn(C4CC4)c3)nc2n1CC(=O)Nc1ccc(C(F)(F)F)cc1Cl] | 740.21 |
| I-10a | 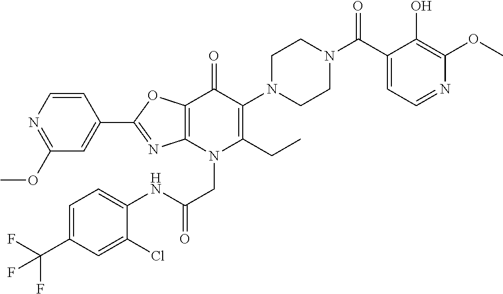 | 741.19 |
| I-11a | 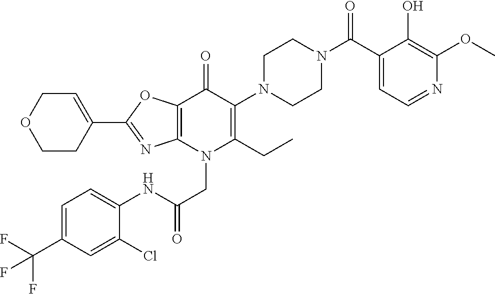 [SMILES: CCc1c(N2CCN(C(=O)c3ccnc(OC)c3O)CC2)c(=O)c2oc(C3=CCOCC3)nc2n1CC(=O)Nc1ccc(C(F)(F)F)cc1Cl] | 716.20 |
| I-12a | 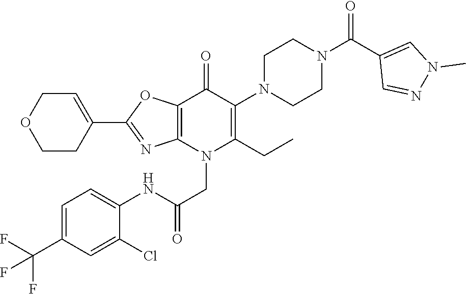 [SMILES: CCc1c(N2CCN(C(=O)c3cnn(C)c3)CC2)c(=O)c2oc(C3=CCOCC3)nc2n1CC(=O)Nc1ccc(C(F)(F)F)cc1Cl] | 673.20 |
| I-13a | 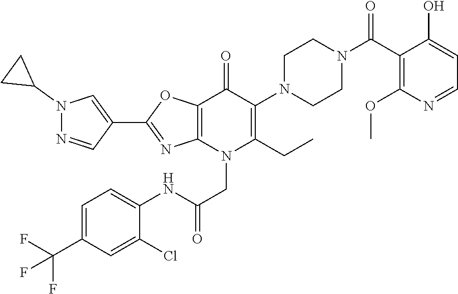 [SMILES: CCc1c(N2CCN(C(=O)c3c(O)ccnc3OC)CC2)c(=O)c2oc(-c3cnn(C4CC4)c3)nc2n1CC(=O)Nc1ccc(C(F)(F)F)cc1Cl] | 740.21 |
| I-14a | 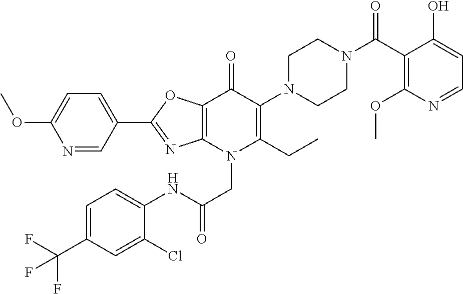 [SMILES: CCc1c(N2CCN(C(=O)c3c(O)ccnc3OC)CC2)c(=O)c2oc(-c3ccc(OC)nc3)nc2n1CC(=O)Nc1ccc(C(F)(F)F)cc1Cl] | 741.19 |
| I-15a | 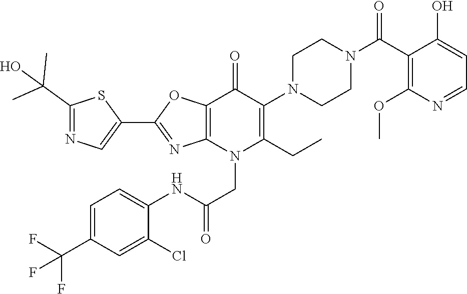 [SMILES: CCc1c(N2CCN(C(=O)c3c(O)ccnc3OC)CC2)c(=O)c2oc(-c3cnc(C(C)(C)O)s3)nc2n1CC(=O)Nc1ccc(C(F)(F)F)cc1Cl] | 775.18 |
| I-16a | 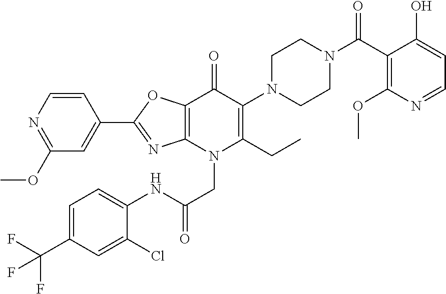 [SMILES: CCc1c(N2CCN(C(=O)c3c(O)ccnc3OC)CC2)c(=O)c2oc(-c3ccnc(OC)c3)nc2n1CC(=O)Nc1ccc(C(F)(F)F)cc1Cl] | 741.19 |
| I-17a | 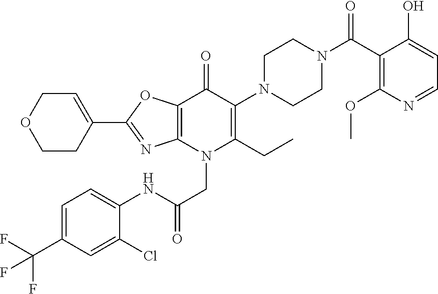 [SMILES: CCc1c(N2CCN(C(=O)c3c(O)ccnc3OC)CC2)c(=O)c2oc(C3=CCOCC3)nc2n1CC(=O)Nc1ccc(C(F)(F)F)cc1Cl] | 716.20 |
| I-18a | 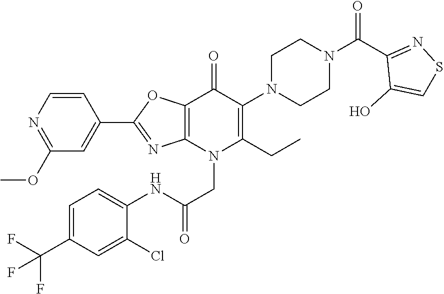 [SMILES: CCc1c(N2CCN(C(=O)c3nscc3O)CC2)c(=O)c2oc(-c3ccnc(OC)c3)nc2n1CC(=O)Nc1ccc(C(F)(F)F)cc1Cl] | 717.14 |
| I-19a | 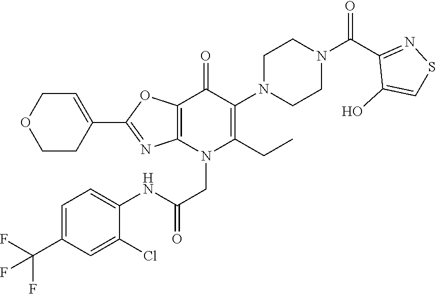 [SMILES: CCc1c(N2CCN(C(=O)c3nscc3O)CC2)c(=O)c2oc(C3=CCOCC3)nc2n1CC(=O)Nc1ccc(C(F)(F)F)cc1Cl] | 692.14 |
| I-20a | 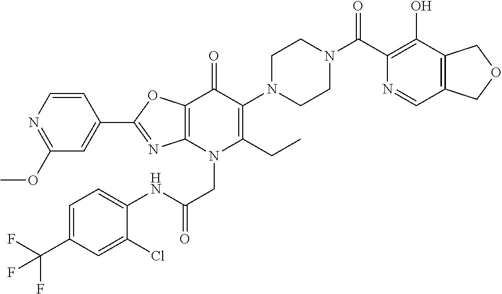 [SMILES: CCc1c(N2CCN(C(=O)c3ncc4c(c3O)COC4)CC2)c(=O)c2oc(-c3ccnc(OC)c3)nc2n1CC(=O)Nc1ccc(C(F)(F)F)cc1Cl] | 753.19 |
| I-21a | 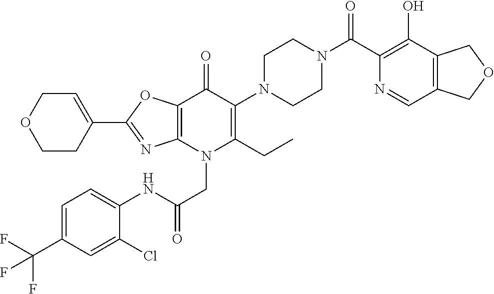 [SMILES: CCc1c(N2CCN(C(=O)c3ncc4c(c3O)COC4)CC2)c(=O)c2oc(C3=CCOCC3)nc2n1CC(=O)Nc1ccc(C(F)(F)F)cc1Cl] | 728.20 |
| I-22a | 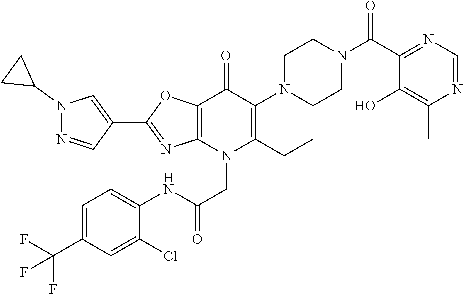 [SMILES: CCc1c(N2CCN(C(=O)c3ncnc(C)c3O)CC2)c(=O)c2oc(-c3cnn(C4CC4)c3)nc2n1CC(=O)Nc1ccc(C(F)(F)F)cc1Cl] | 725.21 |
| I-23a | 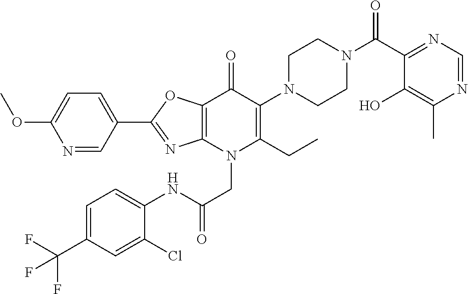 [SMILES: CCc1c(N2CCN(C(=O)c3ncnc(C)c3O)CC2)c(=O)c2oc(-c3ccc(OC)nc3)nc2n1CC(=O)Nc1ccc(C(F)(F)F)cc1Cl] | 726.19 |
| I-24a | 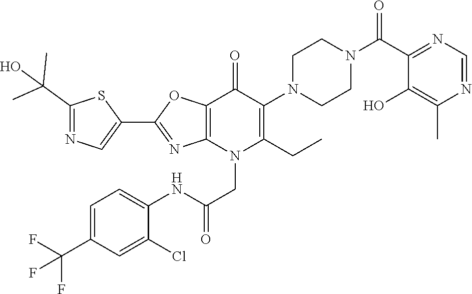 [SMILES: CCc1c(N2CCN(C(=O)c3ncnc(C)c3O)CC2)c(=O)c2oc(-c3cnc(C(C)(C)O)s3)nc2n1CC(=O)Nc1ccc(C(F)(F)F)cc1Cl] | 760.18 |
| I-25a | 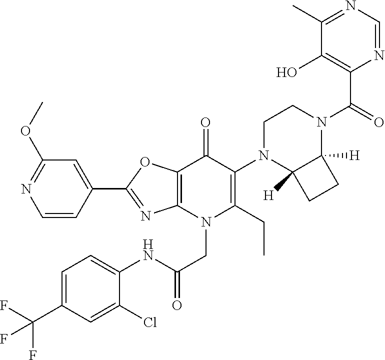 [SMILES: CCc1c(N2CCN(C(=O)c3ncnc(C)c3O)[C@H]3CC[C@@H]32)c(=O)c2oc(-c3ccnc(OC)c3)nc2n1CC(=O)Nc1ccc(C(F)(F)F)cc1Cl] | 752.21 |
| I-26a | 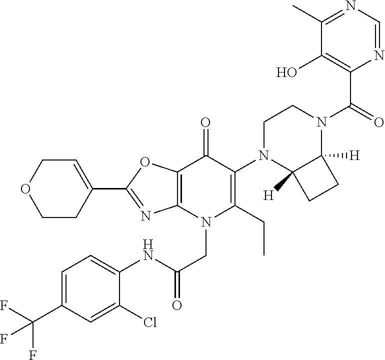 [SMILES: CCc1c(N2CCN(C(=O)c3ncnc(C)c3O)[C@H]3CC[C@@H]32)c(=O)c2oc(C3=CCOCC3)nc2n1CC(=O)Nc1ccc(C(F)(F)F)cc1Cl] | 727.21 |
| I-27a | 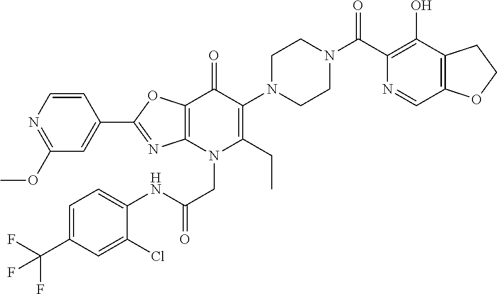 [SMILES: CCc1c(N2CCN(C(=O)c3ncc4c(c3O)CCO4)CC2)c(=O)c2oc(-c3ccnc(OC)c3)nc2n1CC(=O)Nc1ccc(C(F)(F)F)cc1Cl] | 753.19 |
| I-28a | 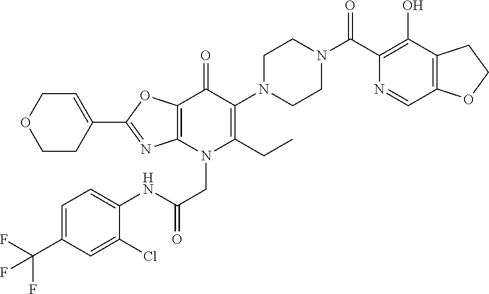 [SMILES: CCc1c(N2CCN(C(=O)c3ncc4c(c3O)CCO4)CC2)c(=O)c2oc(C3=CCOCC3)nc2n1CC(=O)Nc1ccc(C(F)(F)F)cc1Cl] | 728.20 |
| I-29a | 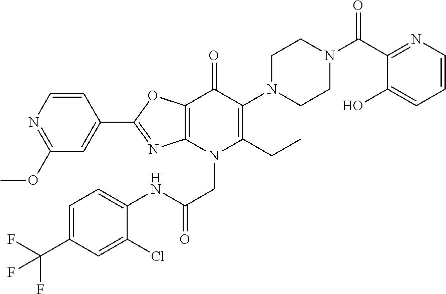 [SMILES: CCc1c(N2CCN(C(=O)c3ncccc3O)CC2)c(=O)c2oc(-c3ccnc(OC)c3)nc2n1CC(=O)Nc1ccc(C(F)(F)F)cc1Cl] | 711.18 |
| I-30a | 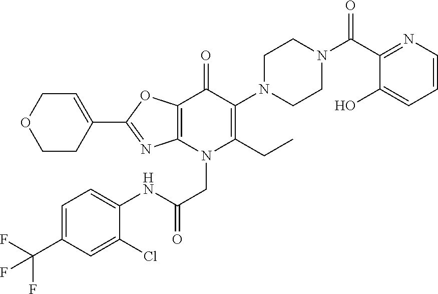 [SMILES: CCc1c(N2CCN(C(=O)c3ncccc3O)CC2)c(=O)c2oc(C3=CCOCC3)nc2n1CC(=O)Nc1ccc(C(F)(F)F)cc1Cl] | 686.19 |
| I-31a | 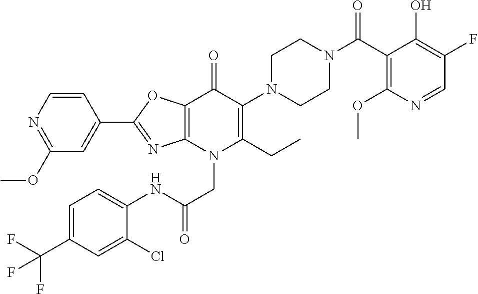 [SMILES: CCc1c(N2CCN(C(=O)c3c(OC)ncc(F)c3O)CC2)c(=O)c2oc(-c3ccnc(OC)c3)nc2n1CC(=O)Nc1ccc(C(F)(F)F)cc1Cl] | |
| I-32a | 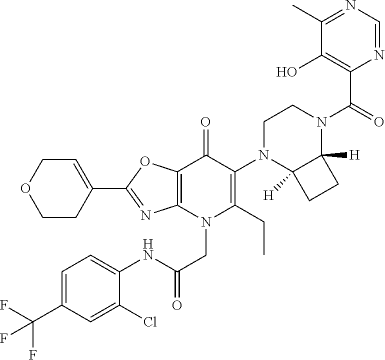 [SMILES: CCc1c(N2CCN(C(=O)c3ncnc(C)c3O)[C@@H]3CC[C@H]32)c(=O)c2oc(C3=CCOCC3)nc2n1CC(=O)Nc1ccc(C(F)(F)F)cc1Cl] | 727.21 |
| I-33a | 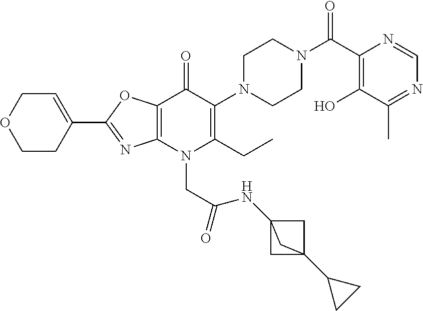 [SMILES: CCc1c(N2CCN(C(=O)c3ncnc(C)c3O)CC2)c(=O)c2oc(C3=CCOCC3)nc2n1CC(=O)NC12CC(C3CC3)(C1)C2] | 629.30 |
| I-34a | 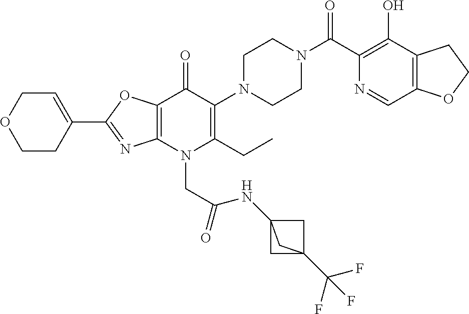 [SMILES: CCc1c(N2CCN(C(=O)c3ncc4c(c3O)CCO4)CC2)c(=O)c2oc(C3=CCOCC3)nc2n1CC(=O)NC12CC(C(F)(F)F)(C1)C2] | 684.25 |
| I-35a | 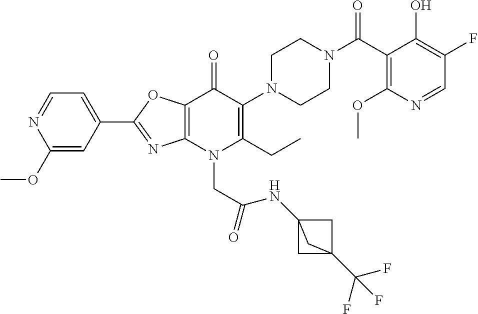 [SMILES: CCc1c(N2CCN(C(=O)c3c(OC)ncc(F)c3O)CC2)c(=O)c2oc(-c3ccnc(OC)c3)nc2n1CC(=O)NC12CC(C(F)(F)F)(C1)C2] | 715.24 |
| I-36a | 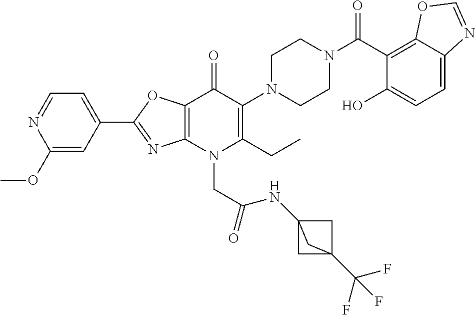 [SMILES: CCc1c(N2CCN(C(=O)c3c(O)ccc4ncoc34)CC2)c(=O)c2oc(-c3ccnc(OC)c3)nc2n1CC(=O)NC12CC(C(F)(F)F)(C1)C2] | 707.23 |
| I-37a | 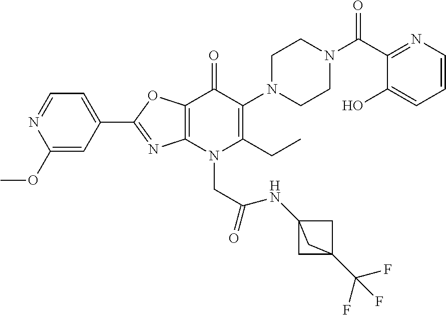 [SMILES: CCc1c(N2CCN(C(=O)c3ncccc3O)CC2)c(=O)c2oc(-c3ccnc(OC)c3)nc2n1CC(=O)NC12CC(C(F)(F)F)(C1)C2] | 667.24 |
| I-38a | 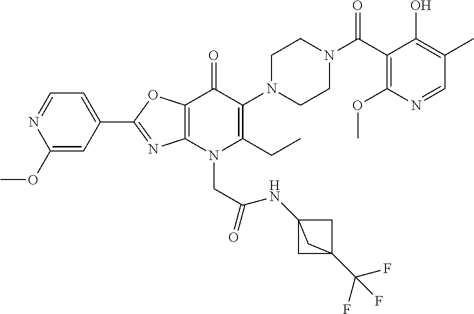 | 711.26 |
| I-39a | 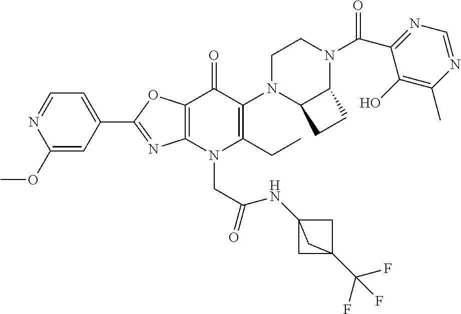 [SMILES: CCc1c(N2CCN(C(=O)c3ncnc(C)c3O)[C@@H]3CC[C@H]32)c(=O)c2oc(-c3ccnc(OC)c3)nc2n1CC(=O)NC12CC(C(F)(F)F)(C1)C2] | 708.26 |
| I-40a | 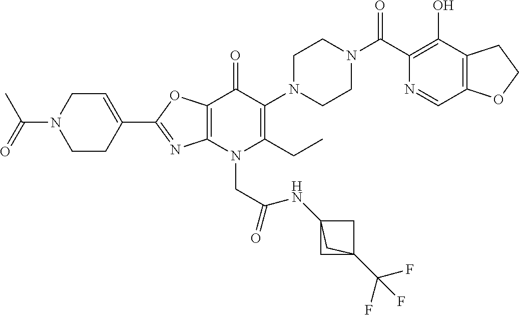 [SMILES: CCc1c(N2CCN(C(=O)c3ncc4c(c3O)CCO4)CC2)c(=O)c2oc(C3=CCN(C(C)=O)CC3)nc2n1CC(=O)NC12CC(C(F)(F)F)(C1)C2] | 725.28 |
| I-41a | 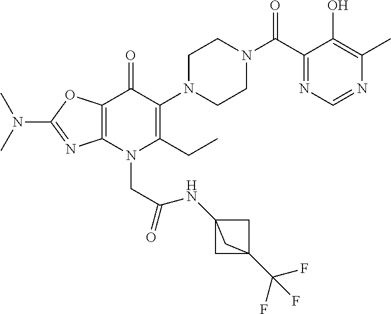 | 618.25 |
| I-42a | 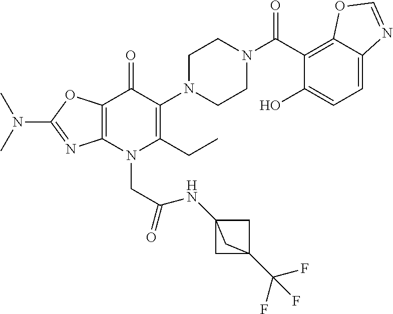 [SMILES: CCc1c(N2CCN(C(=O)c3c(O)ccc4ncoc34)CC2)c(=O)c2oc(N(C)C)nc2n1CC(=O)NC12CC(C(F)(F)F)(C1)C2] | 643.24 |
| I-43a | 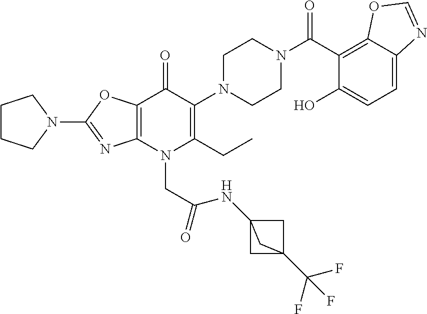 [SMILES: CCc1c(N2CCN(C(=O)c3c(O)ccc4ncoc34)CC2)c(=O)c2oc(N3CCCC3)nc2n1CC(=O)NC12CC(C(F)(F)F)(C1)C2] | 669.25 |
| I-44a | 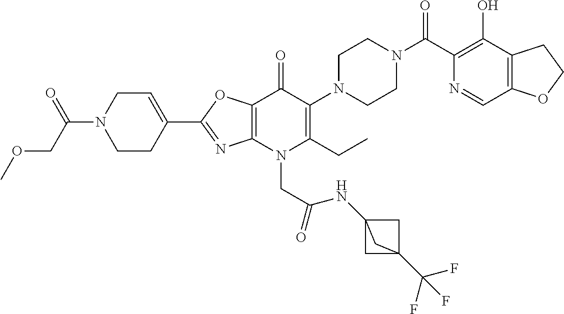 [SMILES: CCc1c(N2CCN(C(=O)c3ncc4c(c3O)CCO4)CC2)c(=O)c2oc(C3=CCN(C(=O)COC)CC3)nc2n1CC(=O)NC12CC(C(F)(F)F)(C1)C2] | 755.29 |
| I-45a | 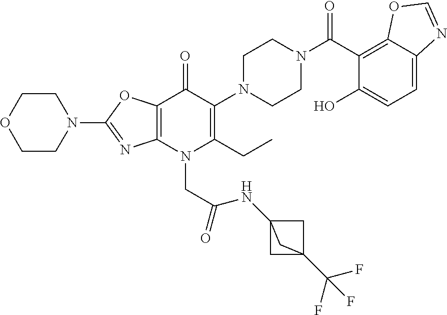 [SMILES: CCc1c(N2CCN(C(=O)c3c(O)ccc4ncoc34)CC2)c(=O)c2oc(N3CCOCC3)nc2n1CC(=O)NC12CC(C(F)(F)F)(C1)C2] | 685.25 |
| I-46a | 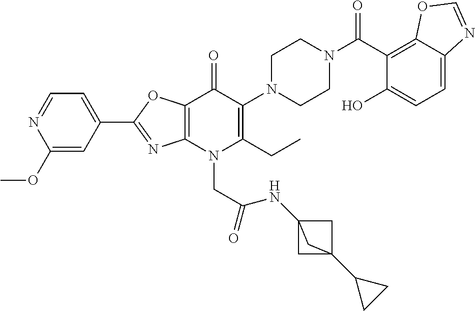 [SMILES: CCc1c(N2CCN(C(=O)c3c(O)ccc4ncoc34)CC2)c(=O)c2oc(-c3ccnc(OC)c3)nc2n1CC(=O)NC12CC(C3CC3)(C1)C2] | 679.28 |
| I-47a | 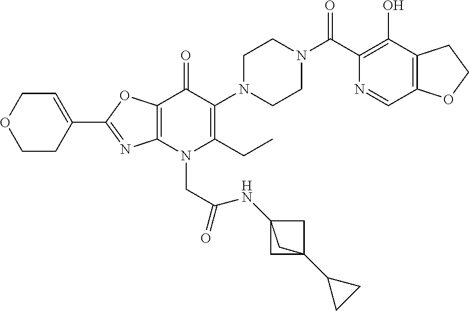 [SMILES: CCc1c(N2CCN(C(=O)c3ncc4c(c3O)CCO4)CC2)c(=O)c2oc(C3=CCOCC3)nc2n1CC(=O)NC12CC(C3CC3)(C1)C2] | |
| I-48a | 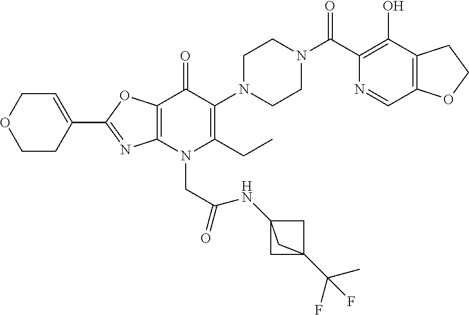 [SMILES: CCc1c(N2CCN(C(=O)c3ncc4c(c3O)CCO4)CC2)c(=O)c2oc(C3=CCOCC3)nc2n1CC(=O)NC12CC(C(C)(F)F)(C1)C2] | 680.28 |
| I-49a | 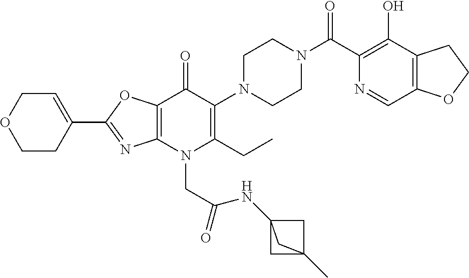 [SMILES: CCc1c(N2CCN(C(=O)c3ncc4c(c3O)CCO4)CC2)c(=O)c2oc(C3=CCOCC3)nc2n1CC(=O)NC12CC(C)(C1)C2] | 630.28 |
| I-50a | 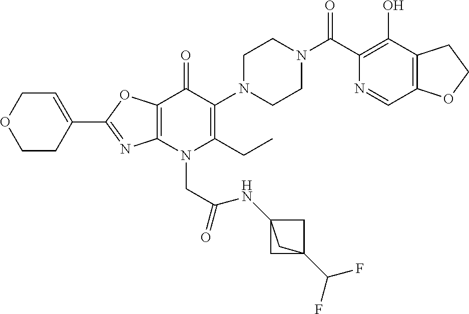 [SMILES: CCc1c(N2CCN(C(=O)c3ncc4c(c3O)CCO4)CC2)c(=O)c2oc(C3=CCOCC3)nc2n1CC(=O)NC12CC(C(F)F)(C1)C2] | 666.26 |
| I-51a | 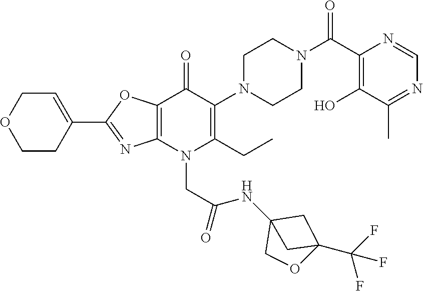 [SMILES: CCc1c(N2CCN(C(=O)c3ncnc(C)c3O)CC2)c(=O)c2oc(C3=CCOCC3)nc2n1CC(=O)NC12COC(C(F)(F)F)(C1)C2] | 673.25 |
| I-52a | 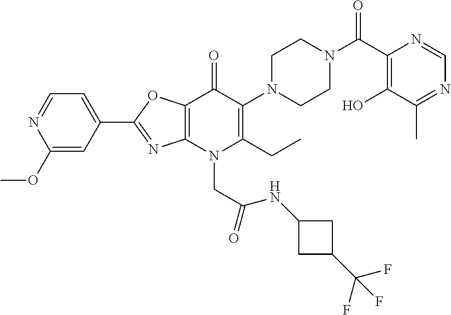 [SMILES: CCc1c(N2CCN(C(=O)c3ncnc(C)c3O)CC2)c(=O)c2oc(-c3ccnc(OC)c3)nc2n1CC(=O)NC1CC(C(F)(F)F)C1] | 670.25 |
| I-53a | 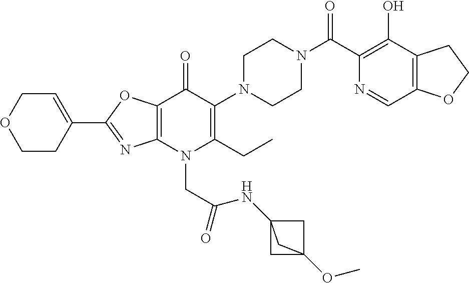 [SMILES: CCc1c(N2CCN(C(=O)c3ncc4c(c3O)CCO4)CC2)c(=O)c2oc(C3=CCOCC3)nc2n1CC(=O)NC12CC(OC)(C1)C2] | 646.28 |
| I-54a | 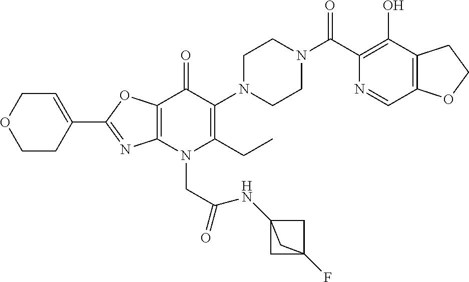 [SMILES: CCc1c(N2CCN(C(=O)c3ncc4c(c3O)CCO4)CC2)c(=O)c2oc(C3=CCOCC3)nc2n1CC(=O)NC12CC(F)(C1)C2] | 634.26 |
| I-55a | 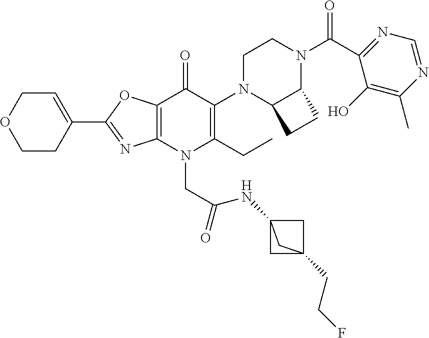 [SMILES: CCc1c(N2CCN(C(=O)c3ncnc(C)c3O)[C@@H]3CC[C@H]32)c(=O)c2oc(C3=CCOCC3)nc2n1CC(=O)N[C@]12C[C@](CCF)(C1)C2] | 661.31 |
| I-56a | 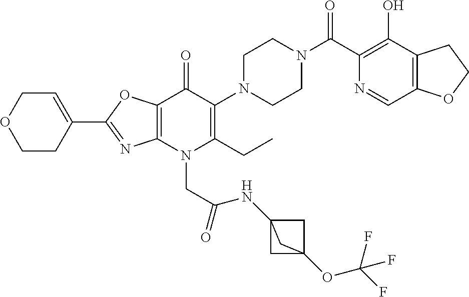 [SMILES: CCc1c(N2CCN(C(=O)c3ncc4c(c3O)CCO4)CC2)c(=O)c2oc(C3=CCOCC3)nc2n1CC(=O)NC12CC(OC(F)(F)F)(C1)C2] | 700.25 |
| I-57a | 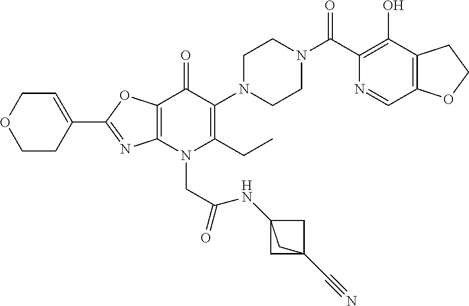 [SMILES: CCc1c(N2CCN(C(=O)c3ncc4c(c3O)CCO4)CC2)c(=O)c2oc(C3=CCOCC3)nc2n1CC(=O)NC12CC(C#N)(C1)C2] | 641.26 |
| I-58a | 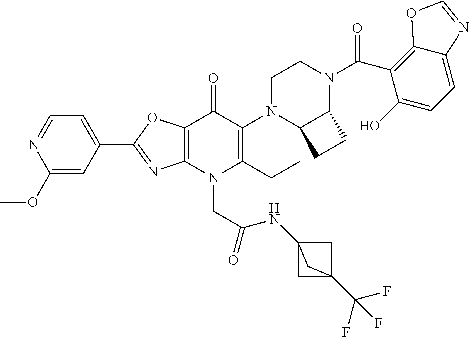 [SMILES: CCc1c(N2CCN(C(=O)c3c(O)ccc4ncoc34)[C@@H]3CC[C@H]32)c(=O)c2oc(-c3ccnc(OC)c3)nc2n1CC(=O)NC12CC(C(F)(F)F)(C1)C2] | 733.25 |
| I-59a | 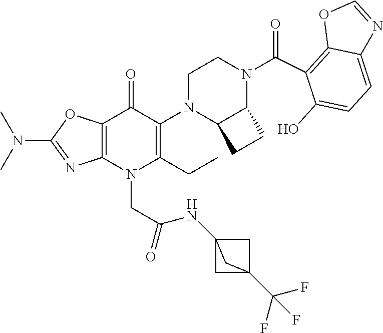 [SMILES: CCc1c(N2CCN(C(=O)c3c(O)ccc4ncoc34)[C@@H]3CC[C@H]32)c(=O)c2oc(N(C)C)nc2n1CC(=O)NC12CC(C(F)(F)F)(C1)C2] | 669.25 |
| I-60a | 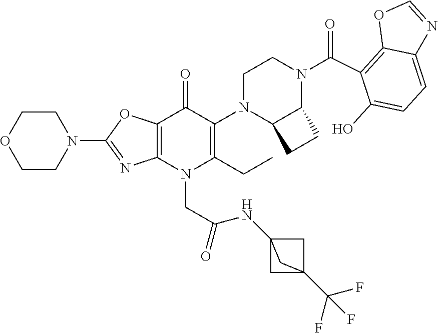 [SMILES: CCc1c(N2CCN(C(=O)c3c(O)ccc4ncoc34)[C@@H]3CC[C@H]32)c(=O)c2oc(N3CCOCC3)nc2n1CC(=O)NC12CC(C(F)(F)F)(C1)C2] | 711.26 |
| I-61a | 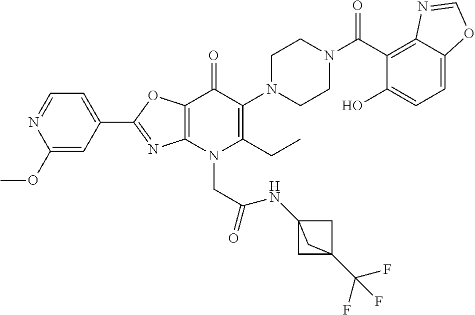 [SMILES: CCc1c(N2CCN(C(=O)c3c(O)ccc4ocnc34)CC2)c(=O)c2oc(-c3ccnc(OC)c3)nc2n1CC(=O)NC12CC(C(F)(F)F)(C1)C2] | 707.23 |
| I-62a | 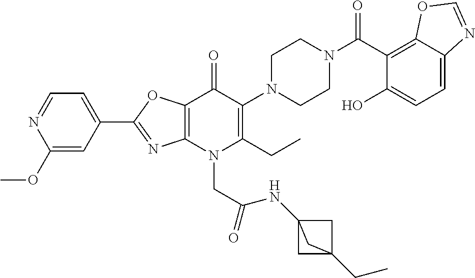 [SMILES: CCc1c(N2CCN(C(=O)c3c(O)ccc4ncoc34)CC2)c(=O)c2oc(-c3ccnc(OC)c3)nc2n1CC(=O)NC12CC(CC)(C1)C2] | 667.28 |
| I-63a | 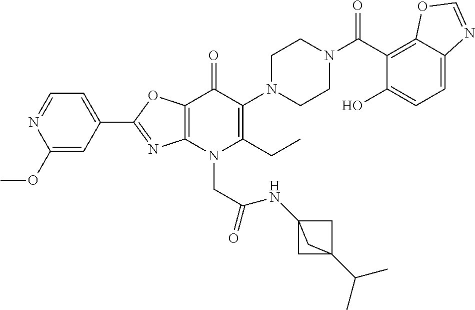 [SMILES: CCc1c(N2CCN(C(=O)c3c(O)ccc4ncoc34)CC2)c(=O)c2oc(-c3ccnc(OC)c3)nc2n1CC(=O)NC12CC(C(C)C)(C1)C2] | 681.29 |
| I-64a | 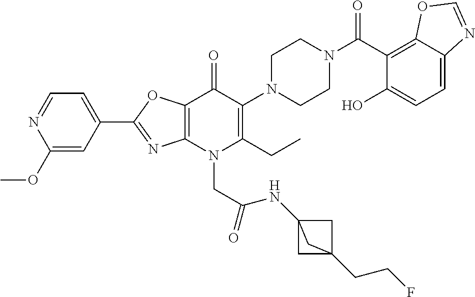 [SMILES: CCc1c(N2CCN(C(=O)c3c(O)ccc4ncoc34)CC2)c(=O)c2oc(-c3ccnc(OC)c3)nc2n1CC(=O)NC12CC(CCF)(C1)C2] | 685.27 |
| I-65a | 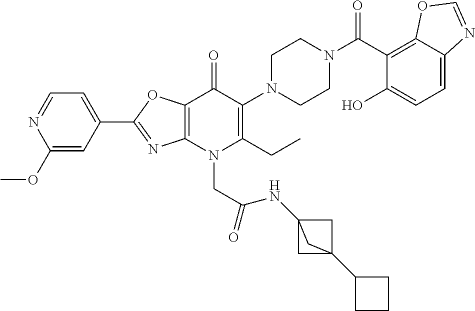 [SMILES: CCc1c(N2CCN(C(=O)c3c(O)ccc4ncoc34)CC2)c(=O)c2oc(-c3ccnc(OC)c3)nc2n1CC(=O)NC12CC(C3CCC3)(C1)C2] | 693.29 |
| I-66a | 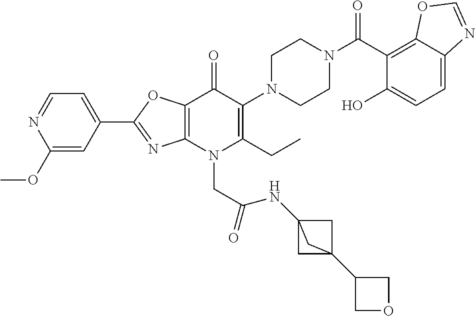 [SMILES: CCc1c(N2CCN(C(=O)c3c(O)ccc4ncoc34)CC2)c(=O)c2oc(-c3ccnc(OC)c3)nc2n1CC(=O)NC12CC(C3COC3)(C1)C2] | 695.27 |
| I-67a | 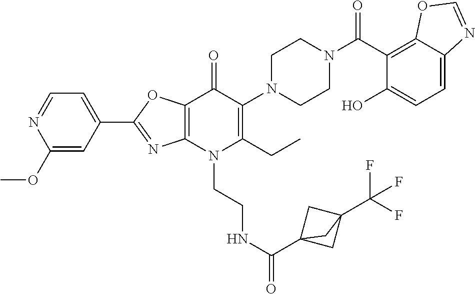 [SMILES: CCc1c(N2CCN(C(=O)c3c(O)ccc4ncoc34)CC2)c(=O)c2oc(-c3ccnc(OC)c3)nc2n1CCNC(=O)C12CC(C(F)(F)F)(C1)C2] | 721.25 |
| I-68a | 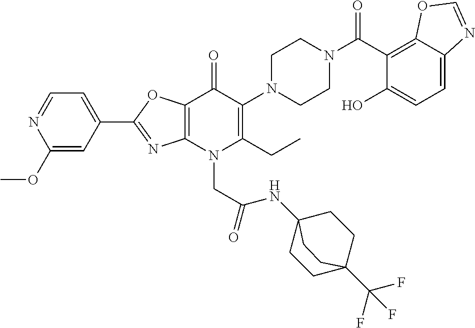 [SMILES: CCc1c(N2CCN(C(=O)c3c(O)ccc4ncoc34)CC2)c(=O)c2oc(-c3ccnc(OC)c3)nc2n1CC(=O)NC12CCC(C(F)(F)F)(CC1)CC2] | 749.28 |
| I-69a | 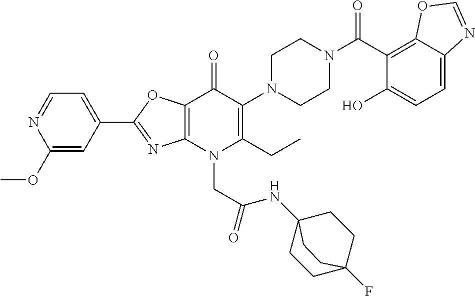 [SMILES: CCc1c(N2CCN(C(=O)c3c(O)ccc4ncoc34)CC2)c(=O)c2oc(-c3ccnc(OC)c3)nc2n1CC(=O)NC12CCC(F)(CC1)CC2] | 699.28 |
| I-70a | 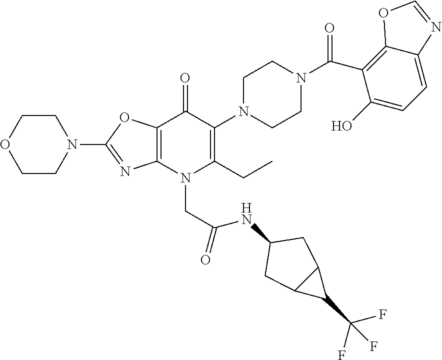 | 699.26 |
| I-71a | 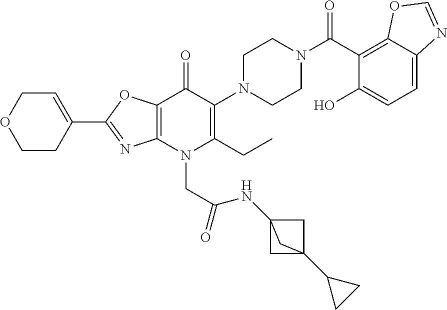 [SMILES: CCc1c(N2CCN(C(=O)c3c(O)ccc4ncoc34)CC2)c(=O)c2oc(C3=CCOCC3)nc2n1CC(=O)NC12CC(C3CC3)(C1)C2] | 654.28 |

As can be appreciated by those skilled in the art, compounds of Table 2b may be accessed through one or more of the general synthetic routes described herein and/or using methods known to one skilled in the art.

TABLE 2b

| | | Mass_ |
|---|---|---|
| | | Theoretical |
| No. | | [g/mol] |
| I-2b | 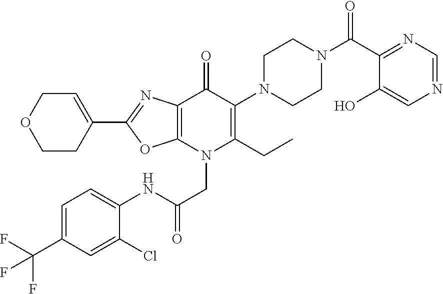 [SMILES: CCc1c(N2CCN(C(=O)c3ncncc3O)CC2)c(=O)c2nc(C3=CCOCC3)oc2n1CC(=O)Nc1ccc(C(F)(F)F)cc1Cl] | 701.20 |
| I-4b | 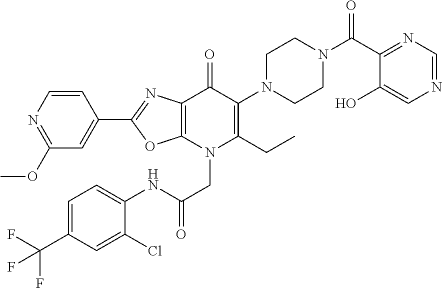 [SMILES: CCc1c(N2CCN(C(=O)c3ncncc3O)CC2)c(=O)c2nc(-c3ccnc(OC)c3)oc2n1CC(=O)Nc1ccc(C(F)(F)F)cc1Cl] | 712.18 |
| I-5b | 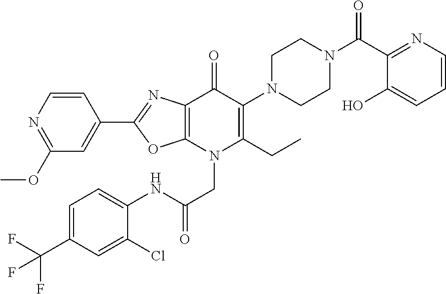 [SMILES: CCc1c(N2CCN(C(=O)c3ncccc3O)CC2)c(=O)c2nc(-c3ccnc(OC)c3)oc2n1CC(=O)Nc1ccc(C(F)(F)F)cc1Cl] | 711.18 |
| I-6b | 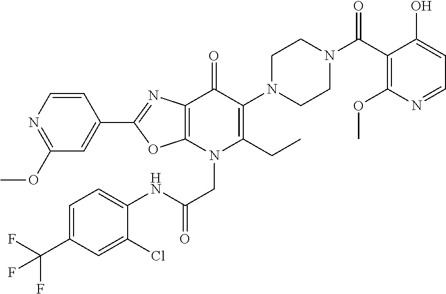 [SMILES: CCc1c(N2CCN(C(=O)c3c(O)ccnc3OC)CC2)c(=O)c2nc(-c3ccnc(OC)c3)oc2n1CC(=O)Nc1ccc(C(F)(F)F)cc1Cl] | 741.19 |
| I-7b | 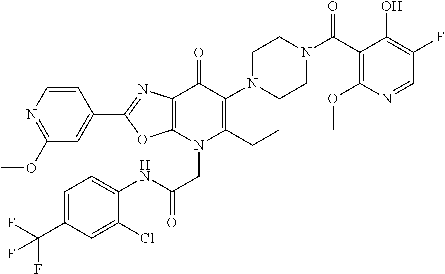 [SMILES: CCc1c(N2CCN(C(=O)c3c(OC)ncc(F)c3O)CC2)c(=O)c2nc(-c3ccnc(OC)c3)oc2n1CC(=O)Nc1ccc(C(F)(F)F)cc1Cl] | 759.18 |
| I-8b | 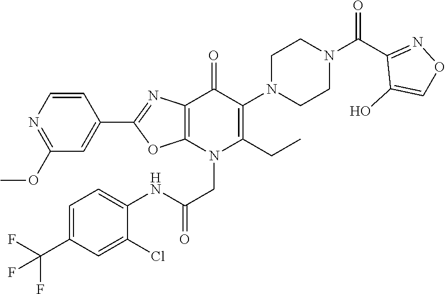 [SMILES: CCc1c(N2CCN(C(=O)c3nocc3O)CC2)c(=O)c2nc(-c3ccnc(OC)c3)oc2n1CC(=O)Nc1ccc(C(F)(F)F)cc1Cl] | 701.16 |
| I-9b | 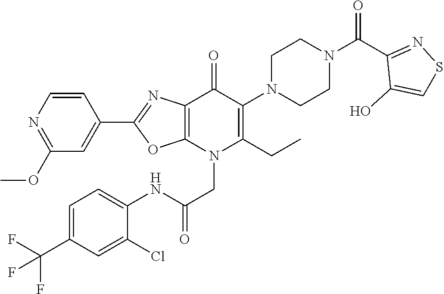 [SMILES: CCc1c(N2CCN(C(=O)c3nscc3O)CC2)c(=O)c2nc(-c3ccnc(OC)c3)oc2n1CC(=O)Nc1ccc(C(F)(F)F)cc1Cl] | 717.14 |
| I-10b | 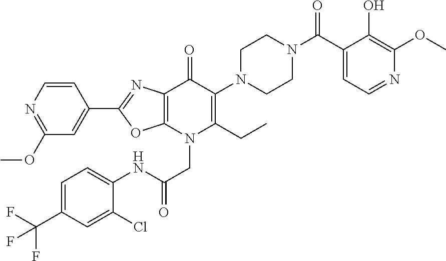 | 741.19 |
| I-11b | 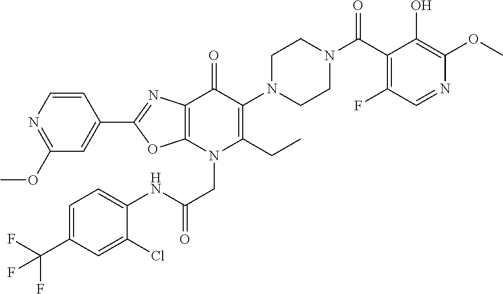 | 759.18 |
| I-12b | 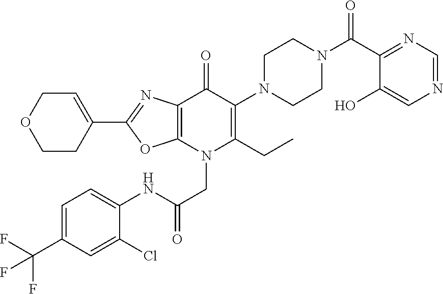 [SMILES: CCc1c(N2CCN(C(=O)c3ncncc3O)CC2)c(=O)c2nc(C3=CCOCC3)oc2n1CC(=O)Nc1ccc(C(F)(F)F)cc1Cl] | 687.18 |
| I-13b | 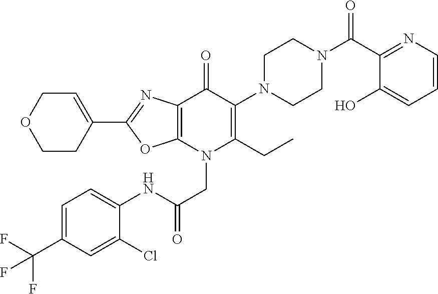 [SMILES: CCc1c(N2CCN(C(=O)c3ncccc3O)CC2)c(=O)c2nc(C3=CCOCC3)oc2n1CC(=O)Nc1ccc(C(F)(F)F)cc1Cl] | 686.19 |
| I-14b | 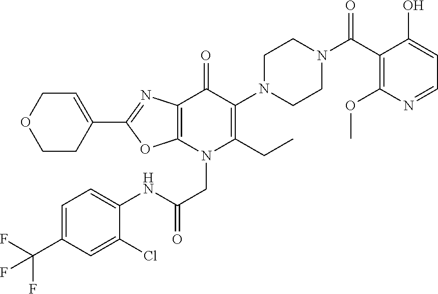 [SMILES: CCc1c(N2CCN(C(=O)c3c(O)ccnc3OC)CC2)c(=O)c2nc(C3=CCOCC3)oc2n1CC(=O)Nc1ccc(C(F)(F)F)cc1Cl] | 716.20 |
| I-15b | 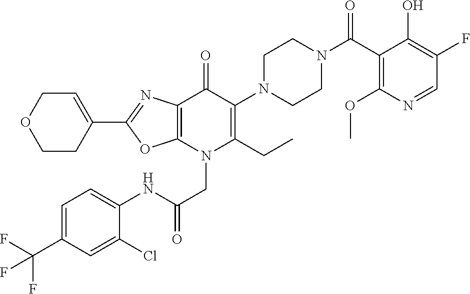 [SMILES: CCc1c(N2CCN(C(=O)c3c(OC)ncc(F)c3O)CC2)c(=O)c2nc(C3=CCOCC3)oc2n1CC(=O)Nc1ccc(C(F)(F)F)cc1Cl] | 734.19 |
| I-16b | 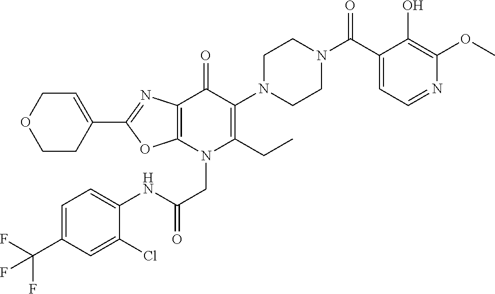 [SMILES: CCc1c(N2CCN(C(=O)c3ccnc(OC)c3O)CC2)c(=O)c2nc(C3=CCOCC3)oc2n1CC(=O)Nc1ccc(C(F)(F)F)cc1Cl] | 716.20 |
| I-17b | 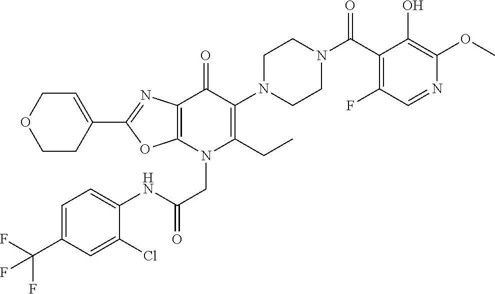 [SMILES: CCc1c(N2CCN(C(=O)c3c(F)cnc(OC)c3O)CC2)c(=O)c2nc(C3=CCOCC3)oc2n1CC(=O)Nc1ccc(C(F)(F)F)cc1Cl] | 734.19 |
| I-18b | 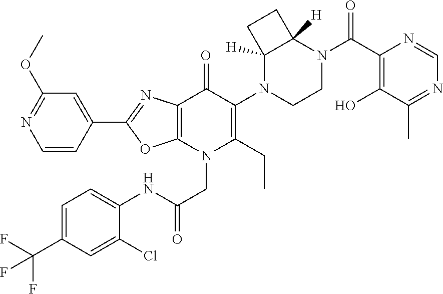 [SMILES: CCc1c(N2CCN(C(=O)c3ncnc(C)c3O)[C@H]3CC[C@@H]32)c(=O)c2nc(-c3ccnc(OC)c3)oc2n1CC(=O)Nc1ccc(C(F)(F)F)cc1Cl] | 752.21 |
| I-19b | 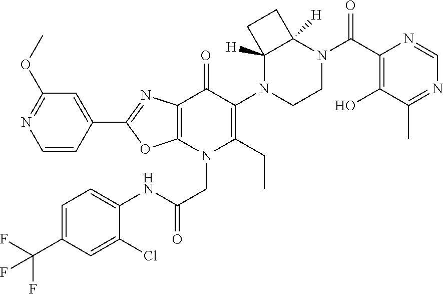 [SMILES: CCc1c(N2CCN(C(=O)c3ncnc(C)c3O)[C@@H]3CC[C@H]32)c(=O)c2nc(-c3ccnc(OC)c3)oc2n1CC(=O)Nc1ccc(C(F)(F)F)cc1Cl] | 752.21 |
| I-20b | 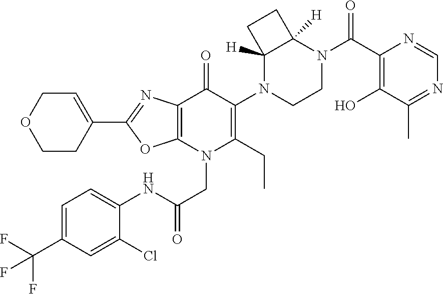 [SMILES: CCc1c(N2CCN(C(=O)c3ncnc(C)c3O)[C@@H]3CC[C@H]32)c(=O)c2nc(C3=CCOCC3)oc2n1CC(=O)Nc1ccc(C(F)(F)F)cc1Cl] | 727.21 |
| I-21b | 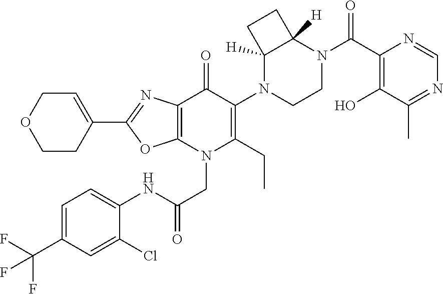 [SMILES: CCc1c(N2CCN(C(=O)c3ncnc(C)c3O)[C@H]3CC[C@@H]32)c(=O)c2nc(C3=CCOCC3)oc2n1CC(=O)Nc1ccc(C(F)(F)F)cc1Cl] | 727.21 |
| I-22b | 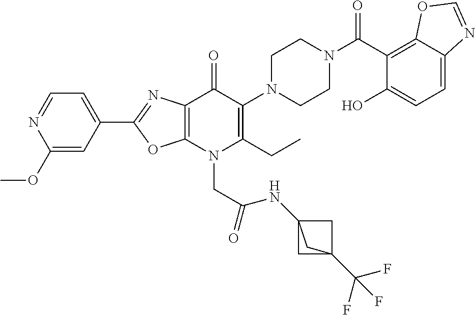 [SMILES: CCc1c(N2CCN(C(=O)c3c(O)ccc4ncoc34)CC2)c(=O)c2nc(-c3ccnc(OC)c3)oc2n1CC(=O)NC12CC(C(F)(F)F)(C1)C2] | 707.23 |
| I-23b | 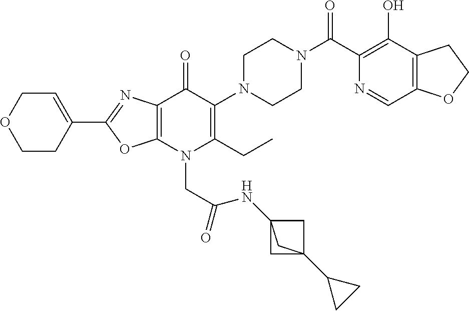 [SMILES: CCc1c(N2CCN(C(=O)c3ncc4c(c3O)CCO4)CC2)c(=O)c2nc(C3=CCOCC3)oc2n1CC(=O)NC12CC(C3CC3)(C1)C2] | 656.30 |
| I-24b | 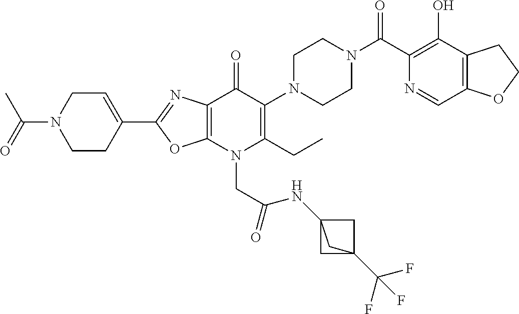 [SMILES: CCc1c(N2CCN(C(=O)c3ncc4c(c3O)CCO4)CC2)c(=O)c2nc(C3=CCN(C(C)=O)CC3)oc2n1CC(=O)NC12CC(C(F)(F)F)(C1)C2] | 725.28 |
| I-25b | 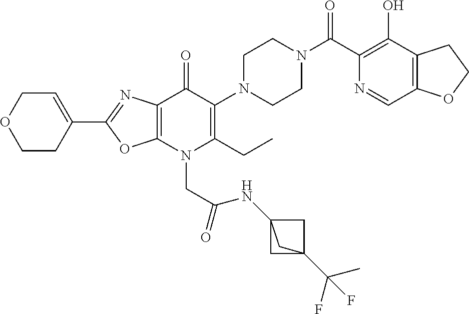 [SMILES: CCc1c(N2CCN(C(=O)c3ncc4c(c3O)CCO4)CC2)c(=O)c2nc(C3=CCOCC3)oc2n1CC(=O)NC12CC(C(C)(F)F)(C1)C2] | 680.28 |
| I-26b | 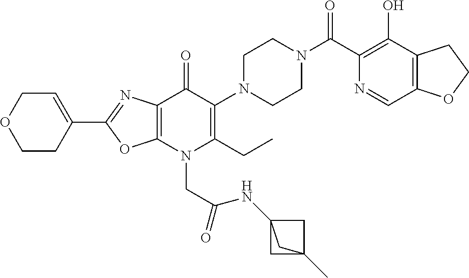 [SMILES: CCc1c(N2CCN(C(=O)c3ncc4c(c3O)CCO4)CC2)c(=O)c2nc(C3=CCOCC3)oc2n1CC(=O)NC12CC(C)(C1)C2] | 630.28 |
| I-27b | 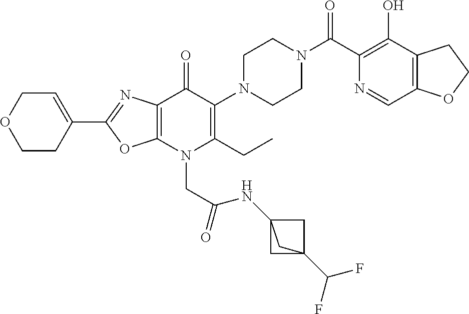 [SMILES: CCc1c(N2CCN(C(=O)c3ncc4c(c3O)CCO4)CC2)c(=O)c2nc(C3=CCOCC3)oc2n1CC(=O)NC12CC(C(F)F)(C1)C2] | 666.26 |
| I-28b | 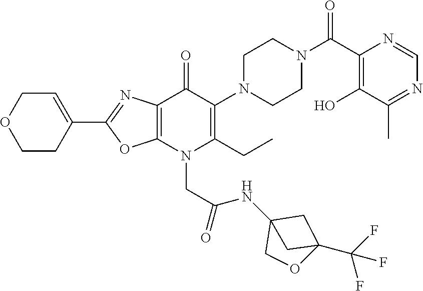 [SMILES: CCc1c(N2CCN(C(=O)c3ncnc(C)c3O)CC2)c(=O)c2nc(C3=CCOCC3)oc2n1CC(=O)NC12COC(C(F)(F)F)(C1)C2] | 673.25 |
| I-29b | 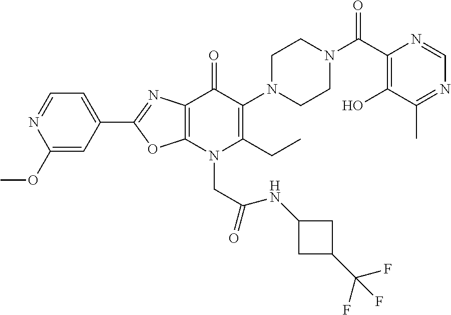 [SMILES: CCc1c(N2CCN(C(=O)c3ncnc(C)c3O)CC2)c(=O)c2nc(-c3ccnc(OC)c3)oc2n1CC(=O)NC1CC(C(F)(F)F)C1] | 670.25 |
| I-30b | 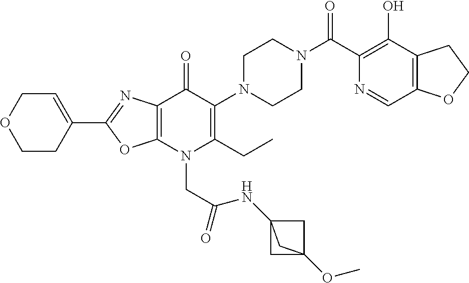 [SMILES: CCc1c(N2CCN(C(=O)c3ncc4c(c3O)CCO4)CC2)c(=O)c2nc(C3=CCOCC3)oc2n1CC(=O)NC12CC(OC)(C1)C2] | 646.28 |
| I-31b | 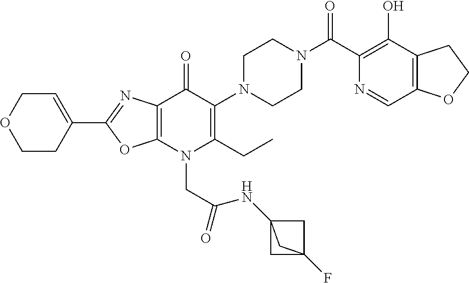 [SMILES: CCc1c(N2CCN(C(=O)c3ncc4c(c3O)CCO4)CC2)c(=O)c2nc(C3=CCOCC3)oc2n1CC(=O)NC12CC(F)(C1)C2] | 634.26 |
| I-32b | 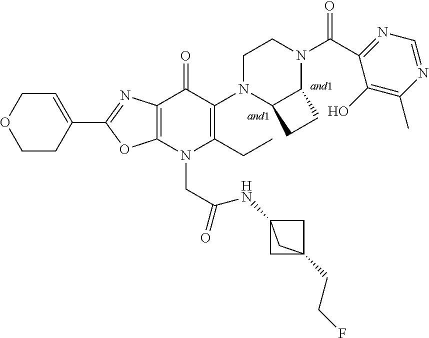 [SMILES: CCc1c(N2CCN(C(=O)c3ncnc(C)c3O)[C@@H]3CC[C@H]32)c(=O)c2nc(C3=CCOCC3)oc2n1CC(=O)N[C@]12C[C@](CCF)(C1)C2] | 661.30 |
| I-33b | 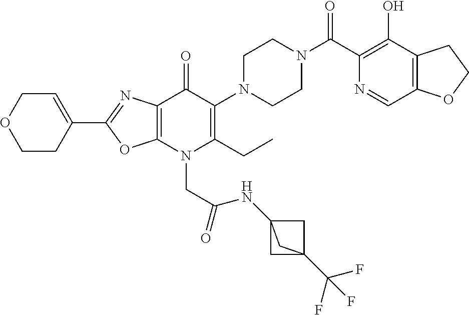 [SMILES: CCc1c(N2CCN(C(=O)c3ncc4c(c3O)CCO4)CC2)c(=O)c2nc(C3=CCOCC3)oc2n1CC(=O)NC12CC(C(F)(F)F)(C1)C2] | 684.25 |
| I-34b | 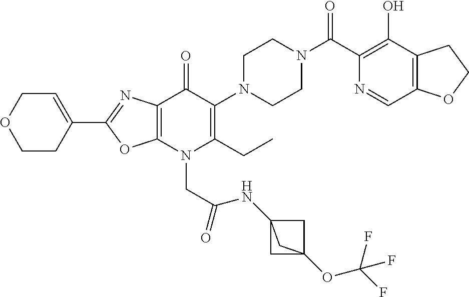 [SMILES: CCc1c(N2CCN(C(=O)c3ncc4c(c3O)CCO4)CC2)c(=O)c2nc(C3=CCOCC3)oc2n1CC(=O)NC12CC(OC(F)(F)F)(C1)C2] | 700.25 |
| I-35b | 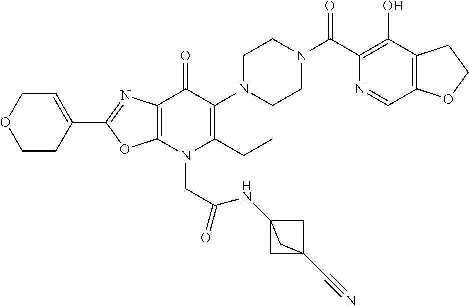 [SMILES: CCc1c(N2CCN(C(=O)c3ncc4c(c3O)CCO4)CC2)c(=O)c2nc(C3=CCOCC3)oc2n1CC(=O)NC12CC(C#N)(C1)C2] | 641.26 |
| I-36b | 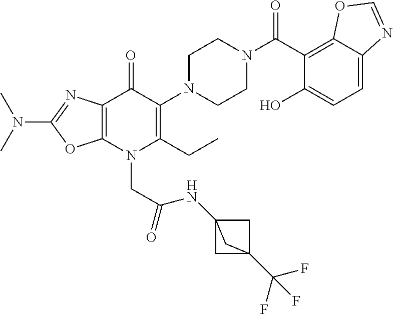 [SMILES: CCc1c(N2CCN(C(=O)c3c(O)ccc4ncoc34)CC2)c(=O)c2nc(N(C)C)oc2n1CC(=O)NC12CC(C(F)(F)F)(C1)C2] | 643.24 |
| I-37b | 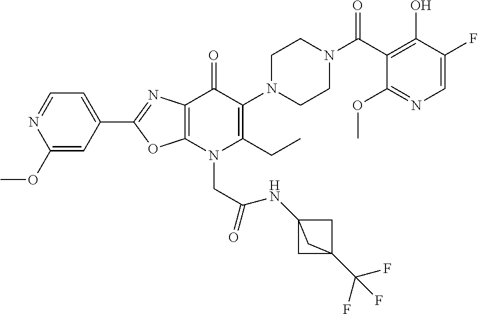 [SMILES: CCc1c(N2CCN(C(=O)c3c(OC)ncc(F)c3O)CC2)c(=O)c2nc(-c3ccnc(OC)c3)oc2n1CC(=O)NC12CC(C(F)(F)F)(C1)C2] | 715.24 |
| I-38b | 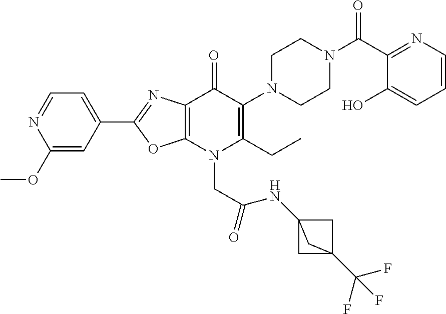 [SMILES: CCc1c(N2CCN(C(=O)c3ncccc3O)CC2)c(=O)c2nc(-c3ccnc(OC)c3)oc2n1CC(=O)NC12CC(C(F)(F)F)(C1)C2] | 667.24 |
| I-39b | 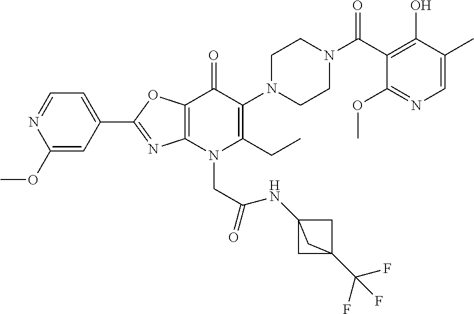 | 711.26 |
| I-40b | 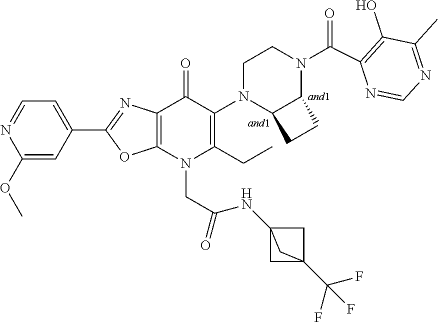 [SMILES: CCc1c(N2CCN(C(=O)c3ncnc(C)c3O)[C@@H]3CC[C@H]32)c(=O)c2nc(-c3ccnc(OC)c3)oc2n1CC(=O)NC12CC(C(F)(F)F)(C1)C2] | 708.26 |
| I-41b | 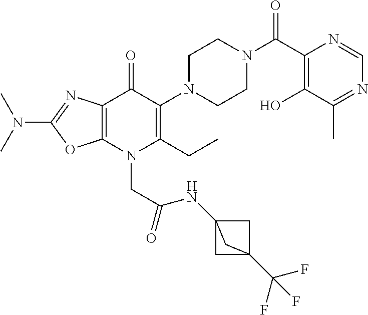 [SMILES: CCc1c(N2CCN(C(=O)c3ncnc(C)c3O)CC2)c(=O)c2nc(N(C)C)oc2n1CC(=O)NC12CC(C(F)(F)F)(C1)C2] | 618.25 |
| I-42b | 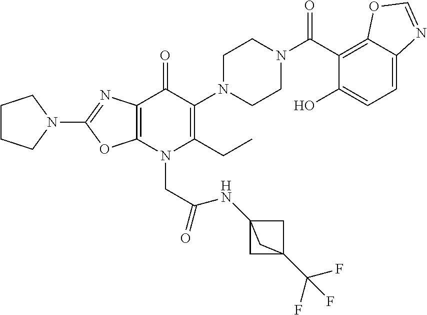 [SMILES: CCc1c(N2CCN(C(=O)c3c(O)ccc4ncoc34)CC2)c(=O)c2nc(N3CCCC3)oc2n1CC(=O)NC12CC(C(F)(F)F)(C1)C2] | 669.25 |
| I-43b | 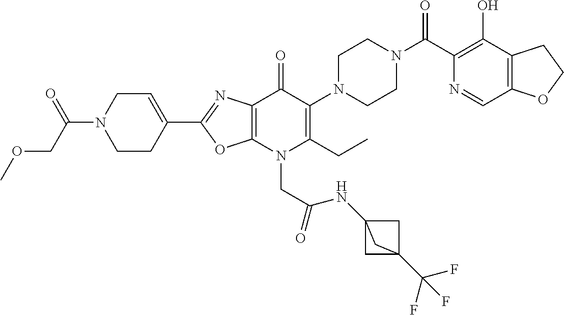 [SMILES: CCc1c(N2CCN(C(=O)c3ncc4c(c3O)CCO4)CC2)c(=O)c2nc(C3=CCN(C(=O)COC)CC3)oc2n1CC(=O)NC12CC(C(F)(F)F)(C1)C2] | 755.29 |
| I-44b | 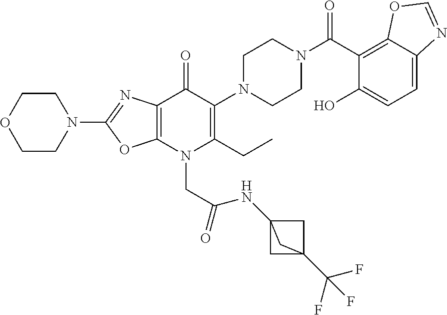 [SMILES: CCc1c(N2CCN(C(=O)c3c(O)ccc4ncoc34)CC2)c(=O)c2nc(N3CCOCC3)oc2n1CC(=O)NC12CC(C(F)(F)F)(C1)C2] | 685.25 |
| I-45b | 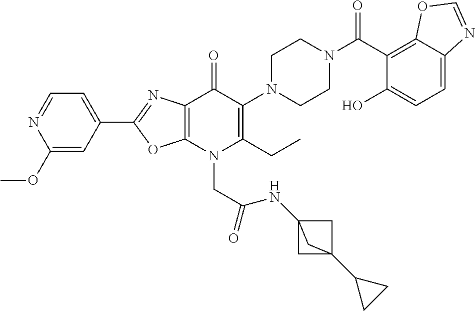 [SMILES: CCc1c(N2CCN(C(=O)c3c(O)ccc4ncoc34)CC2)c(=O)c2nc(-c3ccnc(OC)c3)oc2n1CC(=O)NC12CC(C3CC3)(C1)C2] | 679.28 |
| I-46b | 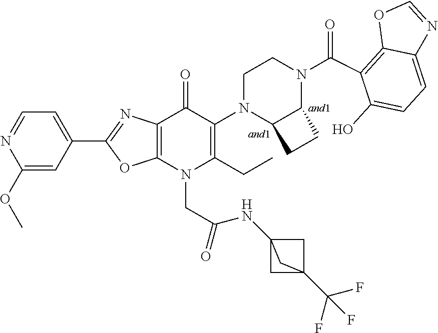 [SMILES: CCc1c(N2CCN(C(=O)c3c(O)ccc4ncoc34)[C@@H]3CC[C@H]32)c(=O)c2nc(-c3ccnc(OC)c3)oc2n1CC(=O)NC12CC(C(F)(F)F)(C1)C2] | |
| I-47b | 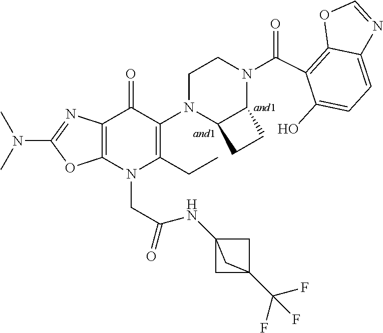 [SMILES: CCc1c(N2CCN(C(=O)c3c(O)ccc4ncoc34)[C@@H]3CC[C@H]32)c(=O)c2nc(N(C)C)oc2n1CC(=O)NC12CC(C(F)(F)F)(C1)C2] | 669.25 |
| I-48b | 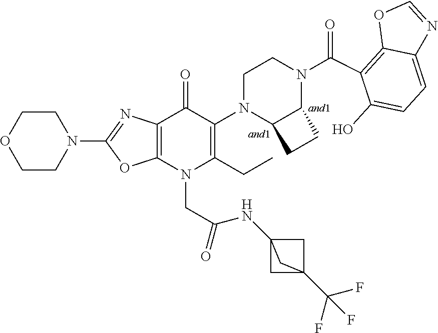 [SMILES: CCc1c(N2CCN(C(=O)c3c(O)ccc4ncoc34)[C@@H]3CC[C@H]32)c(=O)c2nc(N3CCOCC3)oc2n1CC(=O)NC12CC(C(F)(F)F)(C1)C2] | 711.26 |
| I-49b | 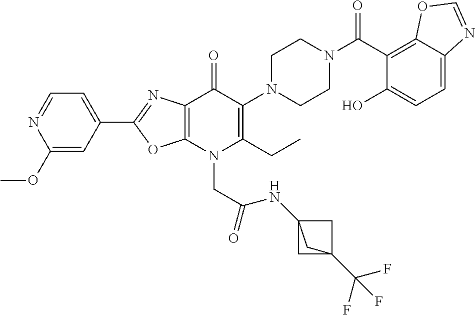 [SMILES: CCc1c(N2CCN(C(=O)c3c(O)ccc4ncoc34)CC2)c(=O)c2nc(-c3ccnc(OC)c3)oc2n1CC(=O)NC12CC(C(F)(F)F)(C1)C2] | 707.23 |
| I-50b | 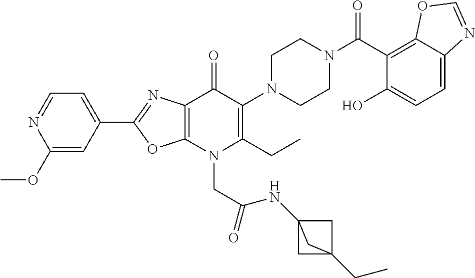 [SMILES: CCc1c(N2CCN(C(=O)c3c(O)ccc4ncoc34)CC2)c(=O)c2nc(-c3ccnc(OC)c3)oc2n1CC(=O)NC12CC(CC)(C1)C2] | 667.28 |
| I-51b | 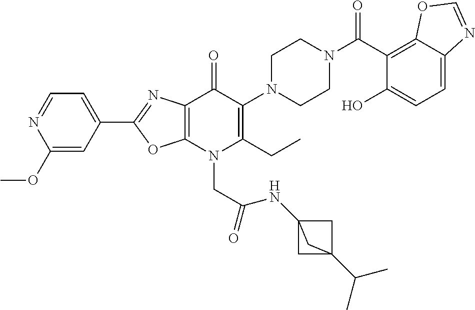 [SMILES: CCc1c(N2CCN(C(=O)c3c(O)ccc4ncoc34)CC2)c(=O)c2nc(-c3ccnc(OC)c3)oc2n1CC(=O)NC12CC(C(C)C)(C1)C2] | 681.29 |
| I-52b | 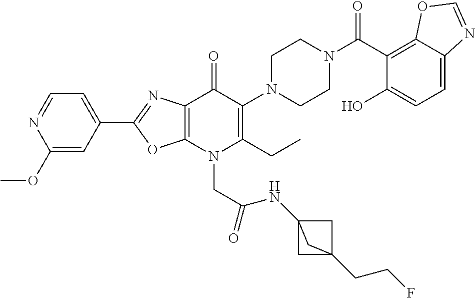 [SMILES: CCc1c(N2CCN(C(=O)c3c(O)ccc4ncoc34)CC2)c(=O)c2nc(-c3ccnc(OC)c3)oc2n1CC(=O)NC12CC(CCF)(C1)C2] | 685.27 |
| I-53b | 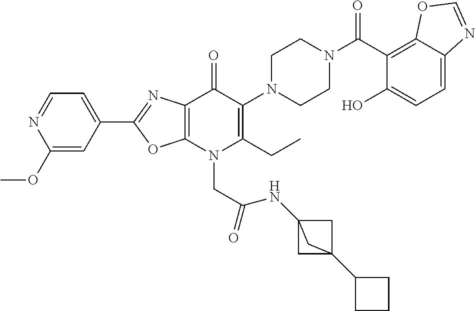 [SMILES: CCc1c(N2CCN(C(=O)c3c(O)ccc4ncoc34)CC2)c(=O)c2nc(-c3ccnc(OC)c3)oc2n1CC(=O)NC12CC(C3CCC3)(C1)C2] | 693.29 |
| I-54b | 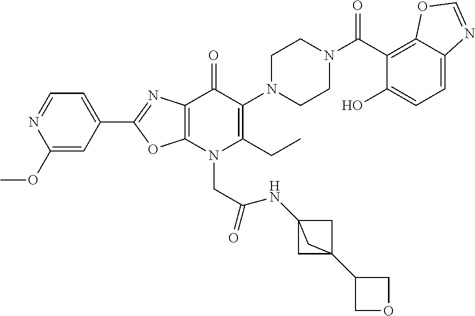 [SMILES: CCc1c(N2CCN(C(=O)c3c(O)ccc4ncoc34)CC2)c(=O)c2nc(-c3ccnc(OC)c3)oc2n1CC(=O)NC12CC(C3COC3)(C1)C2] | 695.27 |
| I-55b | 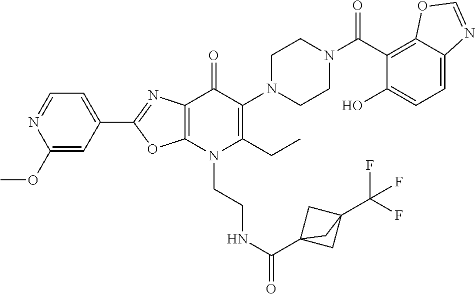 [SMILES: CCc1c(N2CCN(C(=O)c3c(O)ccc4ncoc34)CC2)c(=O)c2nc(-c3ccnc(OC)c3)oc2n1CCNC(=O)C12CC(C(F)(F)F)(C1)C2] | 721.25 |
| I-56b | 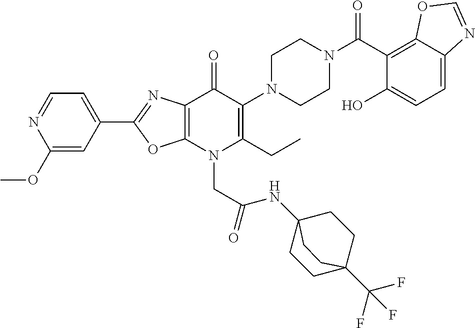 [SMILES: CCc1c(N2CCN(C(=O)c3c(O)ccc4ncoc34)CC2)c(=O)c2nc(-c3ccnc(OC)c3)oc2n1CC(=O)NC12CCC(C(F)(F)F)(CC1)CC2] | 749.28 |
| I-57b | 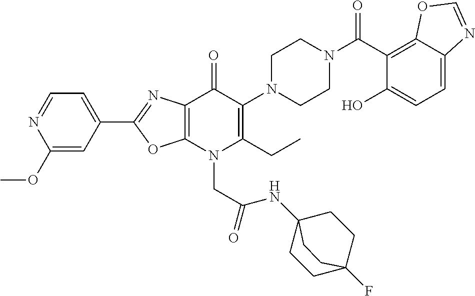 [SMILES: CCc1c(N2CCN(C(=O)c3c(O)ccc4ncoc34)CC2)c(=O)c2nc(-c3ccnc(OC)c3)oc2n1CC(=O)NC12CCC(F)(CC1)CC2] | 699.28 |
| I-58b | 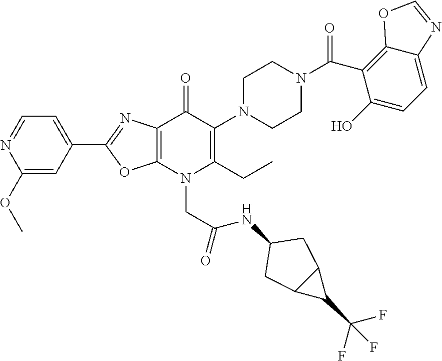 | 721.25 |
| I-59b | 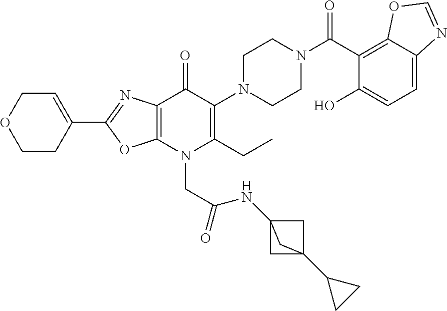 [SMILES: CCc1c(N2CCN(C(=O)c3c(O)ccc4ncoc34)CC2)c(=O)c2nc(C3=CCOCC3)oc2n1CC(=O)NC12CC(C3CC3)(C1)C2] | 654.28 |
| I-60b | 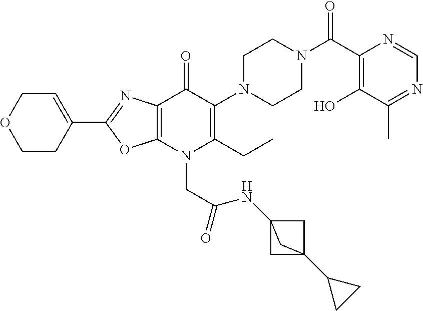 [SMILES: CCc1c(N2CCN(C(=O)c3ncnc(C)c3O)CC2)c(=O)c2nc(C3=CCOCC3)oc2n1CC(=O)NC12CC(C3CC3)(C1)C2] | 629.30 |

### LCMS Methods

### LCMS 1

Instrument Shimadzu LCMS-2020
Stationary Phase Kinetex EVO C18 2.1×30 mm, 5 μm
Mode Binary Gradient
Mobile Phase A 0.025% NH₃·H₂O in water (v/v)
Mobile Phase B Acetonitrile
Gradient 5 to 95% B in 0.8 min, 95% B for 0.42 min, 95 to 5% B in 0.01 min, 5% B for 0.35 min
Flow Rate 1.5 mL/min
Column Temperature 40° C.
Column 2.1×30 mm, 5 μm

### LCMS 2

### Instrument Shimadzu LCMS-2020

### Stationary Phase HALO C18 3.0×30 mm, 5 μm

### Mode Binary Gradient

Mobile Phase A 0.0375% TFA in water (v/v)
Mobile Phase B 0.01875% TFA in Acetonitrile (v/v)

### Gradient

| Time(min) | B(%) | Flow(mL/min) |
|---|---|---|
| 0.0 | 5 | 1.5 |
| 0.50 | 95 | 1.5 |
| 0.80 | 95 | 1.5 |
| 0.81 | 5 | 1.5 |
| 1.05 | 5 | 1.5 |

Flow Rate 1.5 mL/min

### Column Temperature 50° C.

### Column HALO C18 3.0×30 mm, 5 μm

### LCMS 3

### Instrument Agilent HPLC-1290

### Stationary Phase HALO C18, 100×4.6 mm, 2.7 μm

### Mode Binary Gradient

### Mobile Phase A H₂O+0.05% TFA

### Mobile Phase B Acetonitrile+0.05% TFA

Gradient 10% to 95% B in 6 min, hold 95% B in 2 min
Flow Rate 1.5 mL/min

### Column Temperature 40° C.

### Column 100*4.6 mm, 2.7 μm

### Intermediate-1: ethyl 2,4-diiodooxazole-5-carboxylate

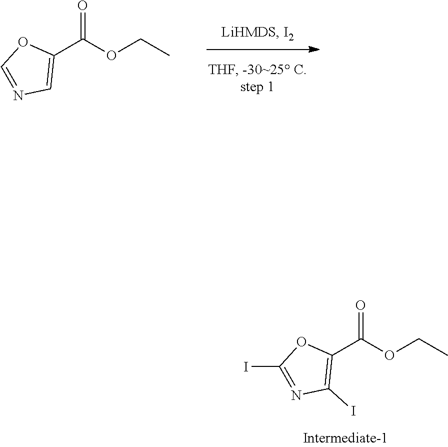

### Step 1. Synthesis of ethyl 2,4-diiodooxazole-5-carboxylate (Intermediate 1)

To a solution of ethyl oxazole-5-carboxylate (10.00 g, 70.86 mmol, 1 eq) in THF (100 mL) was added LiHMDS (1 M, 248.01 mL, 3.5 eq) at −30° C. and the mixture was stirred at −30° C. for 1 h. Then I₂ (53.95 g, 212.58 mmol, 3 eq) was added into the mixture and stirred at 25° C. for 15 h. The mixture was poured into H₂O (500 mL). Then extracted with EtOAc (200 mL*3). The organic phase was washed with brine (500 mL), dried over anhydrous Na₂SO₄ and concentrated under reduced pressure to give a residue. The residue was purified by flash silica gel chromatography (Eluent of EtOAc/PE) and triturated with EtOH (10 mL) to afford the title compound as a yellow solid. ¹H NMR (400 MHz, DMSO-d₆) δ ppm 4.30 (q, 2H), 1.30 (t, 3H). LCMS: 394.0 [M+H]⁺.

### Intermediate 2: tert-butyl 4-(5-ethyl-2-(2-methoxypyridin-4-yl)-7-oxo-4,7-dihydrooxazolo[4,5-b]pyridin-6-yl)piperazine-1-carboxylate

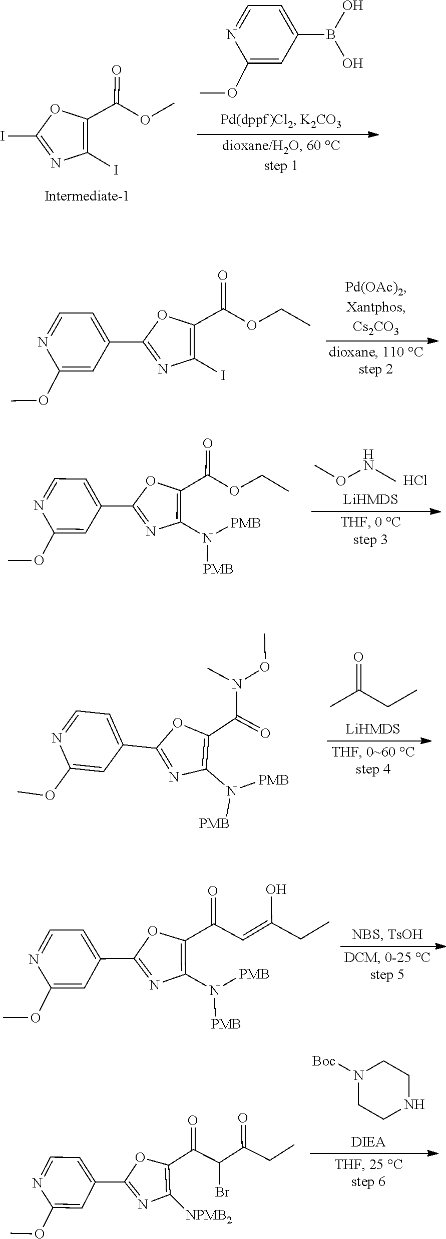

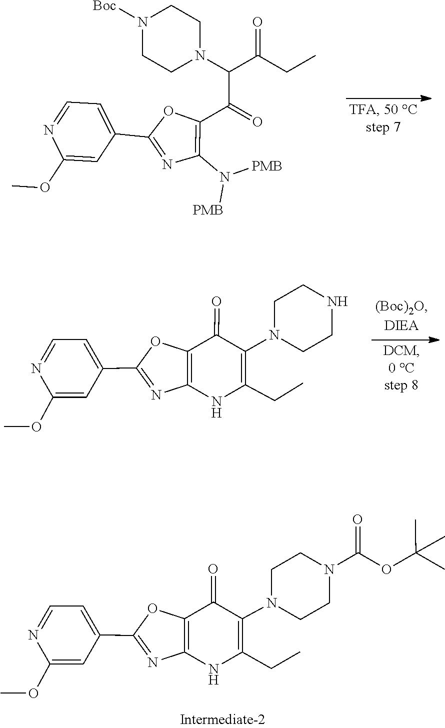

### Step 1. Synthesis of ethyl 4-iodo-2-(2-methoxypyridin-4-yl)oxazole-5-carboxylate

To a solution of ethyl 2,4-diiodooxazole-5-carboxylate (4.90 g, 12.47 mmol, 1 eq), (2-methoxy-4-pyridyl) boronic acid (1.91 g, 12.47 mmol, 1 eq) and K₂CO₃ (3.45 g, 24.94 mmol, 2 eq) in dioxane (100 mL) and H₂O (20 mL) was added Pd(dppf)Cl₂ (913 mg, 1.25 mmol, 0.1 eq). The mixture was degassed and purged with N₂ for 3 times, then stirred at 60° C. for 16 h under N₂ atmosphere. The mixture was poured into H₂O (200 mL), extracted with EtOAc (100 mL*3). The organic phase was washed with brine (200 mL), dried over anhydrous Na₂SO₄ and concentrated under reduced pressure to give a residue. The residue was purified by flash silica gel chromatography (Eluent of EtOAc/PE) and recrystallize with EtOH (50 mL) to afford the title compound as a light yellow solid. ¹H NMR (400 MHz, CDCl₃) δ ppm 8.32 (d, 1H), 7.54 (d, 1H), 7.43 (s, 1H), 4.46 (q, 2H), 4.00 (s, 3H), 1.46 (t, 3H).

LCMS: 375.1 [M+H]⁺.

### Step 2. Synthesis of ethyl 4-(bis(4-methoxybenzyl)amino)-2-(2-methoxypyridin-4-yl)oxazole-5-carboxylate

To a solution of ethyl 4-iodo-2-(2-methoxy-4-pyridyl)oxazole-5-carboxylate (1.00 g, 2.67 mmol, 1 eq) and 1-(4-methoxyphenyl)-N-[(4-methoxyphenyl)methyl]methanamine (1.03 g, 4.01 mmol, 1.5 eq) in dioxane (20 mL) was added Pd(OAc)₂ (60 mg, 267 μmol, 0.1 eq), Xantphos (155 mg, 267 μmol, 0.1 eq) and Cs₂CO₃ (1.31 g, 4.01 mmol, 1.5 eq). The mixture was degassed and purged with N₂ for 3 times, and stirred at 110° C. for 12 h under N₂ atmosphere. It was quenched with H₂O (150 mL), then the mixture was extracted with EtOAc (150 mL*3). The combined organic phase was washed with brine (150 mL*2), dried over anhydrous Na₂SO₄, filtered and concentrated under reduced pressure to give a residue. The residue was purified by flash silica gel chromatography (Eluent of EtOAc/PE) to afford the title compound as a yellow solid. ¹H NMR (400 MHz, CDCl₃) δ ppm 8.30 (d, 1H), 7.53 (d, 1H), 7.42 (s, 1H), 7.19 (d, 4H), 6.86 (d, 4H), 4.77 (s, 4H), 4.31 (q, 2H), 4.02 (s, 3H), 3.81 (s, 6H), 1.33 (t, 3H).

LCMS: 504.3 [M+H]⁺.

### Step 3. Synthesis of 4-(bis(4-methoxybenzyl)amino)-N-methoxy-2-(2-methoxypyridin-4-yl)-N-methyloxazole-5-carboxamide

A mixture of ethyl 4-[bis[(4-methoxyphenyl)methyl]amino]-2-(2-methoxy-4-pyridyl)oxazole-5-carboxylate (500 mg, 993 μmol, 1 eq) and N-methoxymethanamine hydrochloride (484 mg, 4.96 mmol, 5.0 eq) in THF (12 mL) was added LiHMDS (1 M, 7.94 mL, 8 eq) at 0° C. under N₂ atmosphere and the mixture was stirred at 0° C. for 1 h under N₂ atmosphere. The reaction mixture was quenched with saturated NH₄Cl aqueous solution (50 mL) at 0° C. Then the mixture was extracted with EtOAc (50 mL*3). The combined organic phase was washed with brine (50 mL*2), dried over anhydrous Na₂SO₄, filtered and concentrated in vacuum to afford the title compound as a yellow gum, which was used into the next step directly without further purification. ¹H NMR (400 MHz, CDCl₃) δ ppm 8.28 (d, 1H), 7.47 (d, 1H), 7.32 (s, 1H), 7.18 (d, 4H), 6.83 (d, 4H), 4.66 (s, 4H), 4.00 (s, 3H), 3.79 (s, 6H), 3.74 (s, 3H), 3.20 (s, 3H).

LCMS: 519.2 [M+H]⁺.

### Step 4. Synthesis of 1-(4-(bis(4-methoxybenzyl)amino)-2-(2-methoxypyridin-4-yl)oxazol-5-yl)pentane-1,3-dione

A mixture of butan-2-one (556 mg, 7.71 mmol, 690 μL, 5.0 eq) and 4-[bis[(4-methoxyphenyl)methyl]amino]-N-methoxy-2-(2-methoxy-4-pyridyl)-N-methyl-oxazole-5-carboxamide (800 mg, 1.54 mmol, 1 eq) in THF (8 mL) was added LiHMDS (1 M, 7.71 mL, 5.0 eq) at 0° C. under N₂ atmosphere. The mixture was stirred at 60° C. for 1 h. The reaction mixture was quenched with saturated NH₄Cl aqueous solution (100 mL) at 0° C. Then the mixture was extracted with EtOAc (100 mL*3). The combined organic phase was washed with brine (100 mL*2), dried over anhydrous Na₂SO₄, filtered and concentrated under reduced pressure to give a residue. The residue was purified by flash silica gel chromatography (Eluent of EtOAc/PE) to afford the title compound as a yellow gum.

LCMS: 530.2 [M+H]⁺.

### Step 5. Synthesis of 1-(4-(bis(4-methoxybenzyl)amino)-2-(2-methoxypyridin-4-yl)oxazol-5-yl)-2-bromopentane-1,3-dione

A mixture of 1-[4-[bis[(4-methoxyphenyl)methyl]amino]-2-(2-methoxy-4-pyridyl)oxazol-5-yl]pentane-1,3-dione (320 mg, 604 μmol, 1 eq) in DCM (10 mL) was added NBS (86 mg, 483 μmol, 0.8 eq) at 0° C. The reaction was stirred at 0° C. for 1 h. Then TsOH·H₂O (21 mg, 121 μmol, 0.2 eq) was added into the mixture and stirred at 25° C. for another 1 h. The reaction mixture was quenched with H₂O (20 mL) at 25° C. Then the mixture was extracted with DCM (15 mL*3). The combined organic phase was washed with brine (20 mL*2), dried over anhydrous Na₂SO₄, filtered and concentrated in vacuum to afford the title compound as a yellow oil, which was used into the next step directly without purification.

LCMS: 608.1 [M+H]⁺.

### Step 6. Synthesis of tert-butyl 4-(1-(4-(bis(4-methoxybenzyl)amino)-2-(2-methoxypyridin-4-yl)oxazol-5-yl)-1,3-dioxopentan-2-yl)piperazine-1-carboxylate

To a solution of tert-butyl piperazine-1-carboxylate (107 mg, 575 μmol, 1 eq) in THF (7 mL) was added DIEA (149 mg, 1.15 mmol, 200 μL, 2 eq) and 1-[4-[bis[(4-methoxyphenyl)methyl]amino]-2-(2-methoxy-4-pyridyl)oxazol-5-yl]-2-bromo-pentane-1,3-dione (350 mg, 575 μmol, 1 eq). The mixture was stirred at 25° C. for 16 h. It was quenched with H₂O (30 mL), then extracted with EtOAc (30 mL*3). The combined organic phase was washed with brine (30 mL*2), dried over anhydrous Na₂SO₄, filtered and concentrated reduced pressure to give a residue. The residue was purified by flash silica gel chromatography (Eluent of EtOAc/PE) to afford the title compound as a yellow oil.

LCMS: 714.3 [M+H]⁺.

### Step 7. Synthesis of 5-ethyl-2-(2-methoxypyridin-4-yl)-6-(piperazin-1-yl)oxazolo[4,5-b]pyridin-7(4H)-one

A solution of tert-butyl 4-[1-[4-[bis[(4-methoxyphenyl)methyl]amino]-2-(2-methoxy-4-pyridyl)oxazole-5-carbonyl]-2-oxo-butyl]piperazine-1-carboxylate (300 mg, 420 μmol, 1 eq) in TFA (6 mL) was stirred at 50° C. for 1 h. The reaction was cooled to room temperature naturally and concentrated under reduced pressure to afford the title compound as a yellow oil, which was used into the next step directly without purification.

LCMS: 356.1 [M+H]⁺.

### Step 8. Synthesis of tert-butyl 4-(5-ethyl-2-(2-methoxypyridin-4-yl)-7-oxo-4,7-dihydrooxazolo[4,5-b]pyridin-6-yl)piperazine-1-carboxylate

To a solution of 5-ethyl-2-(2-methoxy-4-pyridyl)-6-piperazin-1-yl-4H-oxazolo[4,5-b]pyridin-7-one (150 mg, 422 μmol, 1 eq) in DCM (5 mL) was added DIEA (55 mg, 422 μmol, 74 μL, 1 eq) and (Boc) 20 (184 mg, 844 μmol, 194 μL, 2 eq) at 0° C. The mixture was stirred at 0° C. for 1 h. Then the reaction mixture was diluted with H₂O (20 mL). Then the mixture was extracted with DCM (15 mL*3). The combined organic phase was washed with brine (15 mL*2), dried over anhydrous Na₂SO₄, filtered and concentrated in vacuum to give a residue. The residue was purified by flash silica gel chromatography (Eluent of EtOAc/PE) to afford the title compound as a yellow solid. ¹H NMR (400 MHz, CDCl₃) δ ppm 8.26 (s, 1H), 7.52 (d, 1H), 7.39 (s, 1H), 4.16-4.01 (m, 2H), 3.94 (s, 3H), 3.91-3.80 (m, 2H), 3.02-2.93 (m, 4H), 2.66 (q, 2H), 1.50 (s, 9H), 1.35 (t, 3H).

LCMS: 456.2 [M+H]⁺.

### Intermediate 3: N-(2-chloro-4-(trifluoromethyl)phenyl)-2-(5-ethyl-2-(2-methoxypyridin-4-yl)-7-oxo-6-(piperazin-1-yl)oxazolo[4,5-b]pyridin-4(7H)-yl)acetamide hydrochloride

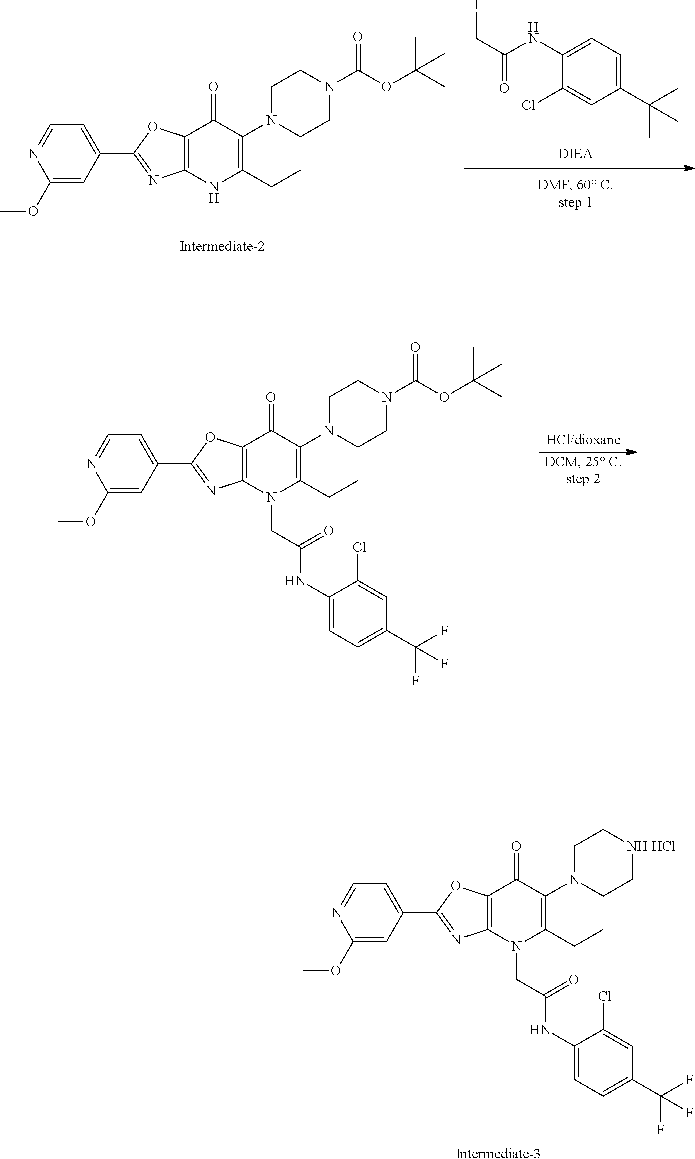

### Step 1. Synthesis of tert-butyl 4-(4-(2-((2-chloro-4-(trifluoromethyl)phenyl)amino)-2-oxoethyl)-5-ethyl-2-(2-methoxypyridin-4-yl)-7-oxo-4,7-dihydrooxazolo[4,5-b]pyridin-6-yl)piperazine-1-carboxylate

To a solution of tert-butyl 4-[5-ethyl-2-(2-methoxy-4-pyridyl)-7-oxo-4H-oxazolo[4,5-b]pyridin-6-yl]piperazine-1-carboxylate (80 mg, 176 μmol, 1 eq) in DMF (2 mL) was added DIEA (68 mg, 527 μmol, 92 μL, 3 eq) and N-[2-chloro-4-(trifluoromethyl)phenyl]-2-iodo-acetamide (Intermediate-12) (83 mg, 228 μmol, 1.3 eq). The mixture was stirred at 60° C. for 1 h. The reaction mixture was cooled to 25° C. and quenched with H₂O (20 mL). Then it was extracted with EtOAc (15 mL*3). The combined organic phase was washed with brine (15 mL*2), dried over anhydrous Na₂SO₄, filtered and concentrated in vacuum to give a residue. The residue was purified by flash silica gel chromatography (Eluent of EtOAc/PE) to afford the title compound as a yellow solid. ¹H NMR (400 MHz, CDCl₃) δ ppm 8.53 (d, 1H), 8.43 (s, 1H), 8.35 (d, 1H), 7.65-7.60 (m, 2H), 7.58-7.54 (m, 1H), 7.51 (s, 1H), 5.23 (s, 2H), 4.19-4.05 (m, 2H), 4.01 (s, 3H), 3.97-3.86 (m, 2H), 3.14 (q, 2H), 3.13-3.11 (m, 2H), 2.69-2.65 (m, 2H), 1.51 (s, 9H), 1.30 (t, 3H).

LCMS: 691.3 [M+H]⁺.

### Step 2. Synthesis of N-(2-chloro-4-(trifluoromethyl)phenyl)-2-(5-ethyl-2-(2-methoxypyridin-4-yl)-7-oxo-6-(piperazin-1-yl)oxazolo[4,5-b]pyridin-4(7H)-yl)acetamide hydrochloride

To a solution of tert-butyl 4-[4-[2-[2-chloro-4-(trifluoromethyl) anilino]-2-oxo-ethyl]-5-ethyl-2-(2-methoxy-4-pyridyl)-7-oxo-oxazolo[4,5-b]pyridin-6-yl]piperazine-1-carboxylate (20 mg, 29 μmol, 1 eq) in DCM (0.4 mL) was added HCl/dioxane (4 M, 0.40 mL, 55 eq). The mixture was stirred at 25° C. for 1 h. The reaction mixture was concentrated under reduced pressure to afford the title compound as a yellow solid, which was used into the next step directly without purification.

LCMS: 591.2 [M+H]⁺.

### Intermediate 4:7-hydroxy-2,3-dihydrofuro[3,2-c]pyridine-6-carboxylic acid

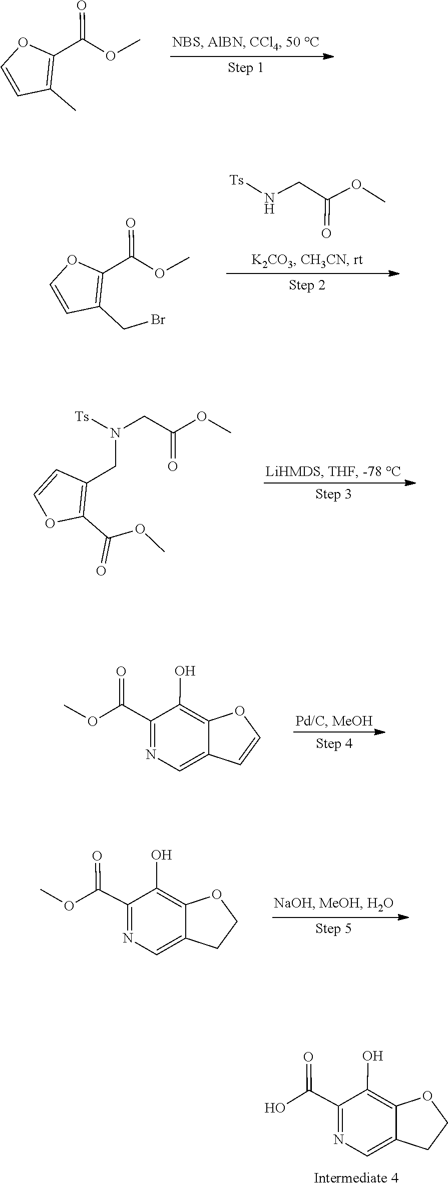

### Step 1. Synthesis of methyl 3-(bromomethyl)furan-2-carboxylate

To a solution of methyl 3-methylfuran-2-carboxylate (5.00 g, 35.7 mmol, 1.00 eq) in CCl₄ (50.0 mL) were added NBS (6.68 g, 37.5 mmol, 1.05 eq) and AIBN (2.35 g, 14.3 mmol, 0.40 eq) at room temperature. The mixture was degassed three times with N₂ and stirred at 50° C. for 16 h. The resulting mixture was concentrated under reduced pressure. The residue was purified by flash silica gel chromatography (eluent of EtOAc/PE) to afford the title compound.

LCMS: 219/221 [M+H]⁺.

### Step 2. Synthesis of methyl 3-(((N-(2-methoxy-2-oxoethyl)-4-methylphenyl)sulfonamido)methyl)furan-2-carboxylate

To a solution of methyl 3-(bromomethyl)furan-2-carboxylate (4.70 g, 21.5 mmol, 1.00 eq) and K₂CO₃ (5.93 g, 43.0 mmol, 2.00 eq) in ACN (47.0 mL) was added methyl 2-(4-methylbenzenesulfonamido)acetate (5.23 g, 21.5 mmol, 1.00 eq) and the mixture was stirred at room temperature for 16 h. The reaction was filtered, and the filtrate was concentrated in vacuum. The residue was purified by column chromatography on silica (Eluent of EtOAc/PE) to afford the title compound. LCMS: 382 [M+H]⁺.

### Step 3. Synthesis of methyl 7-hydroxyfuro[3,2-c]pyridine-6-carboxylate

To a solution of methyl 3-(((N-(2-methoxy-2-oxoethyl)-4-methylphenyl)sulfonamido)methyl)furan-2-carboxylate (1.80 g, 4.72 mmol, 1.00 eq) in THF (18.0 mL) was added a 1 M solution of LiHMDS (14.2 mL, 14.2 mmol, 3.00 eq) in THE dropwise at −78° C. under N₂ atmosphere. After addition, the reaction mixture was allowed to warm to 0° C. and stirred for 5 h under N₂ atmosphere. A saturated NH₄Cl (aq.) solution was added to the reaction mixture and the aq. phase was extracted with EtOAc. The organic layer was dried over anhydrous Na₂SO₄, filtered and concentrated under reduced pressure. The residue was purified by flash silica gel chromatography (Eluent of EtOAc/PE) to afford the title compound.

LCMS: 194 [M+H]⁺.

### Step 4. Synthesis of methyl 7-hydroxy-2,3-dihydrofuro[3,2-c]pyridine-6-carboxylate

To a mixture of methyl 7-hydroxyfuro[3,2-c]pyridine-6-carboxylate (760 mg, 3.93 mmol, 1.00 eq) in MeOH (10.0 mL) was added Pd/C (152 mg, 20%). The mixture was degassed and purged with H₂ gas (40 psi). Then it was stirred at 50° C. for 16 h. The mixture was filtered, and the filtrate was concentrated under reduced pressure. The residue was purified by flash silica gel chromatography (Eluent of DCM/MeOH) to afford the title compound.

LCMS: 196 [M+H]⁺.

### Step 5. Synthesis of 7-hydroxy-2,3-dihydrofuro[3,2-c]pyridine-6-carboxylic acid

To a mixture of methyl 7-hydroxy-2,3-dihydrofuro[3,2-c]pyridine-6-carboxylate (680 mg, 3.48 mmol, 1.00 eq) in H₂O (3.00 mL) and MeOH (3.00 mL) was added NaOH (557 mg, 13.9 mmol, 4.00 eq) at room temperature and the resulting mixture was stirred at 60° C. for 16 h. After completion, the reaction mixture was concentrated under reduced pressure. The residue was diluted with H₂O and acidized by 3 N HCl. The precipitated solids were collected by filtration and dried to afford the title compound, which was used in the next step directly without further purification.

LCMS: 182 [M+H]⁺.

### Intermediate-5: 4-hydroxy-2,3-dihydrofuro[2,3-c]pyridine-5-carboxylic acid

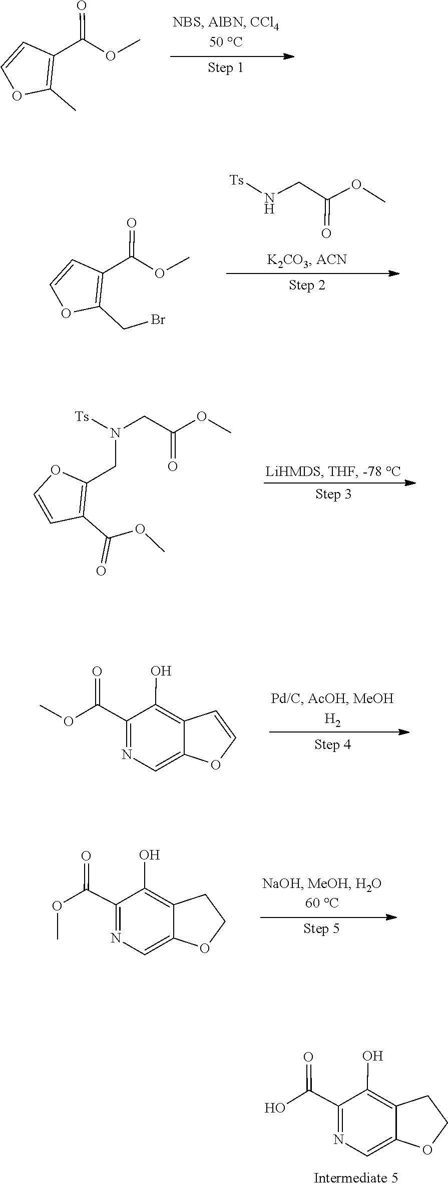

### Step 1. Synthesis of methyl 2-(bromomethyl)furan-3-carboxylate

To a stirred solution of methyl 2-methylfuran-3-carboxylate (10.0 g, 71.4 mmol, 1.00 eq) in CCl₄ (55.0 mL) were added NBS (15.2 g, 85.6 mmol, 1.20 eq) and AIBN (586 mg, 3.57 mmol, 0.05 eq) at room temperature. The resulting mixture was degassed three times with N₂ and stirred overnight at 50° C. under N₂. The reaction mixture was concentrated under reduced pressure. The residue was purified by flash silica gel chromatography (eluent of EtOAc/PE) to afford the title compound. ¹H NMR (400 MHz, DMSO-d₆) δ ppm 7.86 (d, 1H), 6.80 (d, 1H), 4.95 (s, 2H), 3.82 (s, 3H).

### Step 2. Synthesis of methyl 2-(((N-(2-methoxy-2-oxoethyl)-4-methylphenyl)sulfonamido)methyl)furan-3-carboxylate

To a stirred solution of methyl 2-(bromomethyl)furan-3-carboxylate (12.0 g, 54.8 mmol, 1.00 eq) and methyl 2-(4-methylbenzenesulfonamido)acetate (13.3 g, 54.8 mmol, 1.00 eq) in ACN (100 mL) was added K₂CO₃ (15.1 g, 110 mmol, 2.00 eq) at room temperature. The resulting mixture was degassed three times with N₂ and then stirred overnight at room temperature. The reaction mixture was filtered, and the filtrate was concentrated under reduced pressure. The residue was purified by flash silica gel chromatography (eluent of EtOAc/PE) to afford the title compound.

LCMS: 382.1 [M+H]⁺.

### Step 3. Synthesis of methyl 4-hydroxyfuro[2,3-c]pyridine-5-carboxylate

To a stirred solution of methyl 2-([N-(2-methoxy-2-oxoethyl) 4-methylbenzenesulfonamido]methylfuran-3-carboxylate (9.00 g, 23.6 mmol, 1.00 eq) in THF (50.0 mL) was added a 1 M solution of LiHMDS (70.0 mL, 70.0 mmol, 3.00 eq) in THF at −78° C. under N₂. The resulting mixture was stirred for 1 h at room temperature under N₂. The reaction mixture was quenched with saturated NH₄Cl solution at 0° C. and diluted with H₂O (200 mL). The resulting mixture was extracted with EtOAc (2×200 mL). The combined organic layers were washed with brine (3×100 mL), dried over anhydrous Na₂SO₄. After filtration, the filtrate was concentrated under reduced pressure. The residue was purified by flash silica gel chromatography (eluent of EtOAc/PE) to afford a crude product, which was purified by trituration with PE (250 mL) to afford the title compound. LCMS: 194.0 [M+H]⁺.

### Step 4. Synthesis of methyl 4-hydroxy-2,3-dihydrofuro[2,3-c]pyridine-5-carboxylate

To a solution of methyl 4-hydroxyfuro[2,3-c]pyridine-5-carboxylate (1.00 g, 5.18 mmol, 1.00 eq) in AcOH (1.00 mL) and MeOH (10.0 mL) was added Pd/C (1.65 g, 10%). The mixture was degassed three times with H₂, and then stirred at room temperature for 1 h under H₂. The reaction mixture was filtered, and the filter cake was washed with MeOH. The filtrate was concentrated under reduced pressure and the residue was purified by reverse phase HPLC (C18 column, H₂O (10 mmol/L NH₄HCO₃)-ACN) to afford the title compound.

LCMS: 195.9 [M+H]⁺.

### Step 5. Synthesis of 4-hydroxy-2,3-dihydrofuro[2,3-c]pyridine-5-carboxylic acid

To a stirred solution of methyl 4-hydroxy-2H,3H-furo[2,3-c]pyridine-5-carboxylate (300 mg, 1.54 mmol, 1.00 eq) in MeOH (3.00 mL) were added NaOH (246 mg, 6.15 mmol, 4.00 eq) and H₂O (3.00 mL) at room temperature. The reaction mixture was stirred overnight at 60° C. The mixture was acidified to pH=3 with 1 N HCl. The resulting mixture was extracted with EtOAc (3×15 mL). The combined organic layers were washed with brine (2×20 mL), dried over anhydrous Na₂SO₄. After filtration, the filtrate was concentrated under reduced pressure to afford the title compound, which was used in the next step directly without further purification.

LCMS: 181.9 [M+H]⁺.

### Intermediate-11: methyl 5-methoxy-6-methylpyrimidine-4-carboxylate

### Intermediate-6:5-hydroxy-6-methylpyrimidine-4-carboxylic acid

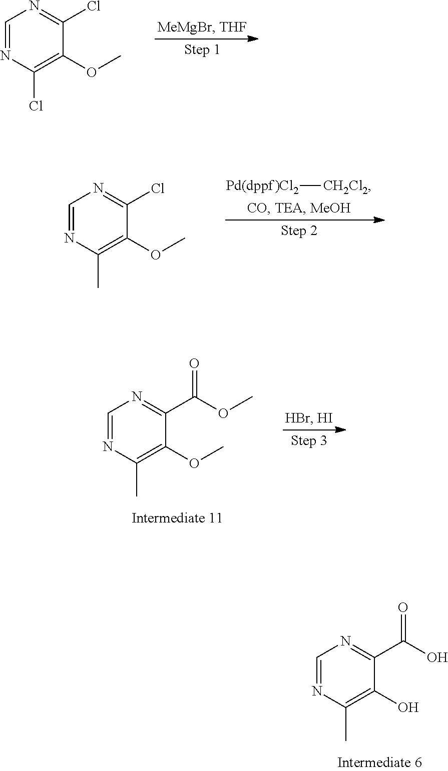

### Step 1: Synthesis of 4-chloro-5-methoxy-6-methylpyrimidine

To a mixture of 4,6-dichloro-5-methoxypyrimidine (30.00 g, 167.6 mmol, 1.0 eq) in THF (300 mL) was added a 3 M solution of MeMgBr (61.45 mL, 184.4 mmol, 1.1 eq) in diethyl ether dropwise at 0° C. and then the mixture was stirred at 5° C. for 1 h. The resulting mixture was poured into H₂O (200 mL) and extracted with EtOAc (100 mL*3). The combined organic phase was washed with brine (100 mL), dried over anhydrous Na₂SO₄, filtered and the filtrate was concentrated in vacuum. The residue was purified by flash silica gel chromatography (eluent of EtOAc/PE) to afford the title compound.

LCMS: 159.1 [M+H]⁺.

### Step 2: Synthesis of methyl 5-methoxy-6-methylpyrimidine-4-carboxylate (Intermediate-11)

To a mixture of 4-chloro-5-methoxy-6-methylpyrimidine (22.00 g, 138.7 mmol, 1.0 eq) in MeOH (250 mL) was added Pd(dppf)Cl₂—CH₂Cl₂ (6.80 g, 8.32 mmol, 0.06 eq) and TEA (28.1 g, 278 mmol, 2.0 eq). The reaction was purged with CO (50 psi) and stirred at 50° C. overnight. The resulting mixture was filtered, and the filtrate was concentrated under reduced pressure. The residue was purified by flash silica gel chromatography (eluent of EtOAc/PE) to afford the title compound.

LCMS: 183.1 [M+H]⁺.

### Step 3: Synthesis of 5-hydroxy-6-methylpyrimidine-4-carboxylic acid

A mixture of methyl 5-methoxy-6-methylpyrimidine-4-carboxylate (16.00 g, 87.83 mmol, 1.0 eq) in HBr solution (aq.) (68.5 mL, 68%) was stirred at 50° C. overnight. Then HI solution (aq.) (67.2 mL, 56%) was added and stirred at 50° C. for 6 h. The reaction mixture was cooled to room temperature and basified with 50% NaOH solution (aq.) to pH 9 at 0° C., then adjusted to pH 7 with 2 M HCl solution (aq.) at 0° C. The mixture was filtered, the filter cake was dried in vacuum to afford the title compound, which was used in the next step without further purification. ¹H NMR (400 MHz, DMSO-d₆) δ ppm 15.96-15.12 (m, 1H), 8.37 (s, 1H), 2.34 (s, 3H).

LCMS: 155.1 [M+H]⁺.

### Intermediate-7: 1-(5-(bis(4-methoxybenzyl)amino)-2-(2-methoxypyridin-4-yl)oxazol-4-yl)-2-bromopentane-1,3-dione

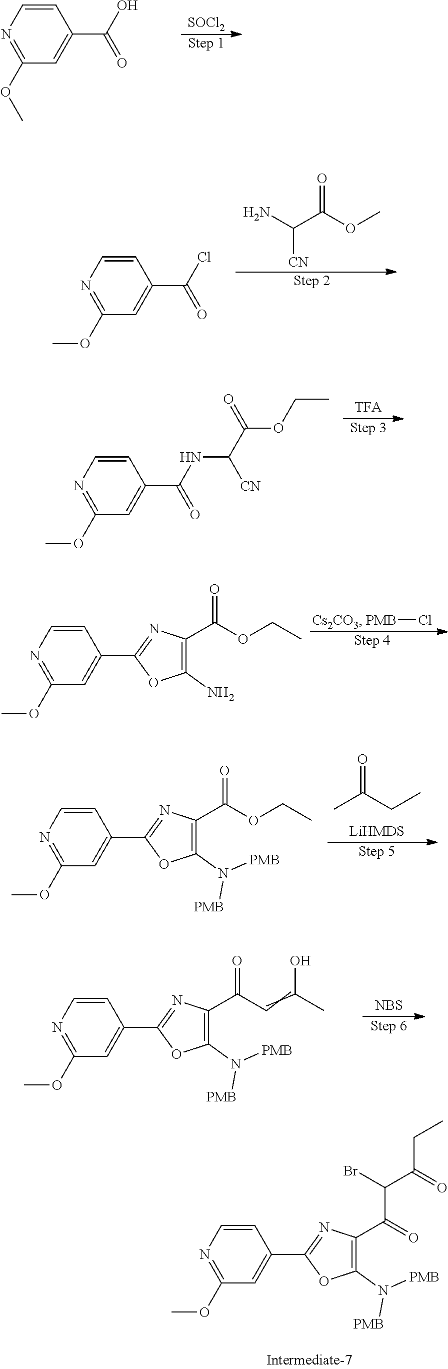

### Step 1. Synthesis of 2-methoxyisonicotinoyl chloride

To a solution of 2-methoxypyridine-4-carboxylic acid (60.00 g, 391.8 mmol, 1 eq) in toluene (500 mL) was added SOCl₂ (140 g, 1.18 mol, 85.4 mL, 3 eq) at 20° C., then the reaction mixture was stirred at 80° C. for 2 h. The reaction mixture was filtered and washed with DCM (25 ml*2), the combined filtrate was concentrated under reduced pressure to afford the title compound, which was used into the next step directly without purification.

### Step 2. Synthesis of ethyl 2-cyano-2-(2-methoxyisonicotinamido)acetate

To a solution of ethyl amino(cyano)acetate 4-methylbenzenesulfonate (40.61 g, 135.2 mmol, 1 eq) in pyridine (70 mL) and DCM (100 mL) was added 2 methoxypyridine-4-carbonyl chloride (23.20 g, 135.2 mmol, 1 eq) in DCM (40 mL) at 0° C. The mixture was stirred at 40° C. for 1 h. The reaction mixture was quenched by H₂O (100 mL) and extracted with DCM (100 mL*3). The combined organic layers were washed with brine (50 mL), dried over anhydrous Na₂SO₄, filtered and concentrated under reduced pressure to give a residue. The residue was purified by flash silica gel chromatography (Eluent of EtOH/DCM) to afford the title compound.

LCMS: 264.1 [M+H]⁺.

### Step 3. Synthesis of ethyl 5-amino-2-(2-methoxypyridin-4-yl)oxazole-4-carboxylate

To ethyl 2-cyano-2-[(2-methoxypyridine-4-carbonyl)amino]acetate (18.00 g, 68.38 mmol, 1 eq) was added TFA (125 g, 1.09 mol, 81.3 mL, 16 eq). The mixture was stirred at 40° C. for 1 h. The reaction mixture was concentrated under reduced pressure to give a residue. The residue was triturated with EtOH (40 ml) at 0° C. for 5 min and filtered, the filter cake was washed with EtOH (20 ml*2) and MTBE (20 ml) and then dried under reduced pressure to afford the title compound.

¹H-NMR (400 MHz, DMSO-d₆) δ ppm 8.26 (d, 1H), 7.67 (s, 2H), 7.31 (d, 1H), 7.01 (s, 1H), 4.24 (q, 2H), 3.90 (s, 3H), 1.28 (t, 3H).

LCMS: 264.0 [M+H]⁺.

### Step 4. Synthesis of ethyl 5-(bis(4-methoxybenzyl)amino)-2-(2-methoxypyridin-4-yl)oxazole-4-carboxylate

To a solution of ethyl 5-amino-2-(2-methoxy-4-pyridyl)oxazole-4-carboxylate (5.00 g, 19.0 mmol, 1 eq) in DMF (50 mL) was added Cs₂CO₃ (27.85 g, 85.47 mmol, 4.5 eq), KI (315 mg, 1.90 mmol, 0.1 eq) and 4-methoxybenzyl chloride (8.92 g, 57.0 mmol, 7.73 mL, 3 eq). The reaction mixture was stirred at 110° C. for 1 h. The reaction mixture was poured into H₂O (300 mL) and was extracted with EtOAc (150 mL*3). The combined organic layers were washed with brine (100 mL*2), dried over anhydrous Na₂SO₄, filtered and concentrated under reduced pressure to give a residue. The residue was purified by flash silica gel chromatography (Eluent of EtOAc/PE) and reverse Phase HPLC (C18 column, water (FA)-ACN) to afford the title compound.

¹H-NMR (400 MHz, CDCl₃) δ ppm 8.20 (d, 1H), 7.39 (d 1H), 7.22-7.14 (m, 5H), 6.87 (d, 4H), 4.70 (s, 4H), 4.38 (q, 2H), 3.96 (s, 3H), 3.81 (s, 6H), 1.39 (t, 3H).

LCMS: 504.2 [M+H]⁺.

### Step 5. Synthesis of 1-(5-(bis(4-methoxybenzyl)amino)-2-(2-methoxypyridin-4-yl)oxazol-4-yl)-3-hydroxypent-2-en-1-one

To a solution of butan-2-one (752 mg, 10.4 mmol, 933 μL, 2.5 eq) in THF (10 mL) was added LiHMDS (1 M in THF, 10.43 mL, 2.5 eq) at 0° C. under N₂ atmosphere. The reaction mixture was stirred at 25° C. for 0.5 h. Then ethyl 5-[bis[(4-methoxyphenyl)methyl]amino]-2-(2-methoxy-4-pyridyl)oxazole-4-carboxylate (2.10 g, 4.17 mmol, 1 eq) dissolved in THF (10 mL) was added into the mixture. The reaction mixture was stirred at 60° C. for 1 h under N₂ atmosphere. The reaction mixture was poured into saturated NH₄Cl solution (200 mL) and extracted with EtOAc (80 mL*3). The combined organic layers were washed with brine (50 mL), dried with anhydrous Na₂SO₄, filtered and concentrated under reduced pressure to give a residue. The residue was purified by flash silica gel chromatography (Eluent of EtOAc/PE) to afford the title compound.

LCMS: 530.2 [M+H]⁺.

### Step 6. Synthesis of 1-(5-(bis(4-methoxybenzyl)amino)-2-(2-methoxypyridin-4-yl)oxazol-4-yl)-2-bromopentane-1,3-dione

To a solution of 1-[5-[bis[(4-methoxyphenyl)methyl]amino]-2-(2-methoxy-4-pyridyl)oxazol-4-yl]-3-hydroxypent-2-en-1-one (1.00 g, 1.89 mmol, 1 eq) in DCM (10 mL) was added NBS (269 mg, 1.51 mmol, 0.8 eq) at 0° C. The reaction mixture was stirred at 25° C. for 1 h. The reaction mixture was poured into H₂O (100 mL) and was extracted with DCM (60 mL*3). The combined organic layers were washed with brine (50 mL), dried over anhydrous Na₂SO₄, filtered and concentrated under reduced pressure to give the title compound, which was used into the next step directly without purification.

LCMS: 610.1 [M+H]⁺.

### Intermediate-8:2-(6-(4-(tert-butoxycarbonyl)piperazin-1-yl)-5-ethyl-2-(2-methoxypyridin-4-yl)-7-oxooxazolo[5,4-b]pyridin-4(7H)-yl)acetic acid

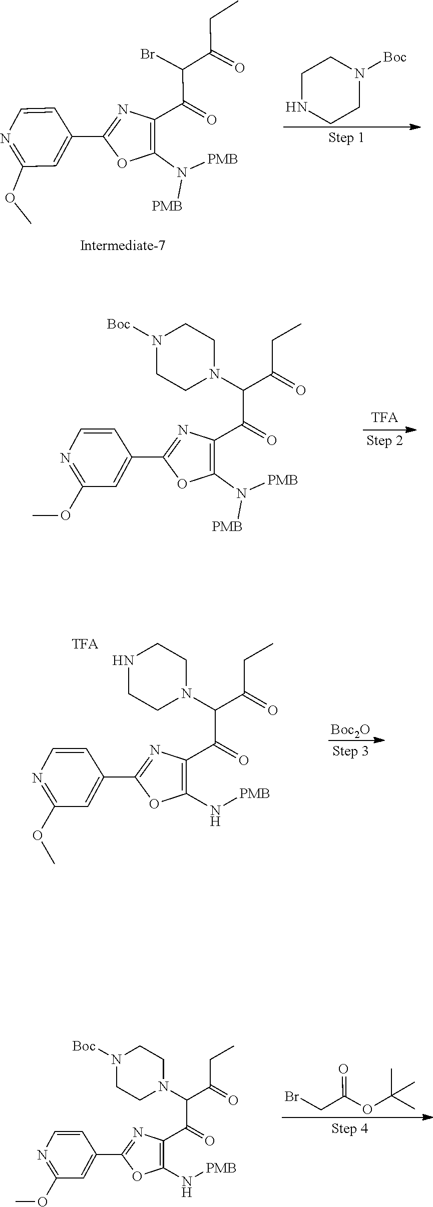

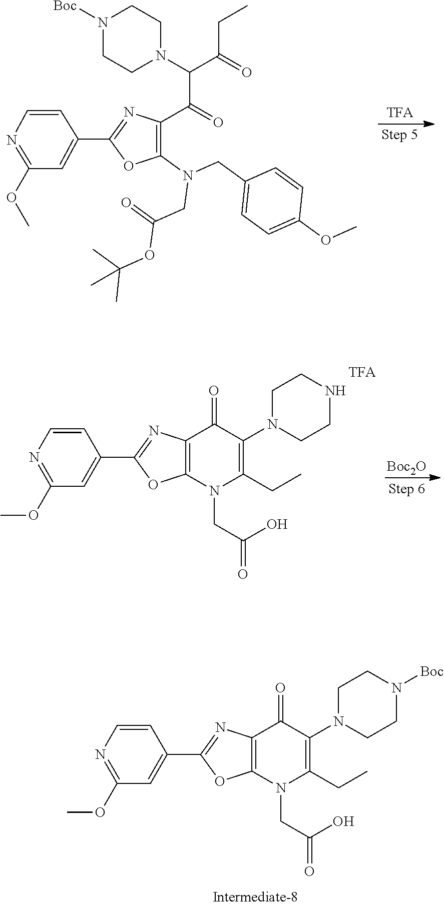

### Step 1. Synthesis of tert-butyl 4-(1-(5-(bis(4-methoxybenzyl)amino)-2-(2-methoxypyridin-4-yl)oxazol-4-yl)-1,3-dioxopentan-2-yl)piperazine-1-carboxylate

To a solution of 1-[5-[bis[(4-methoxyphenyl)methyl]amino]-2-(2-methoxy-4-pyridyl)oxazol-4-yl]-2-bromo-pentane-1,3-dione (Intermediate-7) (700 mg, 1.15 mmol, 1 eq) and tert-butyl piperazine-1-carboxylate (214 mg, 1.15 mmol, 1 eq) in THF (7 mL) was added DIEA (298 mg, 2.30 mmol, 401 μL, 2 eq). The reaction mixture was stirred at 25° C. for 12 h. The reaction mixture was poured into H₂O (100 mL) and extracted with EtOAc (60 mL*3). The combined organic layers was washed with brine (50 mL), dried with anhydrous Na₂SO₄, filtered and concentrated under reduced pressure to give a residue. The residue was purified by flash silica gel chromatography (Eluent of EtOAc/PE) to afford the title compound.

¹H-NMR (400 MHz, CDCl₃) δ ppm 8.22 (d, 1H), 7.26 (d, 1H), 7.17 (d, 4H), 7.06 (s, 1H), 6.88 (d, 4H), 5.28 (s, 1H), 4.97-4.86 (m, 2H), 4.84-4.70 (m, 2H), 3.98 (s, 3H), 3.81 (s, 6H), 3.48-3.44 (m, 4H), 2.90-2.54 (m, 6H), 1.44 (s, 9H), 1.10 (t, 3H).

LCMS: 714.3 [M+H]⁺.

### Step 2. Synthesis of 1-(5-((4-methoxybenzyl)amino)-2-(2-methoxypyridin-4-yl)oxazol-4-yl)-2-(piperazin-1-yl)pentane-1,3-dione trifluoroacetate

To a solution of tert-butyl 4-[1-[5-[bis[(4-methoxyphenyl)methyl]amino]-2-(2-methoxy-4-pyridyl)oxazole-4-carbonyl]-2-oxo-butyl]piperazine-1-carboxylate (700 mg, 981 μmol, 1 eq) in TFA (7 mL) was stirred at 50° C. for 1 h. The reaction was cooled to room temperature and concentrated under reduced pressure to afford the title compound, which was used into the next step directly without purification.

LCMS: 494.2 [M+H]⁺.

### Step 3. Synthesis of tert-butyl 4-(1-(5-((4-methoxybenzyl)amino)-2-(2-methoxypyridin-4-yl)oxazol-4-yl)-1,3-dioxopentan-2-yl)piperazine-1-carboxylate

To a solution of 1-(5-((4-methoxybenzyl)amino)-2-(2-methoxypyridin-4-yl)oxazol-4-yl)-2-(piperazin-1-yl) pentane-1,3-dione trifluoroacetate (590 mg, 971 μmol, 1 eq) in DCM (6 mL) was added DIEA (377 mg, 2.91 mmol, 507 μL, 3 eq) and Boc₂O (254 mg, 1.17 mmol, 267 μL, 1.2 eq). The reaction mixture was stirred at 25° C. for 1 h. The reaction mixture was poured into H₂O (50 mL) and was extracted with DCM (50 mL*3). The combined organic layers were washed with brine (50 mL), dried over anhydrous Na₂SO₄, filtered and concentrated under reduced pressure. The residue was purified by flash silica gel chromatography (Eluent of EtOAc/PE) to afford the title compound.

¹H-NMR (400 MHz, CDCl₃) δ ppm 8.23 (d, 1H), 7.76 (s, 1H), 7.36-7.28 (m, 2H), 7.14 (s, 1H), 6.92 (d, 2H), 5.00 (s, 1H), 4.62 (d, 2H), 3.99 (s, 3H), 3.82 (s, 3H), 3.45-3.49 (m, 4H), 2.79-2.47 (m, 6H), 1.43 (s, 9H), 1.10 (t, 3H).

LCMS: 594.3 [M+H]⁺.

### Step 4. Synthesis of tert-butyl 4-(1-(5-((2-(tert-butoxy)-2-oxoethyl) (4-methoxybenzyl)amino)-2-(2-methoxypyridin-4-yl)oxazol-4-yl)-1,3-dioxopentan-2-yl)piperazine-1-carboxylate

To a solution of tert-butyl 4-(1-(5-((4-methoxybenzyl)amino)-2-(2-methoxypyridin-4-yl)oxazol-4-yl)-1,3-dioxopentan-2-yl)piperazine-1-carboxylate (460 mg, 775 μmol, 1 eq) and tert-butyl 2-bromoacetate (151 mg, 775 μmol, 114 μL, 1 eq) in THF (4.6 mL) was added t-BuOK (348 mg, 3.10 mmol, 4 eq). The reaction mixture was stirred at 50° C. for 0.5 h. Then second batch of tert-butyl 2-bromoacetate (151 mg, 775 μmol, 114 μL, 1 eq) was added to the mixture. The reaction mixture was stirred at 50° C. for another 0.5 h. Then the third batch of tert-butyl 2-bromoacetate (151 mg, 775 μmol, 114 μL, 1 eq) was added into the mixture and stirred at 50° C. for another 0.5 h. The reaction mixture was diluted with H₂O (50 mL) and extracted with DCM (40 mL*3). The combined organic phase was washed with brine (30 mL), dried with anhydrous Na₂SO₄, filtered and concentrated under reduced pressure. The residue was purified by flash silica gel chromatography (Eluent of EtOAc/PE) to afford the title compound.

¹H-NMR (400 MHz, CDCl₃) δ ppm 8.23 (d, 1H), 7.29 (s, 1H), 7.25 (d, 2H), 7.09 (s, 1H), 6.90 (d, 2H), 5.28 (s, 2H), 4.85 (s, 1H), 4.58-4.29 (m, 2H), 3.98 (s, 3H), 3.82 (s, 3H), 3.48-3.44 (m, 4H), 2.76-2.69 (m, 4H), 2.55 (q, 2H), 1.44 (s, 9H), 1.42 (s, 9H), 1.09 (t, 3H).

LCMS: 708.4 [M+H]⁺.

### Step 5. Synthesis of 2-(5-ethyl-2-(2-methoxypyridin-4-yl)-7-oxo-6-(piperazin-1-yl)oxazolo[5,4-b]pyridin-4(7H)-yl)acetic acid Trifluoroacetic acid salt

To a solution of tert-butyl 4-[1-[5-[(2-tert-butoxy-2-oxo-ethyl)-[(4 methoxyphenyl)methyl]amino]-2-(2-methoxy-4-pyridyl)oxazole-4-carbonyl]-2-oxo-butyl]piperazine-1-carboxylate (400 mg, 565 μmol, 1 eq) in TFA (4 mL) was stirred at 120° C. for 3 h. The reaction mixture was concentrated under reduced pressure to afford the title compound, which was used into the next step directly without purification.

LCMS: 414.1 [M+H]⁺.

### Step 6. Synthesis of 2-(6-(4-(tert-butoxycarbonyl)piperazin-1-yl)-5-ethyl-2-(2-methoxypyridin-4-yl)-7-oxooxazolo[5,4-b]pyridin-4(7H)-yl) acetic acid

To a solution of 2-(5-ethyl-2-(2-methoxypyridin-4-yl)-7-oxo-6-(piperazin-1-yl)oxazolo[5,4-b]pyridin-4(7H)-yl) acetic acid Trifluoroacetic acid salt (290 mg, 550 μmol, 1 eq) in DCM (3 mL) was added DIEA (355 mg, 2.75 mmol, 479 μL, 5 eq) and (Boc) 20 (180 mg, 825 μmol, 190 μL, 1.5 eq). The reaction mixture was stirred at 25° C. for 0.5 h. The reaction mixture was poured into H₂O (3 mL) and was extracted with DCM (4 mL*3). The combined organic layers were washed with brine (5 mL), dried with anhydrous Na₂SO₄, filtered and concentrated under reduced pressure to give a residue. The residue was purified by flash silica gel chromatography (Eluent of MeOH/DCM) to afford the title compound.

LCMS: 514.1 [M+H]⁺,

### Intermediate-9: N-(2-chloro-4-(trifluoromethyl)phenyl)-2-(5-ethyl-2-(2-methoxypyridin-4-yl)-7-oxo-6-(piperazin-1-yl)oxazolo[5,4-b]pyridin-4(7H)-yl)acetamide hydrochloride

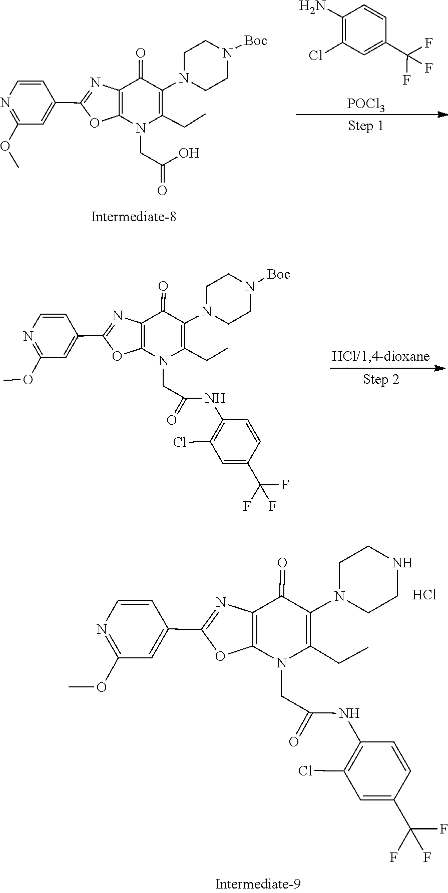

### Step 1. Synthesis of tert-butyl 4-(4-(2-((2-chloro-4-(trifluoromethyl)phenyl)amino)-2-oxoethyl)-5-ethyl-2-(2-methoxypyridin-4-yl)-7-oxo-4,7-dihydrooxazolo[5,4-b]pyridin-6-yl)piperazine-1-carboxylate

To a solution of 2-[6-(4-tert-butoxycarbonylpiperazin-1-yl)-5-ethyl-2-(2-methoxy-4-pyridyl)-7-oxo-oxazolo[5,4-b]pyridin-4-yl]acetic acid (80 mg, 0.16 mmol, 1 eq) and 2-chloro-4-(trifluoromethyl) aniline (40 mg, 0.20 mmol, 28 μL, 1.3 eq) in pyridine (0.8 mL) was added POCl₃ (36 mg, 0.23 mmol, 22 μL, 1.5 eq) at 0° C. The reaction mixture was stirred at 60° C. for 1 h. The reaction mixture was poured into H₂O (10 mL) and was extracted with DCM (10 mL*3). The combined organic layers were washed with brine (10 mL), dried with anhydrous Na₂SO₄, filtered and concentrated under reduced pressure to give a residue. The residue was purified reverse Phase HPLC (C18 column, water (FA)-ACN) to afford the title compound.

LCMS: 691.3 [M+H]⁺.

### Step 2. Synthesis of N-(2-chloro-4-(trifluoromethyl)phenyl)-2-(5-ethyl-2-(2-methoxypyridin-4-yl)-7-oxo-6-(piperazin-1-yl)oxazolo[5,4-b]pyridin-4(7H)-yl)acetamide hydrochloride

To a solution of tert-butyl 4-[4-[2-[2-chloro-4-(trifluoromethyl) anilino]-2-oxo-ethyl]-5-ethyl-2-(2-methoxy-4-pyridyl)-7-oxo-oxazolo[5,4-b]pyridin-6-yl]piperazine-1-carboxylate (50 mg, 72 μmol, 1 eq) in HCl/1,4-dioxane (2 M, 0.5 mL) was stirred at 25° C. for 0.5 h. The reaction mixture was concentrated under reduced pressure to afford the title compound, which was used into the next step directly without purification.

LCMS: 591.2 [M+H]⁺.

### Intermediate-10: sodium 5-hydroxy-6-methylpyrimidine-4-carboxylate

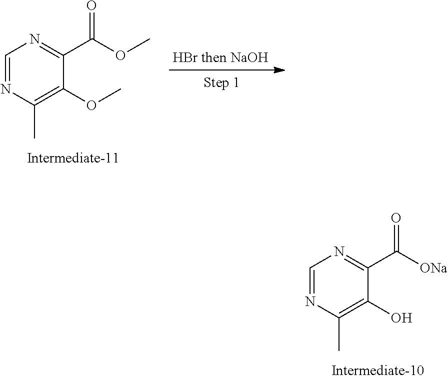

### Step 3. Synthesis of sodium 5-hydroxy-6-methylpyrimidine-4-carboxylate

To a solution of methyl 5-methoxy-6-methyl-pyrimidine-4-carboxylate (Intermediate-11) (210 g, 1.15 mol, 1 eq) was added HBr (850 mL) at 20° C. After addition, the mixture was stirred at 50° C. for 16 h, and then was added HI (600 mL) at 50° C. The resulting mixture was stirred at 50° C. for 6 h. The reaction mixture was filtered. The filtrate was adjusted to pH to 8~9 with aqueous NaOH solution (30% in water) at 0~5° C. The mixture was filtered, the filter cake was dried under reduced pressure to afford the title compound, which was used into next step directly without further purification.

¹H NMR (400 MHz, CD₃OD) δ ppm 8.47 (s, 1H), 2.48 (s, 3H).

### Intermediate-12: N-(2-chloro-4-(trifluoromethyl)phenyl)-2-iodoacetamide

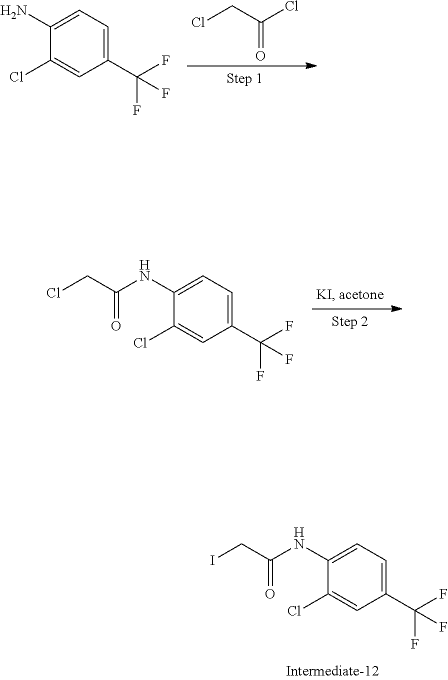 [SMILES: Nc1ccc(C(F)(F)F)cc1Cl ; O=C(Cl)CCl ; O=C(CCl)Nc1ccc(C(F)(F)F)cc1Cl ; O=C(CI)Nc1ccc(C(F)(F)F)cc1Cl]

### Step 1. Synthesis of 2-chloro-N-(2-chloro-4-(trifluoromethyl)phenyl)acetamide

To a solution of 2-chloro-4-(trifluoromethyl) aniline (19.1 g, 97.7 mmol, 1.0 eq) and TEA (19.76 g, 195.3 mmol, 2.0 eq) in DCM (200 mL) was added dropwise a solution of 2-chloroacetyl chloride (11.03 g, 97.7 mmol, 1.0 eq) in DCM (50 mL) at 0° C. After addition, the resulting mixture was warmed to room temperature and stirred at room temperature overnight. The reaction mixture was purified by column chromatography on silica gel (Eluent of EtOAc/PE) to afford the title compound.

LCMS: 273.9 [M+H]⁺.

### Step 2. Synthesis of N-(2-chloro-4-(trifluoromethyl)phenyl)-2-iodoacetamide

To a solution of 2-chloro-N-(2-chloro-4-(trifluoromethyl)phenyl)acetamide (7.70 g, 28.3 mmol, 1.0 eq) in acetone (60 mL) was added KI (5.17 g, 31.1 mmol, 1.1 eq), the resulting mixture was stirred at 60° C. for 2 h. The mixture was cooled to room temperature, filtered and the filtrate was concentrated in vacuum to afford the title compound, which was used in the next step without further purification.

LCMS: 363.9 [M+H]⁺.

### Intermediate-13:2-(5-ethyl-2-(2-methoxypyridin-4-yl)-7-oxo-6-(piperazin-1-yl)oxazolo[5,4-b]pyridin-4(7H)-yl)-N-(3-(trifluoromethyl)bicyclo[1.1.1]pentan-1-yl)acetamide hydrochloride

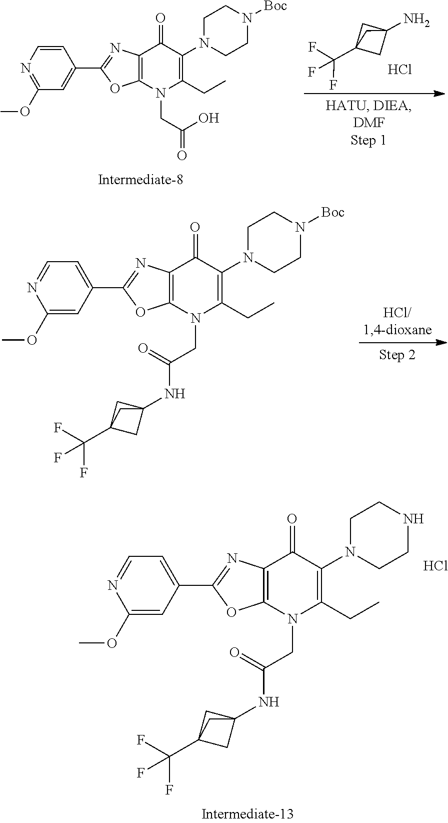 [SMILES: CCc1c(N2CCN(C(=O)OC(C)(C)C)CC2)c(=O)c2nc(-c3ccnc(OC)c3)oc2n1CC(=O)O ; NC12CC(C(F)(F)F)(C1)C2 ; Cl ; CCc1c(N2CCN(C(=O)OC(C)(C)C)CC2)c(=O)c2nc(-c3ccnc(OC)c3)oc2n1CC(=O)NC12CC(C(F)(F)F)(C1)C2 ; CCc1c(N2CCNCC2)c(=O)c2nc(-c3ccnc(OC)c3)oc2n1CC(=O)NC12CC(C(F)(F)F)(C1)C2 ; Cl]

### Step 1. Synthesis of tert-butyl 4-(5-ethyl-2-(2-methoxypyridin-4-yl)-7-oxo-4-(2-oxo-2-((3-(trifluoromethyl)bicyclo[1.1.1]pentan-1-yl)amino)ethyl)-4,7-dihydrooxazolo[5,4-b]pyridin-6-yl)piperazine-1-carboxylate

To a mixture of 2-[6-(4-tert-butoxycarbonylpiperazin-1-yl)-5-ethyl-2-(2-methoxy-4-pyridyl)-7-oxo-oxazolo[5,4-b]pyridin-4-yl]acetic acid (Intermediate-8) (50 mg, 97 μmol, 1 eq) and 3-(trifluoromethyl)bicyclo[1.1.1]pentan-1-amine hydrochloride (37 mg, 195 μmol, 2 eq) in DMF (0.5 mL) were added HATU (74 mg, 195 μmol, 2 eq) and DIEA (38 mg, 292 μmol, 51 μL, 3 eq). The mixture was stirred for 1 h at 25° C. The reaction mixture was poured into water (20 mL) and was extracted with EtOAc (20 mL*3). The combined organic layer was washed with brine (20 mL), dried over anhydrous Na₂SO₄, filtered and concentrated under reduced pressure to give a residue. The residue was purified by flash silica gel chromatography (Eluent of DCM/MeOH) to afford the title compound.

LCMS: 647.3 [M+H]⁺.

### Step 2. Synthesis of 2-(5-ethyl-2-(2-methoxypyridin-4-yl)-7-oxo-6-(piperazin-1-yl)oxazolo[5,4-b]pyridin-4(7H)-yl)-N-(3-(trifluoromethyl)bicyclo[1.1.1]pentan-1-yl)acetamide hydrochloride

A solution of tert-butyl 4-[5-ethyl-2-(2-methoxy-4-pyridyl)-7-oxo-4-[2-oxo-2-[[3-(trifluoromethyl)-1-bicyclo[1.1.1]pentanyl]amino]ethyl]oxazolo[5,4-b]pyridin-6-yl]piperazine-1-carboxylate (25 mg, 39 μmol, 1 eq) in HCl/1,4-dioxane (2 M, 0.2 mL) was stirred at 25° C. for 0.5 h. The reaction mixture was concentrated under reduced pressure directly to afford the title compound, which was used into the next step without further purification.

LCMS: 547.3 [M+H]⁺.

### Intermediate-14:2-(6-(4-(tert-butoxycarbonyl)piperazin-1-yl)-5-ethyl-2-(2-methoxypyridin-4-yl)-7-oxooxazolo[4,5-b]pyridin-4(7H)-yl) acetic acid

### Intermediate-15:2-(5-ethyl-2-(2-methoxypyridin-4-yl)-7-oxo-6-(piperazin-1-yl)oxazolo[4,5-b]pyridin-4(7H)-yl)-N-(3-(trifluoromethyl)bicyclo[1.1.1]pentan-1-yl)acetamide hydrochloride

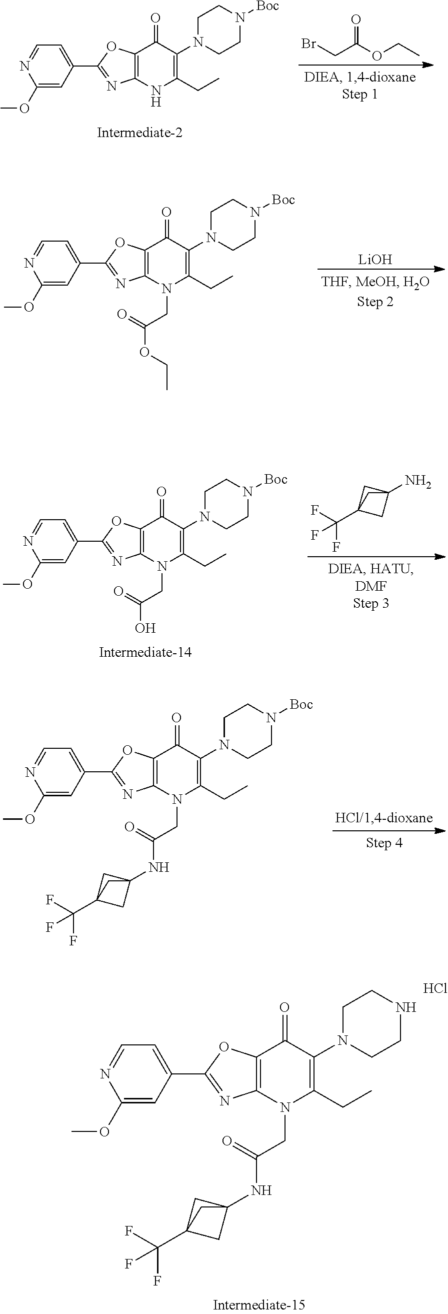

### Step 1. Synthesis of tert-butyl 4-(4-(2-ethoxy-2-oxoethyl)-5-ethyl-2-(2-methoxypyridin-4-yl)-7-oxo-4,7-dihydrooxazolo[4,5-b]pyridin-6-yl)piperazine-1-carboxylate

To a solution of tert-butyl 4-[5-ethyl-2-(2-methoxy-4-pyridyl)-7-oxo-4H-oxazolo[4,5-b]pyridin-6-yl]piperazine-1-carboxylate (Intermediate-2) (1.00 g, 2.20 mmol, 1 eq) and ethyl 2-bromoacetate (440 mg, 2.63 mmol, 292 μL, 1.2 eq) in dioxane (10 mL) was added DIEA (851 mg, 6.59 mmol, 1.15 mL, 3 eq). The mixture was stirred at 100° C. for 0.5 h. The reaction mixture was diluted with H₂O (200 mL) and extracted by EtOAc (250 mL*2). The combined organic phase was washed with brine (100 mL*1), dried over anhydrous Na₂SO₄, filtered and concentrated in vacuum to give a residue. The residue was purified by silica gel chromatography (Eluent of EtOAc/PE) to afford the title compound.

¹H NMR (400 MHz, DMSO-d6) δ ppm 8.43 (d, 1H), 7.60 (d, 1H), 7.37 (s, 1H), 5.23 (s, 2H), 4.21 (q, 2H), 3.94-3.85 (m, 5H), 3.63 (q, 2H), 2.95-2.58 (m, 4H), 2.59-2.56 (m, 2H), 1.43 (s, 9H), 1.23 (t, 3H), 1.11 (t, 3H).

LCMS: 542.2 [M+H]⁺.

### Step 2. Synthesis of 2-(6-(4-(tert-butoxycarbonyl)piperazin-1-yl)-5-ethyl-2-(2-methoxypyridin-4-yl)-7-oxooxazolo[4,5-b]pyridin-4(7H)-yl) acetic acid

To a solution of tert-butyl 4-[4-(2-ethoxy-2-oxo-ethyl)-5-ethyl-2-(2-methoxy-4-pyridyl)-7-oxo-oxazolo[4,5-b]pyridin-6-yl]piperazine-1-carboxylate (180 mg, 332 μmol, 1 eq) in EtOH (2 mL), THF (2 mL) and H₂O (2 mL) was added LiOH·H₂O (21 mg, 499 μmol, 1.5 eq). The mixture was stirred at 25° C. for 0.5 h. The mixture was adjusted pH to about 4 by addition of HCl (1M) dropwise at 25° C. and the mixture was extracted with EtOAc (5 mL*3). The combined organic phase was washed with brine (15 mL*2), dried over anhydrous Na₂SO₄, filtered and concentrated in vacuum to afford the title compound, which was used into next step directly without further purification.

LCMS: 514.2 [M+H]⁺.

### Step 3. Synthesis of tert-butyl 4-(5-ethyl-2-(2-methoxypyridin-4-yl)-7-oxo-4-(2-oxo-2-((3-(trifluoromethyl)bicyclo[1.1.1]pentan-1-yl)amino)ethyl)-4,7-dihydrooxazolo[4,5-b]pyridin-6-yl)piperazine-1-carboxylate

To a solution of 2-[6-(4-tert-butoxycarbonylpiperazin-1-yl)-5-ethyl-2-(2-methoxy-4-pyridyl)-7-oxo-oxazolo[4,5-b]pyridin-4-yl]acetic acid (Intermediate-14) (180 mg, 294 μmol, 1 eq) and 3-(trifluoromethyl)bicyclo[1.1.1]pentan-1-amine hydrochloride salt (110 mg, 589 μmol, 2 eq) in DMF (2 mL) was added DIEA (114 mg, 883 μmol, 154 μL, 3 eq) and HATU (224 mg, 589 μmol, 2 eq). The mixture was stirred at 25° C. for 1 h. To the reaction mixture was added H₂O (15 mL). Then the mixture was extracted with EtOAc (15 mL*3). The combined organic phase was washed with brine (20 mL*2), dried over anhydrous Na₂SO₄, filtered and concentrated in vacuum to give a residue. The residue was purified by silica gel chromatography (Eluent of EtOAc/PE) to afford the title compound.

LCMS: 647.3 [M+H]⁺

### Step 4. Synthesis of 2-(5-ethyl-2-(2-methoxypyridin-4-yl)-7-oxo-6-(piperazin-1-yl)oxazolo[4,5-b]pyridin-4(7H)-yl)-N-(3-(trifluoromethyl)bicyclo[1.1.1]pentan-1-yl)acetamide hydrochloride

A solution of tert-butyl 4-[5-ethyl-2-(2-methoxy-4-pyridyl)-7-oxo-4-[2-oxo-2-[[3-(trifluoromethyl)-1-bicyclo[1.1.1]pentanyl]amino]ethyl]oxazolo[4,5-b]pyridin-6-yl]piperazine-1-carboxylate (180 mg, 278 μmol, 1 eq) in HCl/dioxane (2 M, 3.6 mL, 26 eq) was stirred at 25° C. for 0.2 h. The mixture was concentrated in vacuum directly to afford the title compound, which was used into next step directly without further purification.

LCMS: 547.2 [M+H]⁺.

### Example 2: WRN (BV08) ADP-Glo Assay Protocol

Bovine skin gelatin (BSG), dimethyl sulfoxide (DMSO), Pluronic F-127 and tris(2-carboxyethyl)phosphine hydrochloride solution (TCEP) were purchased from Sigma-Aldrich (St. Louis, MO) at the highest level of purity possible. Bicine buffer solution was purchased from Alfa Aesar (Tewksbury, MA) and compound NSC-617145 was purchased from Tocris (Minneapolis, MN). DNA duplex was synthesized at BGI (Shenzhen, China) and was composed of strand 1 with the sequence 5′-GCACTGGCCGTCGTTTTACGGTCG-3′ (SEQ ID NO.: 1) and strand 2 with the sequence 5′-TCCAAGTAAAACGACGGCCAGTGC-3′ (SEQ ID NO.: 2). DNA strands were annealed by heating to 95° C. for 5 minutes followed by slow cooling to room temperature. Compounds in 100% DMSO (0.1 μl) were spotted into a 384-well white polystyrene Optiplate-384 (Perkin Elmer; Waltham, MA) assay plate using a LabCyte Echo 550 (Agilent; Santa Clara, CA). DMSO (0.1 μl) was added to columns 12, rows A-H and column 24, rows I-P for the maximum signal control. Compound NSC-617145 (0.1 μl) was added to columns 12, rows I-P and 24, rows A-H for the minimum signal control (100% inhibition). Compounds/DMSO were preincubated for 15 minutes at 25° C. with 5 μl 2×WRN (BV08), prepared as described below, in assay buffer containing 20 mM Bicine (pH=7.5), 1 mM MgCl₂, 10 mM KCl, 0.1% Pluronic F-127, 0.005% BSG, 1 mM TCEP. The reaction was initiated by the addition of 5 μl 2× substrate mixture in assay buffer and incubated for 60 minutes at 25° C. The final concentrations of the assay components were 0.15 nM WRN, 5 M ATP, and 0.1 nM DNA duplex. The final DMSO concentration was 1% and the reference compound concentration (NSC-617145) used for the minimal signal control was 20 μM. The reaction was stopped by the addition of the ADP-Glo Kit components (Promega; Madison, WI) as directed and the relative luminescence units (RLU) were read on an Envision 2104 (Perkin Elmer; Waltham, MA).

### % Inhibition Calculation:

$$\% INH = ( RLU MAX - RLU sample ) / ( RLU MAX - RLU MIN ) ) \times 100$$

Where RLU=relative luminescence units, sample=signal in sample well, and MIN and MAX are the respective minimum and maximum signal controls.

### Four-Parameter IC₅₀ Fit Equation:

Y=Bottom+(Top−Bottom)/(1+(IC50/X)\hat{ }Hill Slope)

Where top and bottom are normally allowed to float but may be fixed at 100 or 0 respectively in a 3-parameter fit. Y is the % inhibition and X is the compound concentration.

### WRN Protein Production

Molecular Biology and virus production. The DNA encoding human Werner helicase (Uniprot Q14191, amino acids 517-1235 with L1074F point mutation) was generated with codon-optimization for E. coli expression and subcloned into the pFastBac vector with a TEV cleavable 8×His tag (WRN—BV08). The baculovirus from the expression plasmid WRN—BV08 was generated from transfection and amplification following the manufacturer's instructions.

Gene sequence of WRN-BV08 [pFastBac1-WRN-(517-1235 L1074F)-TEV-8His]
(SEQ ID NO .: 3)
ATGAACGAGGGCGAAGAAGACGACGACAAGGACTTCCTGTGGCCTGCCCCTAACGA
AGAACAAGTGACATGCCTGAAGATGTACTTCGGACACAGTAGCTTCAAGCCTGTGC
AATGGAAGGTCATCCACTCCGTGCTGGAAGAAAGAAGGGACAACGTGGCTGTGATG
GCTACCGGATACGGTAAGTCCCTGTGCTTCCAGTACCCTCCCGTGTACGTGGGCAAG
ATCGGTCTGGTGATCTCCCCTCTGATCTCTCTGATGGAGGACCAGGTGCTGCAATTG
AAGATGTCCAACATCCCCGCTTGCTTCCTGGGTTCCGCTCAAAGTGAGAACGTGCTG
ACAGACATCAAGCTGGGCAAGTACCGCATCGTGTACGTGACCCCTGAGTACTGCTCC
GGTAACATGGGTCTGCTGCAACAGCTGGAGGCTGACATCGGAATCACCCTGATCGCT
GTGGACGAGGCTCACTGCATCTCCGAGTGGGGACACGACTTCCGCGACTCCTTCCGT
AAGCTGGGATCCTTGAAGACCGCTCTCCCTATGGTGCCTATCGTGGCCCTGACCGCC
ACTGCTTCCTCCTCCATCCGCGAGGACATCGTGCGTTGCCTGAACCTGCGCAACCCT
CAGATCACTTGCACCGGTTTCGACCGCCCTAACTTGTACCTCGAGGTGCGTCGCAAG
ACCGGTAACATCCTCCAGGACCTGCAGCCTTTCCTGGTCAAGACCTCCTCCCACTGG
GAATTTGAGGGCCCTACCATCATCTACTGCCCTTCCCGCAAGATGACCCAGCAAGTC
ACCGGCGAGCTGCGCAAGCTCAACCTCTCCTGCGGTACCTACCACGCTGGTATGTCC
TTCTCCACCCGCAAGGACATCCACCACCGCTTCGTCCGTGACGAAATCCAATGCGTC
ATCGCTACCATCGCTTTCGGAATGGGCATCAACAAGGCTGACATCCGCCAGGTGATC
CACTACGGCGCCCCCAAGGACATGGAATCCTACTACCAGGAAATCGGTCGCGCCGG
TCGCGACGGTCTGCAGTCTTCCTGTCACGTGCTGTGGGCCCCCGCTGACATCAACCT
GAACCGCCACCTGCTGACCGAAATCCGCAACGAGAAGTTCCGCCTGTACAAGCTCA
AGATGATGGCTAAGATGGAGAAGTACCTGCACTCCTCCCGCTGTCGCCGTCAGATCA
TCCTCTCCCACTTCGAGGACAAGCAAGTGCAAAAGGCTAGCCTGGGTATCATGGGC
ACCGAAAAGTGTTGTGACAACTGCCGCTCCCGCCTCGACCACTGCTACTCCATGGAC
GACAGCGAGGACACCTCCTGGGACTTCGGTCCTCAAGCTTTCAAGCTCTTGTCCGCT
GTGGACATCCTGGGCGAGAAGTTCGGTATCGGTCTCCCCATCCTCTTCCTGCGTGGT
AGCAACTCCCAACGCCTGGCTGACCAGTACCGCCGCCACTCCCTCTTCGGTACCGGT
AAGGACCAGACCGAGTCCTGGTGGAAGGCTTTCTCTCGCCAACTGATCACCGAAGG
TTTCCTGGTGGAGGTGTCCCGCTACAACAAGTTCATGAAGATCTGCGCTCTCACTAA
GAAGGGAAGGAACTGGCTGCACAAGGCTAACACTGAGTCCCAATCCCTCATCCTGC
AGGCTAACGAGGAGCTGTGCCCTAAGAAGTTCCTGCTGCCTTCCTCCAAGACCGTGT
CCTCCGGAACAAAGGAACACTGCTACAACCAAGTCCCTGTGGAGCTCTCCACCGAG
AAGAAGTCCAACCTGGAGAAGCTGTACAGCTACAAGCCTTGCGACAAGATCAGCTC
CGGTTCCAACATCAGCAAGAAGTCCATCATGGTGCAATCCCCTGAAAAGGCCTACTC
CAGCTCCCAACCTGTCATCTCCGCTCAAGAGCAAGAGACCCAGATCGTGCTGTACGG
TAAGCTGGTCGAAGCCCGCCAAAAGCACGCTAACAAGATGGACGTCCCTCCCGCTA
TCCTCGCCACCAACAAGATCCTCGTGGATATGGCTAAGATGCGCCCCACCACCGTCG
AGAACGTGAAGCGCATCGACGGTGTCTCCGAGGGTAAGGCCGCTATGCTGGCTCCT
CTGCTGGAAGTGATCAAGCACTTCTGCCAGACCAACTCCGTGCAGACCGACCTGTTC
AGTAGTGAGAACCTGTACTTCCAAGGCCACCATCATCATCATCATCACCACTAA
Protein sequence of WRN-BV08 [[1.1]pFastBac1-WRN-(517-1235 L1074F)-TEV-8His][2
(SEQ ID NO .: 4)
MNEGEEDDDKDFLWPAPNEEQVTCLKMYFGHSSFKPVQWKVIHSVLEERRDNVAVMA
TGYGKSLCFQYPPVYVGKIGLVISPLISLMEDQVLQLKMSNIPACFLGSAQSENVLTDIKL
GKYRIVYVTPEYCSGNMGLLQQLEADIGITLIAVDEAHCISEWGHDFRDSFRKLGSLKTA
LPMVPIVALTATASSSIREDIVRCLNLRNPQITCTGFDRPNLYLEVRRKTGNILQDLQPFL
VKTSSHWEFEGPTIIYCPSRKMTQQVTGELRKLNLSCGTYHAGMSFSTRKDIHHRFVRD
EIQCVIATIAFGMGINKADIRQVIHYGAPKDMESYYQEIGRAGRDGLQSSCHVLWAPADI
NLNRHLLTEIRNEKFRLYKLKMMAKMEKYLHSSRCRRQIILSHFEDKQVQKASLGIMGT
EKCCDNCRSRLDHCYSMDDSEDTSWDFGPQAFKLLSAVDILGEKFGIGLPILFLRGSNSQ
RLADQYRRHSLFGTGKDQTESWWKAFSRQLITEGFLVEVSRYNKFMKICALTKKGRNW
LHKANTESQSLILQANEELCPKKFLLPSSKTVSSGTKEHCYNQVPVELSTEKKSNLEKLY
SYKPCDKISSGSNISKKSIMVQSPEKAYSSSQPVISAQEQETQIVLYGKLVEARQKHANK
MDVPPAILATNKILVDMAKMRPTTVENVKRIDGVSEGKAAMLAPLLEVIKHFCQTNSV
QTDLFSSENLYFQGHHHHHHHH

Sf9 cells grown in SF900II media were infected with 1:200 WRN—BV08 P2 virus and incubated for protein expression for 72 h at 27° C. The WRN protein was purified using the following protocol. The cell pellets were thawed and resuspended in buffer A (50 mM Tris, pH 7.5, 500 mM NaCl, 1 mM TCEP, 10% Glycerol) supplemented with 0.5% CHAPS, 1 mM PMSF, 1 μg/ml Leupeptin, 1 μg/ml Pepstatin, and the Pierce Universal Nuclease and cocktail tablet. Cleared lysates were loaded onto a Ni Sepharose™ excel column and washed with buffer A and bound protein was eluted with buffer A supplemented with 300 mM imidazole. The eluted protein was dialyzed against buffer A and digested by His-tagged TEV (1:5 ratio) overnight at 4° C. ZnCl₂ was added into the sample at final 15 μM before loading onto a second Ni Sepharose™ excel column. Untagged WRN protein was eluted from the column with buffer A supplemented with 20 mM imidazole, dialyzed overnight into buffer B (50 mM Tris, pH 7.5, 1 mM TCEP, 10% Glycerol) supplemented with 150 mM NaCl and loaded onto a Heparin column. Proteins were eluted with a step gradient of buffer B supplemented with 150 mM, 200 mM, 300 mM and 500 mM NaCl. WRN containing fractions were pooled and concentrated prior to loading on to size exclusion chromatography using a HiLoad 16/600 Superdex™ 200 μg column (GE Healthcare) in buffer C (20 mM HEPES, pH 7.5, 250 mM NaCl, 0.25 mM TCEP, 2.5% Glycerol).

The resultant IC₅₀ results obtained for the tested compounds are shown below in Table 3. Compounds with an IC₅₀ less than or equal to 0.005 μM are designated as “A.” Compounds with an IC₅₀ greater than 0.005 UM and less than or equal to 0.05 μM are designated as “B.” Compounds with an IC₅₀ greater than 0.05 UM and less than or equal to 0.1 μM are designated as “C.” Compounds with an IC₅₀ greater than 0.1 μM or equal to 0.5 μM are designated as “D.”

TABLE 3

| Cmpd. No. | ADP-Glo_hWRN_IC50 [μM] |
|---|---|
| I-1 | A |
| 1-2 | A |
| 1-3 | B |
| I-4 | B |
| I-5 | A |

### Example 3: Method for Determining Effect on p21 Induction in Cells

The colon carcinoma cell line HCT116 was obtained from ATCC and cultured in growth medium consisting of Mccoy's 5A Medium (Gibco 16600108) supplemented with 10% FBS (Transgene FS201-02) and 100 units/mL penicillin-streptomycin (Gibco 15140122) and maintained at 37° C. under 5% CO₂. On the day of seeding, 2,000 cells in 30 μL of culture media were plated per well to Poly-D-Lysine 384 Well Black Clear Plates (Biocoat 356663) and incubated overnight at 37° C. under 5% CO₂. The following day, compounds were serially diluted in DMSO for a total of 11 test concentrations. The typical starting concentration of cpds was 10 μM with 2-fold dilutions. Next, 150 nL of diluted compound was added in duplicate to the assay plate, using an Echo 655 (Labcyte). The plate was centrifuged at 500 RPM for 1 min and then incubated at 37° C. under 5% CO₂ for 24 h. After 24 h, medium was removed, and cells were fixed by adding 40 μL of 4% paraformaldehyde solution to each well and incubated for 20 min at room temperature. The plate was then washed 4 times with 100 μL per well of wash buffer (PBS with 0.1% Tween-20) using a microplate washer. Next, 30 μL of ice-cold methanol was added to each well and the plate was incubated at −20° C. for 10 min. The plate was washed 4 times with 100 μL per well of wash buffer by a microplate washer, then 30 μL per well of blocking buffer ((Intercept PBS blocking buffer (LI-COR 927-70001) with 0.05% Tween-20)) was added and the plate was incubated at room temperature with shaking for 2h. Next, to each test well, 20 μL of primary antibody solution ((p21 Waf1/CIP (12D1) Rabbit mAb (Cell Signaling Technologies 2947) diluted 1:1000 and GAPDH (D4C6R) Mouse mAb (Cell Signaling Technologies 97166) diluted 1:2000 in blocking buffer)) was added and the plate was placed at 4° C., overnight. The following day, the plate was washed 5 times with 100 μL per well of wash buffer using a microplate washer for 5 min. 20 μL per well of secondary antibody ((IRDye 680CW Goat anti-Mouse IgG (H+L) (LI-COR 926-68070) diluted 1:2000 in Blocking Buffer and IRDye 800CW Goat anti-Rabbit IgG (H+L) (LI-COR 926-32211) diluted 1:2000 in Blocking Buffer)) was then added and the plate was stored for 2 h in the dark at room temperature with shaking. The plate was then washed 4 times with 100 μL per well of wash buffer again using a microplate washer. Finally, the p21 signal and the GAPDH signal were quantified using a LI-COR Odyssey CLx Imager machine reading at 800 nm and 700 nm, respectively. Each plate contained DMSO control (low control) and an internal reference WRN inhibitor (high control) respectively. For quantitation, the 800 nm/700 nm ratio was calculated for each well to give fold p21 induction and then percent activation for each compound well was calculated as follows ((100×(ratio cpd well−ratio low control)/(ratio high control−ratio low control)). EC50 values for each compound was generated after non-linear regression curve fitting using commercially available software.

The resultant EC₅₀ results obtained for the tested compounds are shown below in Table 4. Compounds with an EC₅₀ less than or equal to 0.50 μM are designated as “A.” Compounds with an EC₅₀ greater than 0.50 μM and less than or equal to 2.00 μM are designated as “B.” Compounds with an EC₅₀ greater than 2.00 μM and less than or equal to 5.00 μM are designated as “C.” Compounds with an EC₅₀ greater than 5.00 μM are designated as “D.”

TABLE 4

| Cmpd. No. | P21_EC50 [μM] |
|---|---|
| I-1 | A |
| I-2 | A |
| I-3 | C |
| I-4 | D |
| I-5 | A |

We claim:

1. A compound of Formula I″, or a pharmaceutically acceptable salt thereof: 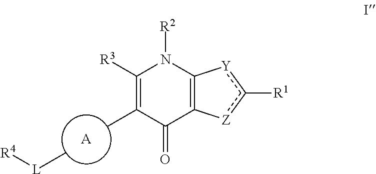
wherein Ring A represents:
a) a 4-7 membered saturated or partially unsaturated bivalent monocyclic ring system selected from carbocyclylene and heterocyclylene (having 1-4 heteroatoms independently selected from nitrogen, oxygen, and sulfur); or
b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
wherein Ring A is substituted with 0-4 independently selected R^{A} substituents;
each of Y and Z is selected from N and O, wherein Y is N and Z is O or Y is O and Z is N, [symbol] denotes a single or double bond, and the 5-membered ring comprising Z and Y is aromatic;
L is a linker selected from —C(O)—, —S(O)—, —S(O)₂—, and 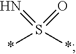
R¹ is selected from one of groups a) to e):
a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A},
b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A};
c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A};
d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}; and
e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹, —CH₂NR¹⁰R¹¹, —SO₂R¹², wherein the C₁-C₆aliphatic, C₃-C₇cycloalkyl, or C₁-C₆alkylene-O—C₁-C₆alkyl is substituted with 0-5 independently selected R^{A};
R¹⁰ is H, C₁-C₆aliphatic, haloC₁-C₆alkyl, C₃-C₆cycloalkyl, haloC₃-C₆cycloalkyl, —C(O)C₃-C₆cycloalkyl, —C(O) C₁-C₆alkyl, or a 5-6 membered heteroaryl (having 1-4 heteroatoms independently selected from nitrogen, oxygen, and sulfur) optionally substituted with 1 or 2 groups independently selected from R^{A};
R¹¹ is H, C₁-C₆aliphatic, or C₃-C₆cycloalkyl, or R¹⁰ and R¹¹ may combine to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R;
R² is selected from C(R^{B})₂C(O)N(R)R^{2A}, C(R^{B})₂C(R^{B})₂C(O)N(R)R^{2A}, C(R^{B})₂C(R^{B})₂N(R)C(O) N(R)R^{2A}, and C(R^{B})₂C(R^{B})₂N(R)C(O)R^{2A},
R^{B} is independently selected at each occurrence from hydrogen, —CH₃, and —CH₂CH₃, or two R^{B} taken together with the carbon to which they are attached form a cyclopropyl ring;
R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring, or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen, and wherein 2 substituents on the same atom of said saturated or partially unsaturated monocyclic ring, or saturated or partially unsaturated bridged, fused, or spirocyclic ring form a cyclic group selected from:
an optionally substituted 3-7 membered saturated or partially unsaturated carbocyclyl, and
an optionally substituted 4-7 membered saturated or partially unsaturated heterocyclyl having 1-3 heteroatoms independently selected from nitrogen, oxygen, and sulfur; or
R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH;
R³ is hydrogen, C₁-C₄aliphatic, C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, —OR, —C(O)NR¹⁰R¹¹, or N(R)C(O)R;
each R^{3A} is independently selected at each occurrence from C₁-C₄alkyl;
R⁴ is phenyl or a first 5-6 membered heteroaryl (having 1-4 heteroatoms independently selected from nitrogen, oxygen, and sulfur) wherein said phenyl or said first 5-6 membered heteroaryl are substituted with 0-5 R^{A} or two substituents on adjacent atoms of said phenyl or said first 5-6 membered heteroaryl together with said adjacent atoms form a 4-7 membered carbocyclyl, a 4-7 membered heterocyclyl, or a second 5-6 membered heteroaryl (having 1-4 heteroatoms independently selected from nitrogen, oxygen, and sulfur) that is fused to the phenyl or first 5-6 membered heteroaryl wherein said 4-7 membered carbocyclyl, 4-7 membered heterocyclyl, or second 5-6 membered heteroaryl are substituted with 0-3 R^{A}; or
R⁴ is a C₁-C₄alkyl, C₁-C₄alkoxy, or C₃-C₆cycloalkyl, each of which is substituted with 0-3 groups independently selected from halogen, —CN, —OH, oxo, NH₂, C₁-C₄alkyl, C₁-C₄alkoxy, optionally substituted 5-6 membered heterocyclyl, and optionally substituted 5-6 membered heterocyclyloxy;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

2. A compound of Formula I, or a pharmaceutically acceptable salt thereof: 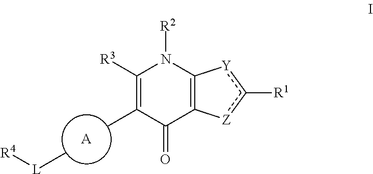
wherein Ring A represents:
a) a 4-7 membered saturated or partially unsaturated bivalent monocyclic ring system selected from carbocyclylene and heterocyclylene (having 1-4 heteroatoms independently selected from nitrogen, oxygen, and sulfur); or
b) a 4-12 membered saturated or partially unsaturated bivalent bicyclic ring system that is fused, bridged, or spirocyclic selected from carbocyclylene and heterocyclylene (having 1-4 heteroatoms independently selected from nitrogen, oxygen, and sulfur);
wherein Ring A is substituted with 0-4 independently selected R^{A} substituents;
each of Y and Z is selected from N and O, wherein Y is N and Z is O or Y is O and Z is N, [symbol] denotes a single or double bond, and the 5-membered ring comprising Z and Y is aromatic;
L is a linker selected from —C(O)—, —S(O)—, —S(O)₂—, and 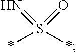
R¹ is selected from one of groups a) to e):
a) a 5-6 membered monocyclic heteroaryl (having 1-4 heteroatoms independently selected from nitrogen, oxygen, and sulfur) optionally substituted with 1-3 groups independently selected from halogen, C₁-C₆alkyl, haloC₁-C₆alkyl, C₃-C₆cycloalkyl, C₁-C₆alkoxy, and C₃-C₆cycloalkoxy, wherein said 5-6 membered monocyclic heteroaryl is further substituted with 0-3 independently selected R^{A};
b) a 9-10 membered bicyclic heteroaryl (having 1-4 heteroatoms independently selected from nitrogen, oxygen, and sulfur) optionally substituted with 1 or 2 groups independently selected from C₁-C₆aliphatic, C₃-C₆cycloalkyl, C₁-C₆alkoxy, and C₃-C₆cycloalkoxy, wherein said 9-10 membered bicyclic heteroaryl is further substituted with 0-3 independently selected R^{A};
c) a 4-7-membered saturated or partially unsaturated monocyclic heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated monocyclic heterocyclyl is further substituted with 0-3 independently selected R^{A};
d) a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0-3 independently selected R^{A}; and
e) H, halogen, C₁-C₆aliphatic, C₃-C₇cycloalkyl, C₁-C₆alkylene-O—C₁-C₆alkyl, —CN, —OR, —NR¹⁰R¹¹, —C(O)NR¹⁰R¹¹—CH₂NR¹⁰R¹¹, —SO₂R₁₂, wherein the C₁-C₆aliphatic, C₃-C₇cycloalkyl, or C₁-C₆alkylene-O—C₁-C₆alkyl is substituted with 0-5 independently selected R^{A};
R¹⁰ is H, C₁-C₆aliphatic, haloC₁-C₆alkyl, C₃-C₆cycloalkyl, haloC₃-C₆cycloalkyl, —C(O)C₃-C₆cycloalkyl, —C(O) C₁-C₆alkyl, or a 5-6 membered heteroaryl (having 1-4 heteroatoms independently selected from nitrogen, oxygen, and sulfur) optionally substituted with 1 or 2 groups independently selected from R^{A};
R¹¹ is H, C₁-C₆aliphatic, or C₃-C₆cycloalkyl, or R¹⁰ and R¹¹ may combine to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{A} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, hydroxy-C₁-C₆alkyl, haloC₁-C₆alkyl, an optionally substituted C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, and —N(R)S(O)₂R;
R² is C(R^{B})₂C(O)N(R)R^{2A};
R^{B} is independently selected at each occurrence from hydrogen, —CH₃, and —CH₂CH₃, or two R^{B} taken together with the carbon to which they are attached form a cyclopropyl ring;
R^{2A} is phenyl or pyridyl, each of which is optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, and —SF₅; or two substituents on adjacent atoms of the phenyl or pyridyl together with said adjacent atoms form a 4-7 membered carbocyclyl fused to the phenyl or pyridyl; or two substituents on adjacent atoms of the phenyl or pyridyl together with said adjacent atoms form a 4-7 membered heterocyclyl (having 1-4 heteroatoms independently selected from nitrogen, oxygen, and sulfur) fused to the phenyl or pyridyl, wherein said 4-7 membered carbocyclyl or 4-7 membered heterocyclyl is substituted with 0-5 independently selected halogen; or
R^{2A} is 2-benzimidazolyl, 2-naphthyl, or 3-quinolinyl, each of which is optionally substituted with 1, 2, or 3 substituents independently selected from halogen, C₁-C₄aliphatic, haloC₁-C₄alkyl, and —OH;
R³ is hydrogen, C₁-C₄aliphatic, C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, —OR, —C(O)NR¹⁰R¹¹, or N(R)C(O)R;
each R^{3A} is independently selected at each occurrence from C₁-C₄alkyl;
R⁴ is phenyl or a first 5-6 membered heteroaryl (having 1-4 heteroatoms independently selected from nitrogen, oxygen, and sulfur) wherein said phenyl or said first 5-6 membered heteroaryl are substituted with 0-5 R^{A} or two substituents on adjacent atoms of said phenyl or said first 5-6 membered heteroaryl together with said adjacent atoms form a 4-7 membered carbocyclyl, a 4-7 membered heterocyclyl, or a second 5-6 membered heteroaryl (having 1-4 heteroatoms independently selected from nitrogen, oxygen, and sulfur) that is fused to the phenyl or first 5-6 membered heteroaryl wherein said 4-7 membered carbocyclyl, 4-7 membered heterocyclyl, or second 5-6 membered heteroaryl are substituted with 0-3 R^{A}; or
R⁴ is a C₁-C₄alkyl, C₁-C₄alkoxy, or C₃-C₆cycloalkyl, each of which is substituted with 0-3 groups independently selected from halogen, —CN, —OH, oxo, NH₂, C₁-C₄alkyl, C₁-C₄alkoxy, optionally substituted 5-6 membered heterocyclyl, and optionally substituted 5-6 membered heterocyclyloxy;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, 4-7 membered partially unsaturated ring, or 5-6 membered heteroaryl ring (wherein said 4-7 membered saturated ring and 4-7 membered partially unsaturated ring has 0-3 heteroatoms independently selected from nitrogen, oxygen, and sulfur and wherein said 5-6 membered heteroaryl ring has 1-3 heteroatoms independently selected from nitrogen, oxygen, and sulfur).

3. The compound of claim 1 or claim 2, or a pharmaceutically acceptable salt thereof, of one of the following formulas: 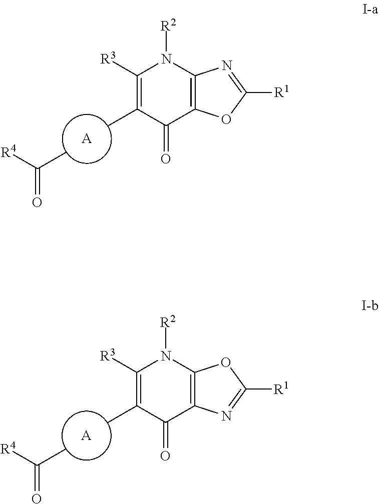
wherein:
R⁴ is selected from one of a), b), and c):
a) R⁴ is a Ring B that is selected from the group consisting of: 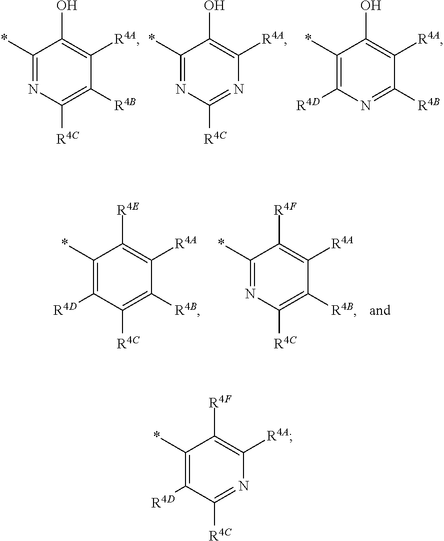
wherein * is a point of attachment to L that is bonded to Ring A in Formula I or I″, wherein L is —C(O)—;
and wherein:
any substituents that are present on Ring B selected from R^{4A}, R^{4B}, R^{4C}, R^{4D}, R^{4E}, and R^{4F} are each independently selected from hydrogen; halogen; —OH; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form 4-7 membered optionally substituted carbocyclyl, 4-7 membered optionally substituted heterocyclyl, or 5-6 membered optionally substituted heteroaryl (having 1-4 heteroatoms independently selected from nitrogen, oxygen, and sulfur) that is fused to Ring B; and any substituents that are present on Ring B selected from R^{4C}, R^{4D}, R^{4E}, and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4B} and R^{4C}, along with their intervening atoms, join to form 4-7 membered optionally substituted carbocyclyl, 4-7 membered optionally substituted heterocyclyl, or 5-6 membered heteroaryl (having 1-4 heteroatoms independently selected from nitrogen, oxygen, and sulfur) that is fused to Ring B; and any substituents that are present on Ring B selected from R⁴, R^{4D}, R^{4E}, and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form 4-7 membered optionally substituted carbocyclyl, 4-7 membered optionally substituted heterocyclyl, or 5-6 membered heteroaryl (having 1-4 heteroatoms independently selected from nitrogen, oxygen, and sulfur) that is fused to Ring B; and any substituents that are present on Ring B selected from R^{4A}, R^{4B}, R^{4E} and R^{4F} are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} and R^{4A}, along with their intervening atoms, join to form 5-6 membered optionally substituted heteroaryl (having 1-4 heteroatoms independently selected from nitrogen, oxygen, and sulfur) that is fused to Ring B; and R^{4B}, R^{4C}, and RAD are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4F} and R⁴⁴, along with their intervening atoms, join to form a 5-6 membered optionally substituted heteroaryl (having 1-4 heteroatoms independently selected from nitrogen, oxygen, and sulfur) that is fused to Ring B; and any substituents that are present on Ring B selected from R^{4B}, R^{4C}, and RAD are each independently selected from hydrogen; halogen; —OH; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen and C₁-C₄alkyl optionally substituted with —OH, —OCH₃, or —OCH₂CH₃; and
R¹⁴ is hydrogen; or NR¹³R¹⁴ forms a heterocyclic ring selected from azetidinyl, pyrrolidinyl, or piperidinyl, said heterocyclic ring optionally substituted with —CH₃; or
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy; or
c) R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl, and optionally substituted 5-6 membered heterocyclyloxy.

4. The compound of any one of claims 1-3, wherein the compound is of one of the following formulas: 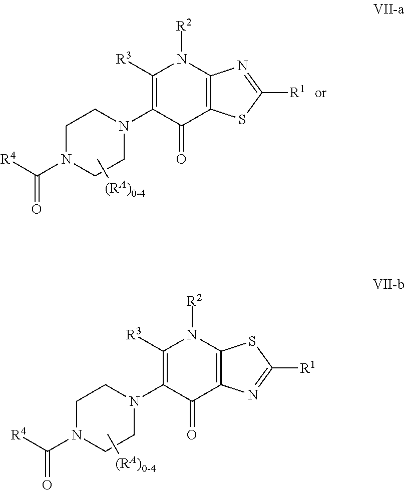
or a pharmaceutically acceptable salt thereof.

5. The compound of any one of claims 1-3, or a pharmaceutically acceptable salt thereof, wherein Ring A is selected from: 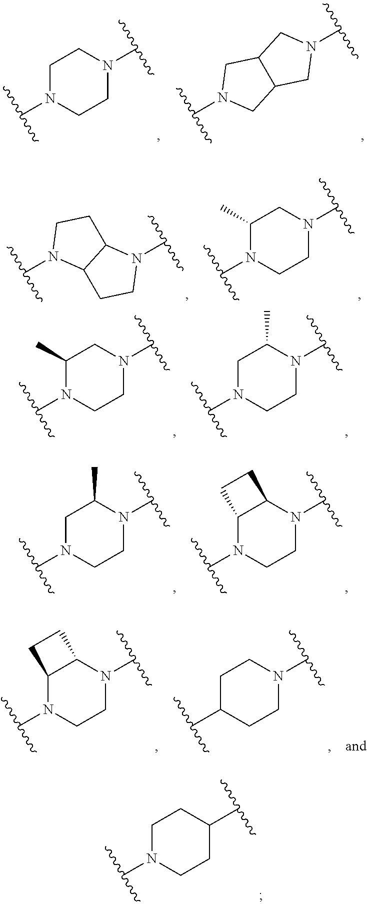
and wherein Ring A is substituted with 0-4 independently selected R^{A} substituents.

6. The compound of any one of claims 1-5, wherein R¹ is halogen, C₁-C₆ alkyl, C₂-C₄ alkene, C₂-C₄ alkyne, CN, —OR¹⁰, —NR¹⁰R¹¹, —C(O)NR¹⁰R¹¹, —CH₂NR¹⁰R¹¹, —SO₂R¹², or a 3-7 membered carbocyclyl, wherein C₁-C₆ alkyl, C₂-C₄ alkene, C₂-C₄alkyne, and 3-7 membered carbocyclyl are substituted with 0-3 substituents independently selected from halogen, C₃-C₆cycloalkyl, haloC₃-C₆cycloalkyl, —OH, —CN, C₁-C₄alkoxy, and haloC₁-C₄alkoxy.

7. The compound of any one of claims 1-5, wherein R¹ is a 5-6 membered heteroaryl (having 1-4 heteroatoms independently selected from nitrogen, oxygen, and sulfur) optionally substituted with 1-3 groups independently selected from halogen, C₁-C₆ alkyl, haloC₁-C₆ alkyl, C₁-C₆alkoxy, and C₃-C₆cycloalkyl, wherein said 5-6 membered heteroaryl is further substituted with 0-3 independently selected R^{A}.

8. The compound of any one of claims 1-5, wherein R¹ is pyridyl substituted with C₁-C₄alkoxy and further substituted with 0-2 R^{A}.

9. The compound of any one of claims 1-5, wherein R¹ is 5-membered heteroaryl (having 1 heteroatom independently selected from nitrogen, oxygen, and sulfur, and 0 or 1 additional ring nitrogen atoms), wherein said 5-membered heteroaryl is substituted with a halogen, C₁-C₆alkyl, haloC₁-C₆alkyl, C₁-C₆alkoxy, or C₃-C₆cycloalkyl, and further substituted with 0-2 independently selected R^{A}.

10. The compound of any one of claims 1-5, wherein R¹ is
c) a 5-6 membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁-C₄aliphatic, —OR, azetidinyl optionally substituted with 1 or 2 independently selected halogen, and pyrrolidinyl optionally substituted with 1 or 2 independently selected halogen; or
d) a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁-C₄aliphatic, —OR, azetidinyl optionally substituted with 1 or 2 independently selected halogen, and pyrrolidinyl optionally substituted with 1 or 2 independently selected halogen.

11. The compound of any one of claims 1-5, wherein R¹ is a 5-6 membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁-C₄aliphatic, —OR, azetidinyl optionally substituted with 1 or 2 independently selected halogen, and pyrrolidinyl optionally substituted with 1 or 2 independently selected halogen.

12. The compound of any one of claims 1-5, wherein R¹ is selected from the group consisting of:

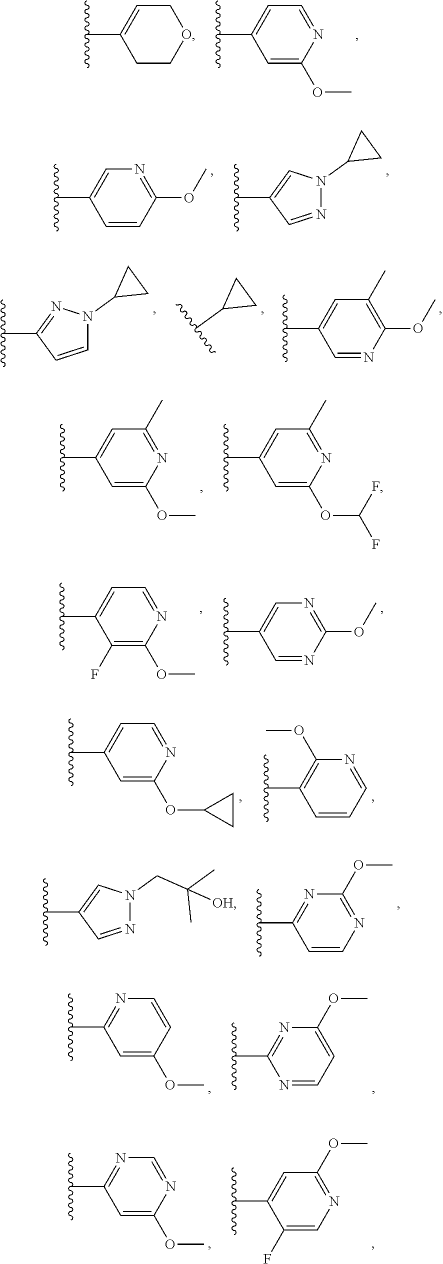

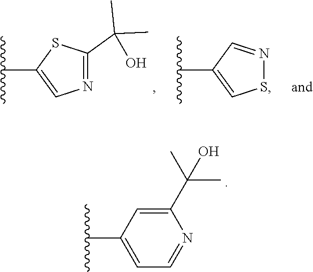

13. The compound of any one of claims 1-12, wherein R⁴ is Ring B of the following structure: 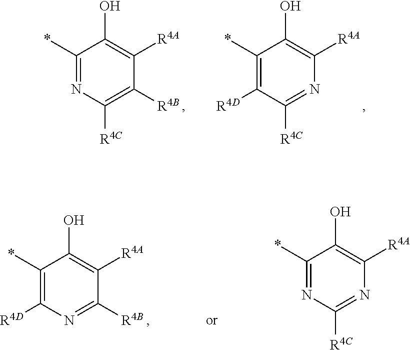
wherein * is a point of attachment to L in Formula I;
R^{4A} is hydrogen, halogen, —CH₃, —CH₂CH₃, —F, —CF₂H, —CF₃, —OCH₃, —OCF₃, —OCH₂CH₃, or —OCHF₂;
R^{4B}, R^{4C} and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴;
R¹³ is independently selected at each occurrence from hydrogen and C₁-C₄alkyl optionally substituted with —OH, —OCH₃, or —OCH₂CH₃; and
R¹⁴ is hydrogen; or R¹³ and R¹⁴ are taken together with the nitrogen atom to which they are attached to form a heterocyclic ring selected from azetidinyl, pyrrolidinyl, and piperidinyl; wherein the heterocyclic ring is optionally substituted with —CH₃; or
R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy.

14. The compound of any one of claims 1-12, wherein R⁴ is: 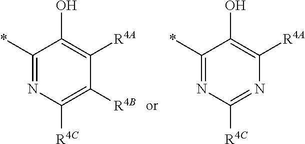
wherein * is a point of attachment to L in Formula I; wherein:
R^{4A} is hydrogen, halogen, —CH₃, —CH₂CH₃, —F, —CF₂H, —CF₃, —OCH₃, —OCF₃, —OCH₂CH₃, or —OCHF₂;
R^{4B} and R^{4C} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴;
R¹³ is independently selected at each occurrence from hydrogen and C₁-C₄alkyl optionally substituted with —OH, —OCH₃, or —OCH₂CH₃; and
R¹⁴ is hydrogen; or R¹³ and R¹⁴ are taken together with the nitrogen atom to which they are attached to form a heterocyclic ring selected from azetidinyl, pyrrolidinyl, and piperidinyl; wherein the heterocyclic ring is optionally substituted with —CH₃.

15. The compound of any one of claims 1-12, wherein R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy.

16. The compound of any one of claims 1-12, wherein R⁴ is a 5-membered heteroaryl (having 1 heteroatom independently selected from nitrogen, oxygen, and sulfur and 0, 1, 2, or 3 additional ring nitrogen atoms) selected from the group consisting of imidazolyl, pyrazolyl, tetrazolyl, thiazolyl, isothiazolyl, 1,2,5-thiadiazolyl, 1,3,4-thiadiazolyl, oxazolyl, isoxazolyl, 1,2,4-oxadiazolyl, 1,2,3-triazolyl, and 1,2,4-triazolyl, wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy.

17. The compound of claim 16, wherein R⁴ is an isoxazolyl substituted with —OH or C₁-C₄alkoxy.

18. The compound of any one of claims 1-12, wherein R⁴ is

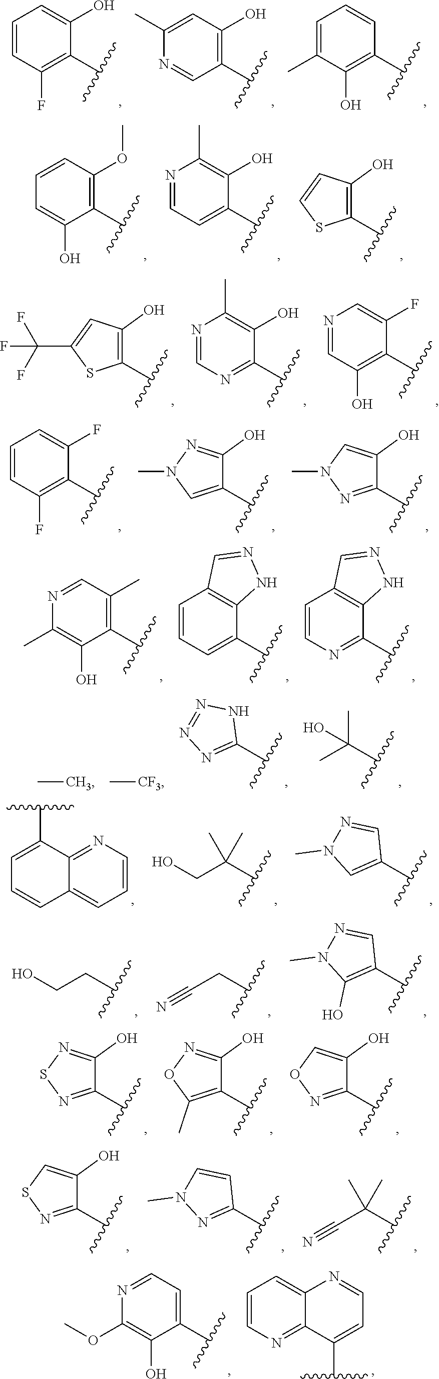

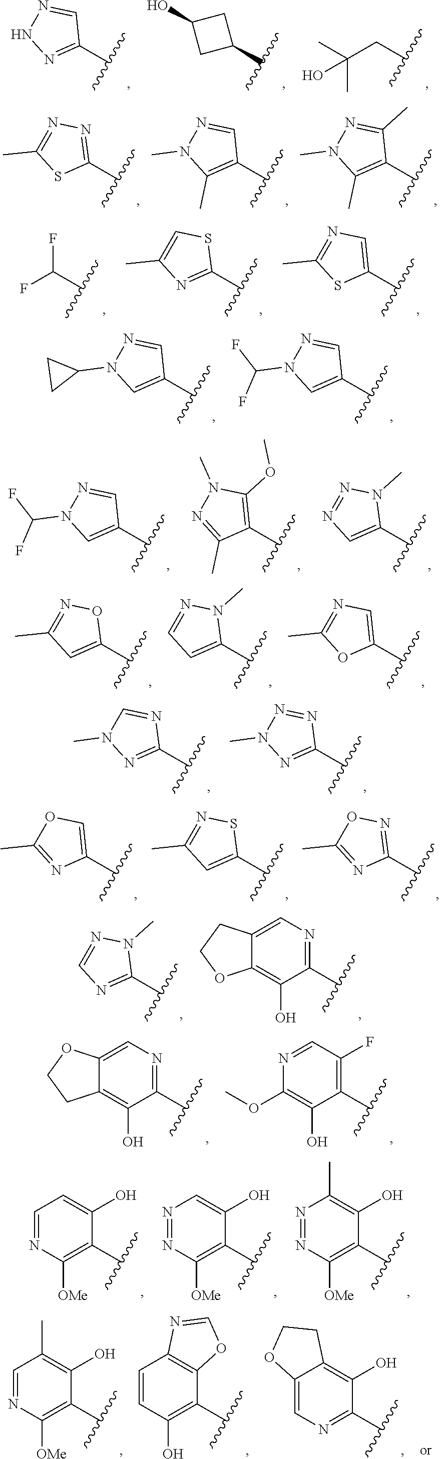

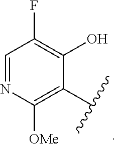 [SMILES: *c1c(OC)ncc(F)c1O]

19. The compound of any one of claims 1-18, wherein R^{2A} is phenyl comprising a —CF₃ substituent or pyridyl comprising a —CF₃ substituent.

20. The compound of any one of claims 1-18, wherein R² is

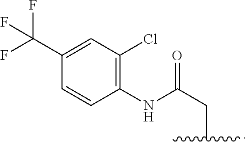 [SMILES: *CC(=O)Nc1ccc(C(F)(F)F)cc1Cl]

21. The compound of claim 1, or any one of claims 3-18, wherein R² is

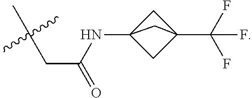 [SMILES: *CC(=O)NC12CC(C(F)(F)F)(C1)C2]

22. The compound of any one of claims 1-21, wherein R³ is C₁-C₄alkyl or C₃-C₅cycloalkyl.

23. The compound of any one of claims 1-3 and 6-22, wherein Ring A and the 0-4 independently selected R^{A} substituents with which Ring A is substituted are selected from

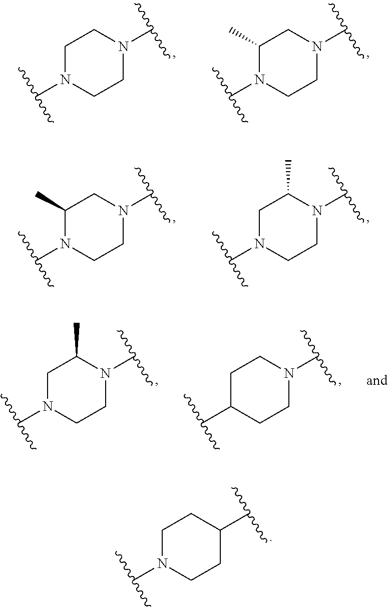

24. The compound of any one of claims 1-3 and 6-22, wherein Ring A and the 0-4 independently selected R^{A} substituents with which Ring A is substituted, is:

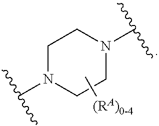

25. The compound of any one of claims 1-3 and 5-22, wherein Ring A is:

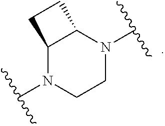 [SMILES: *N1CCN(*)[C@H]2CC[C@@H]21]

26. The compound of any one of claims 1-3 and 5-22, wherein Ring A is:

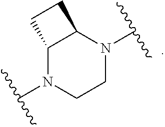 [SMILES: *N1CCN(*)[C@@H]2CC[C@H]21]

27. The compound of claim 1, or a pharmaceutically acceptable salt thereof, wherein the compound is N-(2-chloro-4-(trifluoromethyl)phenyl)-2-(5-ethyl-6-(4-(5-hydroxy-6-methylpyrimidine-4-carbonyl)piperazin-1-yl)-2-(2-methoxypyridin-4-yl)-7-oxooxazolo[4,5-b]pyridin-4(7H)-yl)acetamide.

28. A pharmaceutical composition comprising a compound or pharmaceutically acceptable salt thereof according to any one of claims 1-27, and one or more pharmaceutically acceptable carriers.

29. A method of treating cancer in a subject, wherein the cancer is characterized as microsatellite instability-high (MSI-H) or mismatch repair deficient (dMMR), comprising administering to the subject a therapeutically effective amount of a compound according to any one of claims 1-27, or a pharmaceutically acceptable salt thereof.

30. A method of modulating WRN activity in a subject, wherein the method comprises administering to the subject a therapeutically effective amount of the compound according to any one of claims 1-27, or a pharmaceutically acceptable salt thereof.

31. A method of treating a disorder or disease which can be treated by WRN inhibition in a subject, wherein the method comprises administering to the subject a therapeutically effective amount of the compound according to any one of claims 1-27, or a pharmaceutically acceptable salt thereof.

32. A method of inhibiting WRN in a subject, wherein the method comprises administering to the subject a therapeutically effective amount of the compound according to any one of claims 1-27, or a pharmaceutically acceptable salt thereof.

33. The method of claim 31, wherein the disorder or disease is a cancer characterized as microsatellite instability-high (MSI-H) or mismatch repair deficient (dMMR).

34. The method of claim 33, wherein the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer.
